(19) United States
(12) Patent Application Publication
(10) Pub. No.: US 2023/0241205 A1
(43) Pub. Date: Aug. 3, 2023

(54) SARS-COV-2 IMMUNODOMINANT PEPTIDES AND USES THEREOF

(71) Applicants: TScan Therapeutics, Inc., Waltham, MA (US); AHS Hospital Corp., Morristown, NJ (US)

(72) Inventors: Tomasz Kula, Brookline, MA (US); Gavin MacBeath, Wakefield, MA (US); Andrew P. Ferretti, Waltham, MA (US); Yifan Wang, Arlington, MA (US)

(21) Appl. No.: 18/009,826

(22) PCT Filed: Jun. 17, 2021
(86) PCT No.: PCT/US2021/037731

§ 371 Date: Dec. 12, 2022

Related U.S. Application Data
(60) Provisional application No. 63/040,267, filed on Jun. 17, 2020.
(60) Provisional application No. 63/050,930, filed on Jul. 13, 2020.
(60) Provisional application No. 63/056,462, filed on Jul. 24, 2020.
(60) Provisional application No. 63/056,849, filed on Jul. 27, 2020.

(51) Int. Cl. A61K 39/215; C07K 14/005; A61P 31/14

(52) U.S. Cl. CPC A61K 39/215; A61P 31/14; C07K 14/005; A61K 2039/575

(57) ABSTRACT

Provided herein are methods and compositions for the treatment and/or prevention of COVID-19 through the induction of an immune response against identified SARS-COV-2 immunodominant peptides.

### CROSS-REFERENCE TO RELATED APPLICATIONS

This application claims the benefit of priority to U.S. Provisional Application Serial No. 63/040,267, filed on 17 Jun. 2020; U.S. Provisional Application Serial No. 63/050,930, filed on 13 Jul. 2020; U.S. Provisional Application Serial No. 63/056,462, filed on 24 Jul. 2020; and U.S. Provisional Application Serial No. 63/056,849, filed on 27 Jul. 2020; the entire contents of each of said applications are incorporated herein in their entirety by this reference.

### BACKGROUND OF THE INVENTION

Coronavirus Disease 2019, or COVID-19, is a global pandemic caused by infections with Severe Acute Respiratory Syndrome (SARS)-CoV-2 (SARS-CoV-2) virus that has claimed >500,000 lives world-wide and has affected millions more. SARS-CoV-2 is the seventh coronavirus known to infect humans; SARS-CoV, MERS-CoV and SARS-CoV-2 can cause severe disease, whereas HKU1, NL63, OC43 and 229E are associated with mild symptoms. Developing effective vaccines and therapies requires understanding how the adaptive immune response recognizes and clears the virus and how the interplay between the virus and the immune system affects the pathology of the disease. To date, most efforts have focused on the B cell-mediated antibody response to the virus, but less is understood about how cytotoxic CD8+ T cells recognize and clear infected cells. Notably, the vast majority of current vaccine development efforts are focused on eliciting neutralizing antibodies to the virus, most frequently by immunizing with the spike (S) protein of SARS-CoV-2, or even with just the receptor binding domain (RBD) of the S protein (Vabret et al. (2020) Immunity 52:910-941). Studies of the most closely related coronavirus, SARS-CoV, which caused the 2002/2003 outbreak of SARS, showed that virus-specific memory CD8+ T cells persisted for six to eleven years in individuals who had recovered from SARS, whereas memory B cells and anti-viral antibodies were largely undetectable (Tang et al. (2011) J. Immunol. 186:7264-7268; Peng et al. (2006) Virol. 351:466-475). Similarly, a recent study of COVID-19 convalescent patients showed that although antibody responses to SARS-CoV-2 could be detected in most infected individuals 10-15 days following symptom onset, responses declined to baseline in many patients during the study’s 3-month follow up period (Seow et al. (2020) “Longitudinal evaluation and decline of antibody responses in SARS-CoV-2 infection” medRxiv (doi.org/10.1101/2020.07.09.20148429.2020) available at medrxiv.org/content/10.1101/2020.07.09.20148429v1). These findings suggest that vaccines focused solely on eliciting neutralizing antibodies to the S protein may be insufficient to elicit long-term immunity to coronaviruses. Notably, mouse studies of SARS-CoV showed that virus-specific CD8+ T cells are sufficient to enhance survival and diminish clinical disease (Zhao et al. (2010) J. Virol. 84:9318-9325) and that immunization with a single immunodominant CD8+ T cell epitope confers protection from lethal viral infection (Channappanavar et al. (2014) J. Virol. 88:11034-11044). These studies highlight the importance of understanding the natural CD8+ T cell response to SARS-CoV-2 as a route to designing more durable vaccines.

T cells play a critical role to control acute viral infection and provide durable immune protection from subsequent exposures. In the case of SARS-CoV-2, virus-reactive T cells have been reported, but the specific peptide targets recognized by these T cells remain unknown. Recently, studies using megapools of predicted T cell epitopes revealed that most COVID-19 convalescent patients, including those with severe disease, exhibit SARS-CoV-2-specific CD8+ T cells, and that at least some are directed at the S protein (Grifoni et al. (2020) Cell 181:1489-1501; Le Bert et al. (2020) “SARS-CoV-2-specific T cell immunity in cases of COVID-19 and SARS, and uninfected controls” Nature (doi: 10.1038/s41586-020-2550-z) available at nature.com/articles/s41586-020-2550-z). To date, however, the precise targets of CD8+ T cells in convalescent patients have not been identified, and it is not known how frequently these epitopes are shared among patients, how specific they are to SARS-CoV-2, or how effectively CD8+ T cells protect against severe disease. These peptide targets are important for developing prophylactic or therapeutic vaccines against the SARS-CoV-2 virus. Therefore, there is an urgent need for identifying SARS-CoV-2 virus-specific immunogenic peptides and developing effective vaccines based on these immunogenic peptides.

### SUMMARY OF THE INVENTION

The present invention is based, at least in part, on the discovery of SARS-CoV-2 immunodominant peptides. Importantly, some of these immunogenic peptides can elicit T cell response across patients.

In one aspect, an immunogenic peptide comprising a peptide epitope selected from Table 1A, 1B, 1C, 1D, 1E, and/or 1F, is provided.

In another aspect, an immunogenic peptide consisting of a peptide epitope selected from Table 1A, 1B, 1C, 1D, 1E, and/or 1F, is provided.

Numerous embodiments are further provided that may be applied to any aspect of the present invention and/or combined with any other embodiment described herein. For example, in one embodiment, the immunogenic peptide is derived from a SARS-CoV-2 protein, optionally wherein the immunogenic peptide is 8, 9, 10, 11, 12, 13, 14, or 15 amino acids in length. In another embodiment, the SARS-CoV-2 protein is selected from the group consisting of orfla/b, S protein, N protein, M protein, orf3a, and orf7a. In still another embodiment, the immunogenic peptide is capable of eliciting a T cell response in a subject.

In still another aspect, an immunogenic composition comprising at least one immunogenic peptide described herein (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, or more, or any range in between, inclusive, such as 1-5 peptides), is provided.

Numerous embodiments are further provided that may be applied to any aspect of the present invention and/or combined with any other embodiment described herein. For example, in one embodiment, the composition further comprises an adjuvant. In another embodiment, the immunogenic composition is capable of eliciting a T cell response in a subject.

In yet another aspect, composition comprising a peptide epitope selected from Table 1A, 1B, 1C, 1D, 1E, and/or 1F, and an MHC molecule, is provided.

Numerous embodiments are further provided that may be applied to any aspect of the present invention and/or combined with any other embodiment described herein. For example, in one embodiment, the MHC molecule is a MHC multimer, optionally wherein the MHC multimer is a tetramer. In another embodiment, the MHC molecule is an MHC class I molecule. In still another embodiment, the MHC molecule comprises an MHC alpha chain that is an HLA serotype selected from the group consisting of HLA-A*02, HLA-A*03, HLA-A*01, HLA-A* 11, HLA-A*24, and HLA-B*07, optionally wherein the HLA allele is selected from the group consisting of HLA-A* 0201, HLA-A* 0202, HLA-A*0203, HLA-A*0204, HLA-A*0205, HLA-A*0206, HLA-A*0207, HLA-A*0210, HLA-A*0211, HLA-A*0212, HLA-A*0213, HLA-A*0214, HLA-A*0216, HLA-A*0217, HLA-A*0219, HLA-A*0220, HLA-A*0222, HLA-A*0224, HLA-A*0230, HLA-A*0242, HLA-A*0253, HLA-A*0260, HLA-A*0274 allele, HLA-A*0301, HLA-A*0302, HLA-A*0305, HLA-A*0307, HLA-A*0101, HLA-A*0102, HLA-A*0103, HLA-A*0116 allele, HLA-A*1101, HLA-A*1102, HLA-A*1103, HLA-A*1104, HLA-A*1105, HLA-A*1119 allele, HLA-A*2402, HLA-A*2403, HLA-A*2405, HLA-A*2407, HLA-A*2408, HLA-A*2410, HLA-A*2414, HLA-A*2417, HLA-A*2420, HLA-A*2422, HLA-A*2425, HLA-A*2426, HLA-A*2458 allele, HLA-B*0702, HLA-B*0704, HLA-B*0705, HLA-B*0709, HLA-B*0710, HLA-B*0715, and HLA-B*0721 allele. Sequences, characteristics, structural information, functional information, binding partners, and the like for these and other HLA alleles are well-known in the art (see, e.g., the World Wide Web at hla.alleles.org/nomenclature/index.html, hla.alleles.org/data/hla-a.html, and hla.alleles.org/data/hla-b.html).

In another aspect, a stable MHC-peptide complex, comprising a peptide epitope selected from Table 1A, 1B, 1C, 1D, 1E, and/or 1F in the context of an MHC molecule, is provided.

Numerous embodiments are further provided that may be applied to any aspect of the present invention and/or combined with any other embodiment described herein. For example, in one embodiment, the MHC molecule is a MHC multimer, optionally wherein the MHC multimer is a tetramer. In another embodiment, the MHC molecule is a MHC class I molecule. In still another embodiment, the MHC molecule comprises an MHC alpha chain that is an HLA serotype selected from the group consisting of HLA-A* 02, HLA-A*03, HLA-A*01, HLA-A*11, HLA-A*24, and HLA-B*07, optionally wherein the HLA allele is selected from the group consisting of HLA-A* 0201, HLA-A* 0202, HLA-A* 0203, HLA-A*0204, HLA-A*0205, HLA-A*0206, HLA-A*0207, HLA-A*0210, HLA-A*0211, HLA-A*0212, HLA-A*0213, HLA-A*0214, HLA-A*0216, HLA-A*0217, HLA-A*0219, HLA-A*0220, HLA-A*0222, HLA-A*0224, HLA-A*0230, HLA-A*0242, HLA-A*0253, HLA-A*0260, HLA-A*0274 allele, HLA-A*0301, HLA-A*0302, HLA-A*0305, HLA-A*0307, HLA-A*0101, HLA-A*0102, HLA-A*0103, HLA-A*0116 allele, HLA-A*1101, HLA-A*1102, HLA-A*1103, HLA-A*1104, HLA-A*1105, HLA-A*1119 allele, HLA-A*2402, HLA-A*2403, HLA-A*2405, HLA-A*2407, HLA-A*2408, HLA-A*2410, HLA-A*2414, HLA-A*2417, HLA-A*2420, HLA-A*2422, HLA-A*2425, HLA-A*2426, HLA-A*2458 allele, HLA-B*0702, HLA-B*0704, HLA-B*0705, HLA-B*0709, HLA-B*0710, HLA-B*0715, and HLA-B*0721 allele. In yet another embodiment, the peptide epitope and the MHC molecule are covalently linked and/or wherein the alpha and beta chains of the MHC molecule are covalently linked. In another embodiment, the stable MHC-peptide complex comprises a detectable label, optionally wherein the detectable label is a fluorophore.

In still another aspect, an immunogenic composition comprising a stable MHC-peptide complex described herein, and an adjuvant, is provided.

In yet another aspect, an isolated nucleic acid that encodes an immunogenic peptide described herein, or a complement thereof, is provided.

In another aspect, a vector comprising an isolated nucleic acid described herein, is provided.

In still another aspect, a cell that a) comprises an isolated nucleic acid described herein, b) comprises a vector described herein, and/or c) produces one or more immunogenic peptides described herein and/or presents at the cell surface one or more stable MHC-peptide complexes described herein, optionally wherein the cell is genetically engineered, is provided.

In still another aspect, a binding moiety that specifically binds an immunogenic peptide described herein and/or a stable MHC-peptide complex described herein, optionally wherein the binding moiety is an antibody, an antigen-binding fragment of an antibody, a TCR, an antigen-binding fragment of a TCR, a single chain TCR (scTCR), a chimeric antigen receptor (CAR), or a fusion protein comprising a TCR and an effector domain (optionally further comprising a transmembrane domain and an effector domain that is intracellular), is provided.

In yet another aspect, a device or kit comprising a) one or more immunogenic peptides described herein and/or b) one or more stable MHC-peptide complexes described herein, said device or kit optionally comprising a reagent to detect binding of a) and/or b) to a T cell receptor, is provided.

In another aspect, a method of detecting T cells that bind a stable MHC-peptide complex comprising: (a) contacting a sample comprising T cells with a stable MHC-peptide complex described herein; and (b) detecting binding of T cells to the stable MHC-peptide complex, optionally further determining the percentage of stable MHC-peptide-specific T cells that bind to the stable MHC-peptide complex, is provided.

Numerous embodiments are further provided that may be applied to any aspect of the present invention and/or combined with any other embodiment described herein. For example, in one embodiment, the sample comprises peripheral blood mononuclear cells (PBMCs). In another embodiment, the T cells are CD8+ T cells. In still another embodiment, the detecting and/or determining is performed using fluorescence activated cell sorting (FACS), enzyme linked immunosorbent assay (ELISA), radioimmune assay (RIA), immunochemically, Western blot, or intracellular flow assay. In still another embodiment, the sample comprises T cells contacted with, or suspected of having been contacted with, one or more SARS-CoV-2 proteins or fragments thereof.

In still another aspect, a method of determining whether a subject has exposure to and/or protection from SARS-CoV-2 comprising a) incubating a cell population comprising T cells obtained from the subject with an immunogenic peptide described herein or a stable MHC-peptide complex described herein; and b) detecting the presence or level of reactivity, wherein the presence of or a higher level of reactivity compared to a control level indicates that the subject has exposure to and/or protection from SARS-CoV-2, is provided.

In yet another aspect, a method for predicting the clinical outcome of a subject afflicted with SARS-CoV-2 infection comprising a) determining the presence or level of reactivity between T cells obtained from the subject and one more immunogenic peptides described herein or one or more stable MHC-peptide complexes described herein; and b) comparing the presence or level of reactivity to that from aa control, wherein the control is obtained from a subject having a good clinical outcome, wherein the presence or a higher level of reactivity in the subject sample as compared to the control indicates that the subject has a good clinical outcome, is provided.

In another aspect, a method of assessing the efficacy of a SARS-CoV-2 therapy comprising a) determining the presence or level of reactivity between T cells obtained from the subject and one more immunogenic peptides described herein or one or more stable MHC-peptide complexes described herein, in a first sample obtained from the subject prior to providing at least a portion of the SARS-CoV-2 therapy to the subject, and b) determining the presence or level of reactivity between the one more immunogenic peptides described herein, or the one or more stable MHC-peptide complexes described herein, and T cells obtained from the subject present in a second sample obtained from the subject following provision of the portion of the SARS-CoV-2 therapy, wherein the presence or a higher level of reactivity in the second sample, relative to the first sample, is an indication that the therapy is efficacious for treating SARS-CoV-2 in the subject, is provided.

Numerous embodiments are further provided that may be applied to any aspect of the present invention and/or combined with any other embodiment described herein. For example, in one embodiment, the level of reactivity is indicated by a) the presence of binding and/or b) T cell activation and/or effector function, optionally wherein the T cell activation or effector function is T cell proliferation, killing, or cytokine release. In another embodiment, the method further comprises repeating steps a) and b) at a subsequent point in time, optionally wherein the subject has undergone treatment to ameliorate SARS-CoV-2 infection between the first point in time and the subsequent point in time. In still another embodiment, the T cell binding, activation, and/or effector function is detected using fluorescence activated cell sorting (FACS), enzyme linked immunosorbent assay (ELISA), radioimmune assay (RIA), immunochemically, Western blot, or intracellular flow assay. In yet another embodiment, the control level is a reference number. In another embodiment, the control level is a level of a subject without exposure to SARS-CoV-2.

In still another aspect, a method of preventing and/or treating SARS-CoV-2 infection in a subject comprising administering to the subject a therapeutically effective amount of an immunogenic composition comprising one or more immunogenic peptides, wherein the immunogenic peptides comprise a peptide epitope selected from Table 1A, 1B, 1C, 1D, 1E, and/or 1F, is provided.

Numerous embodiments are further provided that may be applied to any aspect of the present invention and/or combined with any other embodiment described herein. For example, in one embodiment, the immunogenic peptide consists of a peptide epitope selected from Table 1A, 1B, 1C, 1D, 1E, and/or 1F. In another embodiment, the immunogenic peptide is derived from a SARS-CoV-2 protein, optionally wherein the immunogenic peptide is 8, 9, 10, 11, 12, 13, 14, or 15 amino acids in length. In still another embodiment, the SARS-CoV-2 protein is selected from the group consisting of orfla/b, S protein, N protein, M protein, orf3a, and orf7a. In yet another embodiment, the immunogenic peptide is capable of eliciting a T cell response in a subject. In another embodiment, the immunogenic composition comprises more than one immunogenic peptide. In still another embodiment, the immunogenic composition further comprises an adjuvant. In yet another embodiment, the immunogenic composition is capable of eliciting a T cell response in a subject. In another embodiment, the administered immunogenic composition induces an immune response against the SARS-CoV-2 in the subject. In still another embodiment, the administered immunogenic composition induces a T cell immune response against the SARS-CoV-2 in the subject. In yet another embodiment, the T cell immune response is a CD8+ T cell immune response.

In yet another aspect, a method of identifying a peptide-binding molecule, or antigen-binding fragment thereof, that binds to a peptide epitope selected from Table 1A, 1B, 1C, 1D, 1E, and/or 1F comprising a) providing a cell presenting a peptide epitope selected from Table 1A, 1B, 1C, 1D, 1E, and/or 1F in the context of a MHC molecule on the surface of the cell; b) determining binding of a plurality of candidate peptide-binding molecules or antigen-binding fragments thereof to the peptide epitope in the context of the MHC molecule on the cell; and c) identifying one or more peptide-binding molecules or antigen-binding fragments thereof that bind to the peptide epitope in the context of the MHC molecule, is provided.

Numerous embodiments are further provided that may be applied to any aspect of the present invention and/or combined with any other embodiment described herein. For example, in one embodiment, the step a) comprises contacting the MHC molecule on the surface of the cell with a peptide epitope selected from Table 1A, 1B, 1C, 1D, 1E, and/or 1F. In another embodiment, the e step a) comprises transfecting the cell with a vector comprising a heterologous sequence encoding a peptide epitope selected from Table 1A, 1B, 1C, 1D, 1E, and/or 1F.

In another aspect, a method of identifying a peptide-binding molecule or antigen-binding fragment thereof that binds to a peptide epitope selected from Table 1A, 1B, 1C, 1D, 1E, and/or 1F comprising a) providing a peptide epitope either alone or in a stable MHC-peptide complex, comprising a peptide epitope selected from Table 1A, 1B, 1C, 1D, 1E, and/or 1F either alone or in the context of an MHC molecule; b) determining binding of a plurality of candidate peptide-binding molecules or antigen-binding fragments thereof to the peptide or stable MHC-peptide complex; and c) identifying one or more peptide-binding molecules or antigen-binding fragments thereof that bind to the peptide epitope or the stable MHC-peptide complex, is provided.

Numerous embodiments are further provided that may be applied to any aspect of the present invention and/or combined with any other embodiment described herein. For example, in one embodiment, the MHC molecule is a MHC multimer, optionally wherein the MHC multimer is a tetramer. In another embodiment, the MHC molecule is a MHC class I molecule. In still another embodiment, the MHC molecule comprises an MHC alpha chain that is an HLA serotype selected from the group consisting of HLA-A*02, HLA-A*03, HLA-A*01, HLA-A*11, HLA-A*24, and HLA-B*07, optionally wherein the HLA allele is selected from the group consisting of HLA-A*0201, HLA-A*0202, HLA-A*0203, HLA-A*0204, HLA-A*0205, HLA-A*0206, HLA-A*0207, HLA-A*0210, HLA-A*0211, HLA-A*0212, HLA-A*0213, HLA-A*0214, HLA-A*0216, HLA-A*0217, HLA-A*0219, HLA-A*0220, HLA-A*0222, HLA-A*0224, HLA-A*0230, HLA-A*0242, HLA-A*0253, HLA-A*0260, HLA-A*0274 allele, HLA-A*0301, HLA-A*0302, HLA-A*0305, HLA-A*0307, HLA-A*0101, HLA-A*0102, HLA-A*0103, HLA-A*0116 allele, HLA-A*1101, HLA-A*1102, HLA-A*1103, HLA-A*1104, HLA-A*1105, HLA-A*1119 allele, HLA-A*2402, HLA-A*2403, HLA-A*2405, HLA-A*2407, HLA-A*2408, HLA-A*2410, HLA-A*2414, HLA-A*2417, HLA-A*2420, HLA-A*2422, HLA-A*2425, HLA-A*2426, HLA-A*2458 allele, HLA-B*0702, HLA-B*0704, HLA-B*0705, HLA-B*0709, HLA-B*0710, HLA-B*0715, and HLA-B*0721 allele. In yet another embodiment, the peptide epitope and the MHC molecule are covalently linked and/or wherein the alpha and beta chains of the MHC molecule are covalently linked. In another embodiment, the stable MHC-peptide complex comprises a detectable label, optionally wherein the detectable label is a fluorophore. In still another embodiment, the plurality of candidate peptide binding molecules comprises one or more T cell receptors (TCRs), or one or more antigen-binding fragments of a TCR. In yet another embodiment, the plurality of candidate peptide binding molecules comprises at least 2, 5, 10, 100, 10³, 10⁴, 10⁵, 10⁶, 10⁷, 10⁸, 10⁹, or more, different candidate peptide binding molecules. In another embodiment, the plurality of candidate peptide binding molecules comprises one or more candidate peptide binding molecules that are obtained from a sample from a subject or a population of subjects; or the plurality of candidate peptide binding molecules comprises one or more candidate peptide binding molecules that comprise mutations in a parent scaffold peptide binding molecule obtained from a sample from a subject. In still another embodiment, the subject or population of subjects are a) not infected with SARS-CoV-2 and/or have recovered from COVID-19 or b) infected with SARS-CoV-2 and/or have COVID-19. In yet another embodiment, the subject or population of subjects has been vaccinated with one or more immunogenic peptides, wherein the immunogenic peptides comprise a peptide epitope selected from Table 1A, 1B, 1C, 1D, 1E, and/or 1F. In another embodiment, the subject is a mammal, optionally wherein the mammal is a human, a primate, or a rodent. In still another embodiment, the subject is an HLA-transgenic mouse and/or is a human TCR transgenic mouse. In yet another embodiment, the sample comprises T cells. In another embodiment, the sample comprises peripheral blood mononuclear cells (PBMCs) or CD8+ memory T cells. In still another embodiment, the antigen-binding fragment of a TCR is a single chain TCR (scTCR).

In another aspect, the peptide-binding molecule or antigen-binding fragment thereof identified according to a method described herein, optionally wherein the binding moiety is an antibody, an antigen-binding fragment of an antibody, a TCR, an antigen-binding fragment of a TCR, a single chain TCR (scTCR), a chimeric antigen receptor (CAR), or a fusion protein comprising a TCR and an effector domain, is provided.

In still another aspect, a method of treating SARS-CoV-2 infection in a subject comprising administering to the subject a therapeutically effective amount of genetically engineered T cells that express a TCR identified by a method described herein, is provided..

In yet another aspect, a method of treating SARS-CoV-2 infection in a subject comprising administering to the subject a therapeutically effective amount of genetically engineered T cells that express a TCR that binds to a peptide epitope selected from Table 1A, 1B, 1C, 1D, 1E, and/or 1F, is provided.

In another aspect, a method of treating SARS-CoV-2 infection in a subject comprising administering to the subject a therapeutically effective amount of genetically engineered T cells that express a TCR that binds to a stable MHC-peptide complex comprising a peptide epitope selected from Table 1A, 1B, 1C, 1D, 1E, and/or 1F in the context of an MHC molecule, is provided.

Numerous embodiments are further provided that may be applied to any aspect of the present invention and/or combined with any other embodiment described herein. For example, in one embodiment, the MHC molecule is a MHC multimer, optionally wherein the MHC multimer is a tetramer. In another embodiment, the MHC molecule is a MHC class I molecule. In still another embodiment, the MHC molecule comprises an MHC alpha chain that is an HLA serotype selected from the group consisting of HLA-A*02, HLA-A*03, HLA-A*01, HLA-A*11, HLA-A*24, and HLA-B*07, optionally wherein the HLA allele is selected from the group consisting of HLA-A*0201, HLA-A*0202, HLA-A*0203, HLA-A*0204, HLA-A*0205, HLA-A*0206, HLA-A*0207, HLA-A*0210, HLA-A*0211, HLA-A*0212, HLA-A*0213, HLA-A*0214, HLA-A*0216, HLA-A*0217, HLA-A*0219, HLA-A*0220, HLA-A*0222, HLA-A*0224, HLA-A*0230, HLA-A*0242, HLA-A*0253, HLA-A*0260, HLA-A*0274 allele, HLA-A*0301, HLA-A*0302, HLA-A*0305, HLA-A*0307, HLA-A*0101, HLA-A*0102, HLA-A*0103, HLA-A*0116 allele, HLA-A*1101, HLA-A*1102, HLA-A*1103, HLA-A*1104, HLA-A*1105, HLA-A*1119 allele, HLA-A*2402, HLA-A*2403, HLA-A*2405, HLA-A*2407, HLA-A*2408, HLA-A*2410, HLA-A*2414, HLA-A*2417, HLA-A*2420, HLA-A*2422, HLA-A*2425, HLA-A*2426, HLA-A*2458 allele, HLA-B*0702, HLA-B*0704, HLA-B*0705, HLA-B*0709, HLA-B*0710, HLA-B*0715, and HLA-B*0721 allele. In yet another embodiment, the peptide epitope and the MHC molecule are covalently linked and/or wherein the alpha and beta chains of the MHC molecule are covalently linked. In another embodiment, the stable MHC-peptide complex comprises a detectable label, optionally wherein the detectable label is a fluorophore. In still another embodiment, the T cells are isolated from a) the subject, b) a donor not infected with SARS-CoV-2, or c) a donor recovered from COVID-19.

In still another aspect, a method of treating SARS-CoV-2 infection in a subject comprising transfusing antigen-specific T cells to the subject, wherein the antigen-specific T cells are generated by a) stimulating PBMCs or T cells from a subject with a peptide epitope selected from Table 1A, 1B, 1C, 1D, 1E, and/or 1F, a stable MHC-peptide complex comprising a peptide epitope selected from Table 1A, 1B, 1C, 1D, 1E, and/or 1F in the context of an MHC molecule, or a cell that presents a peptide epitope selected from Table 1A, 1B, 1C, 1D, 1E, and/or 1F in the context of a MHC molecule on its cell surface; and b) expanding antigen-specific T cells in vitro, optionally isolating PBMCs or T cells from the subject before stimulating the PBMCs or T cells, is provided.

Numerous embodiments are further provided that may be applied to any aspect of the present invention and/or combined with any other embodiment described herein. For example, in one embodiment, the T cell is a naive T cell, a central memory T cell, or an effector memory T cell. In another embodiment, the T cell is a CD8+ memory T cell. In still another embodiment, the agents are placed in contact under conditions and for a time suitable for the formation of at least one immune complex between the peptide epitope, immunogenic peptide, stable MHC-peptide complex, T cell receptor, and/or T cell. In yet another embodiment, the peptide epitope, immunogenic peptide, stable MHC-peptide complex, and/or T cell receptor are expressed by cells and the cells are expanded and/or isolated during one or more steps. In another embodiment, the subject is a mammal, optionally wherein the mammal is a human, a primate, or a rodent.

### BRIEF DESCRIPTION OF THE DRAWINGS

FIG. 1 shows sample a representative list of exemplary COVID functional epitope targets identified from patients. Sample screen data illustrate the identification of common shared epitopes and epitopes from individual patients. The x-axis shows target enrichment in patient 01-01-001. The y-axis shows target enrichment in patient 01-01-004. The dotted line indicates the enrichment threshold for selecting particularly strong targets.

FIG. 2A and FIG. 2B show that identified T cell epitopes are shared across multiple patients. FIG. 2A shows an enrichment of target epitope KLWAQCVQL across multiple patients harboring HLA-A*02:01 or HLA-A*03:01 alleles. FIG. 2B shows enrichment of target epitope KTFPPTEPKK across patients. Patients who were hospitalized are highlighted in brown and more severe patients that needed ventilators are shown in red.

FIG. 3A and FIG. 3B show a summary of identified T cell epitopes. The x-axis shows a representative list of exemplary functional epitopes identified in screens. The y-axis shows a log2-fold enrichment for each patient.

FIG. 4A - FIG. 4C show the T-Scan approach for comprehensive mapping of the memory CD8+ T cell response to SARS-CoV-2. FIG. 4A shows an overview of the T-Scan antigen discovery screen. FIG. 4B shows the design of the ORFeome-wide SARS-CoV-2 antigen library. FIG. 4C shows an example SARS-CoV-2 ORFeome-wide screen data for a convalescent COVID 19 patient (top panel) and healthy control (bottom panel). Each circle represents a single 61aa SARS-CoV-2 protein fragment, with the X-axis showing the position of the fragment in the concatenated SARS-CoV-2 ORFeome. The Y-axis shows the performance of the fragment in the screen, calculated as the enrichment of target cells expressing the fragment in the sorted target cells expressing the protein fragment relative to the unsorted library. For the calculation, the ten internal nucleotide barcodes for each fragment were combined and the performance of the four technical screen replicates was averaged using a modified geometric mean. The right panels show the performance of the 60 positive control protein fragments derived from CMV, EBV, and Influenza.

FIG. 5A - FIG. 5H show results of discovering and validating immunodominant SARS-CoV-2 epitopes presented on HLA-A*02:01. FIG. 5A shows SARS-CoV-2 ORFeome-wide screen data for nine HLA-A*02:01 COVID19 patients. Each circle corresponds to a 20 amino acid (aa) stretch of the SARS-CoV-2 ORFeome, with the X-axis indicating the position of the stretch in the SARS-CoV-2 genome. The Y-axis shows the mean performance of all of the library fragments spanning the given 20aa stretch, calculated as the enrichment of target cells expressing the fragment in the sorted pool (T-cell recognized) compared to the unsorted library (see FIG. 4C). For the calculation, the ten internal nucleotide barcodes for each fragment were combined and the performance of the four technical screen replicates was averaged using a modified geometric mean. The screen results for nine HLA-A*02:01 patients are marked with different colors. FIG. 5B shows screen data for identified KLW epitope (KLWAQCVQL). The boxplots represent the screen enrichments of all of the fragments in the library that contain the KLW epitope. For this calculation, the ten internal nucleotide barcodes for each fragment were combined and the performance of the four technical screen replicates was averaged using a modified geometric mean. The data for the nine HLA-A*0201 COVID 19 patient screens are shown in blue, two healthy control HLA-A*0201 screens shown in grey, and five HLA-A*0301 COVID19 patient screens shown in red. FIG. 5C shows the collapsed screen data for six identified shared epitopes. Each boxplot shows the aggregate enrichment of one epitope in each of the nine screened HLA-A*0201 COVID19 patients (black dots) and two healthy controls (blue dots). The Y-axis shows the mean enrichment of all fragments in the library containing the given epitope, with the ten internal nucleotide barcodes combined and the performance of the four technical screen replicates averaged. Full epitope sequences are listed in Table 5. FIG. 5D shows the IFNg ELISA validation of identified epitopes. Memory CD8+ T cells from four HLA-A*02:01 COVID19 patients were incubated with HLA-A*02:01 target cells and 1uM of each described peptide for 16 hr. The Y-axis shows the concentration of IFNg secreted by T cells from each patient (black dot) in the presence of each peptide compared to a no-peptide control. Data are the means of two technical replicates and representative of two independent experiments. FIG. 5E shows the tetramer staining quantification of memory CD8+ T cells reactive to six shared HLA-A*02:01 epitopes. Memory CD8+ T cells from 27 HLA-A*02:01 COVID19 patients (black dots) and one healthy control (blue dots) were stained using tetramers loaded with each of the six identified epitopes. The Y-axis indicates the percentage of tetramer-positive cells among all CD8+ cells. FIG. 5F shows the correlation of screen performance and cognate T cell frequency as determined by tetramer staining. Each circle indicates the performance of one epitope in one of the nine screened HLA-A*0201 COVID 19 patients. The X-axis shows the aggregate performance of the epitope in the T-Scan screen, calculated as the average enrichment of all fragments containing that epitope. The Y-axis shows the frequency of tetramer-positive memory CD8+ T cells recognizing that epitope. FIG. 5G and FIG. 5H show recognition of the three most common HLA-A*02:01 epitopes across COVID19 patients based on screening data (n=9) (FIG. 5G) or tetramer staining (n=27) (FIG. 5H). For FIG. 5G, patients were considered positive for an epitope if the aggregate performance of the epitope in the screen data exceeded a set threshold (mean + 2SD of the enrichment of all of the SARS-CoV-2 fragments in the healthy controls). For FIG. 5H, patients were considered positive for an epitope if >0.05% of memory CD8+ T cells were positive by tetramer staining. Patients with no detectable reactivity to any of the three epitopes (4/27) are shown outside the Venn diagram.

FIG. 6A - FIG. 6F show screen data for all validated epitopes. The boxplots represent the screen enrichments of all fragments in the library that contain each described epitope. Samples are colored based on the MHC restriction on which the screen was performed.

FIG. 7A - FIG. 7F show genome-wide screen hits are enriched for high-affinity MHC binding epitopes. The boxplots represent the predicted MHC binding affinity for each fragment of the library (Entire Library) compared to the predicted MHC binding affinity for the top scoring fragments in each set of screens on a single MHC allele. The MHC binding affinity for each tile was calculated by taking the strongest binder as predicted by NetMHC4.0.

FIG. 8 shows validation of epitopes using activation-induced surface markers. Peptides identified by the T-Scan screen were validated by measuring the frequency of activated T cells when co-cultured with target cells pulsed with the identified peptide (1 µM). Each plot depicts the correlation of screen performance (X-axis) and the frequency of CD8+, CD 137+, and CD69+ T cells (Y-axis) when pulsed with the indicated peptide (color of dots) for the indicated HLA. Each dot represents the mean frequency of activated cells for T cells from an individual patient as a fold change over un-pulsed controls.

FIG. 9 shows validation of epitopes using IFNγ secretion peptides identified by the T-Scan screen were validated by measuring IFNγ secretion of T cells co-cultured with target cells pulsed with the identified peptide (1 µM). Each plot depicts the correlation of screen performance (X-axis) and the concentration of IFNγ (Y-axis) when pulsed with the indicated peptide (color of dots). Each dot represents the mean fold change of IFNγ concentration over un-pulsed controls for T cells from an individual patient.

FIG. 10 shows T-Scan screen data for HLA-A*01:01 (n=5), HLA-A*03:01 (n=5), HLA-A*11:01 (n=5), HLA-A*24:02 (n=5), and HLA-B*07:02 (n=5) COVID-19 patients. Each circle corresponds to a 20aa stretch of the SARS-CoV-2 ORFeome, with the X-axis indicating the position of the stretch in the SARS-CoV-2 genome. The Y-axis shows the mean performance of all library fragments spanning the given 20aa stretch, calculated as described in FIG. 4C. Results for each patient are marked with different colors.

FIG. 11A - FIG. 11C show the discovery and validation of immunodominant SARS-CoV-2 epitopes presented on HLA-A*01:01, HLA-A*03:01, HLA-A*11:01, HLA-A*24:02, and HLA-B*07:02. FIG. 11A shows collapsed screen data for shared epitopes identified for each analyzed MHC allele. Each boxplot shows the aggregate enrichment of one epitope in each of the five COVID19 patients (black dots) screened for the listed allele. The Y-axis shows the mean enrichment of all fragments in the library containing the given epitope, with the ten internal nucleotide barcodes combined and the performance of the four technical screen replicates averaged. Full epitope sequences are listed in Table 5. FIG. 11B shows IFNg ELISA validation of identified epitopes. Memory CD8+ T cells from four COVID 19 patients positive for each MHC allele were incubated with MHC-matched target cells and 1 uM of each described peptide for 16 hr. The Y-axis shows the concentration of IFNg secreted by T cells from each patient (black dot) in the presence of each peptide compared to a no-peptide control. Data are the means of two technical replicates and representative of two independent experiments. Validation included some patients that had not been used in the original screening experiments. FIG. 11C shows recognition of the three most common epitopes for each MHC allele across five COVID19 patients. Patients were considered positive for an epitope if the aggregate performance of the epitope in the screen data exceeded a threshold (mean + 2SD of the enrichment of all of the SARS-CoV-2 fragments in the healthy controls).

FIG. 12A - FIG. 12C show the immunodominant epitopes span the SARS-CoV-2 ORFeome and are recognized by TCRs with shared features. FIG. 12A shows a distribution of immunodominant CD8+ T cell epitopes across the SARS-CoV-2 genome. Each bar represents one validated immunodominant epitope, with the X-axis showing its position in the SARS-CoV-2 ORFeome, the color indicating its MHC restriction, and the height of the bar indicating the fraction of MHC-matched patients recognizing the epitope. Patients were considered positive for an epitope if the aggregate performance of the epitope in the screen data exceeded a threshold (mean + 2 standard deviations (SD) of the enrichment of all of the SARS-CoV-2 fragments in the healthy controls). For clarity, overlapping epitopes are plotted as adjacent bars. FIG. 12B shows immunodominant CD8+ T-cell epitopes by SARS-CoV-2 ORF. The stacked bar graphs show the number of immunodominant epitopes per ORF, with the colors indicating the MHC restriction of each epitope. The MHC color-coding is the same as shown in FIG. 12A. FIG. 12C shows TCR alpha variable (TRAV) gene usage in tetramer-positive T cells across patients. Height of each box corresponds to the number of T cells within the clonotype. Blue corresponds to conserved TRAV gene for a specific epitope and red corresponds to all other TRAV genes.

FIG. 13A - FIG. 13C show the minimal cross-reactivity of SARS-CoV-2-reactive memory T cells with other coronaviruses. FIG. 13A shows screen data compared across coronavirus ORFeomes. Each panel shows the collective reactivity to one coronavirus genome (SARS-CoV-2, SARS-CoV-1, OC43, HKU1, NL63, or 229E) detected in the 34 T-Scan screens performed. Each circle corresponds to a 20aa stretch of the coronavirus ORFeome, with the X-axis indicating the position of the stretch in the ORFeome. The Y-axis shows the mean performance of all of the library fragments spanning the given 20aa stretch, calculated as the enrichment of target cells expressing the fragment in the sorted pool (T-cell recognized) compared to the unsorted library. For the calculation, the ten internal nucleotide barcodes for each fragment were combined and the performance of the four technical screen replicates was averaged using a modified geometric mean (see methods and FIG. 4C). Results for nine HLA-A*02:01 screens are marked in blue, five HLA-A*03:01 screens are marked in red, five HLA-A*01:01 screens are marked in yellow, five HLA-A* 11:01 screens are marked in green, five HLA-A*24:02 screens are marked in cyan, and five HLA-B*07:02 screens are marked in magenta. For visualization, the positions of the conserved ORF1ab, S, M, E, and N proteins was aligned across all ORFeomes. FIG. 13B shows an alignment of the KLW epitope across coronavirus genomes. The alignment shows the region of each coronavirus genome corresponding to the SARS-CoV-2 HLA-A*02:01 KLW epitope. The boxplots show the aggregate screen performance of all of the fragments containing each epitope variant for nine HLA-A*02:01-positive COVID19 patients (black dots) and two HLA-A*02:01-positive healthy controls (blue dots). FIG. 13C shows an alignment of the SPR epitope across coronavirus genomes. The alignment shows the region of each coronavirus genome corresponding to the SARS-CoV-2 HLA-B*07:02 epitope. The boxplots show the aggregate screen performance of all of the fragments containing each epitope variant for five HLA-B*07:02-positive COVID19 patients (black dots).

### DETAILED DESCRIPTION OF THE INVENTION

The present invention is based, at least in part, on the discovery of SARS-CoV-2 virus-specific immunogenic peptides. A systematic, comprehensive survey was carried out to map the precise T cell targets recognized by convalescent COVID-19 patients. Strikingly, the study revealed a limited set of highly immunodominant peptide antigens that are recurrently recognized across patients, including several that appear to be universally recognized. For example, it was determined herein that the CD8+ T cell response is dominated by a few (3-8) highly antigenic (immunodominant) epitopes in SARS-CoV-2 that are shared among patients with the same HLA type. These epitopes are largely unique to SARS-CoV-2 (i.e., do not occur in “common cold” coronaviruses), are invariant among viral isolates, and are frequently targeted by multiple clonotypes within each patient. At least twenty-nine shared epitopes were identified across the six HLA types studied. Notably, only ~10% (3 of 29) of the epitopes occur in the S protein, highlighting the need for new classes of vaccines that are designed to elicit a broader CD8+ T cell response. Remarkably, it was determined that 94% of screened patients had T cells that recognized at least one of the three most dominant epitopes for a given HLA and 53% of patients had T cells that recognized all three of the most dominant epitopes for a given HLA. Additional confirmatory analyses in 18 additional A*02:01 patients reiterated the presence of memory CD8+ T cells specific for the top six identified A*02:01 epitopes, and single-cell sequencing revealed that patients often have >5 different T cell clones targeting each epitope, but that the same T cell receptor Va and Vb regions are predominantly used to recognize these epitopes, even across patients. T cells that target most of these immunodominant epitopes (27 of 29) do not cross-react with the endemic coronaviruses that cause the common cold, and the epitopes do not occur in regions with high mutational variation. These results provide useful tools to better understand the CD8+ T cell response in COVID-19 and have significant implications for vaccine design and development.

Accordingly, the present invention relates, in part, to the identified immunogenic peptides, compositions comprising these immunogenic peptides alone or with MHC molecules, stable MHC-peptide complexes, methods of diagnosing, prognosing, and monitoring T cell response to SARS-CoV-2, and methods for preventing and/or treating SARS-CoV-2 infection by administering immunogenic compositions comprising the identified immunogenic peptides.

### I. Definitions

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

As used herein, the term “administering” means providing a pharmaceutical agent or composition to a subject, and includes, but is not limited to, administering by a medical professional and self-administering.

The term “immune response” includes T cell mediated and/or B cell mediated immune responses. Exemplary immune responses include T cell responses, e.g., cytokine production and cellular cytotoxicity. In addition, the term immune response includes immune responses that are indirectly effected by T cell activation, e.g., antibody production (humoral responses) and activation of cytokine responsive cells, e.g., macrophages.

Conventional T cells, also known as Tconv or Teffs, have effector functions (e.g., cytokine secretion, cytotoxic activity, anti-self-recognition, and the like) to increase immune responses by virtue of their expression of one or more T cell receptors. Tcons or Teffs are generally defined as any T cell population that is not a Treg and include, for example, naive T cells, activated T cells, memory T cells, resting Tcons, or Tcons that have differentiated toward, for example, the Th1 or Th2 lineages. In some embodiments, Teffs are a subset of non-Treg T cells. In some embodiments, Teffs are CD4+ Teffs or CD8+ Teffs, such as CD4+ helper T lymphocytes (e.g., Th0, Th1, Tfh, or Th17) and CD8+ cytotoxic T lymphocytes. As described further herein, cytotoxic T cells are CD8+ T lymphocytes. “Naïve Tcons” are CD4⁺ T cells that have differentiated in bone marrow, and successfully underwent a positive and negative processes of central selection in a thymus, but have not yet been activated by exposure to an antigen. Naive Tcons are commonly characterized by surface expression of L-selectin (CD62L), absence of activation markers such as CD25, CD44 or CD69, and absence of memory markers such as CD45RO. Naïve Tcons are therefore believed to be quiescent and non-dividing, requiring interleukin-7 (IL-7) and interleukin-15 (IL-15) for homeostatic survival (see, at least WO 2010/101870). The presence and activity of such cells are undesired in the context of suppressing immune responses. Unlike Tregs, Tcons are not anergic and can proliferate in response to antigen-based T cell receptor activation (Lechler et al. (2001) Philos. Trans. R. Soc. Lond. Biol. Sci. 356:625-637). In tumors, exhausted cells can present hallmarks of anergy.

The term “vaccine” refers to a pharmaceutical composition that elicits an immune response to an antigen of interest. The vaccine may also confer protective immunity upon a subject.

“Vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops, which, in their vector form are not bound to the chromosome. In the present specification, “plasmid” and “vector” are used interchangeably as the plasmid is the most commonly used form of vector. However, as will be appreciated by those skilled in the art, the invention is intended to include such other forms of expression vectors which serve equivalent functions and which become subsequently known in the art.

The term “immunotherapeutic agent” may include any molecule, peptide, antibody or other agent which can stimulate a host immune system to generate an immune response to a viral infection in the subject. Various immunotherapeutic agents are useful in the compositions and methods described herein.

An “isolated protein” refers to a protein that is substantially free of other proteins, cellular material, separation medium, and culture medium when isolated from cells or produced by recombinant DNA techniques, or chemical precursors or other chemicals when chemically synthesized. An “isolated” or “purified” protein or biologically active portion thereof is substantially free of cellular material or other contaminating proteins from the cell or tissue source from which the antibody, polypeptide, peptide or fusion protein is derived, or substantially free from chemical precursors or other chemicals when chemically synthesized. The language “substantially free of cellular material” includes preparations of a biomarker polypeptide or fragment thereof, in which the protein is separated from cellular components of the cells from which it is isolated or recombinantly produced. In one embodiment, the language “substantially free of cellular material” includes preparations of a biomarker protein or fragment thereof, having less than about 30% (by dry weight) of non-biomarker protein (also referred to herein as a “contaminating protein”), more preferably less than about 20% of non-biomarker protein, still more preferably less than about 10% of non-biomarker protein, and most preferably less than about 5% non-biomarker protein. When antibody, polypeptide, peptide or fusion protein or fragment thereof, e.g., a biologically active fragment thereof, is recombinantly produced, it is also preferably substantially free of culture medium, i.e., culture medium represents less than about 20%, more preferably less than about 10%, and most preferably less than about 5% of the volume of the protein preparation.

As used herein, the term “isotype” refers to the antibody class (e.g., IgM, IgG1, IgG2C, and the like) that is encoded by heavy chain constant region genes.

As used herein, the term “K_{D}” is intended to refer to the dissociation equilibrium constant of a particular antibody-antigen interaction. The binding affinity of antibodies of the disclosed invention may be measured or determined by standard antibody-antigen assays, for example, competitive assays, saturation assays, or standard immunoassays such as ELISA or RIA.

A “kit” is any manufacture (e.g., a package or container) comprising at least one reagent, e.g., a probe or small molecule, for specifically detecting and/or affecting the expression of a marker encompassed by the present invention. The kit may be promoted, distributed, or sold as a unit for performing the methods encompassed by the present invention. The kit may comprise one or more reagents necessary to express a composition useful in the methods encompassed by the present invention. In certain embodiments, the kit may further comprise a reference standard, e.g., a nucleic acid encoding a protein that does not affect or regulate signaling pathways controlling cell growth, division, migration, survival or apoptosis. One skilled in the art can envision many such control proteins, including, but not limited to, common molecular tags (e.g., green fluorescent protein and beta-galactosidase), proteins not classified in any of pathway encompassing cell growth, division, migration, survival or apoptosis by GeneOntology reference, or ubiquitous housekeeping proteins. Reagents in the kit may be provided in individual containers or as mixtures of two or more reagents in a single container. In addition, instructional materials which describe the use of the compositions within the kit may be included.

The terms “prevent,” “preventing,” “prevention,” “prophylactic treatment,” and the like refer to reducing the probability of developing a disease, disorder, or condition in a subject, who does not have, but is at risk of or susceptible to developing a disease, disorder, or condition.

The term “prognosis” includes a prediction of the probable course and outcome of a viral infection or the likelihood of recovery from the disease. In some embodiments, the use of statistical algorithms provides a prognosis of a viral infection in an individual. For example, the prognosis may be surgery, development of a clinical subtype of a viral infection, development of one or more clinical factors, or recovery from the disease.

The term “sample” used for detecting or determining the presence or level of at least one biomarker is typically brain tissue, cerebrospinal fluid, whole blood, plasma, serum, saliva, urine, stool (e.g., feces), tears, and any other bodily fluid (e.g., as described above under the definition of “body fluids”), or a tissue sample (e.g., biopsy) such as a small intestine, colon sample, or surgical resection tissue. In certain instances, the method encompassed by the present invention further comprises obtaining the sample from the individual prior to detecting or determining the presence or level of at least one marker in the sample.

The term “small molecule” is a term of the art and includes molecules that are less than about 1000 molecular weight or less than about 500 molecular weight. In one embodiment, small molecules do not exclusively comprise peptide bonds. In another embodiment, small molecules are not oligomeric. Exemplary small molecule compounds which may be screened for activity include, but are not limited to, peptides, peptidomimetics, nucleic acids, carbohydrates, small organic molecules (e.g., polyketides) (Cane et al. (1998) Science 282:63), and natural product extract libraries. In another embodiment, the compounds are small, organic non-peptidic compounds. In a further embodiment, a small molecule is not biosynthetic.

The term “specific binding” refers to antibody binding to a predetermined antigen. Typically, the antibody binds with an affinity (K_{D}) of approximately less than 10⁻⁷ M, such as approximately less than 10⁻⁸ M, 10⁻⁹ M or 10⁻¹⁰ M or even lower when determined by surface plasmon resonance (SPR) technology in a BIACORE® assay instrument using an antigen of interest as the analyte and the antibody as the ligand, and binds to the predetermined antigen with an affinity that is at least 1.1-, 1.2-, 1.3-, 1.4-, 1.5-, 1.6-, 1.7-, 1.8-, 1.9-, 2.0-, 2.5-, 3.0-, 3.5-, 4.0-, 4.5-, 5.0-, 6.0-, 7.0-, 8.0-, 9.0-, or 10.0-fold or greater than its affinity for binding to a non-specific antigen (e.g., BSA, casein) other than the predetermined antigen or a closely-related antigen. The phrases “an antibody recognizing an antigen” and “an antibody specific for an antigen” are used interchangeably herein with the term “an antibody which binds specifically to an antigen.” Selective binding is a relative term referring to the ability of an antibody to discriminate the binding of one antigen over another.

The term “subject” refers to any healthy animal, mammal or human, or any animal, mammal or human afflicted with a viral infection, e.g., SARS-CoV-2 infection. The term “subject” is interchangeable with “patient.”

As used herein, “percent identity” between amino acid sequences is synonymous with “percent homology,” which can be determined using the algorithm of Karlin and Altschul (Proc. Natl. Acad. Sci. USA 87, 2264-2268, 1990), modified by Karlin and Altschul (Proc. Natl. Acad. Sci. USA 90, 5873-5877, 1993). The noted algorithm is incorporated into the NBLAST and XBLAST programs of Altschul et al. (J. Mol. Biol. 215, 403-410, 1990). BLAST nucleotide searches are performed with the NBLAST program, score=100, wordlength=12, to obtain nucleotide sequences homologous to a polynucleotide described herein. BLAST protein searches are performed with the XBLAST program, score=50, wordlength=3, to obtain amino acid sequences homologous to a reference polypeptide. To obtain gapped alignments for comparison purposes, Gapped BLAST is utilized as described in Altschul et al. (Nucleic Acids Res. 25, 3389-3402, 1997). When utilizing BLAST and Gapped BLAST programs, the default parameters of the respective programs (e.g., XBLAST and NBLAST) are used.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body.

A “transcribed polynucleotide” or “nucleotide transcript” is a polynucleotide (e.g., an mRNA, hnRNA, a cDNA, or an analog of such RNA or cDNA) which is complementary to or homologous with all or a portion of a mature mRNA made by transcription of a biomarker nucleic acid and normal post-transcriptional processing (e.g., splicing), if any, of the RNA transcript, and reverse transcription of the RNA transcript.

The term “T cell” includes CD4⁺ T cells and CD8⁺ T cells. The term T cell also includes both T helper 1 type T cells and T helper 2 type T cells. The term “antigen presenting cell” includes professional antigen presenting cells (e.g., B lymphocytes, monocytes, dendritic cells, Langerhans cells), as well as other antigen presenting cells (e.g., keratinocytes, endothelial cells, astrocytes, fibroblasts, and oligodendrocytes).

The term “T cell receptor” or “TCR” should be understood to encompass full TCRs as well as antigen-binding portions or antigen-binding fragments thereof. In some embodiments, the TCR is an intact or full-length TCR, including TCRs in the αβ form or γδ form. In some embodiments, the TCR is an antigen-binding portion that is less than a full-length TCR but that binds to a specific peptide bound in an MHC molecule, such as binds to an MHC-peptide complex. In some cases, an antigen-binding portion or fragment of a TCR may contain only a portion of the structural domains of a full-length or intact TCR, but yet is able to bind the peptide epitope, such as MHC-peptide complex, to which the full TCR binds. In some cases, an antigen-binding portion contains the variable domains of a TCR, such as variable α chain and variable β chain of a TCR, sufficient to form a binding site for binding to a specific MHC-peptide complex. Generally, the variable chains of a TCR contain complementarity determining regions (CDRs) involved in recognition of the peptide, MHC and/or MHC-peptide complex.

The term “therapeutic effect” refers to a local or systemic effect in animals, particularly mammals, and more particularly humans, caused by a pharmacologically active substance. The term thus means any substance intended for use in the diagnosis, cure, mitigation, treatment or prevention of disease or in the enhancement of desirable physical or mental development and conditions in an animal or human. The phrase “therapeutically-effective amount” means that amount of such a substance that produces some desired local or systemic effect at a reasonable benefit/risk ratio applicable to any treatment. In certain embodiments, a therapeutically effective amount of a compound will depend on its therapeutic index, solubility, and the like. For example, certain compounds discovered by the methods encompassed by the present invention may be administered in a sufficient amount to produce a reasonable benefit/risk ratio applicable to such treatment.

The terms “therapeutically-effective amount” and “effective amount” as used herein means that amount of a compound, material, or composition comprising a compound encompassed by the present invention which is effective for producing some desired therapeutic effect in at least a sub-population of cells in an animal at a reasonable benefit/risk ratio applicable to any medical treatment. Toxicity and therapeutic efficacy of subject compounds may be determined by standard pharmaceutical procedures in cell cultures or experimental animals, e.g., for determining the LD₅₀ and the ED₅₀. Compositions that exhibit large therapeutic indices are preferred. In some embodiments, the LD₅₀ (lethal dosage) may be measured and may be, for example, at least 10%, 20%, 30%, 40%, 50%, 60%, 70%, 80%, 90%, 100%, 200%, 300%, 400%, 500%, 600%, 700%, 800%, 900%, 1000% or more reduced for the agent relative to no administration of the agent. Similarly, the ED₅₀ (i.e., the concentration which achieves a half-maximal inhibition of symptoms) may be measured and may be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. Also, Similarly, the IC₅₀ may be measured and may be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. In some embodiments, T cell immune response in an assay may be increased by at least about 10%, 15%, 20%, 25%, 30%, 35%, 40%, 45%, 50%, 55%, 60%, 65%, 70%, 75%, 80%, 85%, 90%, 95%, or even 100%. In another embodiment, at least about a 10%, 15%, 20%, 25%, 30%, 35%, 40%, 45%, 50%, 55%, 60%, 65%, 70%, 75%, 80%, 85%, 90%, 95%, or even 100% decrease in a viral load may be achieved.

“Treating” a disease in a subject or “treating” a subject having a disease refers to subjecting the subject to a pharmaceutical treatment, e.g., the administration of a drug, such that at least one symptom of the disease is decreased or prevented from worsening.

The term “body fluid” refers to fluids that are excreted or secreted from the body as well as fluids that are normally not (e.g., amniotic fluid, aqueous humor, bile, blood and blood plasma, cerebrospinal fluid, cerumen and earwax, cowper’s fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit).

The term “coding region” refers to regions of a nucleotide sequence comprising codons which are translated into amino acid residues, whereas the term “noncoding region” refers to regions of a nucleotide sequence that are not translated into amino acids (e.g., 5′ and 3′ untranslated regions).

The term “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

As used herein, the term “costimulate” with reference to activated immune cells includes the ability of a costimulatory molecule to provide a second, non-activating receptor mediated signal (a “costimulatory signal”) that induces proliferation or effector function. For example, a costimulatory signal may result in cytokine secretion, e.g., in a T cell that has received a T cell-receptor-mediated signal. Immune cells that have received a cell-receptor mediated signal, e.g., via an activating receptor are referred to herein as “activated immune cells.”

The term “determining a suitable treatment regimen for the subject” is taken to mean the determination of a treatment regimen (i.e., a single therapy or a combination of different therapies that are used for the prevention and/or treatment of the viral infection in the subject) for a subject that is started, modified and/or ended based or essentially based or at least partially based on the results of the analysis according to the present invention. One example is starting an adjuvant therapy after surgery whose purpose is to decrease the risk of recurrence, another would be to modify the dosage of a particular chemotherapy. The determination can, in addition to the results of the analysis according to the present invention, be based on personal characteristics of the subject to be treated. In most cases, the actual determination of the suitable treatment regimen for the subject will be performed by the attending physician or doctor.

The term “adjuvant” as used herein refers to substances, which when administered prior, together or after administration of an antigen accelerates, prolong and/or enhances the quality and/or strength of an immune response to the antigen in comparison to the administration of the antigen alone. Adjuvants can increase the magnitude and duration of the immune response induced by vaccination.

“Homologous” as used herein, refers to nucleotide sequence similarity between two regions of the same nucleic acid strand or between regions of two different nucleic acid strands. When a nucleotide residue position in both regions is occupied by the same nucleotide residue, then the regions are homologous at that position. A first region is homologous to a second region if at least one nucleotide residue position of each region is occupied by the same residue. Homology between two regions is expressed in terms of the proportion of nucleotide residue positions of the two regions that are occupied by the same nucleotide residue. By way of example, a region having the nucleotide sequence 5′-ATTGCC-3′ and a region having the nucleotide sequence 5′-TATGGC-3′ share 50% homology. Preferably, the first region comprises a first portion and the second region comprises a second portion, whereby, at least about 50%, and preferably at least about 75%, at least about 90%, or at least about 95% of the nucleotide residue positions of each of the portions are occupied by the same nucleotide residue. More preferably, all nucleotide residue positions of each of the portions are occupied by the same nucleotide residue.

The term “immune cell” refers to cells that play a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes.

The term “SARS-CoV-2” or “Severe Acute Respiratory Syndrome Coronavirus 2” refers to the causative agent of coronavirus disease 2019 (COVID-19). SARS-CoV-2 was identified as a pandemic by the World Health Organization (WHO) on Mar. 11, 2020. In supporting the process of entry of the virus into the host cell, SARS-CoV2 binds to the ACE2 receiver that is highly expressed in the lower respiratory tract such as type II alveolar cells (AT2) of the lungs, upper esophagus and stratified epithelial cells, and other cells such as absorptive enterocytes from the ileum and colon, cholangiocytes, myocardial cells, kidney proximal tubule cells, and bladder urothelial cells. Therefore, patients who are infected with this virus not only experience respiratory problems such as pneumonia leading to Acute Respiratory Distress Syndrome (ARDS), but also experience disorders of heart, kidneys, and digestive tract.

There is no specific treatment for eradication of the SARS-CoV2 virus in patients. Therapeutic approaches for another β-coronavirus approach such as SARS-CoV or MERS-CoV treatments may be used. Some of these approaches including lopinavir/ritonavir, chloroquine, and hydroxychloroquine. Aerosol inhalation of interferon α twice per night also could be used. In some cases, combinations of interferon-α combined with ribavirin have commonly used coronaviruses (such as MERS-CoV). It was also found that the combination of interferon with steroid drugs can accelerate lung repair and increase oxygen survival levels. However, inconsistent results have been shown for therapy using interferon α.

SARS-CoV-2 virus is an enveloped, non-segmented, positive sense RNA virus that is included in the sarbecovirus, ortho corona virinae subfamily which is broadly distributed in humans and other mammals. Its diameter is about 65-125 nm, containing single strands of RNA and provided with crown-like spikes on the outer surface. SARS-CoV2 is a novel β-coronavirus after the previously identified SARS-CoV and MERS-CoV which led to pulmonary failure and potentially fatal respiratory tract infection and caused outbreaks mainly in Guandong, China and Saudi Arabia.

The genome size of the SARS-CoV-2 varies from 29.8 kb to 29.9 kb and its genome structure followed the specific gene characteristics to known CoVs. The 5′ more than two-thirds of the genome comprises orf1a/b encoding orfla/b polyproteins, while the 3′ one third consists of genes encoding four main structural proteins including spike (S) glycoprotein, small envelope (E) glycoprotein, membrane (M) glycoprotein, and nucleocapsid (N) protein. Additionally, the SARS-CoV-2 contains 6 accessory proteins, encoded by ORF3a, ORF6, ORF7a, ORF7b, and ORF8 genes (Khailany et al. (2020) Gene Rep 19:100682).

The ORF1ab gene is the largest gene segment of the coronavirus and it constitutes two ORF, i.e., ORF1a and ORF1b, to produce two large overlapping polyproteins, pp1a (orf1a polyprotein) and pp1ab (orflab polyprotein) by contributing a ribosomal frame shifting event. The polyproteins are supplemented by protease enzymes namely papain-like proteases (PLpro) and a serine type Mpro (chymotrypsin-like protease (3CLpro)) protease that are encoded in nsp3 and nsp 5. Subsequently, cleavage occurs between pp1a and pp1ab into nonstructural proteins (nsps) 1-11 and 1-16, respectively. The nsps play an important role in many processes in viruses and host cells. Representative sequences of orf1a polyprotein and orf1ab polyprotein are presented below in Table 1G.

ORF3a is one of the accessory proteins encoded by SARS-CoV-2 genome. Recent studies have showed that the functional domains of SARS-CoV-2 ORF3a protein are linked to virulence, infectivity, ion channel formation, and virus release (Issa et al. (2020) mSystems 5:e00266-20). Representative sequences of ORF3a are presented below in Table 1G.

ORF7a is another SARS-CoV-2 genome-encoded accessory protein that is composed of a type I transmembrane protein that localizes primarily to the Golgi apparatus but can be found on the cell surface. SARS-CoV ORF7a overlaps ORF7b in the viral genome, where they share a transcriptional regulatory sequence (TRS). In some embodiments, ORF7a has a 15-amino-acid (aa) N-terminal signal peptide, an 81-aa luminal domain, a 21-aa transmembrane domain, and a 5-aa cytoplasmic tail (Taylor et al. (2015) J. Virol. 89:11820-11833). Representative sequences of ORF7a are presented below in Table 1G.

The spike or S glycoprotein is a transmembrane protein with a molecular weight of about 150 kDa found in the outer portion of the virus. S protein has an RBD located in the S1 subunit of the virus that facilitates entry of the virus into the host cell by binding to its receptors on the host cell, ACE2. S protein forms homotrimers protruding in the viral surface and facilitates binding of envelope viruses to host cells by attraction with angiotensin-converting enzyme 2 (ACE2) expressed in lower respiratory tract cells. This glycoprotein is cleaved by the host cell furin-like protease into 2 sub units namely S1 and S2. Part S1 is responsible for the determination of the host virus range and cellular tropism with the receptor binding domain make-up while S2 functions to mediate virus fusion in transmitting host cells. Representative sequences of S glycoprotein are presented below in Table 1G.

The nucleocapsid known as N protein is the structural component of CoV localizing in the endoplasmic reticulum-Golgi region that structurally is bound to the nucleic acid material of the virus. Because the protein is bound to RNA, the protein is involved in processes related to the viral genome, the viral replication cycle, and the cellular response of host cells to viral infections. N protein is also heavily phosphorylated and suggested to lead to structural changes enhancing the affinity for viral RNA. Representative sequences of N glycoprotein are presented below in Table 1G.

Another important part of this virus is the membrane or M protein, which is the most structurally structured protein and plays a role in determining the shape of the virus envelope. This protein can bind to all other structural proteins. Binding with M protein helps to stabilize nucleocapsids or N proteins and promotes completion of viral assembly by stabilizing N protein-RNA complex, inside the internal virion. Representative sequences of M protein are presented below in Table 1G.

The last component is the envelope or E protein which is the smallest protein in the SARS-CoV-2 structure that plays a role in the production and maturation of this virus.

The genomic information of SARS-CoV-2 is publicly available and can be obtained from, for example, the NCBI Severe acute respiratory syndrome coronavirus 2 database (available on the World Wide Web at ncbi.nlm.nih.gov/sars-cov-2/) and NGDC Genome Warehouse (available at bigd.big.ac.cn/gwh/), together with epidemiological data for the sequenced isolates. There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

GENETIC CODE

| | |
|---|---|
| Alanine (Ala, A) | GCA, GCC, GCG, GCT |
| Arginine (Arg, R) | AGA, ACG, CGA, CGC, CGG, CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well-known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA encoding a biomarker nucleic acid (or any portion thereof) may be used to derive the polypeptide amino acid sequence, using the genetic code to translate the DNA or RNA into an amino acid sequence. Likewise, for polypeptide amino acid sequence, corresponding nucleotide sequences that can encode the polypeptide can be deduced from the genetic code (which, because of its redundancy, will produce multiple nucleic acid sequences for any given amino acid sequence). Thus, description and/or disclosure herein of a nucleotide sequence which encodes a polypeptide should be considered to also include description and/or disclosure of the amino acid sequence encoded by the nucleotide sequence. Similarly, description and/or disclosure of a polypeptide amino acid sequence herein should be considered to also include description and/or disclosure of all possible nucleotide sequences that can encode the amino acid sequence.

### II. Peptides

In certain aspects, provided herein are methods and compositions for the treatment and/or prevention of COIVD-19 through the induction of an immune response against SARS-CoV-2 through the administration of identified SARS-COV-2 immunodominant peptides or nucleic acids encoding identified SARS-COV-2 immunodominant peptides.

In certain embodiments, the SARS-COV-2 immunodominant peptide comprises (e.g., consists of) a peptide epitope selected from Table 1A, 1B, 1C, 1D, 1E, and/or 1F. Peptide epitopes described herein may be combined with MHC molecules, such as particular HLA molecules having particular alpha chain alleles. For example, Table 1A peptides were identified in association with an MHC whose alpha chain had an HLA-A*02 serotype, such as that encoded by an HLA-A*0201, HLA-A*0202, HLA-A*0203, HLA-A*0204, HLA-A*0205, HLA-A*0206, HLA-A*0207, HLA-A*0210, HLA-A*0211, HLA-A*0212, HLA-A*0213, HLA-A*0214, HLA-A*0216, HLA-A*0217, HLA-A*0219, HLA-A*0220, HLA-A*0222, HLA-A*0224, HLA-A*0230, HLA-A*0242, HLA-A*0253, HLA-A*0260, and/or HLA-A*0274 allele; Table 1B peptides were identified in association with an MHC whose alpha chain had an HLA-A*03 serotype, such as that encoded by an HLA-A*0301, HLA-A*0302, HLA-A*0305, and/or HLA-A*0307; Table 1C peptides were identified in association with an MHC whose alpha chain had an HLA-A*01 serotype, such as that encoded by an HLA-A*0101, HLA-A*0102, HLA-A*0103, and/or HLA-A*0116 allele; Table 1D peptides were identified in association with an MHC whose alpha chain had an HLA-A*11 serotype, such as that encoded by an HLA-A*1101, HLA-A*1102, HLA-A*1103, HLA-A*1104, HLA-A*1105, and/or HLA-A*1119 allele; Table 1E peptides were identified in association with an MHC whose alpha chain had an HLA-A*24 serotype, such as that encoded by an HLA-A*2402, HLA-A*2403, HLA-A*2405, HLA-A*2407, HLA-A*2408, HLA-A*2410, HLA-A*2414, HLA-A*2417, HLA-A*2420, HLA-A*2422, HLA-A*2425, HLA-A*2426, and/or HLA-A*2458 allele; and Table 1F peptides were identified in association with an MHC whose alpha chain had an HLA-B*07 serotype, such as that encoded by an HLA-B*0702, HLA-B*0704, HLA-B*0705, HLA-B*0709, HLA-B*0710, HLA-B*0715, and/or HLA-B*0721 allele, as described further in the working examples. In some embodiments, the SARS-COV-2 immunodominant peptides are derived from a SARS-COV-2 protein selected from Table 1G. In some embodiments, one or more SARS-COV-2 immunodominant peptides are administered alone or in combination with an adjuvant.

In certain aspects, provided herein are compositions comprising one or more SARS-CoV-2 immunogenic peptides described herein and an adjuvant.

TABLE 1A

HLA-A02

| | |
|---|---|
| Peptide Epitopes | Derived From SARS-CoV-2 Protein |
| ALWEIQQVV | ORF1ab |
| YLQPRTFLLK | S |
| SALWEIQQVV | ORF1ab |
| ATYYLFDESGEFKL | ORF1ab |
| PLLYDANYFL | ORF3a |
| LLYDANYFL | ORF3a |
| RLANECAQV | ORF1ab |
| QLSSYSLFDM | ORF1ab |
| YLFDESGEFKL | ORF1ab |
| FLIVAAIVFI | ORF7a |
| YANSVFNI | ORF1ab |
| FLCWHTNCYDYCI | ORF3a |
| SMWALIISV | ORF1ab |
| LLLDRLNQL | N |
| FAFACPDGV | ORF7a |
| YRLANECAQV | ORF1ab |
| GYLQPRTFLL | S |
| YLQPRTFLL | S |
| KLWAQCVQL | ORF1ab |
| ALWEIQQV | ORF1ab |
| ALDQAISMWA | ORF1ab |
| SLFDMSKFPL | ORF1ab |
| LLAKDTTEA | ORF1ab |
| MDLFMRIFTI | ORF3a |
| KILGLPTQTV | ORF1ab |
| SLQTYVTQQL | S |
| ALSKGVHFV | ORF3a |
| VMCGGSLYV | ORF1ab |
| TYASALWEIQQVV | ORF1ab |
| LLYDANYFLC | ORF3a |
| FDMSKFPLKL | ORF1ab |
| TYYLFDESGEFKL | ORF1ab |
| YSLFDMSKFPL | ORF1ab |
| YASALWEIQQVV | ORF1ab |
| FLLKYNENGTI | S |
| FTYASALWEI | ORF1ab |
| YYLFDESGEFKL | ORF1ab |
| RLWLCWKCRSKNPL | ORF3a |

TABLE 1B

HLA-A03

| | |
|---|---|
| Peptide Epitopes | Derived From SARS-CoV-2 Protein |
| TVIEVQGYK | ORF1ab |
| QIAPGQTGK | S |
| MMVTNNTFTLK | ORF1ab |
| RLFRKSNLK | S |
| YNSASFSTFK | S |
| VTNNTFTLK | ORF1ab |
| RQIAPGQTGK | S |
| KLFDRYFKY | ORF1ab |
| KTIQPRVEK | ORF1ab |
| CVADYSVLY | S |
| RLKLFDRYFK | ORF1ab |
| KTFPPTEPK | N |
| STFKCYGVSPTK | S |
| KCYGVSPTK | S |
| VLYNSASFSTFK | S |
| MVTNNTFTLK | ORF1ab |
| KTFPPTEPKK | N |
| KLFDRYFK | ORF1ab |
| QLPQGTTLPK | N |

TABLE 1C

HLA-A01

| | |
|---|---|
| Peptide Epitopes | Derived From SARS-CoV-2 Protein |
| VPTDNYITTY | ORF1ab |
| FTSDYYQLYS | ORF3a |
| CTDDNALAY | ORF1ab |
| SSPDDQIGYY | N |
| HTTDPSFLGRY | ORF1ab |
| TACTDDNALAYY | ORF1ab |
| TDDNALAY | ORF1ab |
| GTDLEGNFY | ORF1ab |
| PTDNYITTY | ORF1ab |
| TCDGTTFTY | ORF1ab |
| SMDNSPNLA | ORF1ab |
| YHTTDPSFLGRY | ORF1ab |
| LTTAAKLMVVIPDY | ORF1ab |
| VDTDFVNEFY | ORF1ab |
| ACTDDNALAYY | ORF1ab |
| FTSDYYQLY | ORF3a |
| YFTSDYYQLY | ORF3a |
| DTDFVNEFY | ORF1ab |
| SSDNIALLV | M |
| CTDDNALAYY | ORF1ab |
| TTDPSFLGRY | ORF1ab |
| LSPRWYFYY | N |
| YYHTTDPSFLGRY | ORF1ab |
| EYYHTTDPSFLGRY | ORF1ab |
| TSDYYQLY | ORF3a |
| ACTDDNALAY | ORF1ab |
| VATSRTLSYY | M |
| ATSRTLSYY | M |
| NTCDGTTFTY | ORF1ab |

TABLE 1D

HLA-A11

| | |
|---|---|
| Peptide Epitopes | Derived From SARS-CoV-2 Protein |
| VTDTPKGPK | ORF1ab |
| VTNNTFTLK | ORF1ab |
| TVATSRTLSYYK | M |
| ASAFFGMSR | N |
| LIRQGTDYK | N |
| LLNKHIDAYK | N |
| AVILRGHLR | M |
| QDLKWARFPK | ORF1ab |
| VTLACFVLAAVYR | M |
| KVKYLYFIK | ORF1ab |
| STMTNRQFHQKLLK | ORF1ab |
| KTFPPTEPK | N |
| QQQGQTVTK | N |
| ATSRTLSYYK | M |
| ATEGALNTPK | N |
| KSAAEASKK | N |
| KAYNVTQAFGR | N |

TABLE 1E

HLA-A24

| | |
|---|---|
| Peptide Epitopes | Derived From SARS-CoV-2 Protein |
| QYIKWPWYI | S |
| VYIGDPAQL | ORF1ab |
| VYFLQSINF | ORF3a |
| YYRRATRRI | N |
| RWYFYYLGTG | N |
| QYIKWPWYIW | S |
| KYEQYIKWPW | S |
| KWPWYIWLGF | S |
| LYLYALVYF | ORF3a |
| LYALVYFLQSINFV | ORF3a |
| YLYALVYFLQSINF | ORF3a |
| QYIKWPWYIWLGF | S |
| LYALVYFLQSINF | ORF3a |

TABLE 1F

HLA-B07

| | |
|---|---|
| Peptide Epitopes | Derived From SARS-CoV-2 Protein |
| SPRWYFYYLG | N |
| IPRRNVATL | ORF1ab |
| RPDTRYVL | ORF1ab |
| SPRWYFYYL | N |
| RPDTRYVLM | ORF1ab |
| IPRRNVATLQ | ORF1ab |
| EIPRRNVATL | ORF1ab |
| PRWYFYYL | N |
| LSPRWYFYYL | N |
| RIRGGDGKM | N |
| SLEIPRRNVATLQA | ORF1ab |

TABLE 1G

>YP_009724389 (SARS-CoV-2 ORF1a/b protein)
MESLVPGFNEKTHVQLSLPVLQVRDVLVRGFGDSVEEVLSEARQHLKDGTCGLVEVEKGVLPQLEQPYVFIKRSDARTAPHGHVMVELVAELEGIQYGRSGETLGVLVPHVGEIPVAYRKVLLRKNGNKGAGGHSYGADLKSFDLGDLGTDPYEDFQENWNTKHSSGVTRELMRELNGGAYTRYVDNNFCGPDGYPECIKDLLARAGKASCTLSEQLDFIDTKRGVYCCREHEHEIAWYTERSEKYELQTPFEIKLAKKFDTFNGECPNFVFPLNSIIKTIQPRVEKKKLDGFMGRIRSVYPVASPNECNQMCLSTLMKCDHCGETSWQTGDFVKATCEFCGTENLTKEGATTCGYLPQNAVVKIYCPACHNSEVGPEHSLAEYHNESGLKTILRKGGRTIAFGGCVFSYVGCHNKCAYWVPRASANIGCNHTGVVGEGSEGLNDNLLEILQKEKVNINIVGDFKLNEEIAIILASFSASTSAFVETVKGLDYKAFKQIVESCGNFKVTKGKAKKGAWNIGEQKSILSPLYAFASEAARWRSIFSRTLETAQNSVRVLQKAAITILDGISQYSLRLIDAMMFTSDLATNNLVVMAYITGGVVQLTSQWLTNIFGTVYEKLKPVLDWLEEKFKEGVEFLRDGWEIVKFISTCACEIVGGQIVTCAKEIKESVQTFFKLVNKFLALCADSIIIGGAKLKALNLGETFVTHSKGLYRKCVKSREETGLLMPLKAPKEIIFLEGETLPTEVLTEEVVLKTGDLQPLEQPTSEAVEAPLVGTPVCINGLMLLEIKDTEKYCALAPNMMVTNNTFTLKGGAPTKVTFGDDTVIEVQGYKSVNITFELDERIDKVLNEKCSAYTVELGTEVNEFACVVADAVIKTLQPVSELLTPLGIDLDEWSMATYYLFDESGEFKLASHMYCSFYPPDEDEEEGDCEEEEFEPSTQYEYGTEDDYQGKPLEFGATSAALQPEEEQEEDWLDDDSQQTVGQQDGSEDNQTTTIQTIVEVQPQLEMELTPVVQTIEVNSFSGYLKLTDNVYIKNADIVEEAKKVKPTVVVNAANVYLKHGGGVAGALNKATNNAMQVESDDYIATNGPLKVGGSCVLSGHNLAKHCLHVVGPNVNKGEDIQLLKSAYENFNQHEVLLAPLLSAGIFGADPIHSLRVCVDTVRTNVYLAVFDKNLYDKLVSSFLEMKSEKQVEQKIAEIPKEEVKPFITESKPSVEQRKQDDKKIKACVEEVTTTLEETKFLTENLLLYIDINGNLHPDSATLVSDIDITFLKKDAPYIVGDVVQEGVLTAVVIPTKKAGGTTEMLAKALRKVPTDNYITTYPGQGLNGYTVEEAKTVLKKCKSAFYILPSIISNEKQEILGTVSWNLREMLAHAEETRKLMPVCVETKAIVSTIQRKYKGIKIQEGVVDYGARFYFYTSKTTVASLINTLNDLNETLVTMPLGYVTHGLNLEEAARYMRSLKVPATVSVSSPDAVTAYNGYLTSSSKTPEEHFIETISLAGSYKDWSYSGQSTQLGIEFLKRGDKSVYYTSNPTTFHLDGEVITFDNLKTLLSLREVRTIKVFTTVDNINLHTQVVDMSMTYGQQFGPTYLDGADVTKIKPHNSHEGKTFYVLPNDDTLRVEAFEYYHTTDPSFLGRYMSALNHTKKWKYPQVNGLTSIKWADNNCYLATALLTLQQIELKFNPPALQDAYYRARAGEAANFCALILAYCNKTVGELGDVRETMSYLFQHANLDSCKRVLNVVCKTCGQQQTTLKGVEAVMYMGTLSYEQFKKGVQIPCTCGKQATKYLVQQESPFVMMSAPPAQYELKHGTFTCASEYTGNYQCGHYKHITSKETLYCIDGALLTKSSEYKGPITDVFYKENSYTTTIKPVTYKLDGVVCTEIDPKLDNYYKKDNSYFTEQPIDLVPNQPYPNASFDNFKFVCDNIKFADDLNQLTGYKKPASRELKVTFFPDLNGDVVAIDYKHYTPSFKKGAKLLHKPIVWHVNNATNKATYKPNTWCIRCLWSTKPVETSNSFDVLKSEDAQGMDNLACEDLKPVSEEVVENPTIQKDVLECNVKTTEVVGDIILKPANNSLKITEEVGHTDLMAAYVDNSSLTIKKPNELSRVLGLKTLATHGLAAVNSVPWDTIANYAKPFLNKVVSTTTNIVTRCLNRVCTNYMPYFFTLLLQLCTFTRSTNSRIKASMPTTIAKNTVKSVGKFCLEASFNYLKSPNFSKLINIIIWFLLLSVCLGSLIYSTAALGVLMSNLGMPSYCTGYREGYLNSTNVTIATYCTGSIPCSVCLSGLDSLDTYPSLETIQITISSFKWDLTAFGLVAEWFLAYILFTRFFYVLGLAAIMQLFFSYFAVHFISNSWLMWLIINLVQMAPISAMVRMYIFFASFYYVWKSYVHVVDGCNSSTCMMCYKRNRATRVECTTIVNGVRRSFYVYANGGKGFCKLHNWNCVNCDTFCAGSTFISDEVARDLSLQFKRPINPTDQSSYIVDSVTVKNGSIHLYFDKAGQKTYERHSLSHFVNLDNLRANNTKGSLPINVIVFDGKSKCEESSAKSASVYYSQLMCQPILLLDQALVSDVGDSAEVAVKMFDAYVNTFSSTFNVPMEKLKTLVATAEAELAKNVSLDNVLSTFISAARQGFVDSDVETKDVVECLKLSHQSDIEVTGDSCNNYMLTYNKVENMTPRDLGACIDCSARHINAQVAKSHNIALIWNVKDFMSLSEQLRKQIRSAAKKNNLPFKLTCATTRQVVNVVTTKIALKGGKIVNNWLKQLIKVTLVFLFVAAIFYLITPVHVMSKHTDFSSEIIGYKAIDGGVTRDIASTDTCFANKHADFDTWFSQRGGSYTNDKACPLIAAVITREVGFVVPGLPGTILRTTNGDFLHFLPRVFSAVGNICYTPSKLIEYTDFATSACVLAAECTIFKDASGKPVPYCYDTNVLEGSVAYESLRPDTRYVLMDGSIIQFPNTYLEGSVRVVTTFDSEYCRHGTCERSEAGVCVSTSGRWVLNNDYYRSLPGVFCGVDAVNLLTNMFTPLIQPIGALDISASIVAGGIVAIVVTCLAYYFMRFRRAFGEYSHVVAFNTLLFLMSFTVLCLTPVYSFLPGVYSVIYLYLTFYLTNDVSFLAHIQWMVMFTPLVPFWITIAYIICISTKHFYWFFSNYLKRRVVFNGVSFSTFEEAALCTFLLNKEMYLKLRSDVLLPLTQYNRYLALYNKYKYFSGAMDTTSYREAACCHLAKALNDFSNSGSDVLYQPPQTSITSAVLQSGFRKMAFPSGKVEGCMVQVTCGTTTLNGLWLDDVVYCPRHVICTSEDMLNPNYEDLLIRKSNHNFLVQAGNVQLRVIGHSMQNCVLKLKVDTANPKTPKYKFVRIQPGQTFSVLACYNGSPSGVYQCAMRPNFTIKGSFLNGSCGSVGFNIDYDCVSFCYMHHMELPTGVHAGTDLEGNFYGPFVDRQTAQAAGTDTTITVNVLAWLYAAVINGDRWFLNRFTTTLNDFNLVAMKYNYEPLTQDHVDILGPLSAQTGIAVLDMCASLKELLQNGMNGRTILGSALLEDEFTPFDVVRQCSGVTFQSAVKRTIKGTHHWLLLTILTSLLVLVQSTQWSLFFFLYENAFLPFAMGIIAMSAFAMMFVKHKHAFLCLFLLPSLATVAYFNMVYMPASWVMRIMTWLDMVDTSLSGFKLKDCVMYASAVVLLILMTARTVYDDGARRVWTLMNVLTLVYKVYYGNALDQAISMWALIISVTSNYSGVVTTVMFLARGIVFMCVEYCPIFFITGNTLQCIMLVYCFLGYFCTCYFGLFCLLNRYFRLTLGVYDYLVSTQEFRYMNSQGLLPPKNSIDAFKLNIKLLGVGGKPCIKVATVQSKMSDVKCTSVVLLSVLQQLRVESSSKLWAQCVQLHNDILLAKDTTEAFEKMVSLLSVLLSMQGAVDINKLCEEMLDNRATLQAIASEFSSLPSYAAFATAQEAYEQAVANGDSEVVLKKLKKSLNVAKSEFDRDAAMQRKLEKMADQAMTQMYKQARSEDKRAKVTSAMQTMLFTMLRKLDNDALNNIINNARDGCVPLNIIPLTTAAKLMVVIPDYNTYKNTCDGTTFTYASALWEIQQVVDADSKIVQLSEISMDNSPNLAWPLIVTALRANSAVKLQNNELSPVALRQMSCAAGTTQTACTDDNALAYYNTTKGGRFVLALLSDLQDLKWARFPKSDGTGTIYTELEPPCRFVTDTPKGPKVKYLYFIKGLNNLNRGMVLGSLAATVRLQAGNATEVPANSTVLSFCAFAVDAAKAYKDYLASGGQPITNCVKMLCTHTGTGQAITVTPEANMDQESFGGASCCLYCRCHIDHPNPKGFCDLKGKYVQIPTTCANDPVGFTLKNTVCTVCGMWKGYGCSCDQLREPMLQSADAQSFLNRVCGVSAARLTPCGTGTSTDVVYRAFDIYNDKVAGFAKFLKTNCCRFQEKDEDDNLIDSYFVVKRHTFSNYQHEETIYNLLKDCPAVAKHDFFKFRIDGDMVPHISRQRLTKYTMADLVYALRHFDEGNCDTLKEILVTYNCCDDDYFNKKDWYDFVENPDILRVYANLGERVRQALLKTVQFCDAMRNAGIVGVLTLDNQDLNGNWYDFGDFIQTTPGSGVPVVDSYYSLLMPILTLTRALTAESHVDTDLTKPYIKWDLLKYDFTEERLKLFDRYFKYWDQTYHPNCVNCLDDRCILHCANFNVLFSTVFPPTSFGPLVRKIFVDGVPFVVSTGYHFRELGVVHNQDVNLHSSRLSFKELLVYAADPAMHAASGNLLLDKRTTCFSVAALTNNVAFQTVKPGNFNKDFYDFAVSKGFFKEGSSVELKHFFFAQDGNAAISDYDYYRYNLPTMCDIRQLLFVVEVVDKYFDCYDGGCINANQVIVNNLDKSAGFPFNKWGKARLYYDSMSYEDQDALFAYTKRNVIPTITQMNLKYAISAKNRARTVAGVSICSTMTNRQFHQKLLKSIAATRGATVVIGTSKFYGGWHNMLKTVYSDVENPHLMGWDYPKCDRAMPNMLRIMASLVLARKHTTCCSLSHRFYRLANECAQVLSEMVMCGGSLYVKPGGTSSGDATTAYANSVFNICQAVTANVNALLSTDGNKIADKYVRNLQHRLYECLYRNRDVDTDFVNEFYAYLRKHFSMMILSDDAVVCFNSTYASQGLVASIKNFKSVLYYQNNVFMSEAKCWTETDLTKGPHEFCSQHTMLVKQGDDYVYLPYPDPSRILGAGCFVDDIVKTDGTLMIERFVSLAIDAYPLTKHPNQEYADVFHLYLQYIRKLHDELTGHMLDMYSVMLTNDNTSRYWEPEFYEAMYTPHTVLQAVGACVLCNSQTSLRCGACIRRPFLCCKCCYDHVISTSHKLVLSVNPYVCNAPGCDVTDVTQLYLGGMSYYCKSHKPPISFPLCANGQVFGLYKNTCVGSDNVTDFNAIATCDWTNAGDYILANTCTERLKLFAAETLKATEETFKLSYGIATVREVLSDRELHLSWEVGKPRPPLNRNYVFTGYRVTKNSKVQIGEYTFEKGDYGDAVVYRGTTTYKLNVGDYFVLTSHTVMPLSAPTLVPQEHYVRITGLYPTLNISDEFSSNVANYQKVGMQKYSTLQGPPGTGKSHFAIGLALYYPSARIVYTACSHAAVDALCEKALKYLPIDKCSRIIPARARVECFDKFKVNSTLEQYVFCTVNALPETTADIVVFDEISMATNYDLSVVNARLRAKHYVYIGDPAQLPAPRTLLTKGTLEPEYFNSVCRLMKTIGPDMFLGTCRRCPAEIVDTVSALVYDNKLKAHKDKSAQCFKMFYKGVITHDVSSAINRPQIGVVREFLTRNPAWRKAVFISPYNSQNAVASKILGLPTQTVDSSQGSEYDYVIFTQTTETAHSCNVNRFNVAITRAKVGILCIMSDRDLYDKLQFTSLEIPRRNVATLQAENVTGLFKDCSKVITGLHPTQAPTHLSVDTKFKTEGLCVDIPGIPKDMTYRRLISMMGFKMNYQVNGYPNMFITREEAIRHVRAWIGFDVEGCHATREAVGTNLPLQLGFSTGVNLVAVPTGYVDTPNNTDFSRVSAKPPPGDQFKHLIPLMYKGLPWNVVRIKIVQMLSDTLKNLSDRVVFVLWAHGFELTSMKYFVKIGPERTCCLCDRRATCFSTASDTYACWHHSIGFDYVYNPFMIDVQQWGFTGNLQSNHDLYCQVHGNAHVASCDAIMTRCLAVHECFVKRVDWTIEYPIIGDELKINAACRKVQHMVVKAALLADKFPVLHDIGNPKAIKCVPQADVEWKFYDAQPCSDKAYKIEELFYSYATHSDKFTDGVCLFWNCNVDRYPANSIVCRFDTRVLSNLNLPGCDGGSLYVNKHAFHTPAFDKSAFVNLKQLPFFYYSDSPCESHGKQVVSDIDYVPLKSATCITRCNLGGAVCRHHANEYRLYLDAYNMMISAGFSLWVYKQFDTYNLWNTFTRLQSLENVAFNVVNKGHFDGQQGEVPVSIINNTVYTKVDGVDVELFENKTTLPVNVAFELWAKRNIKPVPEVKILNNLGVDIAANTVIWDYKRDAPAHISTIGVCSMTDIAKKPTETICAPLTVFFDGRVDGQVDLFRNARNGVLITEGSVKGLQPSVGPKQASLNGVTLIGEAVKTQFNYYKKVDGVVQQLPETYFTQSRNLQEFKPRSQMEIDFLELAMDEFIERYKLEGYAFEHIVYGDFSHSQLGGLHLLIGLAKRFKESPFELEDFIPMDSTVKNYFITDAQTGSSKCVCSVIDLLLDDFVEIIKSQDLSVVSKVVKVTIDYTEISFMLWCKDGHVETFYPKLQSSQAWQPGVAMPNLYKMQRMLLEKCDLQNYGDSATLPKGIMMNVAKYTQLCQYLNTLTLAVPYNMRVIHFGAGSDKGVAPGTAVLRQWLPTGTLLVDSDLNDFVSDADSTLIGDCATVHTANKWDLIISDMYDPKTKNVTKENDSKEGFFTYICGFIQQKLALGGSVAIKITEHSWNADLYKLMGHFAWWTAFVTNVNASSSEAFLIGCNYLGKPREQIDGYVMHANYIFWRNTNPIQLSSYSLFDMSKFPLKLRGTAVMSLKEGQINDMILSLLSKGRLIIRENNRVVISSDVLVNN
>YP_009724390 (SARS-CoV-2 S protein)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGWFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT
>YP_009724397 (SARS-CoV-2 N protein)
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA
>YP_009724391 (SARS-CoV-2 orf3a protein)
MDLFMRIFTIGTVTLKQGEIKDATPSDFVRATATIPIQASLPFGWLIVGVALLAVFQSASKIITLKKRWQLALSKGVHFVCNLLLLFVTVYSHLLLVAAGLEAPFLYLYALVYFLQSINFVRIIMRLWLCWKCRSKNPLLYDANYFLCWHTNCYDYCIPYNSVTSSIVITSGDGTTSPISEHDYQIGGYTEKWESGVKDCVVLHSYFTSDYYQLYSTQLSTDTGVEHVTFFIYNKIVDEPEEHVQIHTIDGSSGVVNPVMEPIYDEPTTTTSVPL
> YP_009724393.1 (SARS-CoV-2 M protein)
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ
> YP_009724395.1 (SARS-CoV-2 orf7a protein)
MKIILFLALITLATCELYHYQECVRGTTVLLKEPCSSGTYEGNSPFHPLADNKFALTCFSTQFAFACPDGVKHVYQLRARSVSPKLFIRQEEVQELYSPIFLIVAAIVFITLCFTLKRKTE

* Included in Tables 1A-1G are peptide epitopes, as well as polypeptide molecules comprising an amino acid sequence having at least 80%, 81%, 82%, 83%, 84%, 85%, 86%, 87%, 88%, 89%, 90%, 91%, 92%, 93%, 94%, 95%, 96%, 97%, 98%, 99%, 99.5%, or more identity across their full length with an amino acid sequence of any SEQ ID NO listed in Tables 1A-1G, or a portion thereof. Such polypeptides may have a function of the full-length peptide or polypeptide as described further herein.

In some embodiments, provided herein are orf1a/b polypeptides and/or nucleic acids encoding orf1a/b polypeptides. Orf1a/b polypeptides are polypeptides that include an amino acid sequence that corresponds to the amino acid sequence of an orf1a/b polyprotein, and/or a portion of the orf1a/b amino acid sequence of sufficient length to elicit an orf1a/b-specific immune response. In certain embodiments, the orf1a/b polypeptide also includes amino acids that do not correspond to the amino acid sequence (e.g., a fusion protein comprising an orf1a/b amino acid sequence and an amino acid sequence corresponding to a non-orf1a/b protein or polypeptide). In some embodiments, the orf1a/b polypeptide only includes amino acid sequence corresponding to an orf1a/b polyprotein or fragment thereof.

In some embodiments, the orf1a/b polypeptide has an amino acid sequence that comprises, consists essentially of, or consists of at least 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 200, 300, 400, 500, 600, 700, 800, 900, 1000, 1500, 2000, 2500, 3000, 3500, 4000, 4500, 5000, 5500, 6000, 6500, or 7000 consecutive amino acids of an orf1a/b protein amino acid sequence set forth in Table 1G. In some embodiments, the consecutive amino acids are identical to an amino acid sequence of orf1a/b set forth in Table 1G. In some embodiments, orf1a/b polypeptides comprise, consist essentially of, or consist of one or more peptide epitopes selected from the group consisting of orf1a/b peptide epitopes listed in Table 1A, 1B, 1C, 1D, 1E, and/or 1F.

In some embodiments, provided herein are S protein polypeptides and/or nucleic acids encoding S protein polypeptides. S protein polypeptides are polypeptides that include an amino acid sequence that corresponds to the amino acid sequence of an S protein polyprotein, and/or a portion of the S protein amino acid sequence of sufficient length to elicit an S protein-specific immune response. In certain embodiments, the S protein polypeptide also includes amino acids that do not correspond to the amino acid sequence (e.g., a fusion protein comprising an S protein amino acid sequence and an amino acid sequence corresponding to a non-S protein or polypeptide). In some embodiments, the S protein polypeptide only includes amino acid sequence corresponding to an S protein polyprotein or fragment thereof.

In certain embodiments, the S protein polypeptide has an amino acid sequence that comprises, consists essentially of, or consists of at least 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950, 1000, 1050, 1100, 1150, 1200, or 1250 consecutive amino acids of an S protein amino acid sequence set forth in Table 1G. In some embodiments, the consecutive amino acids are identical to an amino acid sequence of S protein set forth in Table 1G. In some embodiments, S polypeptides comprise, consist essentially of, or consist of one or more peptide epitopes selected from the group consisting of S peptide epitopes listed in Table 1A, 1B, 1C, 1D, 1E, and/or 1F.

In some embodiments, provided herein are N protein polypeptides and/or nucleic acids encoding N protein polypeptides. N protein polypeptides are polypeptides that include an amino acid sequence that corresponds to the amino acid sequence of an N protein polyprotein, and/or a portion of the N protein amino acid sequence of sufficient length to elicit an N protein-specific immune response. In certain embodiments, the N protein polypeptide also includes amino acids that do not correspond to the amino acid sequence (e.g., a fusion protein comprising an N protein amino acid sequence and an amino acid sequence corresponding to a non-N protein or polypeptide). In some embodiments, the N protein polypeptide only includes amino acid sequence corresponding to an N protein polyprotein or fragment thereof.

In certain embodiments, the N protein polypeptide has an amino acid sequence that comprises, consists essentially of, or consists of at least 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, or 300 consecutive amino acids of an N protein amino acid sequence set forth in Table 1G. In some embodiments, the consecutive amino acids are identical to an amino acid sequence of an N protein set forth in Table 1G. In some embodiments, N polypeptides comprise, consist essentially of, or consist of one or more peptide epitopes selected from the group consisting of N peptide epitopes listed in Table 1A, 1B, 1C, 1D, 1E, and/or 1F.

In some embodiments, provided herein are M protein polypeptides and/or nucleic acids encoding M protein polypeptides. M protein polypeptides are polypeptides that include an amino acid sequence that corresponds to the amino acid sequence of an M protein polyprotein, and/or a portion of the M protein amino acid sequence of sufficient length to elicit an M protein-specific immune response. In certain embodiments, the M protein polypeptide also includes amino acids that do not correspond to the amino acid sequence (e.g., a fusion protein comprising an M protein amino acid sequence and an amino acid sequence corresponding to a non-M protein or polypeptide). In some embodiments, the M protein polypeptide only includes amino acid sequence corresponding to an N protein polyprotein or fragment thereof.

In certain embodiments, the M protein polypeptide has an amino acid sequence that comprises, consists essentially of, or consists of at least 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 210, or 220 consecutive amino acids of an M protein amino acid sequence set forth in Table 1G. In some embodiments, the consecutive amino acids are identical to an amino acid sequence of an M protein set forth in Table 1G. In some embodiments, M polypeptides comprise, consist essentially of, or consist of one or more peptide epitopes selected from the group consisting of M peptide epitopes listed in Table 1A, 1B, 1C, 1D, 1E, and/or 1F.

In some embodiments, provided herein are orf3a polypeptides and/or nucleic acids encoding orf3a polypeptides. Orf3a polypeptides are polypeptides that include an amino acid sequence that corresponds to the amino acid sequence of an orf3a polyprotein, and/or a portion of the orf3a amino acid sequence of sufficient length to elicit an orf3a-specific immune response. In certain embodiments, the orf3a polypeptide also includes amino acids that do not correspond to the amino acid sequence (e.g., a fusion protein comprising an orf3a amino acid sequence and an amino acid sequence corresponding to a non-orf3a protein or polypeptide). In some embodiments, the orf3a polypeptide only includes amino acid sequence corresponding to an orf3a polyprotein or fragment thereof.

In certain embodiments, the orf3a polypeptide has an amino acid sequence that comprises, consists essentially of, or consists of at least 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, or 270 consecutive amino acids of an orf3a amino acid sequence set forth in Table 1G. In some embodiments, the consecutive amino acids are identical to an amino acid sequence of an orf3a protein set forth in Table 1G. In some embodiments, orf3a polypeptides comprise, consist essentially of, or consist of one or more peptide epitopes selected from the group consisting of orf3a peptide epitopes listed in Table 1A, 1B, 1C, 1D, 1E, and/or 1F.

In some embodiments, provided herein are orf7a polypeptides and/or nucleic acids encoding orf7a polypeptides. Orf7a polypeptides are polypeptides that include an amino acid sequence that corresponds to the amino acid sequence of an orf7a polyprotein, and/or a portion of the orf7a amino acid sequence of sufficient length to elicit an orf7a-specific immune response. In certain embodiments, the orf7a polypeptide also includes amino acids that do not correspond to the amino acid sequence (e.g., a fusion protein comprising an orf7a amino acid sequence and an amino acid sequence corresponding to a non-orf7a protein or polypeptide). In some embodiments, the orf7a polypeptide only includes amino acid sequence corresponding to an orf7a polyprotein or fragment thereof.

In certain embodiments, the orf7a polypeptide has an amino acid sequence that comprises, consists essentially of, or consists of at least 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, or 120 consecutive amino acids of an orf7a amino acid sequence set forth in Table 1G. In some embodiments, the consecutive amino acids are identical to an amino acid sequence of an orf7a protein set forth in Table 1G. In some embodiments, orf7a polypeptides comprise, consist essentially of, or consist of one or more peptide epitopes selected from the group consisting of orf7a peptide epitopes listed in Table 1A, 1B, 1C, 1D, 1E, and/or 1F.

As is well-known to those skilled in the art, polypeptides having substantial sequence similarities can cause identical or very similar immune reaction in a host animal. Accordingly, in some embodiments, a derivative, equivalent, variant, fragment, or mutant of a SARS-CoV-2 immunogenic peptide described herein or fragment thereof may also suitable for the methods and compositions provided herein.

In some embodiments, variations or derivatives of the SARS-CoV-2 immunogenic polypeptides are provided herein. The altered polypeptide may have an altered amino acid sequence, for example by conservative substitution, yet still elicits immune responses which react with the unaltered protein antigen, and are considered functional equivalents. As used herein, the term “conservative substitution” denotes the replacement of an amino acid residue by another, biologically similar residue. It is well known in the art that the amino acids within the same conservative group may typically substitute for one another without substantially affecting the function of a protein. According to certain embodiments, the derivative, equivalents, variants, or mutants of the ligand-binding domain of a SARS-CoV-2 immunogenic peptide are polypeptides that are at least 85% homologous to the sequence of a SARS-CoV-2 immunogenic peptide described herein or fragment thereof. In some embodiments, the homology is at least 90%, at least 95%, at least 98%, or more.

Immunogenic peptides encompassed by the present invention may comprise a peptide epitope derived from a SARS-CoV-2 protein, such as those listed in Table 1A, 1B, 1C, 1D, 1E, and/or 1F. In some embodiments, the immunogenic peptide is 8, 9, 10, 11, 12, 13, 14, or 15 amino acids in length. In some embodiments, the peptide amino acid sequences is modified, which may include conservative or non-conservative mutations. A peptide may comprise at most 1, 2, 3, 4, or more mutations. In some embodiments, a peptide may comprise at least 1, 2, 3, 4, or more mutations.

In some embodiments, a peptide may be chemically modified. For example, a peptide can be mutated to modify peptide properties such as detectability, stability, biodistribution, pharmacokinetics, half-life, surface charge, hydrophobicity, conjugation sites, pH, function, and the like. N-methylation is one example of methylation that can occur in a peptide of the disclosure. In some embodiments, a peptide may be modified by methylation on free amines such as by reductive methylation with formaldehyde and sodium cyanoborohydride.

A chemical modification may comprise a polymer, a polyether, polyethylene glycol, a biopolymer, a zwitterionic polymer, a polyamino acid, a fatty acid, a dendrimer, an Fc region, a simple saturated carbon chain such as palmitate or myristolate, or albumin. The chemical modification of a peptide with an Fc region may be a fusion Fc-peptide. A polyamino acid may include, for example, a poly amino acid sequence with repeated single amino acids (e.g., poly glycine), and a poly amino acid sequence with mixed poly amino acid sequences that may or may not follow a pattern, or any combination of the foregoing. In some embodiments, the peptides of the present disclosure may be modified such that the modification increases the stability and/or the half-life of the peptides. In some embodiments, the attachment of a hydrophobic moiety, such as to the N-terminus, the C-terminus, or an internal amino acid, can be used to extend half-life of a peptide of the present disclosure. In other embodiments, a peptide may include post-translational modifications (e.g., methylation and/or amidation), which can affect, for example, serum half-life. In some embodiments, simple carbon chains (e.g., by myristoylation and/or palmitylation) can be conjugated to the fusion proteins or peptides. In some embodiments, the simple carbon chains may render the fusion proteins or peptides easily separable from the unconjugated material. For example, methods that may be used to separate the fusion proteins or peptides from the unconjugated material include, but are not limited to, solvent extraction and reverse phase chromatography. The lipophilic moieties can extend half-life through reversible binding to serum albumin. The conjugated moieties may be lipophilic moieties that extend half-life of the peptides through reversible binding to serum albumin. In some embodiments, the lipophilic moiety may be cholesterol or a cholesterol derivative, including cholestenes, cholestanes, cholestadienes and oxysterols. In some embodiments, the peptides may be conjugated to myristic acid (tetradecanoic acid) or a derivative thereof. In other embodiments, a peptide may be coupled (e.g., conjugated) to a half-life modifying agent. Examples of half-life modifying agents include but are not limited to: a polymer, a polyethylene glycol (PEG), a hydroxyethyl starch, polyvinyl alcohol, a water soluble polymer, a zwitterionic water soluble polymer, a water soluble poly(amino acid), a water soluble polymer of proline, alanine and serine, a water soluble polymer containing glycine, glutamic acid, and serine, an Fc region, a fatty acid, palmitic acid, or a molecule that binds to albumin. In some embodiments, a spacer or linker may be coupled to a peptide, such as 1, 2, 3, 4, or more amino acid residues that serve as a spacer or linker in order to facilitate conjugation or fusion to another molecule, as well as to facilitate cleavage of the peptide from such conjugated or fused molecules. In some embodiments, fusion proteins or peptides may be conjugated to other moieties that, for example, can modify or effect changes to the properties of the peptides.

A peptide may be conjugated to an agent used in imaging, research, therapeutics, theranostics, pharmaceuticals, chemotherapy, chelation therapy, targeted drug delivery, and radiotherapy. In some embodiments, a peptide may be conjugated to or fused with detectable agents, such as a fluorophore, a near-infrared dye, a contrast agent, a nanoparticle, a metal-containing nanoparticle, a metal chelate, an X-ray contrast agent, a PET agent, a metal, a radioisotope, a dye, radionuclide chelator, or another suitable material that can be used in imaging. In some embodiments, 1, 2, 3, 4, 5, 6, 7, 8, 9, 10, or more detectable moieties may be linked to a peptide. Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212. In some embodiments, the near-infrared dyes are not easily quenched by biological tissues and fluids. In some embodiments, the fluorophore is a fluorescent agent emitting electromagnetic radiation at a wavelength between 650 nm and 4000 nm, such emissions being used to detect such agent. Non-limiting examples of fluorescent dyes that may be used as a conjugating molecule include DyLight-680, DyLight-750, VivoTag-750, DyLight-800, IRDye-800, VivoTag-680, Cy5.5, ZQ800, or indocyanine green (ICG). In some embodiments, near infrared dyes often include cyanine dyes (e.g., Cy7, Cy5.5, and Cy5). Additional non-limiting examples of fluorescent dyes for use as a conjugating molecule in the present disclosure include acradine orange or yellow, Alexa Fluors (e.g., Alexa Fluor 790, 750, 700, 680, 660, and 647) and any derivative thereof, 7-actinomycin D, 8-anilinonaphthalene-1-sulfonic acid, ATTO dye and any derivative thereof, auramine-rhodamine stain and any derivative thereof, bensantrhone, bimane, 9-10-bis(phenylethynyl)anthracene, 5,12-bis(phenylethynyl)naththacene, bisbenzimide, brainbow, calcein, carbodyfluorescein and any derivative thereof, 1-chloro-9,10-bis(phenylethynyl)anthracene and any derivative thereof, DAPI, DiOC6, DyLight Fluors and any derivative thereof, epicocconone, ethidium bromide, FlAsH-EDT2, Fluo dye and any derivative thereof, FluoProbe and any derivative thereof, Fluorescein and any derivative thereof, Fura and any derivative thereof, GelGreen and any derivative thereof, GelRed and any derivative thereof, fluorescent proteins and any derivative thereof, m isoform proteins and any derivative thereof such as for example mCherry, hetamethine dye and any derivative thereof, hoeschst stain, iminocoumarin, indian yellow, indo-1 and any derivative thereof, laurdan, lucifer yellow and any derivative thereof, luciferin and any derivative thereof, luciferase and any derivative thereof, mercocyanine and any derivative thereof, nile dyes and any derivative thereof, perylene, phloxine, phyco dye and any derivative thereof, propium iodide, pyranine, rhodamine and any derivative thereof, ribogreen, RoGFP, rubrene, stilbene and any derivative thereof, sulforhodamine and any derivative thereof, SYBR and any derivative thereof, synapto-pHluorin, tetraphenyl butadiene, tetrasodium tris, Texas Red, Titan Yellow, TSQ, umbelliferone, violanthrone, yellow fluroescent protein and YOYO-1. Other Suitable fluorescent dyes include, but are not limited to, fluorescein and fluorescein dyes (e.g., fluorescein isothiocyanine or FITC, naphthofluorescein, 4′,5′-dichloro-2′,7′-dimethoxyfluorescein, 6-carboxyfluorescein or FAM, etc.), carbocyanine, merocyanine, styryl dyes, oxonol dyes, phycoerythrin, erythrosin, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Oregon Green Dyes (e.g., Oregon Green 488, Oregon Green 500, Oregon Green 514., etc.), Texas Red, Texas Red-X, SPECTRUM RED, SPECTRUM GREEN, cyanine dyes (e.g., CY-3, Cy-5, CY-3.5, CY-5.5, etc.), ALEXA FLUOR dyes (e.g., ALEXA FLUOR 350, ALEXA FLUOR 488, ALEXA FLUOR 532, ALEXA FLUOR 546, ALEXA FLUOR 568, ALEXA FLUOR 594, ALEXA FLUOR 633, ALEXA FLUOR 660, ALEXA FLUOR 680, etc.), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665, etc.), IRDyes (e.g., IRD40, IRD 700, IRD 800, etc.), and the like. Additional suitable detectable agents are described in PCT/US14/56177. Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212.

A peptide may be conjugated to a radiosensitizer or photosensitizer. Examples of radiosensitizers include but are not limited to: ABT-263, ABT-199, WEHI-539, paclitaxel, carboplatin, cisplatin, oxaliplatin, gemcitabine, etanidazole, misonidazole, tirapazamine, and nucleic acid base derivatives (e.g., halogenated purines or pyrimidines, such as 5-fluorodeoxyuridine). Examples of photosensitizers include but are not limited to: fluorescent molecules or beads that generate heat when illuminated, nanoparticles, porphyrins and porphyrin derivatives (e.g., chlorins, bacteriochlorins, isobacteriochlorins, phthalocyanines, and naphthalocyanines), metalloporphyrins, metallophthalocyanines, angelicins, chalcogenapyrrillium dyes, chlorophylls, coumarins, flavins and related compounds such as alloxazine and riboflavin, fullerenes, pheophorbides, pyropheophorbides, cyanines (e.g., merocyanine 540), pheophytins, sapphyrins, texaphyrins, purpurins, porphycenes, phenothiaziniums, methylene blue derivatives, naphthalimides, nile blue derivatives, quinones, perylenequinones (e.g., hypericins, hypocrellins, and cercosporins), psoralens, quinones, retinoids, rhodamines, thiophenes, verdins, xanthene dyes (e.g., eosins, erythrosins, rose bengals), dimeric and oligomeric forms of porphyrins, and prodrugs such as 5-aminolevulinic acid. Advantageously, this approach allows for highly specific targeting of cells of interest (e.g., immune cells) using both a therapeutic agent (e.g., drug) and electromagnetic energy (e.g., radiation or light) concurrently. In some embodiments, the peptide is fused with, or covalently or non-covalently linked to the agent, for example, directly or via a linker.

A peptide may be produced recombinantly or synthetically, such as by solid-phase peptide synthesis or solution-phase peptide synthesis. Peptide synthesis may be performed by known synthetic methods, such as using fluorenylmethyloxycarbonyl (Fmoc) chemistry or by butyloxycarbonyl (Boc) chemistry. Peptide fragments may be joined together enzymatically or synthetically.

In some embodiments, provided herein is a nucleic acid encoding a SARS-CoV-2 immunogenic peptide described herein or fragment thereof, such as a DNA molecule encoding a SARS-CoV-2 immunogenic peptide. In some embodiments, the composition comprises an expression vector comprising an open reading frame encoding a SARS-CoV-2 immunogenic peptide described herein or fragment thereof. In some embodiments, the nucleic acid includes regulatory elements necessary for expression of the open reading frame. Such elements may include, for example, a promoter, an initiation codon, a stop codon, and a polyadenylation signal. In addition, enhancers may be included. These elements may be operably linked to a sequence that encodes the SARS-CoV-2 immunogenic polypeptide or fragment thereof.

Examples of promoters include but are not limited to promoters from Simian Virus 40 (SV40), Mouse Mammary Tumor Virus (MMTV) promoter, Human Immunodeficiency Virus (HIV) such as the HIV Long Terminal Repeat (LTR) promoter, Moloney virus, Cytomegalovirus (CMV) such as the CMV immediate early promoter, Epstein Barr Virus (EBV), Rous Sarcoma Virus (RSV) as well as promoters from human genes such as human actin, human myosin, human hemoglobin, human muscle creatine, and human metalothionein. Examples of suitable polyadenylation signals include but are not limited to SV40 polyadenylation signals and LTR polyadenylation signals.

In addition to the regulatory elements required for expression, other elements may also be included in the nucleic acid molecule. Such additional elements include enhancers. Enhancers include the promoters described hereinabove. Preferred enhancers/promoters include, for example, human actin, human myosin, human hemoglobin, human muscle creatine and viral enhancers such as those from CMV, RSV and EBV.

In some embodiments, the nucleic acid may be operably incorporated in a carrier or delivery vector as described further below. Useful delivery vectors include but are not limited to biodegradable microcapsules, immuno-stimulating complexes (ISCOMs) or liposomes, and genetically engineered attenuated live carriers such as viruses or bacteria.

In some embodiments, the vector is a viral vector, such as lentiviruses, retroviruses, herpes viruses, adenoviruses, adeno-associated viruses, vaccinia viruses, baculoviruses, Fowl pox, AV-pox, modified vaccinia Ankara (MVA) and other recombinant viruses. For example, a lentivirus vector may be used to infect T cells.

### III. Nucleic Acids, Vectors, and Cells

A further object of the present invention relates to nucleic acid sequences encoding SARS-CoV-2 immunogenic peptides and fragments thereof, MHC molecules, and TCRs and fragments thereof of the present invention.

In a particular embodiment, the present invention relates to a nucleic acid sequence encoding the SARS-CoV-2 immunogenic peptides described herein.

Typically, said nucleic acid is a DNA or RNA molecule, which may be included in any suitable vector, such as a plasmid, cosmid, episome, artificial chromosome, phage or a viral vector.

The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. Thus, a further object of the invention relates to a vector comprising a nucleic acid of the present invention.

Such vectors may comprise regulatory elements, such as a promoter, enhancer, terminator and the like, to cause or direct expression of said polypeptide upon administration to a subject. Examples of promoters and enhancers used in the expression vector for animal cell include early promoter and enhancer of SV40 (Mizukami T. et al. 1987), LTR promoter and enhancer of Moloney mouse leukemia virus (Kuwana Y et al. 1987), promoter (Mason J O et al. 1985) and enhancer (Gillies S D et al. 1983) of immunoglobulin H chain and the like.

Any expression vector for animal cell can be used. Examples of suitable vectors include pAGE107 (Miyaji H et al. 1990), pAGE103 (Mizukami T et al. 1987), pHSG274 (Brady G et al. 1984), pKCR (O′Hare K et al. 1981), pSG1 beta d2-4-(Miyaji H et al. 1990) and the like. Other representative examples of plasmids include replicating plasmids comprising an origin of replication, or integrative plasmids, such as for instance pUC, pcDNA, pBR, and the like. Representative examples of viral vector include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv-positive cells, 293 cells, etc. Detailed protocols for producing such replication-defective recombinant viruses may be found for instance in WO 95/14785, WO 96/22378, U.S. Pat. No. 5,882,877, U.S. Pat. No. 6,013,516, U.S. Pat. No. 4,861,719, U.S. Pat. No. 5,278,056 and WO 94/19478.

A further object of the present invention relates to a cell which has been transfected, infected or transformed by a nucleic acid and/or a vector according to the invention. The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed.”

The nucleic acids of the present invention may be used to produce a recombinant polypeptide of the invention in a suitable expression system. The term “expression system” means a host cell and compatible vector under suitable conditions, e.g. for the expression of a protein coded for by foreign DNA carried by the vector and introduced to the host cell.

Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, 3T3 cells, COS cells, etc.) as well as primary or established mammalian cell cultures (e.g., produced from lymphoblasts, fibroblasts, embryonic cells, epithelial cells, nervous cells, adipocytes, etc.). Examples also include mouse SP2/0-Ag14 cell (ATCC CRL1581), mouse P3X63-Ag8.653 cell (ATCC CRL1580), CHO cell in which a dihydrofolate reductase gene (hereinafter referred to as “DHFR gene”) is defective (Urlaub G et al; 1980), rat YB2/3HL.P2.G11.16Ag.20 cell (ATCC CRL 1662, hereinafter referred to as “YB2/0 cell”), and the like. The YB2/0 cell is preferred, since ADCC activity of chimeric or humanized antibodies is enhanced when expressed in this cell.

The present invention also relates to a method of producing a recombinant host cell expressing SARS-CoV-2 immunogenic peptides and fragments thereof, MHC molecules, and TCRs and fragments thereof of the invention according to the invention, said method comprising the steps consisting of (i) introducing in vitro or ex vivo a recombinant nucleic acid or a vector as described above into a competent host cell, (ii) culturing in vitro or ex vivo the recombinant host cell obtained and (iii), optionally, selecting the cells which express said SARS-CoV-2 immunogenic peptides and fragments thereof, MHC molecules, and TCRs and fragments thereof. Such recombinant host cells can be used for the diagnostic, prognostic, and/or therapeutic method of the invention.

In another aspect, the present invention provides isolated nucleic acids that hybridize under selective hybridization conditions to a polynucleotide disclosed herein. Thus, the polynucleotides of this embodiment can be used for isolating, detecting, and/or quantifying nucleic acids comprising such polynucleotides. For example, polynucleotides of the present invention can be used to identify, isolate, or amplify partial or full-length clones in a deposited library. In some embodiments, the polynucleotides are genomic or cDNA sequences isolated, or otherwise complementary to, a cDNA from a human or mammalian nucleic acid library. Preferably, the cDNA library comprises at least 80% full-length sequences, preferably, at least 85% or 90% full-length sequences, and, more preferably, at least 95% full-length sequences. The cDNA libraries can be normalized to increase the representation of rare sequences. Low or moderate stringency hybridization conditions are typically, but not exclusively, employed with sequences having a reduced sequence identity relative to complementary sequences. Moderate and high stringency conditions can optionally be employed for sequences of greater identity. Low stringency conditions allow selective hybridization of sequences having about 70% sequence identity and can be employed to identify orthologous or paralogous sequences. Optionally, polynucleotides of this invention will encode at least a portion of an antibody encoded by the polynucleotides described herein. The polynucleotides of this invention embrace nucleic acid sequences that can be employed for selective hybridization to a polynucleotide encoding an antibody of the present invention. See, e.g., Ausubel, supra; Colligan, supra, each entirely incorporated herein by reference.

### IV. MHC-Peptide Complexes

In certain aspects, provided herein are compositions comprising a SARS-CoV-2 immunogenic peptide described herein and a MHC molecule. In some embodiments, the SARS-CoV-2 immunogenic peptide forms a stable complex with the MHC molecule.

The MHC proteins provided and used in the compositions and methods of the present disclosure may be any suitable MHC molecules known in the art. Generally, they have the formula (α-β-P)ₙ, where n is at least 2, for example between 2-10, e.g., 4. α is an α chain of a class I or class II MHC protein. β is a β chain, herein defined as the β chain of a class II MHC protein or β₂ microglobulin for a MHC class I protein. P is a peptide antigen.

In some embodiments, the MHC proteins are MHC class I complexes, such as HLA I complexes.

The MHC proteins may be from any mammalian or avian species, e.g., primate sp., particularly humans; rodents, including mice, rats and hamsters; rabbits; equines, bovines, canines, felines; etc. For instance, the MHC protein may be derived the human HLA proteins or the murine H-2 proteins. HLA proteins include the class II subunits HLA-DPα, HLA-DPβ, HLA-DQα, HLA-DQβ, HLA-DRα and HLA-DRβ, and the class I proteins HLA-A, HLA-B, HLA-C, and β2 -microglobulin. H-2 proteins include the class I subunits H-2K, H-2D, H-2L, and the class II subunits I-Aα, I-Aβ, I-Eα and I-Eβ, and β2-microglobulin. Sequences of some representative MHC proteins may be found in Kabat et al. Sequences of Proteins of Immunological Interest, NIH Publication No. 91-3242, pp724-815. MHC protein subunits suitable for use in the present invention are a soluble form of the normally membrane-bound protein, which is prepared as known in the art, for instance by deletion of the transmembrane domain and the cytoplasmic domain.

For class I proteins, the soluble form may include the α1, α2 and α3 domain. Soluble class II subunits may include the α1 and α2 domains for the α subunit, and the β1 and β2 domains for the β subunit.

The α and β subunits may be separately produced and allowed to associate in vitro to form a stable heteroduplex complex, or both of the subunits may be expressed in a single cell. Methods for producing MHC subunits are known in the art.

In certain embodiments, the MHC-peptide complex comprises a peptide epitope selected from Table 1A and an MHC whose alpha chain has an HLA-A*02 serotype, such as that encoded by an HLA-A*0201, HLA-A*0202, HLA-A*0203, HLA-A*0204, HLA-A*0205, HLA-A*0206, HLA-A*0207, HLA-A*0210, HLA-A*0211, HLA-A*0212, HLA-A*0213, HLA-A*0214, HLA-A*0216, HLA-A*0217, HLA-A*0219, HLA-A*0220, HLA-A*0222, HLA-A*0224, HLA-A*0230, HLA-A*0242, HLA-A*0253, HLA-A*0260, and/or HLA-A*0274 allele. In other embodiments, the MHC-peptide complex comprises a peptide epitope selected from Table 1B and an MHC whose alpha chain has an HLA-A*03 serotype, such as that encoded by an HLA-A*0301, HLA-A*0302, HLA-A*0305, and/or HLA-A*0307 allele. In still other embodiments, the MHC-peptide complex comprises a peptide epitope selected from Table 1C and an MHC whose alpha chain has an HLA-A*01 serotype, such as that encoded by an HLA-A*0101, HLA-A*0102, HLA-A*0103, and/or HLA-A*0116 allele. In yet other embodiments, the MHC-peptide complex comprises a peptide epitope selected from Table 1D and an MHC whose alpha chain has an HLA-A*11 serotype, such as that encoded by an HLA-A*1101, HLA-A*1102, HLA-A*1103, HLA-A* 1104, HLA-A*1105, and/or HLA-A*1119 allele. In other embodiments, the MHC-peptide complex comprises a peptide epitope selected from Table 1E and an MHC whose alpha chain has an HLA-A*24 serotype, such as that encoded by an HLA-A*2402, HLA-A*2403, HLA-A*2405, HLA-A*2407, HLA-A*2408, HLA-A*2410, HLA-A*2414, HLA-A*2417, HLA-A*2420, HLA-A*2422, HLA-A*2425, HLA-A*2426, and/or HLA-A*2458 allele. In still other embodiments, the MHC-peptide complex comprises a peptide epitope selected from Table 1F and an MHC whose alpha chain has an HLA-B*07 serotype, such as that encoded by an HLA-B*0702, HLA-B*0704, HLA-B*0705, HLA-B*0709, HLA-B*0710, HLA-B*0715, and/or HLA-B*0721 allele.

To prepare the MHC-peptide complex, the subunits may be combined with an antigenic peptide and allowed to fold in vitro to form a stable heterodimer complex with intrachain disulfide bonded domains. The peptide may be included in the initial folding reaction, or may be added to the empty heterodimer in a later step. In the compositions and methods encompassed by the present invention, this is a SARS-CoV-2 immunogenic peptide or fragment thereof. Conditions that permit folding and association of the subunits and peptide are known in the art. As one example, roughly equimolar amounts of solubilized α and β subunits may be mixed in a solution of urea. Refolding is initiated by dilution or dialysis into a buffered solution without urea. Peptides may be loaded into empty class II heterodimers at about pH 5 to 5.5 for about 1 to 3 days, followed by neutralization, concentration and buffer exchange. However, the specific folding conditions are not critical for the practice of the invention.

The monomeric complex (α-β-P) (herein monomer) may be multimerized, for example, for a MHC tetramer. The resulting multimer is stable over long periods of time. Preferably, the multimer may be formed by binding the monomers to a multivalent entity through specific attachment sites on the α or β subunit, as known in the art (e.g., as described in U.S. Pat. No. 5,635,363). The MHC proteins, in either their monomeric or multimeric forms, may also be conjugated to beads or any other support.

The multimeric complex may be labeled, so as to be directly detectable when used in immunostaining or other methods known in the art, or may be used in conjunction with secondary labeled immunoreagents which specifically bind the complex (e.g., bind to a MHC protein subunit) as known in the art. For example, the detectable label may be a fluorophore, such as fluorescein isothiocyanate (FITC), rhodamine, Texas Red, phycoerythrin (PE), allophycocyanin (APC), Brilliant Violet™ 421, Brilliant UV™ 395, Brilliant Violet™ 480, Brilliant Violet™ 421 (BV421), Brilliant Blue™ 515, APC-R700, or APC-Fire750. In some embodiments, the multimeric complex is labeled by a moiety that is capable of specifically binding another moiety. For instance, the label may be biotin, streptavidin, an oligonucleotide, or a ligand. Other labels of interest may include fluorochromes, dyes, enzymes, chemiluminescers, particles, radioisotopes, or other directly or indirectly detectable agent.

In some embodiments, a cell presenting an immunogenic peptides in context of an MHC molecule on the cell surface is generated by transfecting or transducing the cell with a vector (e.g., a viral vector) that comprising nucleic acid that encodes a recombinant or heterologous antigen into a cell. In some embodiments, the vector is introduced into the cell under conditions in which one or more peptide antigens, including, in some cases, one or more peptide antigens of the expressed heterologous protein, are expressed by the cell, processed and presented on the surface of the cell in the context of a major histocompatibility complex (MHC) molecule.

Generally, the cell to which the vector is contacted is a cell that expresses MHC, i.e., MHC-expressing cells. The cell may be one that normally expresses an MHC on the cell surface, that is induced to express and/or upregulate expression of MHC on the cell surface or that is engineered to express an MHC molecule on the cell surface. In some embodiments, the MHC contains a polymorphic peptide binding site or binding groove that can, in some cases, complex with peptide antigens of polypeptides, including peptide antigens processed by the cell machinery. In some cases, MHC molecules may be displayed or expressed on the cell surface, including as a complex with peptide, i.e., MHC-peptide complex, for presentation of an antigen in a conformation recognizable by TCRs on T cells, or other peptide binding molecules.

In some embodiments, the cell is a nucleated cell. In some embodiments, the cell is an antigen-presenting cell. In some embodiments, the cell is a macrophage, dendritic cell, B cell, endothelial cell or fibroblast. In some embodiments, the cell is an endothelial cell, such as an endothelial cell line or primary endothelial cell. In some embodiments, the cell is a fibroblast, such as a fibroblast cell line or a primary fibroblast cell.

In some embodiments, the cell is an artificial antigen presenting cell (aAPC). Typically, aAPCs include features of natural APCs, including expression of an MHC molecule, stimulatory and costimulatory molecule(s), Fc receptor, adhesion molecule(s) and/or the ability to produce or secrete cytokines (e.g., IL-2). Normally, an aAPC is a cell line that lacks expression of one or more of the above, and is generated by introduction (e.g., by transfection or transduction) of one or more of the missing elements from among an MHC molecule, a low affinity Fc receptor (CD32), a high affinity Fc receptor (CD64), one or more of a co-stimulatory signal (e.g., CD7, B7-1 (CD80), B7-2 (CD86), PD-L1, PD-L2, 4-1BBL, OX40L, ICOS-L, ICAM, CD30L, CD40, CD70, CD83, HLA-G, MICA, MICB, HVEM, lymphotoxin beta receptor, ILT3, ILT4, 3/TR6 or a ligand of B7-H3; or an antibody that specifically binds to CD27, CD28, 4-1BB, OX40, CD30, CD40, PD-1, ICOS, LFA-1, CD2, CD7, LIGHT, NKG2C, B7-H3, Toll ligand receptor or a ligand of CD83), a cell adhesion molecule (e.g., ICAM-1 or LFA-3) and/or a cytokine (e.g., IL-2, IL-4, IL-6, IL-7, IL-10, IL-12, IL-15, IL-21, interferon-alpha (IFN.alpha.), interferon-beta (IFN.beta.), interferon-gamma (IFN.gamma.), tumor necrosis factor-alpha (TNF.alpha.), tumor necrosis factor-beta (TNF.beta.), granulocyte macrophage colony stimulating factor (GM-CSF), and granulocyte colony stimulating factor (GCSF)). In some cases, an aAPC does not normally express an MHC molecule, but may be engineered to express an MHC molecule or, in some cases, is or may be induced to express an MHC molecule, such as by stimulation with cytokines. In some cases, aAPCs also may be loaded with a stimulatory ligand, which may include, for example, an anti-CD3 antibody, an anti-CD28 antibody or an anti-CD2 antibody. An exemplary cell line that may be used as a backbone for generating an aAPC is a K562 cell line or a fibroblast cell line. Various aAPCs are known in the art, see e.g., U.S. Pat. No. 8,722,400, published application No. US2014/0212446; Butler and Hirano (2014) Immunol Rev., 257(1):10. 1111/imr.12129; Suhoshki et al. (2007) Mol. Ther., 15:981-988).

It is well within the level of a skilled artisan to determine or identify the particular MHC or allele expressed by a cell. In some embodiments, prior to contacting cells with a vector, expression of a particular MHC molecule may be assessed or confirmed, such as by using an antibody specific for the particular MHC molecule. Antibodies to MHC molecules are known in the art, such as any described below.

In some embodiments, the cells may be chosen to express an MHC allele of a desired MHC restriction. In some embodiments, the MHC typing of cells, such as cell lines, are well known in the art. In some embodiments, the MHC typing of cells, such as primary cells obtained from a subject, may be determined using procedures well known in the art, such as by performing tissue typing using molecular haplotype assays (BioTest ABC SSPtray, BioTest Diagnostics Corp., Denville, N.J.; SeCore Kits, Life Technologies, Grand Island, N.Y.). In some cases, it is well within the level of a skilled artisan to perform standard typing of cells to determine the HLA genotype, such as by using sequence-based typing (SBT) (Adams et al. (2004) J. Transl. Med., 2:30; Smith (2012) Methods Mol Biol., 882:67-86). In some cases, the HLA typing of cells, such as fibroblast cells, are known. For example, the human fetal lung fibroblast cell line MRC-5 is HLA-A*0201, A29, B13, B44 Cw7 (C*0702); the human foreskin fibroblast cell line Hs68 is HLA-A1, A29, B8, B44, Cw7, Cw16; and the WI-38 cell line is A*6801, B*0801, (Solache et al. (1999) J Immunol, 163:5512-5518; Ameres et al. (2013) PloS Pathog. 9:e1003383). The human transfectant fibroblast cell line M1DR1/Ii/DM express HLA-DR and HLA-DM (Karakikes et al. (2012) FASEB J., 26:4886-96).

In some embodiments, the cells to which the vector is contacted or introduced are cells that are engineered or transfected to express an MHC molecule. In some embodiments, cell lines may be prepared by genetically modifying a parental cells line. In some embodiments, the cells are normally deficient in the particular MHC molecule and are engineered to express such particular MHC molecule. In some embodiments, the cells are genetically engineered using recombinant DNA techniques.

In some embodiments, the stable MHC-peptide complexes described herein are used to detect T cells that bind a stable MHC-peptide complex. In some embodiments, the stable MHC-peptide complexes described herein are used to monitor T cell response in a subject, for example, by detecting the amount and/or percentage of T cells (e.g., CD8+ T cells) that specifically bind to the MHC-peptide complexes that are fluorescently labeled. Methods of generating, labeling, and using MHC-peptide complexes (e.g., MHC-peptide tetramers) for detecting MHC-peptide complex-specific T cells are well known in the art. Additional description can be found in for example, U.S. Pat. US 7,776,562, U. S. Pat. US 8,268,964, and U.S. Pat. applications US2019/0085048, each of which is incorporated herein by reference in its entirety.

### V. Immunogenic Compositions

In some aspects, provided herein are pharmaceutical compositions (e.g., a vaccine composition) comprising a SARS-CoV-2 immunogenic peptide and/or a nucleic acid encoding a SARS-CoV-2 immunogenic peptide and an adjuvant. In some aspects, provided herein are pharmaceutical compositions (e.g., a vaccine composition) comprising a stable MHC-peptide complex comprising a SARS-CoV-2 immunogenic peptide in the context of a MHC molecule and an adjuvant. In some embodiments, the composition includes a combination of multiple (e.g., two or more) SARS-CoV-2 immunogenic peptides or nucleic acids and an adjuvant. In some embodiments, the composition includes a combination of multiple (e.g., two or more) stable MHC-peptide complexes comprising a SARS-CoV-2 immunogenic peptide in the context of a MHC molecule and an adjuvant. In some embodiments, the compositions described above further comprises a pharmaceutically acceptable carrier.

The pharmaceutical compositions disclosed herein may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; or (2) parenteral administration, for example, by subcutaneous, intramuscular, intravenous or epidural injection as, for example, a sterile solution or suspension, or sustained-release formulation.

Methods of preparing these formulations or compositions include the step of bringing into association a SARS-CoV-2 immunogenic peptide and/or nucleic acid described herein with the adjuvant, carrier and, optionally, one or more accessory ingredients. In general, the formulations are prepared by uniformly and intimately bringing into association an agent described herein with liquid carriers, or finely divided solid carriers, or both, and then, if necessary, shaping the product.

Pharmaceutical compositions suitable for parenteral administration comprise SARS-CoV-2 immunogenic peptides and/or nucleic acids described herein in combination with a adjuvant, as well as one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity may be maintained, for example, by the use of coating materials, such as lecithin, by the maintenance of the required particle size in the case of dispersions, and by the use of surfactants.

Regardless of the route of administration selected, the agents provided herein, which may be used in a suitable hydrated form, and/or the pharmaceutical compositions disclosed herein, are formulated into pharmaceutically-acceptable dosage forms by conventional methods known to those of skill in the art.

In some embodiments, the pharmaceutical composition described, when administered to a subject, can elicit an immune response against a cell that is infected by SARS-CoV-2. Such pharmaceutical compositions may be useful as vaccine compositions for prophylactic and/or therapeutic treatment of COIVD-19.

In some embodiments, the pharmaceutical composition further comprises a physiologically acceptable adjuvant. In some embodiments, the adjuvant employed provides for increased immunogenicity of the pharmaceutical composition. Such a further immune response stimulating compound or adjuvant may be (i) admixed to the pharmaceutical composition according to the invention after reconstitution of the peptides and optional emulsification with an oil-based adjuvant as defined above, (ii) may be part of the reconstitution composition of the invention defined above, (iii) may be physically linked to the peptide(s) to be reconstituted or (iv) may be administered separately to the subject, mammal or human, to be treated. The adjuvant may be one that provides for slow release of antigen (e.g., the adjuvant may be a liposome), or it may be an adjuvant that is immunogenic in its own right thereby functioning synergistically with antigens (i.e., antigens present in the SARS-CoV-2 immunogenic peptide). For example, the adjuvant may be a known adjuvant or other substance that promotes antigen uptake, recruits immune system cells to the site of administration, or facilitates the immune activation of responding lymphoid cells. Adjuvants include, but are not limited to, immunomodulatory molecules (e.g., cytokines), oil and water emulsions, aluminum hydroxide, glucan, dextran sulfate, iron oxide, sodium alginate, Bacto-Adjuvant, synthetic polymers such as poly amino acids and co-polymers of amino acids, saponin, paraffin oil, and muramyl dipeptide. In some embodiments, the adjuvant is Adjuvant 65, α-GalCer, aluminum phosphate, aluminum hydroxide, calcium phosphate, β-Glucan Peptide, CpG DNA, GM-CSF, GPI-0100, IFA, IFN-γ, IL-17, lipid A, lipopolysaccharide, Lipovant, Montanide, N-acetyl-muramyl-L-alanyl-D-isoglutamine, Pam3CSK4, quil A, trehalose dimycolate or zymosan.

In some embodiments, the adjuvant is an immunomodulatory molecule. For example, the immunomodulatory molecule may be a recombinant protein cytokine, chemokine, or immunostimulatory agent or nucleic acid encoding cytokines, chemokines, or immunostimulatory agents designed to enhance the immunologic response.

Examples of immunomodulatory cytokines include interferons (e.g., IFNα, IFNβ and IFNγ), interleukins (e.g., IL-1, IL-2, IL-3, IL-4, IL-5, IL-6, IL-7, IL-8, IL-9, IL-10, IL-12, IL-17 and IL-20), tumor necrosis factors (e.g., TNFα and TNFβ), erythropoietin (EPO), FLT-3 ligand, gIp10, TCA-3, MCP-1, MIF, MIP-1.alpha., MIP-1β, Rantes, macrophage colony stimulating factor (M-CSF), granulocyte colony stimulating factor (G-CSF), and granulocyte-macrophage colony stimulating factor (GM-CSF), as well as functional fragments of any of the foregoing.

In some embodiments, an immunomodulatory chemokine that binds to a chemokine receptor, i.e., a CXC, CC, C, or CX3C chemokine receptor, also may be included in the compositions provided here. Examples of chemokines include, but are not limited to, Mip1α, Mip-1β, Mip-3α (Larc), Mip-3β, Rantes, Hcc-1, Mpif-1, Mpif-2, Mcp-1, Mcp-2, Mcp-3, Mcp-4, Mcp-5, Eotaxin, Tarc, Elc, I309, IL-8, Gcp-2 Gro-α, Gro-β, Gro-γ, Nap-2, Ena-78, Gcp-2, Ip-10, Mig, I-Tac, Sdf-1, and Bca-1 (Blc), as well as functional fragments of any of the foregoing.

In some embodiments, the composition comprises a nucleic acid encoding an SARS-CoV-2 immunogenic polypeptide described herein, such as a DNA molecule encoding a SARS-CoV-2 immunogenic peptide. In some embodiments the composition comprises an expression vector comprising an open reading frame encoding a SARS-CoV-2 immunogenic peptide.

When taken up by a cell (e.g., muscle cell, an antigen-presenting cell (APC) such as a dendritic cell, macrophage, etc.), a DNA molecule may be present in the cell as an extrachromosomal molecule and/or may integrate into the chromosome. DNA may be introduced into cells in the form of a plasmid which may remain as separate genetic material. Alternatively, linear DNAs that may integrate into the chromosome may be introduced into the cell. Optionally, when introducing DNA into a cell, reagents which promote DNA integration into chromosomes may be added.

### VI. Binding Moieties

In some aspects, a binding moiety that binds a peptide described herein and/or a stable MHC-peptide complex described herein are provided. For example, binding proteins like T cell receptors (TCRs), antibodies, and the like that specifically bind to the peptide and/or the stable MHC-peptide complex, such as with a K_{d} less than or equal to about 10⁻⁷ M (e.g., about 10⁻⁷, about 10⁻⁸, about 10⁻⁹, about 10¹⁰, about 10⁻¹¹, about 10⁻¹², about 10⁻¹³, about 10⁻¹⁴), are provided.

In some embodiments, the MHC molecule comprises an MHC alpha chain that is an HLA serotype selected from the group consisting of HLA-A*02, HLA-A*03, HLA-A*01, HLA-A*11, HLA-A*24, and/or HLA-B*07. In some embodiments, the HLA allele is selected from the group consisting of HLA-A*0201, HLA-A*0202, HLA-A*0203, HLA-A*0204, HLA-A*0205, HLA-A*0206, HLA-A*0207, HLA-A*0210, HLA-A*0211, HLA-A*0212, HLA-A*0213, HLA-A*0214, HLA-A*0216, HLA-A*0217, HLA-A*0219, HLA-A*0220, HLA-A*0222, HLA-A*0224, HLA-A*0230, HLA-A*0242, HLA-A*0253, HLA-A*0260, and HLA-A*0274 allele. In a specific embodiment, the HLA allele is HLA-A*0201. In some embodiments, the binding proteins are genetically engineered, isolated, and/or purified.

In some embodiments, the binding proteins provided herein comprise a constant region that is chimeric, humanized, human, primate, or rodent (e.g., rat or mouse). For example, a human variable region may be chimerized with a murine constant region or a murine variable region may be humanized with a human constant region and/or human framework regions. In some embodiments, the constant regions may be mutated to modify functionality (e.g., introduction of non-naturally occurring cysteine substitutions in opposing residue locations in TCR alpha and beta chains to provide disulfide bonds useful for increasing affinity between the TCR alpha and beta chains). Similarly, mutations may be made in the transmembrane domain of the constant region to modify functionality (e.g., increase hydrophobicity by introducing a non-naturally occurring substitution of a residue with a hydrophobic amino acid).

In some embodiments, each CDR of the binding protein has up to five amino acid substitutions, insertions, deletions, or a combination thereof as compared to a reference CDR sequence.

In some embodiments, the binding proteins disclosed herein may comprise a T cell receptor (TCR), an antigen-binding fragment of a TCR, or a chimeric antigen receptor (CAR). In some embodiments, the binding protein disclosed herein may comprise two polypeptide chains, each of which comprises a variable region comprising a CDR3 of a TCR alpha chain and a CDR3 of a TCR beta chain, or a CDR1, CDR2, and CDR3 of both a TCR alpha chain and a TCR beta chain. In some embodiments, a binding protein comprises a single chain TCR (scTCR), which comprises both the TCR V_{α} and TCR V_{β} domains, but only a single TCR constant domain (C_{α} or C_{β}). The term “chimeric antigen receptor” (CAR) refers to a fusion protein that is engineered to contain two or more naturally-occurring amino acid sequences linked together in a way that does not occur naturally or does not occur naturally in a host cell, which fusion protein can function as a receptor when present on a surface of a cell. CARs encompassed by the present invention may include an extracellular portion comprising an antigen-binding domain (i.e., obtained or derived from an immunoglobulin or immunoglobulin-like molecule, such as an antibody or TCR, or an antigen binding domain derived or obtained from a killer immunoreceptor from an NK cell) linked to a transmembrane domain and one or more intracellular signaling domains (optionally containing co-stimulatory domain(s)) (see, e.g., Sadelain et al. (2013) Cancer Discov. 3:388, Harris and Kranz (2016) Trends Pharmacol. Sci. 37:220, and Stone et al. (2014) Cancer Immunol. Immunother. 63:1163).

In some embodiments, the binding proteins (e.g., the TCR, antigen-binding fragment of a TCR, or chimeric antigen receptor (CAR)) disclosed herein is chimeric (e.g., comprises amino acid residues or motifs from more than one donor or species), humanized (e.g., comprises residues from a non-human organism that are altered or substituted so as to reduce the risk of immunogenicity in a human), or human.

Methods for producing engineered binding proteins, such as TCRs, CARs, and antigen-binding fragments thereof, are well-known in the art (e.g., Bowerman et al. (2009) Mol. Immunol. 5:3000, U.S. Pat. No. 6,410,319, U.S. Pat. No. 7,446,191, U.S. Pat. Publ. No. 2010/065818; U.S. Pat. No. 8,822,647, PCT Publ. No. WO 2014/031687, U.S. Pat. No. 7,514,537, and Brentjens et al. (2007) Clin. Cancer Res. 73:5426).

In some embodiments, the binding protein described herein is a TCR, or antigen-binding fragment thereof, expressed on a cell surface, wherein the cell surface-expressed TCR is capable of more efficiently associating with a CD3 protein as compared to endogenous TCR A binding protein encompassed by the present invention, such as a TCR, when expressed on the surface of a cell like a T cell, may also have higher surface expression on the cell as compared to an endogenous binding protein, such as an endogenous TCR In some embodiments, provided herein is a CAR, wherein the binding domain of the CAR comprises an antigen-specific TCR binding domain (see, e.g., Walseng et al. (2017) Scientific Reports 7:10713).

Also provided are modified binding proteins (e.g., TCRs, antigen-binding fragments of TCRs, or CARs) that may be prepared according to well-known methods using a binding protein having one or more of the V_{α} and/or V_{β} sequences disclosed herein as starting material to engineer a modified binding protein that may have altered properties from the starting binding protein. A binding protein may be engineered by modifying one or more residues within one or both variable regions (i.e., V_{α} and/or V_{β}), for example within one or more CDR regions and/or within one or more framework regions. Additionally or alternatively, a binding protein may be engineered by modifying residues within the constant region(s).

Another type of variable region modification is to mutate amino acid residues within the V_{α} and/or V_{β} CDR1, CDR2 and/or CDR3 regions to thereby improve one or more binding properties (e.g., affinity) of the binding protein of interest. Site-directed mutagenesis or PCR-mediated mutagenesis may be performed to introduce the mutation(s) and the effect on protein binding, or other functional property of interest, may be evaluated in in vitro or in vivo assays as described herein and provided in the Examples. In some embodiments, conservative modifications (as discussed above) may be introduced. The mutations may be amino acid substitutions, additions or deletions. In some embodiments, the mutations are substitutions. Moreover, typically no more than one, two, three, four or five residues within a CDR region are modified.

In some embodiments, binding proteins (e.g., TCRs, antigen-binding fragments of TCRs, or CARs) described herein may possess one or more amino acid substitutions, deletions, or additions relative to a naturally occurring TCR In some embodiments, each CDR of the binding protein has up to five amino acid substitutions, insertions, deletions, or a combination thereof as compared to a reference CDR sequence. Conservative substitutions of amino acids are well-known and may occur naturally or may be introduced when the binding protein is recombinantly produced. Amino acid substitutions, deletions, and additions may be introduced into a protein using mutagenesis methods known in the art (see, e.g., Sambrook et al. (2001) Molecular Cloning: A Laboratory Manual, 3d ed., Cold Spring Harbor Laboratory Press, NY). Oligonucleotide-directed site-specific (or segment specific) mutagenesis procedures may be employed to provide an altered polynucleotide that has particular codons altered according to the substitution, deletion, or insertion desired. Alternatively, random or saturation mutagenesis techniques, such as alanine scanning mutagenesis, error prone polymerase chain reaction mutagenesis, and oligonucleotide-directed mutagenesis may be used to prepare immunogen polypeptide variants (see, e.g., Sambrook et al. supra).

A variety of criteria known to the ordinarily skilled artisan indicate whether an amino acid that is substituted at a particular position in a peptide or polypeptide is conservative (or similar). For example, a similar amino acid or a conservative amino acid substitution is one in which an amino acid residue is replaced with an amino acid residue having a similar side chain. Similar amino acids may be included in the following categories: amino acids with basic side chains (e.g., lysine, arginine, histidine); amino acids with acidic side chains (e.g., aspartic acid, glutamic acid); amino acids with uncharged polar side chains (e.g., glycine, asparagine, glutamine, serine, threonine, tyrosine, cysteine, histidine); amino acids with nonpolar side chains (e.g., alanine, valine, leucine, isoleucine, proline, phenylalanine, methionine, tryptophan); amino acids with beta-branched side chains (e.g., threonine, valine, isoleucine), and amino acids with aromatic side chains (e.g., tyrosine, phenylalanine, tryptophan). Proline, which is considered more difficult to classify, shares properties with amino acids that have aliphatic side chains (e.g., leucine, valine, isoleucine, and alanine). In some embodiments, substitution of glutamine for glutamic acid or asparagine for aspartic acid may be considered a similar substitution in that glutamine and asparagine are amide derivatives of glutamic acid and aspartic acid, respectively. As understood in the art “similarity” between two polypeptides is determined by comparing the amino acid sequence and conserved amino acid substitutes thereto of the polypeptide to the sequence of a second polypeptide (e.g., using GENEWORKS™, Align, the BLAST algorithm, or other algorithms described herein and practiced in the art).

In any of the embodiments described herein, an encoded binding protein (e.g., TCR, antigen-binding fragment of a TCR, or CAR) may comprise a “signal peptide” (also known as a leader sequence, leader peptide, or transit peptide). Signal peptides target newly synthesized polypeptides to their appropriate location inside or outside the cell. A signal peptide may be removed from the polypeptide during or once localization or secretion is completed. Polypeptides that have a signal peptide are referred to herein as a “pre-protein” and polypeptides having their signal peptide removed are referred to herein as “mature” proteins or polypeptides. In some embodiments, a binding protein (e.g., TCR, antigen-binding fragment of a TCR, or CAR) described herein comprises a mature V_{α} domain, a mature V_{β} domain, or both. In some embodiments, a binding protein (e.g., TCR, antigen-binding fragment of a TCR, or CAR) described herein comprises a mature TCR β-chain, a mature TCR α-chain, or both.

In some embodiments, the binding proteins are fusion proteins comprising: (a) an extracellular component comprising a TCR or antigen-binding fragment thereof; (b) an intracellular component comprising an effector domain or a functional portion thereof; and (c) a transmembrane domain connecting the extracellular and intracellular components. In some embodiments, the fusion protein is capable of specifically binding to a MHC-peptide antigen complex comprising a peptide epitope described herein in the context of an MHC molecule (e.g., a MHC class I molecule).

As used herein, an “effector domain” or “immune effector domain” is an intracellular portion or domain of a fusion protein or receptor that can directly or indirectly promote an immune response in a cell when receiving an appropriate signal. In some embodiments, an effector domain is from an immune cell protein or portion thereof or immune cell protein complex that receives a signal when bound (e.g., CD3ζ), or when the immune cell protein or portion thereof or immune cell protein complex binds directly to a target molecule and triggers signal transduction from the effector domain in an immune cell.

An effector domain may directly promote a cellular response when it contains one or more signaling domains or motifs, such as an intracellular tyrosine-based activation motif (ITAM), such as those found in costimulatory molecules. Without wishing to be bound by theory, it is believed that ITAMs are useful for T cell activation following ligand engagement by a T cell receptor or by a fusion protein comprising a T cell effector domain. In some embodiments, the intracellular component or functional portion thereof comprises an ITAM. Exemplary immune effector domains include but are not limited to those from, CD3ε, CD3δ, CD3ζ, CD25, CD79A, CD79B, CARD11, DAP10, FcRα, FcRβ, FcRγ, Fyn, HVEM, ICOS, Lck, LAG3, LAT, LRP, NKG2D, NOTCH1, NOTCH2, NOTCH3, NOTCH4, Wnt, ROR2, Ryk, SLAMF1, Slp76, pTα, TCRα, TCRβ, TRIM, Zap70, PTCH2, or any combination thereof. In some embodiments, an effector domain comprises a lymphocyte receptor signaling domain (e.g., CD3ζ or a functional portion or variant thereof).

In further embodiments, the intracellular component of the fusion protein comprises a costimulatory domain or a functional portion thereof selected from CD27, CD28, 4-1BB (CD137), OX40 (CD134), CD2, CD5, ICAM-1 (CD54), LFA-1 (CD11a/CD18), ICOS (CD278), GITR, CD30, CD40, BAFF-R, HVEM, LIGHT, MKG2C, SLAMF7, NKp80, CD160, B7-H3, a ligand that specifically binds with CD83, or a functional variant thereof, or any combination thereof. In some embodiments, the intracellular component comprises a CD28 costimulatory domain or a functional portion or variant thereof (which may optionally include a LL- GG mutation at positions 186-187 of the native CD28 protein (e.g., Nguyen et al. (2003) Blood 702:4320), a 4-1BB costimulatory domain or a functional portion or variant thereof, or both.

In some embodiments, an effector domain comprises a CD3ε endodomain or a functional (e.g., signaling) portion thereof, or a functional variant thereof. In further embodiments, an effector domain comprises a CD27 endodomain or a functional (e.g., signaling) portion thereof, or a functional variant thereof. In further embodiments, an effector domain comprises a CD28 endodomain or a functional (e.g., signaling) portion thereof, or a functional variant thereof. In still further embodiments, an effector domain comprises a 4-1BB endodomain or a functional (e.g., signaling) portion thereof, or a functional variant thereof. In further embodiments, an effector domain comprises an OX40 endodomain or a functional (e.g., signaling) portion thereof, or a functional variant thereof. In further embodiments, an effector domain comprises a CD2 endodomain or a functional (e.g., signaling) portion thereof, or a functional variant thereof. In further embodiments, an effector domain comprises a CD5 endodomain or a functional (e.g., signaling) portion thereof, or a functional variant thereof. In further embodiments, an effector domain comprises an ICAM-1 endodomain or a functional (e.g., signaling) portion thereof, or a functional variant thereof. In further embodiments, an effector domain comprises a LFA-1 endodomain or a functional (e.g., signaling) portion thereof, or a functional variant thereof. In further embodiments, an effector domain comprises an ICOS endodomain or a functional (e.g., signaling) portion thereof, or a functional variant thereof.

An extracellular component and an intracellular component encompassed by the present invention are connected by a transmembrane domain. A “transmembrane domain,” as used herein, is a portion of a transmembrane protein that can insert into or span a cell membrane. Transmembrane domains have a three-dimensional structure that is thermodynamically stable in a cell membrane and generally range in length from about 15 amino acids to about 30 amino acids. The structure of a transmembrane domain may comprise an alpha helix, a beta barrel, a beta sheet, a beta helix, or any combination thereof. In some embodiments, the transmembrane domain comprises or is derived from a known transmembrane protein (e.g., a CD4 transmembrane domain, a CD8 transmembrane domain, a CD27 transmembrane domain, a CD28 transmembrane domain, or any combination thereof).

In some embodiments, the extracellular component of the fusion protein further comprises a linker disposed between the binding domain and the transmembrane domain. As used herein when referring to a component of a fusion protein that connects the binding and transmembrane domains, a “linker” may be an amino acid sequence having from about two amino acids to about 500 amino acids, which can provide flexibility and room for conformational movement between two regions, domains, motifs, fragments, or modules connected by the linker. For example, a linker encompassed by the present invention can position the binding domain away from the surface of a host cell expressing the fusion protein to enable proper contact between the host cell and a target cell, antigen binding, and activation (Patel et al. (1999) Gene Therapy 6:412-419). Linker length may be varied to maximize antigen recognition based on the selected target molecule, selected binding epitope, or antigen binding domain seize and affinity (see, e.g., Guest et al. (2005) Immunother. 28:203-11 and PCT Publ. No. WO 2014/031687). Exemplary linkers include those having a glycine-serine amino acid chain having from one to about ten repeats of GlyₓSer_{y}, wherein x and y are each independently an integer from 0 to 10, provided that x and y are not both 0 (e.g., (Gly₄Ser)₂, (Gly₃Ser)₂, Gly₂Ser, or a combination thereof, such as ((Gly₃Ser)₂Gly₂Ser)).

In some embodiments, binding moeities encompassed by the present invention may be engineered protein scaffolds, an antibody or an antigen-binding fragment thereof, TCR-mimic antibodies, and the like. Such binding moieties may be designed and/or generated against peptides and/or MHC-peptide complexes described herein using routine immunological methods, such as immunizing a host, obtaining antibody-producing cells and/or antibodies thereof, and generating hybridomas useful for producing monoclonal antibodies (e.g., Watt et al. (2006) Nat. Biotechnol. 24:177-183; Gebauer and Skerra (2009) Curr. Opin. Chem Biol. 13:245-255; Skerra et al. (2008) FEBS J. 275:2677-2683; Nygren et al. (2008) FEBS J. 275:2668-2676; Dana et al. (2012) Exp. Rev. Mol. Med. 14:e6; Sergeva et al. (2011) Blood 117:4262-4272; PCT Publ. Nos. WO 2007/143104, PCT/US86/02269, and WO 86/01533; U.S. Pat. No. 4,816,567; Better et al. (1988) Science 240:1041-1043; Liu et al. (1987) Proc. Natl. Acad. Sci. U.S.A. 84:3439-3443; Liu et al. (1987) J. Immunol. 139:3521-3526; Sun et al. (1987) Proc. Natl. Acad. Sci. 84:214-218; Nishimura et al. (1987) Cancer Res. 47:999-1005; Wood et al. (1985) Nature 314:446-449; Shaw et al. (1988) J. Natl. Cancer Inst. 80: 1553-1559); Morrison, S. L. (1985) Science 229:1202-1207; Oi et al. (1986) Biotechniques 4:214; U.S. Pat. No. 5,225,539; Jones et al. (1986) Nature 321:552-525; Verhoeyan et al. (1988) Science 239: 1534; and Beidler et al. (1988) J. Immunol. 141:4053-4060. If desired, binding moieties may be isolated or purified using conventional procedures such as, for example, protein A-Sepharose, hydroxylapatite chromatography, gel electrophoresis, dialysis, affinity chromatography, ammonium sulfate or ethanol precipitation, acid extraction, anion or cation exchange chromatography, phosphocellulose chromatography, hydrophobic interaction chromatography, hydroxylapatite chromatography, lectin chromatography, and high performance liquid chromatography (HPLC) (e.g., Current Protocols in Immunology, or Current Protocols in Protein Science, John Wiley & Sons, NY, N.Y.).

The terms “antibody” and “antibodies” broadly encompass naturally-occurring forms of antibodies (e.g. IgG, IgA, IgM, IgE) and recombinant antibodies, such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody.

In addition, intrabodies are well-known antigen-binding molecules having the characteristic of antibodies, but that are capable of being expressed within cells in order to bind and/or inhibit intracellular targets of interest (Chen et al. (1994) Human Gene Ther. 5:595-601). Methods are well-known in the art for adapting antibodies to target (e.g., inhibit) intracellular moieties, such as the use of single-chain antibodies (scFvs), modification of immunoglobulin VL domains for hyperstability, modification of antibodies to resist the reducing intracellular environment, generating fusion proteins that increase intracellular stability and/or modulate intracellular localization, and the like. Intracellular antibodies can also be introduced and expressed in one or more cells, tissues or organs of a multicellular organism, for example for prophylactic and/or therapeutic purposes (e.g., as a gene therapy) (see, at least PCT Publ. Nos. WO 08/020079, WO 94/02610, WO 95/22618, and WO 03/014960; U.S. Pat. No. 7,004,940; Cattaneo and Biocca (1997) Intracellular Antibodies: Development and Applications (Landes and Springer-Verlag publs.); Kontermann (2004) Methods 34:163-170; Cohen et al. (1998) Oncogene 17:2445-2456; Auf der Maur et al. (2001) FEBS Lett. 508:407-412; Shaki-Loewenstein et al. (2005) J. Immunol. Meth. 303:19-39).

The term “antibody” as used herein also includes an “antigen-binding portion” of an antibody (or simply “antibody portion”). The term “antigen-binding portion”, as used herein, refers to one or more fragments of an antibody that retain the ability to specifically bind to an antigen (e.g., a peptide and/or an MHC-peptide complex described herein). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent polypeptides (known as single chain Fv (scFv); see e.g., Bird et al. (1988) Science 242:423-426; and Huston et al. (1988) Proc. Natl. Acad. Sci. USA 85:5879-5883; and Osbourn et al. 1998, Nature Biotechnology 16: 778). Such single chain antibodies are also intended to be encompassed within the term “antigen-binding portion” of an antibody. Any VH and VL sequences of specific scFv can be linked to human immunoglobulin constant region cDNA or genomic sequences, in order to generate expression vectors encoding complete IgG polypeptides or other isotypes. VH and VL can also be used in the generation of Fab, Fv or other fragments of immunoglobulins using either protein chemistry or recombinant DNA technology. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger et al. (1993) Proc. Natl. Acad. Sci. U.S.A. 90:6444-6448; Poljak et al. (1994) Structure 2:1121-1123).

Still further, an antibody or antigen-binding portion thereof may be part of larger immunoadhesion polypeptides, formed by covalent or noncovalent association of the antibody or antibody portion with one or more other proteins or peptides. Examples of such immunoadhesion polypeptides include use of the streptavidin core region to make a tetrameric scFv polypeptide (Kipriyanov et al. (1995) Human Antibodies and Hybridomas 6:93-101) and use of a cysteine residue, protein subunit peptide and a C-terminal polyhistidine tag to make bivalent and biotinylated scFv polypeptides (Kipriyanov et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion polypeptides can be obtained using standard recombinant DNA techniques, as described herein.

Antibodies may be polyclonal or monoclonal; xenogeneic, allogeneic, or syngeneic; or modified forms thereof (e.g. humanized, chimeric, etc.). Antibodies may also be fully human. Preferably, antibodies of the invention bind specifically or substantially specifically to a peptide and/or an MHC-peptide complex described herein. The terms “monoclonal antibodies” and “monoclonal antibody composition”, as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

Similar to other binding moieties described herein, antibodies may also be “humanized,” which is intended to include antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. The humanized antibodies of the invention may include amino acid residues not encoded by human germline immunoglobulin sequences (e.g., mutations introduced by random or site-specific mutagenesis in vitro or by somatic mutation in vivo), for example in the CDRs. The term “humanized antibody”, as used herein, also includes antibodies in which CDR sequences derived from the germline of another mammalian species, have been grafted onto human framework sequences.

Binding proteins encompassed by the present invention may, in some embodiments, be covalently linked to a moiety. In some embodiments, the covalently linked moiety comprises an affinity tag or a label. The affinity tag may be selected from the group consisting of Glutathione-S-Transferase (GST), calmodulin binding protein (CBP), protein C tag, Myc tag, HaloTag, HA tag, Flag tag, His tag, biotin tag, and V5 tag. The label may be a fluorescent protein. In some embodiments, the covalently linked moiety is selected from the group consisting of an inflammatory agent, an anti-inflammatory agent, a cytokine, a toxin, a cytotoxic molecule, a radioactive isotope, or an antibody such as a single-chain Fv.

A binding protein may be conjugated to an agent used in imaging, research, therapeutics, theranostics, pharmaceuticals, chemotherapy, chelation therapy, targeted drug delivery, and radiotherapy. In some embodiments, a binding protein may be conjugated to or fused with detectable agents, such as a fluorophore, a near-infrared dye, a contrast agent, a nanoparticle, a metal-containing nanoparticle, a metal chelate, an X-ray contrast agent, a PET agent, a metal, a radioisotope, a dye, radionuclide chelator, or another suitable material that can be used in imaging. In some embodiments, 1, 2, 3, 4, 5, 6, 7, 8, 9, 10, or more detectable moieties may be linked to a binding protein. Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212. In some embodiments, the near-infrared dyes are not easily quenched by biological tissues and fluids. In some embodiments, the fluorophore is a fluorescent agent emitting electromagnetic radiation at a wavelength between 650 nm and 4000 nm, such emissions being used to detect such agent Non-limiting examples of fluorescent dyes that may be used as a conjugating molecule include DyLight-680, DyLight-750, VivoTag-750, DyLight-800, IRDye-800, VivoTag-680, Cy5.5, ZQ800, or indocyanine green (ICG). In some embodiments, near infrared dyes often include cyanine dyes (e.g., Cy7, Cy5.5, and Cy5). Additional, non-limiting examples of fluorescent dyes for use as a conjugating molecule in accordance with present invention include acradine orange or yellow, Alexa Fluors® (e.g., Alexa Fluor® 790, 750, 700, 680, 660, and 647) and any derivative thereof, 7-actinomycin D, 8-anilinonaphthalene-1-sulfonic acid, ATTO® dye and any derivative thereof, auramine-rhodamine stain and any derivative thereof, bensantrhone, bimane, 9-10-bis(phenylethynyl)anthracene, 5,12-bis(phenylethynyl)naththacene, bisbenzimide, brainbow, calcein, carbodyfluorescein and any derivative thereof, 1-chloro-9,10-bis(phenylethynyl)anthracene and any derivative thereof, DAPI, DiOC6, DyLight® Fluors® and any derivative thereof, epicocconone, ethidium bromide, F1AsH-EDT2®, Fluo dye and any derivative thereof, FluoProbe® and any derivative thereof, fluorescein and any derivative thereof, Fura® and any derivative thereof, GelGreen® and any derivative thereof, GelRed® and any derivative thereof, fluorescent proteins and any derivative thereof, m isoform proteins and any derivative thereof such as for example mCherry, hetamethine dye and any derivative thereof, hoeschst stain, iminocoumarin, indian yellow, indo-1 and any derivative thereof, laurdan, lucifer yellow and any derivative thereof, luciferin and any derivative thereof, luciferase and any derivative thereof, mercocyanine and any derivative thereof, nile dyes and any derivative thereof, perylene, phloxine, phyco dye and any derivative thereof, propium iodide, pyranine, rhodamine and any derivative thereof, ribogreen, RoGFP, rubrene, stilbene and any derivative thereof, sulforhodamine and any derivative thereof, SYBR and any derivative thereof, synapto-pHluorin, tetraphenyl butadiene, tetrasodium tris, Texas Red, Titan Yellow, TSQ, umbelliferone, violanthrone, yellow fluorescent protein and YOYO-1. Other suitable fluorescent dyes include, but are not limited to, fluorescein and fluorescein dyes (e.g., fluorescein isothiocyanine or FITC, naphthofluorescein, 4′, 5′-dichloro-2′,7′-dimethoxyfluorescein, 6-carboxyfluorescein or FAM, etc.), carbocyanine, merocyanine, styryl dyes, oxonol dyes, phycoerythrin, erythrosin, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Oregon Green™ dyes (e.g., Oregon Green™ 488, 500, 514., etc.), Texas Red®, Texas Red®-X, SPECTRUM RED®, SPECTRUM GREEN®, cyanine dyes (e.g., CY-3, Cy-5, CY-3.5, CY-5.5, etc.), Alexa Fluor® dyes (e.g., Alexa Fluor® 350, 488, 532, 546, 568, 594, 633, 660, 680, etc.), BODIPY® dyes (e.g., BODIPY® FL, R6G, TMR, TR, 530/550, 558/568, 564/570, 576/589, 581/591, 630/650, 650/665, etc.), IRD dyes (e.g., IRD40™, IRD700™, IRD800™, etc.), and the like. Additional suitable detectable agents are well-known in the art (e.g., PCT Publ. No. PCT/US14/56177). Non-limiting examples of radioisotopes include alpha emitters, beta emitters, positron emitters, and gamma emitters. In some embodiments, the metal or radioisotope is selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. In some embodiments, the metal is actinium, bismuth, lead, radium, strontium, samarium, or yttrium. In some embodiments, the radioisotope is actinium-225 or lead-212.

Binding proteins may be conjugated to a radiosensitizer or photosensitizer. Examples of radiosensitizers include but are not limited to: ABT-263, ABT-199, WEHI-539, paclitaxel, carboplatin, cisplatin, oxaliplatin, gemcitabine, etanidazole, misonidazole, tirapazamine, and nucleic acid base derivatives (e.g., halogenated purines or pyrimidines, such as 5-fluorodeoxyuridine). Examples of photosensitizers include but are not limited to: fluorescent molecules or beads that generate heat when illuminated, nanoparticles, porphyrins and porphyrin derivatives (e.g., chlorins, bacteriochlorins, isobacteriochlorins, phthalocyanines, and naphthalocyanines), metalloporphyrins, metallophthalocyanines, angelicins, chalcogenapyrrillium dyes, chlorophylls, coumarins, flavins and related compounds such as alloxazine and riboflavin, fullerenes, pheophorbides, pyropheophorbides, cyanines (e.g., merocyanine 540), pheophytins, sapphyrins, texaphyrins, purpurins, porphycenes, phenothiaziniums, methylene blue derivatives, naphthalimides, nile blue derivatives, quinones, perylenequinones (e.g., hypericins, hypocrellins, and cercosporins), psoralens, quinones, retinoids, rhodamines, thiophenes, verdins, xanthene dyes (e.g., eosins, erythrosins, rose bengals), dimeric and oligomeric forms of porphyrins, and prodrugs such as 5-aminolevulinic acid. Advantageously, this approach allows for highly specific targeting of cells of interest (e.g., immune cells) using both a therapeutic agent (e.g., drug) and electromagnetic energy (e.g., radiation or light) concurrently. In some embodiments, the binding protein is fused with, or covalently or non-covalently linked to the agent, for example, directly or via a linker.

In some embodiments, the binding protein may be chemically modified. For example, a binding protein may be mutated to modify peptide properties such as detectability, stability, biodistribution, pharmacokinetics, half-life, surface charge, hydrophobicity, conjugation sites, pH, function, and the like. N-methylation is one example of methylation that can occur in a binding protein encompassed by the present invention. In some embodiments, a binding protein may be modified by methylation on free amines such as by reductive methylation with formaldehyde and sodium cyanoborohydride.

A chemical modification may comprise a polymer, a polyether, polyethylene glycol, a biopolymer, a zwitterionic polymer, a polyamino acid, a fatty acid, a dendrimer, an Fc region, a simple saturated carbon chain such as palmitate or myristolate, or albumin. The chemical modification of a binding protein with an Fc region may be a fusion Fc-protein. A polyamino acid may include, for example, a poly amino acid sequence with repeated single amino acids (e.g., poly glycine), and a poly amino acid sequence with mixed poly amino acid sequences that may or may not follow a pattern, or any combination of the foregoing.

In some embodiments, the binding proteins encompassed by the present invention may be modified. In some embodiments, the modifications having substantial or significant sequence identity to a parent binding protein to generate a functional variant that maintains one or more biophysical and/or biological activities of the parent binding protein (e.g., maintain binding specificity). In some embodiments, the mutation is a conservative amino acid substitution.

In some embodiments, binding proteins encompassed by the present invention may comprise synthetic amino acids in place of one or more naturally-occurring amino acids. Such synthetic amino acids are well-known in the art, and include, for example, aminocyclohexane carboxylic acid, norleucine, a-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, a-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, a-aminocyclopentane carboxylic acid, oc-aminocyclohexane carboxylic acid, a-aminocycloheptane carboxylic acid, a-(2-amino-2-norbornane)-carboxylic acid, α,γ-diaminobutyric acid,β-diaminopropionic acid, homophenylalanine, and oc-tert-butylglycine.

Binding proteins encompassed by the present invention may be modified, such as glycosylated, amidated, carboxylated, phosphorylated, esterified, N-acylated, cyclized (e.g., via a disulfide bridge), or converted into an acid addition salt and/or optionally dimerized or polymerized, or conjugated.

In some embodiments, the attachment of a hydrophobic moiety, such as to the N-terminus, the C-terminus, or an internal amino acid, may be used to extend half-life of a peptide encompassed by the present invention. In other embodiments, a binding protein may include post-translational modifications (e.g., methylation and/or amidation), which can affect, for example, serum half-life. In some embodiments, simple carbon chains (e.g., by myristoylation and/or palmitylation) may be conjugated to the binding proteins. In some embodiments, the simple carbon chains may render the binding proteins easily separable from the unconjugated material. For example, methods that may be used to separate the binding proteins from the unconjugated material include, but are not limited to, solvent extraction and reverse phase chromatography. The lipophilic moieties can extend half-life through reversible binding to serum albumin. The conjugated moieties may be lipophilic moieties that extend half-life of the peptides through reversible binding to serum albumin. In some embodiments, the lipophilic moiety may be cholesterol or a cholesterol derivative, including cholestenes, cholestanes, cholestadienes and oxysterols. In some embodiments, the binding proteins may be conjugated to myristic acid (tetradecanoic acid) or a derivative thereof. In other embodiments, a binding protein may be coupled (e.g., conjugated) to a half-life modifying agent Examples of half-life modifying agents include but are not limited to: a polymer, a polyethylene glycol (PEG), a hydroxyethyl starch, polyvinyl alcohol, a water soluble polymer, a zwitterionic water soluble polymer, a water soluble poly(amino acid), a water soluble polymer of proline, alanine and serine, a water soluble polymer containing glycine, glutamic acid, and serine, an Fc region, a fatty acid, palmitic acid, or a molecule that binds to albumin. In some embodiments, a spacer or linker may be coupled to a binding protein, such as 1, 2, 3, 4, or more amino acid residues that serve as a spacer or linker in order to facilitate conjugation or fusion to another molecule, as well as to facilitate cleavage of the peptide from such conjugated or fused molecules. In some embodiments, binding proteins may be conjugated to other moieties that, for example, can modify or effect changes to the properties of the binding proteins.

A binding protein may be produced recombinantly or synthetically, such as by solid-phase peptide synthesis or solution-phase peptide synthesis. Polypeptide synthesis may be performed by known synthetic methods, such as using fluorenylmethyloxycarbonyl (Fmoc) chemistry or by butyloxycarbonyl (Boc) chemistry. Polypeptide fragments may be joined together enzymatically or synthetically.

In an aspect encompassed by the present invention, provided herein are methods of producing a binding protein described herein, comprising the steps of: (i) culturing a transformed host cell which has been transformed by a nucleic acid comprising a sequence encoding a binding protein described herein under conditions suitable to allow expression of said binding protein; and (ii) recovering the expressed binding protein.

Methods useful for isolating and purifying recombinantly produced binding protein, by way of example, may include obtaining supernatants from suitable host cell/vector systems that secrete the binding protein into culture media and then concentrating the media using a commercially available filter. Following concentration, the concentrate may be applied to a single suitable purification matrix or to a series of suitable matrices, such as an affinity matrix or an ion exchange resin. One or more reverse phase HPLC steps may be employed to further purify a recombinant polypeptide. These purification methods may also be employed when isolating an immunogen from its natural environment. Methods for large scale production of one or more of binding proteins described herein include batch cell culture, which is monitored and controlled to maintain appropriate culture conditions. Purification of the binding protein may be performed according to methods described herein and known in the art.

A variety of assays are well-known for assessing binding affinity and/or determining whether a binding molecule specifically binds to a particular ligand (e.g., peptide antigen-MHC complex). It is within the level of a skilled artisan to determine the binding affinity of a binding protein for a target, such as a T cell peptide epitope of a target polypeptide, such as by using any of a number of binding assays that are well-known in the art. For example, in some embodiments, a Biacore™ machine may be used to determine the binding constant of a complex between two proteins. The dissociation constant (K_{D}) for the complex may be determined by monitoring changes in the refractive index with respect to time as buffer is passed over the chip. Other suitable assays for measuring the binding of one protein to another include, for example, immunoassays such as enzyme linked immunosorbent assays (ELISA) and radioimmunoas says (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, ELISA, analytical ultracentrifugation, spectroscopy and surface plasmon resonance (Biacore™) analysis (see, e.g., Scatchard et al. (1949) Ann. N.Y. Acad. Sci. 51:660, Wilson (2002) Science 295:2103, Wolff et al. (1993) Cancer Res. 53:2560, and U.S. Pat. Nos. 5,283,173 and 5,468,614), flow cytometry, sequencing and other methods for detection of expressed nucleic acids. In one example, apparent affinity for a target is measured by assessing binding to various concentrations of tetramers, for example, by flow cytometry using labeled multimers, such as MHC-antigen peptide tetramers. In one representative example, apparent K_{D} of a binding protein is measured using 2-fold dilutions of labeled tetramers at a range of concentrations, followed by determination of binding curves by non-linear regression, apparent K_{D} being determined as the concentration of ligand that yielded half-maximal binding.

### VII. Uses and Methods

### A. Diagnostic Methods

In some aspects, provided herein are diagnostic methods for determining whether a subject has exposure to and/or protection from SARS-CoV-2 comprising: (a) incubating a sample (e.g., blood, isolated PBMCs or isolated T cells) obtained from the subject with a SARS-CoV-2 immunogenic peptides described herein (e.g., a peptide epitope selected from Table 1A, 1B, 1C, 1D, 1E, and/or 1F), a MHC-peptide complex described herein, or a cell presenting a MHC-peptide complex described herein; and (b) detecting the level of reactivity; wherein a higher level of reactivity compared to a control level indicates that the subject has exposure to and/or protection from SARS-CoV-2.

In some embodiments, the level of reactivity is indicated by T cell activation or effector function, such as, but not limited to, T cell proliferation, killing, or cytokine release. The control level may be a reference number or a level of a healthy subject who has no exposure to SARS-CoV-2.

### B. Therapeutic Methods

In some aspects, provided herein are methods for preventing and/or treating COVID-19 (i.e., a SARS-CoV-2 infection), and/or for inducing an immune response against a SARS-CoV-2 protein or fragment thereof. In certain embodiments, the method comprises administering to a subject an immunogenic composition described herein.

The methods described herein may be used to treat any subject in need thereof. As used herein, a “subject in need thereof” includes any subject who has COVID-19, who has had COVID-19 and/or who is predisposed to COVID-19. For example, in some embodiments, the subject has a COVID-19. In some embodiments, the subject has undergone treatments for COVID-19. In some embodiments, the subject is predisposed to COVID-19 due to age, or having a compromised immune system or other serious underlying medical conditions that predisposes the subject to COVID-19.

The pharmaceutical compositions disclosed herein may be delivered by any suitable route of administration, including orally and parenterally. In certain embodiments the pharmaceutical compositions are delivered generally (e.g., via oral or parenteral administration). In specific embodiments, the pharmaceutical compositions is administered by subcutaneous injection.

The dosage of the subject agent may be determined by reference to the plasma concentrations of the agent. For example, the maximum plasma concentration (Cmax) and the area under the plasma concentration-time curve from time 0 to infinity (AUC (0-4)) may be used. Dosages include those that produce the above values for Cmax and AUC (0-4) and other dosages resulting in larger or smaller values for those parameters.

Actual dosage levels of the active ingredients in the pharmaceutical compositions may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular agent employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could prescribe and/or administer doses of the agents employed in the pharmaceutical composition at levels lower than that required in order to achieve the desired therapeutic effect and gradually increase the dosage until the desired effect is achieved.

In general, a suitable daily dose of an agent described herein will be that amount of the agent which is the lowest dose effective to produce a therapeutic effect. Such an effective dose will generally depend upon the factors described above.

In some embodiments, the immunogenic composition comprises an amount of a SRS-CoV-2 immunogenic peptide in combination with an adjuvant that constitutes a pharmaceutical dosage unit. A pharmaceutical dosage unit is defined herein as the amount of active ingredients (e.g., SRS-CoV-2 immunogenic peptides and/or adjuvant) that is applied to a subject at a given time point. A pharmaceutical dosage unit may be applied to a subject in a single volume, e.g., a single shot, or may be applied in 2, 3, 4, 5 or more separate volumes or shots that are applied at different locations of the body, for instance in the right and the left limb. Reasons for applying a single pharmaceutical dosage unit in separate volumes may be multiples, such as avoid negative side effects, avoiding antigenic competition and/or composition analytics considerations. It is to be understood herein that the separate volumes of a pharmaceutical dosage may differ in composition, i.e., may comprise different kinds or composition of active ingredients and/or adjuvants.

A pharmaceutical dosage unit may be an effective amount or part of an effective amount. An “effective amount” is to be understood herein as an amount or dose of active ingredients required to prevent and/or reduce the symptoms of a disease (e.g., COVID-19) relative to an untreated patient. The effective amount of active compound(s) used to practice the present invention for preventive and/or therapeutic treatment of COVID-19 varies depending upon the manner of administration, the age, body weight, and general health of the subject. Ultimately, the attending physician or veterinarian will decide the appropriate amount and dosage regimen. Such amount is referred to as an “effective” amount. This effective amount may also be the amount that is able to induce an effective cellular T cell response in the subject to be treated, or more preferably an effective systemic cellular T cell response.

In one aspect, provided herein is a method of eliciting in a subject an immune response to a cell that is infected with SARS-CoV-2 virus. The method comprises: administering to the subject a pharmaceutical composition described herein, wherein the pharmaceutical composition, when administered to the subject, elicits an immune response to the cell that is infected with SARS-CoV-2 virus.

Generally, the immune response may include a humoral immune response, a cell-mediated immune response, or both.

A humoral response may be determined by a standard immunoassay for antibody levels in a serum sample from the subject receiving the pharmaceutical composition. A cellular immune response is a response that involves T cells and may be determined in vitro or in vivo. For example, a general cellular immune response may be determined as the T cell proliferative activity in cells (e.g., peripheral blood leukocytes (PBLs)) sampled from the subject at a suitable time following the administering of a pharmaceutical composition. Following incubation of e.g., PBMCs with a stimulator for an appropriate period, [³H]thymidine incorporation may be determined. The subset of T cells that is proliferating may be determined using flow cytometry.

In certain aspects, the methods provided herein include administering to both human and non-human mammals. Veterinary applications also are contemplated. In some embodiments, the subject may be any living organism in which an immune response may be elicited. Examples of subjects include, without limitation, humans, livestock, dogs, cats, mice, rats, and transgenic species thereof.

In some embodiments, the pharmaceutical composition may be administered at any time that is appropriate. For example, the administering may be conducted before or during treatment of a subject having a COVID-19, and continued after the SARS-CoV-2 infection becomes clinically undetectable. The administering also may be continued in a subject showing signs of recurrence.

In some embodiments, the pharmaceutical composition may be administered in a therapeutically or a prophylactically effective amount. Administering the pharmaceutical composition to the subject may be carried out using known procedures, and at dosages and for periods of time sufficient to achieve a desired effect.

In some embodiments, the pharmaceutical composition may be administered to the subject at any suitable site. The route of administering may be parenteral, intramuscular, subcutaneous, intradermal, intraperitoneal, intranasal, intravenous (including via an indwelling catheter), via an afferent lymph vessel, or by any other route suitable in view of the subject’s condition. Preferably, the dose will be administered in an amount and for a period of time effective in bringing about a desired response, be it eliciting the immune response or the prophylactic or therapeutic treatment of the SARS-CoV-2 infection and/or symptoms associated therewith.

The pharmaceutical composition may be given subsequent to, preceding, or contemporaneously with other therapies including therapies that also elicit an immune response in the subject. For example, the subject may previously or concurrently be treated by other forms of immunomodulatory agents, such other therapies preferably provided in such a way so as not to interfere with the immunogenicity of the compositions described herein.

Administering may be properly timed by the care giver (e.g., physician, veterinarian), and may depend on the clinical condition of the subject, the objectives of administering, and/or other therapies also being contemplated or administered. In some embodiments, an initial dose may be administered, and the subject monitored for an immunological and/or clinical response. Suitable means of immunological monitoring include using patient’s peripheral blood lymphocyte (PBL) as responders and immunogenic peptides or MHC-peptide complexes described herein as stimulators. An immunological reaction also may be determined by a delayed inflammatory response at the site of administering. One or more doses subsequent to the initial dose may be given as appropriate, typically on a monthly, semimonthly, or a weekly basis, until the desired effect is achieved. Thereafter, additional booster or maintenance doses may be given as required, particularly when the immunological or clinical benefit appears to subside.

### C. Methods of Identifying Molecules That Bind to a Peptide in the Context of an MHC Molecule

In some aspect, provide herein are methods of identifying a peptide-binding molecule or antigen-binding fragment thereof that binds to a peptide epitope selected from Table 1A, 1B, 1C, 1D, 1E, and/or 1F.

In some embodiments, the peptide binding molecule, i.e., MHC-peptide binding molecule, is a molecule or portion thereof that possesses the ability to bind, e.g., specifically bind, to a peptide epitope that is presented or displayed in the context of an MHC molecule (MHC-peptide complex), such as on the surface of a cell. Exemplary peptide binding molecules include T cell receptors or antibodies, or antigen-binding portions thereof, including single chain immunoglobulin variable regions (e.g., scTCR, scFv) thereof, that exhibit specific ability to bind to an MHC-peptide complex. In some embodiments, the peptide binding molecule is a TCR or antigen-binding fragment thereof. In some embodiments, the peptide binding molecule is an antibody, such as a TCR-like antibody or antigen-binding fragment thereof. In some embodiments, the peptide binding molecule is a TCR-like CAR that contains an antibody or antigen binding fragment thereof, such as a TCR-like antibody, such as one that has been engineered to bind to MHC-peptide complexes. In some embodiments, the peptide binding molecule may be derived from natural sources, or it may be partly or wholly synthetically or recombinantly produced.

In some embodiments, a binding molecule that binds to a peptide epitope may be identified by contacting one or more candidate peptide binding molecules, such as one or more candidate TCR molecules, antibodies or antigen-binding fragments thereof, with an MHC-peptide complex, and assessing whether each of the one or more candidate binding molecules binds, such as specifically binds, to the MHC-peptide complex. The methods may be performed in vitro, ex vivo or in vivo. Methods are well-known in the art for screening, such as described in U.S. Pat. Publ. 2020/0102553.

In some embodiments, the methods include contacting a plurality or library of binding molecules, such as a plurality or library of TCRs or antibodies, with an MHC-restricted epitope and identifying or selecting molecules that specifically bind such an epitope. In some embodiments, a library or collection containing a plurality of different binding molecules, such as a plurality of different TCRs or a plurality of different antibodies, may be screened or assessed for binding to an MHC-restricted epitope. In some embodiments, such as for selecting an antibody molecule that specifically binds an MHC-restricted peptide, hybridoma methods may be employed.

In some embodiments, screening methods may be employed in which a plurality of candidate binding molecules, such as a library or collection of candidate binding molecules, are individually contacted with an peptide binding molecule, either simultaneously or sequentially. Library members that specifically bind to a particular MHC-peptide complex may be identified or selected. In some embodiments, the library or collection of candidate binding molecules may contain at least 2, 5, 10, 100, 10³, 10⁴, 10⁵, 10⁶, 10⁷, 10⁸, 10⁹, or more different peptide binding molecules.

In some embodiments, the methods may be employed to identify a peptide binding molecule, such as a TCR or an antibody, that exhibits binding for more than one MHC haplotype or more than one MHC allele. In some embodiments, the peptide binding molecule, such as a TCR or antibody, specifically binds or recognizes a peptide epitope presented in the context of a plurality of MHC class I haplotypes or alleles. In some embodiments, the peptide binding molecule, such as a TCR or antibody, specifically binds or recognizes a peptide epitope presented in the context of a plurality of MHC class II haplotypes or alleles.

A variety of assays are known for assessing binding affinity and/or determining whether a binding molecule specifically binds to a particular ligand (e.g., MHC-peptide complex). It is within the level of a skilled artisan to determine the binding affinity of a TCR for a T cell epitope of a target polypeptide, such as by using any of a number of binding assays that are well known in the art. For example, in some embodiments, a BIAcore machine may be used to determine the binding constant of a complex between two proteins. The dissociation constant (K_{D}) for the complex may be determined by monitoring changes in the refractive index with respect to time as buffer is passed over the chip. Other suitable assays for measuring the binding of one protein to another include, for example, immunoassays such as enzyme linked immunosorbent assays (ELISA) and radioimmunoas says (RIA), or determination of binding by monitoring the change in the spectroscopic or optical properties of the proteins through fluorescence, UV absorption, circular dichroism, or nuclear magnetic resonance (NMR). Other exemplary assays include, but are not limited to, Western blot, ELISA, analytical ultracentrifugation, spectroscopy and surface plasmon resonance (Biacore®) analysis (see, e.g., Scatchard et al. (1949) Ann. N.Y. Acad. Sci. 51:660; Wilson (2002) Science 295:2103; Wolff et al. (1993) Cancer Res. 53:2560; and U.S. Pat. Nos. 5,283,173, 5,468,614, or the equivalent), flow cytometry, sequencing and other methods for detection of expressed nucleic acids. In one example, apparent affinity for a TCR is measured by assessing binding to various concentrations of tetramers, for example, by flow cytometry using labeled tetramers. In one example, apparent K_{D} of a TCR is measured using 2-fold dilutions of labeled tetramers at a range of concentrations, followed by determination of binding curves by non-linear regression, apparent K_{D} being determined as the concentration of ligand that yielded half-maximal binding.

In some embodiments, the methods may be used to identify binding molecules that bind only if the particular peptide is present in the complex, and not if the particular peptide is absent or if another, non-overlapping or unrelated peptide is present. In some embodiments, the binding molecule does not substantially bind the MHC in the absence of the bound peptide, and/or does not substantially bind the peptide in the absence of the MHC. In some embodiments, the binding molecules are at least partially specific. In some embodiments, an exemplary identified binding molecule may bind to an MHC-peptide complex if the particular peptide is present, and also bind if a related peptide that has one or two substitutions relative to the particular peptide is present.

In some embodiments, an identified antibody, such as a TCR-like antibody, may be used to produce or generate a chimeric antigen receptors (CARs) containing a non-TCR antibody that specifically binds to a MHC-peptide complex.

In some embodiments, the methods of identifying a peptide binding molecule, such as a TCR or TCR-like antibody or TCR-like CAR, may be used to engineer cells expressing or containing an peptide binding molecule. In some embodiments, a cell or engineered cell is a T cell. In some embodiments, the T cell is a CD4+ or CD8+ T cell. In some embodiments, the peptide binding molecule recognizes a MHC class I peptide complex, an MHC class II peptide complex and/or an MHC-E peptide complex. In some embodiments, an peptide binding molecule, such as a TCR or antibody or CAR, that specifically recognizes a peptide in the context of an MHC class I may be used to engineer CD8+ T cells. In some embodiments, also provided is a composition of engineered CD8+ T cells expressing or containing the TCR, antibody or CAR, for recognition of a peptide presented in the context of MHC class I. In any of such embodiments, the cells may be used in methods of adoptive cell therapy.

In some embodiments, TCR libraries may be generated by amplification of the repertoire of Vα and Vβ from T cells isolated from a subject, including cells present in PBMCs, spleen or other lymphoid organ. In some cases, T cells may be amplified from tumor-infiltrating lymphocytes (TILs). In some embodiments, TCR libraries may be generated from CD4+ or CD8+ cells. In some embodiments, the TCRs may be amplified from a T cell source of a normal of healthy subject, i.e., normal TCR libraries. In some embodiments, the TCRs may be amplified from a T cell source of a diseased subject, i.e., diseased TCR libraries. In some embodiments, degenerate primers are used to amplify the gene repertoire of Vα and VP, such as by RT-PCR in samples, such as T cells, obtained from humans. In some embodiments, scTv libraries may be assembled from naive Vα and Vβ libraries in which the amplified products are cloned or assembled to be separated by a linker. Depending on the source of the subject and cells, the libraries may be HLA allele-specific.

Alternatively, in some embodiments, TCR libraries may be generated by mutagenesis or diversification of a parent or scaffold TCR molecule. For example, in some aspects, a subject, e.g., human or other mammal such as a rodent, may be vaccinated with a peptide, such as a peptide identified by the present methods. In some embodiments, a sample may be obtained from the subject, such as a sample containing blood lymphocytes. In some instances, binding molecules, e.g., TCRs, may be amplified out of the sample, e.g., T cells contained in the sample. In some embodiments, antigen-specific T cells may be selected, such as by screening to assess CTL activity against the peptide. In some aspects, TCRs, e.g., present on the antigen-specific T cells, may be selected, such as by binding activity, e.g., particular affinity or avidity for the antigen. In some aspects, the TCRs are subjected to directed evolution, such as by mutagenesis, e.g., of the α or β chain. In some aspects, particular residues within CDRs of the TCR are altered. In some embodiments, selected TCRs may be modified by affinity maturation. In some aspects, a selected TCR may be used as a parent scaffold TCR against the antigen.

In some embodiments, the subject is a human, such as a human with COVID-19. In some embodiments, the subject is a rodent, such as a mouse. In some such embodiments, the mouse is a transgenic mouse, such as a mouse expressing human MHC (i.e., HLA) molecules, such as HLA-A2. See Nicholson et al. Adv Hematol. 2012; 2012: 404081.

In some embodiments, the subject is a transgenic mouse expressing human TCRs or is an antigen-negative mouse. See Li et al. (2010) Nat Med. 161029-1034; Obenaus et al. (2015) Nat Biotechnol. 33:402-407. In some aspects the subject is a transgenic mouse expressing human HLA molecules and human TCRs.

In some embodiments, such as where the subject is a transgenic HLA mouse, the identified TCRs are modified, e.g., to be chimeric or humanized. In some aspects, the TCR scaffold is modified, such as analogous to known antibody humanizing methods.

In some embodiments, such a scaffold molecule is used to generate a library of TCRs.

For example, in some embodiments, the library includes TCRs or antigen-binding portions thereof that have been modified or engineered compared to the parent or scaffold TCR molecule. In some embodiments, directed evolution methods may be used to generate TCRs with altered properties, such as with higher affinity for a specific MHC-peptide complex. In some embodiments, display approaches involve engineering, or modifying, a known, parent or reference TCR. For example, in some cases, a wild-type TCR may be used as a template for producing mutagenized TCRs in which in one or more residues of the CDRs are mutated, and mutants with an desired altered property, such as higher affinity for a desired target antigen, are selected. In some embodiments, directed evolution is achieved by display methods including, but not limited to, yeast display (Holler et al. (2003) Nat Immunol 4:55-62; Holler et al. (2000) Proc Natl Acad Sci USA 97:5387-5392), phage display (Li et al. (2005) Nat Biotechnol 23:349-354), or T cell display (Chervin et al. (2008) J Immunol Methods 339:175-184).

In some embodiments, the libraries may be soluble. In some embodiments, the libraries are display libraries in which the TCR is displayed on the surface of a phage or cell, or attached to a particle or molecule, such as a cell, ribosome or nucleic acid, e.g., RNA or DNA. Typically, the TCR libraries, including normal and disease TCR libraries or diversified libraries, may be generated in any form, including as a heterodimer or as a single chain form. In some embodiments, one or more members of the TCR may be a two-chain heterodimer. In some embodiments, pairing of the Vα and Vβ chains may be promoted by introduction of a disulfide bond. In some embodiments, members of the TCR library may be a TCR single chain (scTv or ScTCR), which, in some cases, may include a Vα and Vβ chain separated by a linker. Further, in some cases, upon screening and selection of a TCR from the library, the selected member may be generated in any form, such as a full-length TCR heterodimer or single-chain form or as antigen-binding fragments thereof.

Other methods of identifying molecules that bind to a peptide in the context of an MHC molecule are also described in U.S. Pat. Application 2020/0182884, which is incorporated by reference herein in its entirety.

### D. Monitoring of Effects During Clinical Trials

Monitoring the influence of a SARS-CoV-2 therapy (e.g., compounds, drugs, vaccines, or cell therapies) on T cell reactivity (e.g., the presence of binding and/or T cell activation and/or effector function), can be applied not only in basic candidate peptide-binding molecule screening, but also in clinical trials. For example, the effectiveness of SARS-CoV-2 immunogenic peptides or compositions, nucleic acids encoding such SARS-CoV-2 immunogenic peptides, MHC-peptide complexes, or cells expressing nucleic acids, vectors, immunogenic peptides or MHC-peptide complexes as described herein to increase immune response (e.g., T cell immune response) against SARS-CoV-2 infection, can be monitored in clinical trials of subjects afflicted with COVID-19. In such clinical trials, the presence of binding and/or T cell activation and/or effector function (e.g., T cell proliferation, killing, or cytokine release), can be used as a “read out” or marker of the phenotype of a particular cell, tissue, or system. Similarly, the effectiveness of an adaptive T cell therapy with T cells engineered to express a TCR determined by a screening assay as described herein, or with T cells that stimulated with immunogenic peptides, MHC-peptide complexes, or cells presenting MHC-peptide complexes as described herein to increase immune response to cells that are infected by SARS-CoV-2, can be monitored in clinical trials of subjects afflicted with COVID-19. In such clinical trials, the presence of binding and/or T cell activation and/or effector function (e.g., T cell proliferation, killing, or cytokine release), can be used as a “read out” or marker of the phenotype of a particular cell, tissue, or system.

In one embodiment, the present invention provides a method for monitoring the effectiveness of treatment of a subject with a SARS-CoV-2 therapy (e.g., compounds, drugs, vaccines, or cell therapies) including the steps of a) determining the presence or level of reactivity between T cells obtained from the subject and one or more immunogenic peptides or one or more stable MHC-peptide complexes described herein, in a first sample obtained from the subject prior to providing at least a portion of the SARS-CoV-2 therapy to the subject, and b) determining the presence or level of reactivity between the one more immunogenic peptides, or the one or more stable MHC-peptide complexes described herein, and T cells obtained from the subject present in a second sample obtained from the subject following provision of the portion of the SARS-CoV-2 therapy, wherein the presence or a higher level of reactivity in the second sample, relative to the first sample, is an indication that the therapy is efficacious for treating SARS-CoV-2 in the subject.

For example, increased administration of the SARS-CoV-2 therapy may be desirable to increase the presence or level of reactivity between T cells obtained from the subject and one or more immunogenic peptides or one or more stable MHC-peptide complexes described herein, i.e., to increase the effectiveness of the SARS-CoV-2 therapy. According to such an embodiment, the presence or level of reactivity between T cells obtained from the subject and one or more immunogenic peptides or one or more stable MHC-peptide complexes described herein may be used as an indicator of the effectiveness of a SARS-CoV-2 therapy, even in the absence of an observable phenotypic response. Similarly, analysis of the presence or level of reactivity between T cell and one or more immunogenic peptides or one or more stable MHC-peptide complexes described herein, such as by a direct binding assay, fluorescence activated cell sorting (FACS), enzyme linked immunosorbent assay (ELISA), radioimmune assay (RIA), immunochemically, Western blot, or intracellular flow assay, can also be used to select patients who will receive SARS-CoV-2 therapy.

For example, in a direct binding assay, immunogenic peptides or MHC-peptide complexes can be coupled with a radioisotope or enzymatic label such that binding can be determined by detecting the labeled immunogenic peptides or MHC-peptide complexes. For example, the immunogenic peptides or MHC-peptide complexes can be labeled with ¹²⁵I, ³⁵S, ¹⁴C, or ³H, either directly or indirectly, and the radioisotope detected by direct counting of radioemmission or by scintillation counting. Alternatively, the immunogenic peptides or MHC-peptide complexes can be enzymatically labeled with, for example, horseradish peroxidase, alkaline phosphatase, or luciferase, and the enzymatic label detected by determination of conversion of an appropriate substrate to product. Determining the interaction between immunogenic peptides or MHC-peptide complexes and T cells can also be accomplished using standard binding or enzymatic analysis assays. In one or more embodiments of the above described assay methods, it may be desirable to immobilize immunogenic peptides or MHC-peptide complexes to accommodate automation of the assay.

Binding of immunogenic peptides or MHC-peptide complexes to T cells can be accomplished in any vessel suitable for containing the reactants. Non-limiting examples of such vessels include microtiter plates, test tubes, and micro-centrifuge tubes. Immobilized forms of the immunogenic peptides or MHC-peptide complexes described herein can also include immunogenic peptides or MHC-peptide complexes bound to a solid phase like a porous, microporous (with an average pore diameter less than about one micron) or macroporous (with an average pore diameter of more than about 10 microns) material, such as a membrane, cellulose, nitrocellulose, or glass fibers; a bead, such as that made of agarose or polyacrylamide or latex; or a surface of a dish, plate, or well, such as one made of polystyrene.

In some embodiments, the reactivity of T cells to one or more immunogenic peptides or one or more stable MHC-peptide complexes described herein the presence of binding and/or T cell activation and/or effector function. The term “T cell activation” refers to T lymphocytes selected from proliferation, differentiation, cytokine secretion, release of cytotoxic effector molecules, cytotoxic activity, and expression of activation markers, particularly refers to one or more cellular responses of cytotoxic T lymphocytes.

The reactivity of T cells to one or more immunogenic peptides or one or more stable MHC-peptide complexes can be measured according to any of the T cell functional parameters described herein (e.g., proliferation, cytokine release, cytotoxicity, changes in cell surface marker phenotype, etc.).

Cytokine production and/or release can be measured by methods well known in the art, for example, ELISA, enzyme-linked immune absorbent spot (ELISPOT), Luminex® assay, intracellular cytokine staining, and flow cytometry, and combinations thereof (e.g., intracellular cytokine staining and flow cytometry). It can be determined according to the method implemented.

The term “cytokine” as used herein refers to a molecule that mediates and/ r regulates a biological or cellular function or process (e.g., immunity, inflammation, and hematopoiesis). The term “cytokine” as used herein includes “lymphokines”, “chemokines”, “monokines”, and “interleukins”. Examples of useful cytokines are GM-CSF, IL-1α, IL-1β, IL-2, IL-3, IL-4, IL-5, IL-6, IL-7, IL-8, IL-10, IL-12, IL-15, IFN-α, IFN-β, IFN-γ, MIP-1α, MIP-1β, TGF-β, TNF-α, and TNF-β.

The proliferation and clonal expansion of T cells resulting from antigen-specific induction or stimulation of an immune response can be determined, for example, through incorporation of a non-radioactive assay such as a tritiated thymidine assay or MTT assay.

Cytotoxicity assays to determine CTL activity can be performed using any one of several techniques and methods routinely practiced in the art (e.g., Henkart et al. (2003) Fundamental Immunology 1127-1150). Additional description of methods for measuring antigen-specific T cell reactivity can be found in, for example, U.S. Pat. 10,208,086 and U.S. Pat. Application 2017/0209573, each of which is incorporated by reference herein in its entirety.

### VIII. Cell Therapy

In certain aspects, the methods include adoptive cell therapy, whereby genetically engineered cells expressing the provided molecules targeting an MHC-restricted epitope (e.g., cells expressing a TCR or TCR-like CAR) are administered to subjects. Such administration may promote activation of the cells (e.g., T cell activation) in an antigen-targeted manner, such that the cells infected with SARS-CoV-2 are targeted for destruction.

Thus, the provided methods and uses include methods and uses for adoptive cell therapy. In some embodiments, the methods include administration of the cells or a composition containing the cells to a subject, tissue, or cell, such as one having, at risk for, or suspected of having the disease, condition or disorder. In some embodiments, the cells, populations, and compositions are administered to a subject having the particular disease or condition to be treated, e.g., via adoptive cell therapy, such as adoptive T cell therapy. In some embodiments, the cells or compositions are administered to the subject, such as a subject having or at risk for the disease or condition. In some aspects, the methods thereby treat, e.g., ameliorate one or more symptom of the disease or condition.

Methods for administration of cells for adoptive cell therapy are known and may be used in connection with the provided methods and compositions. For example, adoptive T cell therapy methods are described, e.g., in U.S. Pat. Application Publication No. 2003/0170238 to Gruenberg et al; U.S. Pat. No. 4,690,915 to Rosenberg; Rosenberg (2011) Nat Rev Clin Oncol. 8:577-585). See, e.g., Themeli et al. (2013) Nat Biotechnol. 31: 928-933; Tsukahara et al. (2013) Biochem Biophys Res Commun 438: 84-89; Davila et al. (2013) PLoS ONE 8:e61338.

In some embodiments, the cell therapy, e.g., adoptive cell therapy, e.g., adoptive T cell therapy, is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject, to whom the cells, cell populations, or compositions are administered is a primate, such as a human. In some embodiments, the primate is a monkey or an ape. The subject may be male or female and may be any suitable age, including infant, juvenile, adolescent, adult, and geriatric subjects. In some embodiments, the subject is a non-primate mammal, such as a rodent. In some examples, the patient or subject is a validated animal model for disease, adoptive cell therapy, and/or for assessing toxic outcomes such as cytokine release syndrome (CRS).

The binding molecules, such as TCRs, TCR-like antibodies and chimeric receptors (e.g., CARs) containing the TCR-like antibodies and cells expressing the same, may be administered by any suitable means, for example, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon’s injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. Dosing and administration may depend in part on whether the administration is brief or chronic. Various dosing schedules include but are not limited to single or multiple administrations over various time-points, bolus administration, and pulse infusion.

For the prevention or treatment of disease, the appropriate dosage of the binding molecule or cell may depend on the type of disease to be treated, the type of binding molecule, the severity and course of the disease, whether the binding molecule is administered for preventive or therapeutic purposes, previous therapy, the patient’s clinical history and response to the binding molecule, and the discretion of the attending physician. The compositions and molecules and cells are in some embodiments suitably administered to the patient at one time or over a series of treatments.

In certain embodiments, the cells, or individual populations of sub-types of cells, are administered to the subject at a range of about one million to about 100 billion cells and/or that amount of cells per kilogram of body weight, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges and/or per kilogram of body weight. Dosages may vary depending on attributes particular to the disease or disorder and/or patient and/or other treatments.

In some embodiments, for example, where the subject is a human, the dose includes fewer than about 1×10⁸ total recombinant receptor (e.g., CAR)-expressing cells, T cells, or peripheral blood mononuclear cells (PBMCs), e.g., in the range of about 1×10⁶ to 1×10⁸ such cells, such as 2×10⁶, 5×10⁶, 1×10⁷, 5×10⁷, or 1×10⁸ or total such cells, or the range between any two of the foregoing values.

In some embodiments, the cells or binding molecules (e.g., TCR or TCR-like antibodies) are administered as part of a combination treatment, such as simultaneously with or sequentially with, in any order, another therapeutic intervention, such as another antibody or engineered cell or receptor or agent, such as a cytotoxic or therapeutic agent.

The cells or binding molecules (e.g., TCR or TCR-like antibodies) in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells or binding molecules (e.g., TCR or TCR-like antibodies) are administered prior to the one or more additional therapeutic agents. In some embodiments, the cells or binding molecules (e.g., TCR or TCR-like antibodies) are administered after to the one or more additional therapeutic agents.

Once the cells are administered to a mammal (e.g., a human), the biological activity of the engineered cell populations and/or binding molecules (e.g., TCR or TCR-like antibodies) in some aspects is measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells may be measured using any suitable method known in the art, such as cytotoxicity assays described in, for example, Kochenderfer et al. (2009) J. Immunotherapy 32: 689-702, and Herman et al. (2004) J. Immunological Methods 285:25-40. In certain embodiments, the biological activity of the cells also may be measured by assaying expression and/or secretion of certain cytokines, such as CD 107a, IFNγ, IL-2, and TNF. In some aspects the biological activity is measured by assessing clinical outcome, such as reduction in tumor burden or load.

In certain embodiments, engineered cells are modified in any number of ways, such that their therapeutic or prophylactic efficacy is increased. For example, the engineered CAR or TCR expressed by the population may be conjugated either directly or indirectly through a linker to a targeting moiety. The practice of conjugating compounds, e.g., the CAR or TCR, to targeting moieties is known in the art. See, for instance, Wadwa et al. (1995) J. Drug Targeting 3: 111, and U.S. Pat. No. 5,087,616.

In certain aspects, the SARS-CoV-2 immunogenic peptides described herein, or a nucleic acid encoding such SARS-CoV-2 immunogenic peptides, may be used in compositions and methods for providing SARS-CoV-2-primed, antigen-presenting cells, and/or SARS-CoV-2-specific lymphocytes generated with these antigen-presenting cells. In some embodiments, such antigen-presenting cells and/or lymphocytes are used in the treatment and/or prevention of COIVD-19 (i.e., SARS-CoV-2 infection).

In some aspects, provided herein are methods for making SARS-CoV-2-primed, antigen-presenting cells by contacting antigen-presenting cells with a SARS-CoV-2 immunogenic polypeptide described herein, or nucleic acids encoding the at least one SARS-CoV-2 immunogenic polypeptide, alone or in combination with an adjuvant, in vitro under a condition sufficient for the at least one SARS-CoV-2 immunogenic polypeptide to be presented by the antigen-presenting cells.

In some embodiments, the SARS-CoV-2 immunogenic polypeptide, or nucleic acid encoding the SARS-CoV-2 immunogenic polypeptide, alone or in combination with an adjuvant, may be contacted with a homogenous, substantially homogenous, or heterogeneous composition comprising antigen-presenting cells. For example, the composition may include but is not limited to whole blood, fresh blood, or fractions thereof such as, but not limited to, peripheral blood mononuclear cells, buffy coat fractions of whole blood, packed red cells, irradiated blood, dendritic cells, monocytes, macrophages, neutrophils, lymphocytes, natural killer cells, and natural killer T cells. If, optionally, precursors of antigen-presenting cells are used, the precursors may be cultured under suitable culture conditions sufficient to differentiate the precursors into antigen-presenting cells. In some embodiments, the antigen-presenting cells (or precursors thereof) are selected from monocytes, macrophages, cells of myeloid lineage, B cells, dendritic cells, or Langerhans cells.

The amount of the SARS-CoV-2 immunogenic polypeptide, or nucleic acid encoding the SARS-CoV-2 immunogenic polypeptide, alone or in combination with an adjuvant, to be placed in contact with antigen-presenting cells may be determined by one of ordinary skill in the art by routine experimentation. Generally, antigen-presenting cells are contacted with the SARS-CoV-2 immunogenic polypeptide, or nucleic acid encoding the SARS-CoV-2 immunogenic polypeptide, alone or in combination with an adjuvant, for a period of time sufficient for cells to present the processed forms of the antigens for the modulation of T cells. In one embodiment, antigen-presenting cells are incubated in the presence of the SARS-CoV-2 immunogenic polypeptide, or nucleic acid encoding the SARS-CoV-2 immunogenic polypeptide, alone or in combination with an adjuvant, for less than about a week, illustratively, for about 1 minute to about 48 hours, about 2 minutes to about 36 hours, about 3 minutes to about 24 hours, about 4 minutes to about 12 hours, about 6 minutes to about 8 hours, about 8 minutes to about 6 hours, about 10 minutes to about 5 hours, about 15 minutes to about 4 hours, about 20 minutes to about 3 hours, about 30 minutes to about 2 hours, and about 40 minutes to about 1 hour. The time and amount of the SARS-CoV-2 immunogenic polypeptide, or nucleic acid encoding the SARS-CoV-2 immunogenic polypeptide, alone or in combination with an adjuvant, necessary for the antigen presenting cells to process and present the antigens may be determined, for example using pulse-chase methods wherein contact is followed by a washout period and exposure to a read-out system e.g., antigen reactive T cells.

In certain embodiments, any appropriate method for delivery of antigens to the endogenous processing pathway of the antigen-presenting cells may be used. Such methods include but are not limited to, methods involving pH-sensitive liposomes, coupling of antigens to adjuvants, apoptotic cell delivery, pulsing cells onto dendritic cells, delivering recombinant chimeric virus-like particles (VLPs) comprising antigen to the MHC class I processing pathway of a dendritic cell line.

In one embodiment, solubilized SARS-CoV-2 immunogenic polypeptide is incubated with antigen-presenting cells. In some embodiments, the SARS-CoV-2 immunogenic polypeptide may be coupled to a cytolysin to enhance the transfer of the antigens into the cytosol of an antigen-presenting cell for delivery to the MHC class I pathway. Exemplary cytolysins include saponin compounds such as saponin-containing Immune Stimulating Complexes (ISCOM5), pore-forming toxins (e.g., an alpha-toxin), and natural cytolysins of gram-positive bacteria such as listeriolysin O (LLO), streptolysin O (SLO), and perfringolysin O (PFO).

In some embodiments, antigen-presenting cells, such as dendritic cells and macrophage, may be isolated according to methods known in the art and transfected with polynucleotides by methods known in the art for introducing a nucleic acid encoding the SARS-CoV-2 immunogenic polypeptide into the antigen-presenting cell. Transfection reagents and methods are known in the art and commercially available. For example, RNA encoding SARS-CoV-2 immunogenic polypeptide may be provided in a suitable medium and combined with a lipid (e.g., a cationic lipid) prior to contact with antigen-presenting cells. Non-limiting examples of such lipids include LIPOFECTIN™ and LIPOFECTAMINE™. The resulting polynucleotide-lipid complex may then be contacted with antigen-presenting cells. Alternatively, the polynucleotide may be introduced into antigen-presenting cells using techniques such as electroporation or calcium phosphate transfection. The polynucleotide-loaded antigen-presenting cells may then be used to stimulate T lymphocyte (e.g., cytotoxic T lymphocyte) proliferation in vivo or ex vivo. In one embodiment, the ex vivo expanded T lymphocyte is administered to a subject in a method of adoptive immunotherapy.

In certain aspects, provided herein is a composition comprising antigen-presenting cells that have been contacted in vitro with a SARS-CoV-2 immunogenic polypeptide, or a nucleic acid encoding a SARS-CoV-2 immunogenic polypeptide, alone or in combination with an adjuvant under a condition sufficient for a SARS-CoV-2 immunogenic epitope to be presented by the antigen-presenting cells.

In some aspects, provided herein is a method for preparing lymphocytes specific for a SARS-CoV-2 protein. The method comprises contacting lymphocytes with the antigen-presenting cells described above under conditions sufficient to produce a SARS-CoV-2 protein-specific lymphocyte capable of eliciting an immune response against a cell that is infected by the SARS-CoV-2 virus. Thus, the antigen-presenting cells also may be used to provide lymphocytes, including T lymphocytes and B lymphocytes, for eliciting an immune response against cell that is infected by the SARS-CoV-2 virus.

In some embodiments, a preparation of T lymphocytes is contacted with the antigen-presenting cells described above for a period of time, (e.g., at least about 24 hours) to priming the T lymphocytes to a SARS-CoV-2 immunogenic epitope presented by the antigen-presenting cells.

In some embodiments, a population of antigen-presenting cells may be co-cultured with a heterogeneous population of peripheral blood T lymphocytes together with a SARS-CoV-2 immunogenic polypeptide, or a nucleic acid encoding a SARS-CoV-2 immunogenic polypeptide, alone or in combination with an adjuvant. The cells may be co-cultured for a period of time and under conditions sufficient for SARS-CoV-2 epitopes included in the SARS-CoV-2 polypeptides to be presented by the antigen-presenting cells and the antigen-presenting cells to prime a population of T lymphocytes to respond to cells is infected by the SARS-CoV-2 virus. In certain embodiments, provided herein are T lymphocytes and B lymphocytes that are primed to respond to cells that is infected by the SARS-CoV-2 virus.

T lymphocytes may be obtained from any suitable source such as peripheral blood, spleen, and lymph nodes. The T lymphocytes may be used as crude preparations or as partially purified or substantially purified preparations, which may be obtained by standard techniques including, but not limited to, methods involving immunomagnetic or flow cytometry techniques using antibodies.

In certain aspects, provided herein is a composition (e.g., a pharmaceutical composition) comprising the antigen-presenting cells or the lymphocytes described above, and a pharmaceutically acceptable carrier and/or diluent. In some embodiments, the composition further comprises an adjuvant as described above.

In certain aspects, provided herein is a method for eliciting an immune response to the cell is infected by the SARS-CoV-2 virus, the method comprising administering to the subject the antigen-presenting cells or the lymphocytes described above in effective amounts sufficient to elicit the immune response. In some embodiments, provided herein is a method for treatment or prophylaxis of COVID-19, the method comprising administering to the subject an effective amount of the antigen-presenting cells or the lymphocytes described above. In one embodiment, the antigen-presenting cells or the lymphocytes are administered systemically, preferably by injection. Alternately, one may administer locally rather than systemically, for example, via injection directly into tissue, preferably in a depot or sustained release formulation.

In certain embodiments, the antigen-primed antigen-presenting cells described herein and the antigen-specific T lymphocytes generated with these antigen-presenting cells may be used as active compounds in immunomodulating compositions for prophylactic or therapeutic treatment of COVID-19. In some embodiments, the SARS-CoV-2 -primed antigen-presenting cells described herein may be used for generating CD8⁺ T lymphocytes, CD4⁺ T lymphocytes, and/or B lymphocytes for adoptive transfer to the subject. Thus, for example, SARS-CoV-2 -specific lymphocyte may be adoptively transferred for therapeutic purposes in subjects afflicted with COVID-19.

In certain embodiments, the antigen-presenting cells and/or lymphocytes described herein may be administered to a subject, either by themselves or in combination, for eliciting an immune response, particularly for eliciting an immune response to cells are infected by the SARS-CoV-2 virus. In some embodiments, the antigen-presenting cells and/or lymphocytes may be derived from the subject (i.e., autologous cells) or from a different subject that is MHC matched or mismatched with the subject (e.g., allogeneic).

Single or multiple administrations of the antigen-presenting cells and lymphocytes may be carried out with cell numbers and treatment being selected by the care provider (e.g., physician). In some embodiments, the antigen-presenting cells and/or lymphocytes are administered in a pharmaceutically acceptable carrier. Suitable carriers may be growth medium in which the cells were grown, or any suitable buffering medium such as phosphate buffered saline. The cells may be administered alone or as an adjunct therapy in conjunction with other therapeutics.

### IX. Kits

The present invention also encompasses kits. For example, the kit may comprise immunogenic peptides, vectors comprising sequences encoding immunogenic peptides, stable MHC-peptide complexes as described herein, adjuvants, and combinations thereof, packaged in a suitable container and may further comprise instructions for using such reagents. The kit may also contain other components, such as administration tools packaged in a separate container.

The disclosure is further illustrated by the following examples which should not be construed as limiting. The contents of all references, patents and published patent applications cited throughout this application, as well as the Figures, are incorporated herein by reference.

### EXAMPLES

### Example 1: Materials and Methods for Examples 2 and 3

### A. Sample Collection Design

The study was approved by local institutional review boards (IRBs) at participating sites. All donors were provided written consent. The study was conducted in accordance with the Declaration of Helsinki (1996), approved by the Atlantic Health System Institutional Review Board and the Ochsner Clinic Foundation institutional Review Board and registered at clinicaltrials.gov #NCT04397900. Patients who had recovered from COVID-19 were eligible for this study. They were required to be >18 years of age and have laboratory-confirmed diagnosis of COVID-19 using CDC or state health labs or at hospitals using an FDA Emergency Use Authorized molecular assay. Time since discontinuation of isolation was required to be > 14 days and discontinuation of isolation followed CDC guidelines (accessed on Mar. 19, 2020) using either test-based or non-test-based criteria for patients either in home isolation or in isolation at hospitals. Patients were also required to have no anti-pyretic use for >17 days and be able to sign informed consent for blood draws for 4 tubes of whole blood with approximately 7.5 mL of blood per tube. Eligible patients were identified by the participating sites through advertising and direct contact. Case report forms did not contain identifying information. Samples were de-identified at the participating sites with an anonymous code assigned to each sample Anonymized blood samples were sent to TScan laboratories with limited demographic and clinical data. Demographics included age, gender and ethnicity. Clinical data included date of diagnosis, specifics of diagnostic testing, duration of symptoms and whether the patient required hospitalization, supplemental oxygen or ICU care/ ventilator support. Comorbidities and current medications were also recorded.

### B. Recruitment and Demographics

Convalescents who met eligibility criteria and consented to described procedures were enrolled and sampled from two sites, Atlantic Health (New Jersey, 51 samples) and Ochsner (New Orleans, 27 samples). These sites were key in treating patients from early epicenters of SARS-CoV-2 outbreaks. Recruitment materials clearly requested patients that had recovered from COVID-19 with the goal of designing effective vaccines and treatments for this indication. As of Jun. 9, 2020, 63 convalescent samples (47 Females, 16 Males) have been received from a variety of ethnic backgrounds with ages ranging from 21 to 76 years old. Average self-reported duration of symptoms was 18 days (1-80 days range) in females and 21 days (0-76 days range) in males. Hospitalizations made up ~32% of total convalescent samples received, with 31% requiring oxygen and 5% placed on a ventilator.

### C. Isolation of PBMCs and CD8 Memory T Cells

Blood samples were collected in four 10 mL VACUETTE® K2 EDTA vacutainer tubes (BD) and processed within 24-30 hours to PBMCs or CD8 memory T cells. Before processing, a 1 mL sample was removed and centrifuged at 500 xg for 10 minutes to obtain plasma. To isolate PBMCs, blood samples were diluted with an equal volume of MACS® separation buffer (phosphate buffered saline, 0.5% bovine serum albumin, 2 mM EDTA), then layered onto lymphocyte separation media (Corning) and centrifuged at 1200xg for 20 minutes. The interface was removed and washed once with MACS® buffer before further processing or cryopreservation. Memory CD8+ T cells were isolated from PBMCs using MACS® microbead kits according to the manufacturer’s instructions (Miltenyi). Following separation, purity was confirmed using antibodies to CD3 (APC-Cy7, HIT3a Biolegend), CD8 (AF647, SK1 Biolegend), CD45RA (BV510, HI100 Biolegend), CD45RO (PE, UCHL1 Biolegend), and CD57 (Pacific Blue, HNK-1 Biolegend). Immediately following isolation, memory CD8+ T cells were expanded by co-culturing with 2×10⁷ mitomycin C-treated (50 ug/mL, 30 minutes) allogenic PBMCs in the presence of 0.1 ug/mL anti-CD3 (OKT3, ebioscience), 50 U/mL recombinant IL-2 (Peprotech), 5 ng/mL IL-7, and 5 ng/mL IL-15 (R&D Systems). After 10 days of expansion, the cells were collected and cryopreserved.

### D. Library Design, Generation, and Cloning

All SARS-CoV-2 genomic sequences were obtained from the NCBI database on Mar. 15, 2020, encoding a total of 1,117 proteins. Additionally, full-genome coding sequences from SARS-CoV-1 (NC_004718.3), HCoV 229E (NC_002645.1), HCoV NL63 (NC_005831.2), HCoV OC43 (NC_006213.1) and HCoV HKU1 (NC_006577.2) were obtained from the NCBI viral database. Each protein encoded by these viral genomes was broken up into 61 amino acid (aa) fragments tiled every 20 aa, resulting in 4,278 unique protein tiles. As positive controls, 32 known antigenic peptides from CMV, EBV, and influenza (flu) were included (the CEF peptide pool, available at pubmed.ncbi.nlm.nih.gov/11792386/) in the context of two overlapping tiles with the surrounding viral sequence identified from the UniProt database, for a total of 64 protein tiles. The combined library of 4,342 protein fragments was reverse translated with 10 unique nucleotide sequences each to serve as internal replicates, for a total of 42,780 oligonucleotide sequences. All protein fragments were reverse translated with ten unique nucleotide sequences each, synthesized on a releasable microarray (Agilent), and cloned into the pHAGE™ CMV NFlagHA DEST vector.

### E. Generation of Reporter Cells

MHC-null HEK293T reporter cells, as described in Kula et al. (2019) Cell 178:P1016-P1028, were transduced to express one of each of the top nine most frequently occurring HLA alleles. Each reporter line was then transduced to express the COVID library described above. Library cells were maintained in culture at 1,500x representation of the antigen library until seeded for TScan screen co-culture.

### F. Screen Co-Culture

To stimulate T cells for antigen screens, 1.5×10⁷ CD8 memory T cells were thawed and re-stimulated as above by co-culturing with 3×10⁸ mitomycin C-treated (50 ug/mL, 30 minutes) allogenic PBMCs in the presence of 0.1 ug/mL anti-CD3 (OKT3, ebioscience), 50 U/mL recombinant IL-2 (Peprotech), 5 ng/mL IL-7 and 5 ng/mL IL-15 (R&D Systems). After expansion for 7 days, the T cells were added to library transduced reporter cell at an effector to target ratio of 1.25:1 and incubated at 37° C. for 4 hours.

### G. Cell Sorting

After incubation, cells were harvested by trypsinization and labeled with Annexin V magnetic microbeads (Miltenyi) according to the manufacturer’s instructions. Annexin-labeled cells were isolated using an AutoMACS Pro (Miltenyi). The antigen-expressing cells targeted by T cell killing sorted using a MoFlo® Astrios EQ cell sorter (Beckman Coulter). Cells that were IFP-positive, indicative of being recognized by T cells due to COVID antigen, were collected for antigen-expression cassette sequencing and subsequent enrichment analysis.

### H. HLA Typing of Patient Samples

Genomic DNA was extracted from sorted cells, such as 2×10⁶ patient cells, using the GeneJET™ genomic DNA purification kit (Thermo Scientific). Both type I and II HLA loci were amplified and Next Generation Sequencing libraries were prepared using the TruSight® HLA kit from CareDx. A pool of 24 samples were sequenced on Illumina MiSeq® sequencer with 150x2 cycles to get around 200x coverage of each locus. Sequence data were then analyzed using Assign TruSight® HLA v2.1 software to get the HLA typing information for each patient.

### I. COVID Peptidome Library Cloning and Lentivirus Packaging

COVID peptidome library was synthesized as 213-mer oligos by Agilent. 1 ng of oligos were PCR amplified and cloned into EcoRI site of pHAGE-CMV-n-FHA-IRES-puro vector using Gibson Assembly. Lentivirus of the library was packaged in Lenti-X cells and concentrated 100x for downstream reporter cell transduction.

### J. Screen Sample Processing and Sequencing

Genomic DNA extraction and next generation sequencing library preparation was done following a standard TScan screen protocol. Libraries of input sample and sorted samples were pooled and sequenced on Illumina MiSeq® sequencer. Reads were mapped to the designed COVID peptidome library to get the counts for each peptide. Specifically, genomic DNA (gDNA) was extracted from sorted cells using the GeneJET™ genomic DNA purification kit (Thermo Scientific). Samples were then subjected to 2 rounds of PCR. In the first round, primers amplified the antigen cassette from the extracted gDNA. Following PCR purification using AMPure™ XP beads, the second round of PCR added sequencing adaptors and sample-specific index sequences to the amplicon. Samples were then purified using AMPure™ XP beads, and pooled to equal quantities of DNA. Amplicons were sequenced on either an Illumina MiSeq® or Illumina NextSeq® sequencer using the standard Illumina sequencing primer. A 150-cycle kit was used for either instrument, and sequencing was performed with read lengths: 110 bpread 1, 8bp- i7 index, 8bp- i5 index.

### K. Data Analysis

The abundance of each peptide in the sorted screen sample was compared to the abundance in the original input library to calculate an enrichment score. Next, the peptide sequences were ranked based on their enrichment across the independent nucleotide barcodes or the screen replicates for each sample. To harness the TScan screen data and delineate the specific MHC-binding epitopes within each fragment, a maximum parsimony approach was applied. For each recognized protein fragment, the NetMHC algorithm was used to identify all predicted candidate MHC-binding epitopes. Next, the collective performance of all of the protein fragments in the library that contained each candidate epitope was analyzed. Finally, the minimum number of high-affinity binding epitopes that could account for the screen results was selected. These epitopes were found in the fragments that enriched, but were absent from fragments that failed to enrich. In this way, the redundancy in the library was leveraged along with what is known about MHC binding to robustly map specific peptide epitopes recognized by each patient.

Nucleotide sequences were mapped to individual nucleotide tiles and read counts for each library entity representing identical amino acid tiles were summed. The proportion of read counts for each tile was calculated for each screen replicate (n=4) and for the input for each pool of transduced reporter cells, and enrichments of each tile were calculated by dividing the proportion of the tile in the screen replicate by the proportion of the tile in the input library. A modified geometric mean of the enrichment of an identical tile across the 4 screen replicates (calculated by adding 0.1 to all enrichment values and taking the geometric mean) and was used to identify reproducible screen hits. Specific MHC-binding epitopes for each tile above the threshold of 2-fold enrichment were predicted using NetMHC4.0. Candidate epitopes for each tile were selected by identifying predicted strong binding epitopes shared across overlapping adjacent and redundant tiles that were enriched in the screen. To collapse data from multiple tiles into a single datapoint for each patient, the arithmetic mean of all the tiles containing the indicated epitope was calculated.

### L. Peptide Validation Assay

5×10⁴ monomeric MHC reporter cells were seeded into 96-well plates and rested for 16 hours, then pulsed with 1 ug/mL of individual peptides (Genscript) for 1 hour. Bulk isolated CD8+ memory T cells were thawed, washed with warm media, added to the plates at a 2:1 effector to target cell ratio, and incubated for 16 hours. The cells were harvested by pipetting, transferred to V-bottom 96-well plates and centrifuged at 500 xg for 2 minutes. The supernatant was removed and IFNγ was immediately measured using an Ella human IFNγ 3^{rd} generation single-plex assay (Protein Simple) following the manufacturer’s instructions. The remaining cell pellets were washed with FACS buffer (phosphate buffered saline, 0.5% bovine serum albumin, 2 mM EDTA) and stained with PE-conjugated anti-CD137 (Miltenyi), AF647-conjugated anti-CD69 (Biolegend), and BV421-conjugated anti-CD8 (Biolegend) antibodies and analyzed by flow cytometry (Cytoflex S, Beckman Coulter).

### M. Tetramer Staining

MHC tetramers were generated by incubating each peptide with PE- or APC-conjugated empty A*02:01 tetramers (Tetramer Shop) at a final peptide concentration of 30 ug/mL for 30 minutes at room temperature. Two tetramer-peptide reagents with contrasting fluorophore conjugates were used in each stain cocktail at a dilution of 1:10 in FACS buffer. Bulk isolated memory CD8+ T cells were thawed, washed with warm media, and plated in V-bottom 96-well plates at 1×10⁶ cells/well. Cells were pelleted and resuspended in the tetramer stain cocktail and incubated at 37° C. for 15 minutes prior to adding a BV421-conjugated anti-human TCR antibody (Biolegend) and incubating for an additional 15 minutes at room temperature. The stained cells were pelleted and washed three times before resuspending in a 5 ug/mL DAPI solution and analyzed by flow cytometry (Cytoflex S, Beckman Coulter). The limit of detection was defined as the mean + 2 SD of the frequency of three MHC-mismatched controls.

### N. Single Cell TCR Sequencing

Single-cell TCR-seq (scTCR-seq) libraries were prepared following the 10x Genomics Single Cell V(D)J Reagent Kit (v1) protocol. Briefly, cells were captured in droplets before undergoing reverse transcription. Following cDNA purification, cDNA was amplified (98° C. for 45 sec; 16 cycles of 98° C. for 20 sec, 67° C. for 30 sec, 72° C. for 1 min; 72° C. for 1 min). Following sample purification, 2 uL of each library was used for TCR sequence enrichment. TCR enriched libraries were subsequently fragmented, end-repaired, and amplified with indexing primers. The scTCR-seq libraries were sequenced on an Illumina NextSeq™ using a High Output v2.5 kit (150 cycles) with read lengths: 26 bp- read 1, 8 bp- i7 index, 98 bp- read 2. scTCR-seq reads were processed using Cellranger 3.1.0. Reads were aligned to the GRCh38 reference genome, Cellranger vdj was used to annotate TCR consensus sequences.

### Example 2: Identification of Highly Immunodominant Peptides for SARS-CoV-2

T cells play a critical role to control acute viral infection and provide durable immune protection from subsequent exposures. In the case of SARS-CoV-2, virus-reactive T cells have been reported, but the specific peptide targets recognized by these T cells remain unknown. A systematic, comprehensive survey was undertaken to map the precise T cell targets recognized by convalescent COVID-19 patients. Table 2 shows HLA alleles corresponding to patient samples analyzed.

TABLE 2

| | | | | | | | | | | | | | | | | |
|---|---|---|---|---|---|---|---|---|---|---|---|---|---|---|---|---|
| Sample | HLA-A | | HLA-B | | HLA-C | | DPA1 | | DPB1 | | DQA1 | | DRB1 | | DRB | |
| cov-1 | A*02:01:01 | A*23:01:01 | B*49:01:01 | B*50:01:01 | C*06:02:01 | C*07:01:01 | DPA1*01:03:01 | X | DPB1*02:01:02 | DPB1*04:01:01 | DQA1*01:03:01 | DQA1*05:05:01 | DRB1*11:04:01 | DRB1*13:01:01 | DRB3*01:01:02 | DRB3*02:02:01 |
| 01-01-002 | A*24:02:01 | A*32:01:01 | B*15:17:01 | B*35:03:01 | C*07:01:02 | C*12:03:01 | DPA1*01:03:01 | X | DPB1*02:01:02 | DPB1*03:01:01 | DQA1*01:02:01 | DQA1 *01:04:01 | DRB1*13:02:01 | DRB1*14:54:01 | DRB3*02:02:01 | DRB3*03:01:01 |
| 01-01-003 | A*01:01:01 | A*11:01:01 | B*40:02:01 | B*57:01:01 | C*02:02:02 | C*06:02:01 | DPA1*01:03:01 | X | DPB1*04:01:01 | DPB1*04:02:01 | DQA1*02:01 | DQA1 *05:05:01 | DRB1*11:01:01 | DRB1*07:01:01 | DRB3*02:02:01 | DRB4*01:03:01N |
| 01-01-004 | A*02:01:01 | A*74:01:01 | B*15:03:01 | B*35:12:01 | C*02:10:01 | C*04:01:01 | DPA1*01:03:01 | X | DPB1*02:01:02 | DPB1*04:02:01 | DQA1*01:02:01 | DQA1 *03:01:01 | DRB1*15:03:01 | DRB1*04:07:01 | DRB4*01:03:01 | DRB5*01:01:01 |
| 01-01-005 | A*01:01:01 | A*32:01:01 | B*08:01:01 | B*35:189 | C*04:01:01 | C*07:01:01 | DPA1*01:03:01 | X | DPB1*04:01:01 | DPB1*04:02:01 | DQA1*01:01:01 | DQA1 *05:01:01 | DRB1*01:01:01 | DRB1*03:01:01 | DRB3*01:01:02 | X |
| 01-01-006 | A*03:01:01 | A*24:02:01 | B*18:01:01 | B*35:01:01 | C*04:01:01 | C*07:01:01 | DPA1*01:03:01 | X | DPB1*04:01:01 | DPB1*04:02:01 | DQA1*01:01:01 | DQA1 *01:02:01 | DRB1*01:01:01 | DRB1*15:01:01 | DRB5*01:01:01 | X |
| 01-01-007 | A*01:01:01 | A*02:01:01 | B*07:04 | B*08:01:01 | C*07:01:01 | C*07:02:01 | DPA1*01:03:01 | DPA1*02:01:02 | DPB1*01:01:01 | DPB1*04:01:01 | DQA1*05:01:01 | X | DRB1*03:01:01 | X | DRB3*01:01:02 | X |
| 01-01-008 | A*02:01:01 | A*03:01:01 | ? | ? | C*03:03:01 | C*12:03:01 | DPA1*01:03:01 | DPA1*02:01:01 | DPB1*04:01:01 | DPB1*23:01:01 | DQA1*01:02:01 | X | DRB1*15:01:01 | X | DRB5*01:01:01 | X |
| 01-01-009 | A*01:01:01 | X | B*37:01:01 | B*57:01:01 | C*06:02:01 | X | ? | ? | DPB1*04:01:01 | X | DQA1*01:05:01 | DQA1 *02:01 | DRB1*10:01:01 | DRB1*07:01:01 | DRB4*01:03:01 | X |
| 01-01-010 | A*01:01:01 | A*24:02:01 | B*49:01:01 | X | C*07:01:01 | X | DPA1*01:03:01 | DPA1*02:01:01 | DPB1*04:01:01 | DPB1*104:01 | DQA1*01:01:02 | DQA1 *01:02:01 | DRB1*01:02:01 | DRB1*15:01:01 | DRB5*01:01:01 | X |
| 01-01-011 | A*24:02:01 | X | B*18:01:01 | B*35:03:01 | C*04:01:01 | C*05:01:01 | DPA1*01:03:01 | X | DPB1*02:02 | DPB1*03:01:01 | DQA1*01:04:01 | DQA1 *05:01:01 | DRB1*03:01:01 | DRB1*14:54:01 | DRB3*02:02:01 | X |
| 01-01-012 | ? | ? | B*15:01:01 | B*40:01:02 | C*03:03:01 | C*03:04:01 | DPA1*01:03:01 | X | DPB1*04:01:01 | X | DQA1*01:02:01 | DQA1 *05:05:01 | DRB1*11:01:01 | DRB1*13:02:01 | DRB3*02:02:01 | DRB3*03:01:01 |
| 01-01-013 | A*24:02:01 | A*26:01:01 | B*15:01:01 | B*40:01:02 | C*03:03:01 | C*03:04:01 | DPA1*01:03:01 | DPA1*01:04 | DPB1*02:01:02 | DPB1*15:01:01 | DQA1*01:01:01 | DQA1 *03:01:01 | DRB1*01:01:01 | DRB1*04:04:01 | DRB4*01:03:01 | X |
| 01-01-014 | A*02:05:01 | A*30:04:01 | B*35:03:01 | B*51:01:01 | C*04:01:01 | C*16:01:01 | DPA1*01:03:01 | X | DPB1*04:01:01 | X | DQA1*01:02:01 | DQA1 *05:05:01 | DRB1*15:01:01 | DRB1*13:03:01 | DRB3*01:01:02 | DRB5*01:01:01 |
| 01-01-015 | A*02:01:01 | A*24:02:01 | B*18:01:01 | B*35:03:01 | C*04:01:01 | C*07:01:01 | DPA1*01:03:01 | X | DPB1*02:01:02 | DPB1*04:01:01 | DQA1*01:02:01 | DQA1 *05:05:01 | DRB1*15:01:01 | DRB1*11:01:01 | DRB3*02:02:01 | DRB5*01:01:01 |
| 01-01-016 | A*02:01:01 | A*32:01:01 | B*18:01:01 | B*50:01:01 | C*06:02:01 | C*12:03:01 | DPA1*01:03:01 | X | DPB1*03:01:01 | DPB1*04:01:01 | DQA1*02:01 | DQA1 *05:05:01 | DRB1*11:04:01 | DRB1*07:01:01 | DRB3*02:02:01 | DRB4*01:03:01 |
| 01-02-001 | A*29:02:01 | A*30:02:01 | B*51:01:01 | B*57:01:01 | C*02:10:01 | C*16:01:01 | DPA1*02:01:08 | DPA1*03:01 | DPB1*01:01:01 | DPB1*105:01 | DQA1*01:01:01 | DQA1 *05:01:01 | DRB1*01:01:01 | DRB1*08:04:01 | | |
| 01-02-002 | A*03:01:01 | A*23:01:01 | B*07:02:01 | B*49:01:01 | C*07:01:01 | C*07:02:01 | DPA1*01:03:01 | DPA1*02:01:01 | DPB1*13:01:01 | DPB1*23:01:01 | DQA1*01:02:01 | X | DRB1*15:01:01 | | DRB5*01:01:01 | |
| 01-02-003 | A*26:01:01 | A*33:01:01 | B*14:02:01 | B*38:01:01 | C*08:02:01 | C*12:03:01 | DPA1*01:03:01 | X | DPB1*04:01:01 | X | DQA1*01:01:02 | DQA1 *03:01:01 | DRB1*01:02:01 | DRB1*04:02:01 | DRB4*01:03:01 | X |
| 01-02-004 | A*03:01:01 | X | B*07:02:01 | B*14:02:01 | C*07:02:01 | C*08:02:01 | DPA1*01:03:01 | X | DPB1*02:01:02 | DPB1*16:01:01 | DQA1*01:02:01 | DQA1 *02:01 | DRB1*15:01:01 | DRB1*07:01:01 | DRB4*01:01:01 | DRB5*01:01:01 |
| 01-02-005 | A*02:01:01 | X | B*41:02:01 | B*44:02:01 | C*05:01:01 | C*17:03 | DPA1*01:03:01 | X | DPB1*04:01:01 | DPB1*04:02:01 | DQA1*03:03:01 | DQA1 *05:05:01 | DRB1*13:03:01 | DRB1*04:01:01 | DRB3*01:01:02 | DRB4*01:03:01 |
| 01-02-006 | A*02:01:01 | A*25:01:01 | B*15:01:01 | B*44:03:01 | C*03:03:01 | C*16:01:01 | DPA1*01:03:01 | X | DPB1*04:01:01 | DPB1*04:02:01 | DQA1*01:01:01 | DQA1 *01:02:01 | DRB1*01:01:01 | DRB1*15:01:01 | DRB5*01:01:01 | X |
| 01-02-007 | A*11:01:01 | A*24:02:01 | B*38:02:01 | X | C*07:02:01 | C*07:27:01 | DPA1*02:02:02 | X | DPB1*01:01:01 | X | DQA1*01:02:01 | X | DRB1*15:02:01 | X | DRB5*01:01:01 | X |
| 01-02-008 | A*02:01:01 | A*11:01:01 | B*44:02:01 | B*52:01:01 | C*03:04:01 | C*12:02:02 | ? | ? | DPB1*04:01:01 | DPB1*17:01 | DQA1*01:03:01 | DQA1 *05:05:01 | DRB1*15:02:01 | DRB1*12:01:01 | DRB3*02:02:01 | DRB5*01:02 |
| 01-01-017 | A*11:01:01 | A*29:02:01 | B*44:03:01 | B*51:01:01 | C*04:01:01 | C*16:01:01 | DPA1*01:03:01 | DPA1*02:01:01 | DPB1*04:01:01 | DPB1*10:01:01 | DQA1*01:02:01 | DQA1 *05:05:01 | DRB1*11:04:01 | DRB1*13:02:01 | DRB3*02:02:01 | DRB3*03:01:01 |
| 01-01-018 | A*24:02:01 | A*26:01:01 | B*35:01:01 | B*55:01:01 | C*01:02:01 | C*04:01:01 | DPA1*01:03:01 | X | DPB1*02:01:02 | DPB1*23:01:01 | DQA1*01:01:01 | DQA1 *02:01 | DRB1*01:03 | DRB1*07:01:01 | DRB4*01:03:01 | X |
| 01-01-019 | A*03:01:01 | A*11:01:01 | B*35:03:01 | B*51:01:01 | C*12:03:01 | C*14:02:01 | DPA1*01:03:01 | X | DPB1*04:01:01 | X | DQA1*01:02:01 | DQA1 *01:02:02 | DRB1*15:01:01 | DRB1*16:01:01 | DRB5*01:01:01 | DRB5*02:02 |
| 01-01-020 | A*02:01:01 | A*03:01:01 | B*07:02:01 | B*27:02:01 | C*02:02:02 | C*07:02:01 | DPA1*01:03:01 | X | DPB1*02:01:02 | DPB1*04:01:01 | DQA1*01:02:01 | DQA1 *01:02:02 | DRB1*15:01:01 | DRB1*16:01:01 | DRB5*01:01:01 | DRB5*02:02 |
| 01-01-021 | A*03:01:01 | A*30:01:01 | B*07:02:01 | B*13:02:01 | C*06:02:01 | C*07:02:01 | DPA1*01:03:01 | DPA1*02:01:02 | DPB1*01:01:01 | DPB1*04:02:01 | DQA1*02:01 | DQA1 *05:01:01 | DRB1*03:01:01 | DRB1*07:01:01 | DRB3*01:01:02 | DRB4*01:03:01 |
| 01-01-022 | A*03:01:01 | A*33:03:01 | B*07:02:01 | B*58:01:01 | C*03:02:02 | C*07:02:01 | DPA1*01:03:01 | X | DPB1*04:01:01 | DPB1*04:02:01 | DQA1*01:02:01 | DQA1 *05:01:01 | DRB1*15:01:01 | DRB1*03:01:01 | DRB3*02:02:01 | DRB5*01:01:01 |
| 01-01-023 | A*11:01:01 | A*68:01:01 | B*35:01:01 | B*51:01:01 | C*04:01:01 | C*15:04:01 | DPA1*01:03:01 | X | DPB1*04:01:01 | X | DQA1*01:03:01 | DQA1 *03:01:01 | DRB1*13:01:01 | DRB1*04:01:01 | DRB3*02:02:01 | DRB4*01:03:01 |
| 01-01-024 | A*24:02:01 | A*33:03:01 | B*35:01:01 | B*40:01:02 | C*03:04:01 | C*04:01:01 | DPA1*01:03:01 | X | DPB1*04:01:01 | X | DQA1*02:01 | DQA1*05:05:01 | DRB1*12:01:01 | DRB1*07:01:01 | DRB3*02:02:01 | DRB4*01:01:01 |
| 01-01-025 | A*01:01:01 | A*02:01:01 | B*08:01:01 | B*39:06:02 | C*07:01:01 | C*07:02:01 | DPA1*01:03:01 | X | DPB1*04:01:01 | X | DQA1*01:02:01 | DQA1*04:01:01 | DRB1*15:01:01 | DRB1*08:01:01 | | DRB5*01:01:01 |
| 01-01-026 | A*02:120 | A*32:01:01 | B*07:02:01 | B*18:01:01 | C*07:02:01 | C*12:03:01 | DPA1*01:03:01 | X | DPB1*04:01:01 | DPB1*04:02:01 | DQA1*01:01:01 | X | DRB1*01:01:01 | X | | |
| 01-01-027 | A*01:01:01 | A*03:01:01 | B*39:06:02 | B*56:01:01 | C*01:02:01 | C*07:02:01 | DPA1*01:03:01 | X | DPB1*04:01:01 | DPB1*04:02:01 | DQA1*01:01:01 | DQA1*04:01:01 | DRB1*01:01:01 | DRB1*08:01:01 | DRB3*01:15 | X |
| 01-01-028 | A*01:01:01 | A*68:02:01 | B*15:17:01 | B*57:01:01 | C*06:02:01 | C*07:01:02 | DPA1*01:03:01 | X | DPB1*04:01:01 | DPB1*06:01 | DQA1*01:02:01 | DQA1*02:01 | DRB1*13:02:01 | DRB1*07:01:01 | DRB3*03:01:01 | DRB4*01:03:01N |
| 01-01-029 | A*02:01:01 | A*33:01:01 | B*14:02:01 | B*15:01:01 | C*03:04:01 | C*08:02:01 | DPA1*01:03:01 | X | DPB1*02:01:02 | DPB1*04:01:01 | DQA1*01:01:02 | DQA1*02:01 | DRB1*01:02:01 | DRB1*07:01:01 | DRB4*01:03:01 | X |
| 01-01-030 | A*01:01:01 | A*24:02:01 | B*07:02:01 | B*08:01:01 | C*07:01:01 | C*07:02:01 | DPA1*01:03:01 | DPA1*02:01:02 | DPB1*01:01:01 | DPB1*04:01:01 | DQA1*01:02:01 | DQA1*05:01:01 | DRB1*15:01:01 | DRB1*03:01:01 | DRB3*01:01:02 | DRB5*01:01:01 |
| 01-01-031 | A*03:01:01 | A*24:02:01 | B*35:03:01 | B*39:06:02 | C*04:01:01 | C*07:02:01 | DPA1*01:03:01 | X | DPB1*03:01:01 | DPB1*04:01:01 | DQA1*02:01 | DQA1*04:01:01 | DRB1*08:01:01 | DRB1*07:01:01 | | DRB4*01:03:01N |
| 01-01-032 | A*02:01:01 | A*66:01:01 | B*41:02:01 | B*51:01:01 | C*02:02:02 | C*17:03 | DPA1*01:03:01 | DPA1*02:01:01 | DPB1*04:01:01 | DPB1*17:01 | DQA1*01:02:01 | DQA1*05:05:01 | DRB1*15:01:01 | DRB1*13:03:01 | DRB3*01:01:02 | DRB5*01:01:01 |
| 01-01-033 | A*02:01:01 | A*24:02:01 | B*44:03:01 | B*50:01:01 | C*06:02:01 | C*16:01:01 | DPA1*02:01:01 | DPA1*02:02:02 | DPB1*04:01:01 | DPB1*11:01:01 | DQA1*02:01 | X | DRB1*07:01:01 | X | DRB4*01:01:01 | X |
| 01-01-034 | A*01:01:01 | A*11:01:01 | B*18:01:01 | B*35:01:01 | C*04:01:01 | C*07:01:01 | DPA1*01:03:01 | X | DPB1*04:01:01 | X | DQA1*01:02:01 | DQA1*02:01 | DRB1*13:02:01 | DRB1*07:01:01 | DRB3*03:01:01 | DRB4*01:01:01 |
| 01-01-035 | A*02:01:01 | A*30:01:01 | B*13:02:01 | B*35:02:01 | C*04:01:01 | C*06:02:01 | DPA1*01:03:01 | DPA1*02:01:01 | DPB1*06:01 | DPB1*17:01 | DQA1*02:01 | DQA1*05:05:01 | DRB1*11:04:01 | DRB1*07:01:01 | DRB3*02:02:01 | DRB4*01:03:01 |
| 01-01-036 | A*01:01:01 | A*23:01:01 | B*49:01:01 | B*52:01:01 | C*07:01:01 | C*12:02:02 | DPA1*01:03:01 | DPA1*01:04 | DPB1*04:01:01 | DPB1*15:01:01 | DQA1*01:03:01 | DQA1*05:05:01 | DRB1*15:02:01 | DRB1*11:01:01 | DRB3*02:02:01 | DRB5*01:02 |
| 01-01-037 | A*02:01:01 | X | B*07:02:01 | B*13:02:01 | C*06:02:01 | C*07:02:01 | DPA1*01:03:01 | DPA1*02:01:01 | DPB1*02:01:02 | DPB1*17:01 | DQA1*01:02:01 | DQA1*02:01 | DRB1*15:01:01 | DRB1*07:01:01 | DRB4*01:03:01 | DRB5*01:01:01 |
| 01-01-038 | A*02:01:01 | X | B*18:01:01 | B*49:01:01 | C*07:01:01 | X | DPA1*01:03:01 | X | DPB1*02:01:02 | DPB1*04:02:01 | DQA1*03:03:01 | DQA1*05:05:01 | DRB1*11:04:01 | DRB1*04:05:01 | DRB3*02:02:01 | DRB4*01:03:01 |
| 01-01-039 | A*01:01:01 | A*11:01:01 | B*35:01:01 | B*57:01:01 | C*04:01:01 | C*06:02:01 | DPA1*01:03:01 | X | DPB1*04:01:01 | DPB1*04:02:01 | DQA1*01:01:01 | DQA1*05:05:01 | DRB1*01:01:01 | DRB1*13:05:01 | DRB3*02:02:01 | X |
| 01-02-009 | A*01:01:01 | A*24:02:13 | B*40:06:01 | B*44:03:02 | C*07:06 | C*15:02:01 | | | DPB1*02:01:02 | DPB1*04:01:01 | DQA1*01:03:01 | DQA1*02:01 | DRB1*15:01:01 | DRB1*07:01:01 | DRB4*01:03:01 | DRB5*01:01:01 |
| 01-02-010 | A*03:01:01 | A*23:01:01 | B*15:17:01 | B*53:01:01 | C*06:02:01 | C*16:01:01 | DPA1*02:01:08 | DPA1*02:02:02 | DPB1*01:01:01 | X | DQA1*01:02:01 | DQA1*04:01:02 | DRB1*08:04:01 | DRB1*13:02:01 | DRB3*03:01:01 | X |
| 01-02-011 | A*02:02:01 | A*30:02:01 | B*15:16:01 | B*42:01:01 | C*14:02:01 | C*17:01:01 | | | DPB1*01:01:01 | DPB1*85:01 | DQA1*01:03:01 | DQA1*02:01 | DRB1*13:01:01 | DRB1*07:01:01 | DRB3*01:01:02 | DRB4*01:01:01 |
| 01-02-012 | A*01:01:01 | A*11:01:01 | B*35:01:01 | B*35:03:01 | C*04:01:01 | X | DPA1*01:03:01 | X | DPB1*03:01:01 | DPB1*04:01:01 | DQA1*01:01:01 | DQA1*04:01:01 | DRB1*01:03 | DRB1*08:01:01 | | |
| 01-02-013 | A*24:02:01 | A*29:02:01 | B*14:02:01 | B*44:03:01 | C*02:02:02 | C*16:01:01 | DPA1*01:03:01 | DPA1*02:01:01 | DPB1*04:01:01 | DPB1*11:01:01 | DQA1*01:01:02 | DQA1*02:01 | DRB1*01:02:01 | DRB1*07:01:01 | DRB4*01:01:01 | X |
| 01-02-014 | A*30:01:01 | A*74:01:01 | B*15:03:01 | B*42:01:01 | C*02:10:01 | C*17:01:01 | DPA1*02:01:01 | X | DPB1*17:01 | DPB1*131:01 | DQA1*01:02:01 | DQA1*05:01:01 | DRB1*03:01:01 | DRB1*13:02:01 | DRB3*02:02:01 | DRB3*03:01:01 |
| 01-02-015 | A*02:01:01 | A*31:01:02 | B*35:01:01 | B*48:01:01 | C*04:01:01 | C*08:03:01 | DPA1*01:03:01 | X | DPB1*04:02:01 | X | DQA1*03:01:01 | DQA1*04:01:01 | DRB1*08:02:01 | DRB1*04:04:01 | | DRB4*01:03:01 |
| 01-02-016 | A*30:02:01 | A*33:03:01 | B*15:03:01 | B*57:02:01 | C*02:10:01 | C*18:02 | DPA1*02:01:01 | DPA1*03:01 | DPB1*11:01:01 | DPB1*105:0 1 | DQA1*01:05:01 | DQA1*02:01 | DRB1*10:01:01 | DRB1*07:01:01 | DRB4*01:03:01 | X |
| 01-02-017 | A*02:01:01 | A*29:02:01 | B*13:02:01 | B*40:01:02 | C*03:04:01 | C*06:02:01 | DPA1*01:03:01 | X | DPB1*02:01:02 | DPB1*06:01 | DQA1*01:02:02 | DQA1*03:01:01 | DRB1*16:01:01 | DRB1*04:04:01 | DRB4*01:03:01 | DRB5*02:02 |
| 01-02-018 | A*33:03:01 | A*68:02:01 | B*13:02:01 | B*44:03:01 | C*06:02:01 | X | DPA1*02:02:02 | DPA1*03:01 | DPB1*01:01:01 | DPB1*105:01 | DQA1*01:02:01 | DQA1*01:05:01 | DRB1*15:03:01 | DRB1*12:01:01 | DRB3*02:02:01 | DRB5*01:01:01 |
| 01-02-019 | A*01:01:01 | A*02:01:01 | B*40:01:02 | B*57:01:01 | C*03:04:01 | C*06:02:01 | DPA1*01:03:01 | X | DPB1*02:01:02 | DPB1*04:01:01 | DQA1*02:01 | X | DRB1*07:01:01 | X | DRB4*01:01:01 | DRB4*01:03:01N |
| 01-02-020 | A*30:01:01 | A*32:01:01 | B*42:01:01 | B*44:02:01 | C*05:01:01 | C*17:01:01 | DPA1*01:03:01 | DPA1*02:01:08 | DPB1*01:01:01 | DPB1*03:01:01 | DQA1*04:01:01 | DQA1*05:05:01 | DRB1*03:02:01 | DRB1*11:01:01 | DRB3*01:01:02 | DRB3*02:02:01 |
| 01-02-021 | A*02:01:01 | X | B*15:01:01 | B*57:01:01 | C*03:03:01 | C*06:02:01 | DPA1*01:03:01 | X | DPB1*04:01:01 | DPB1*04:02:01 | DQA1*02:01 | DQA1*03:01:01 | DRB1*04:01:01 | DRB1*07:01:01 | DRB4*01:03:01 | DRB4*01:03:01N |
| 01-02-022 | A*02:01:01 | X | B*40:01:02 | B*56:01:01 | C*01:02:01 | C*03:04:01 | DPA1*01:03:01 | X | DPB1*03:01:01 | DPB1*06:01 | DQA1*01:02:01 | DQA1*03:01:01 | DRB1*13:02:01 | DRB1*04:01:01 | DRB3*03:01:01 | DRB4*01:03:01 |
| 01-02-023 | A*02:01:01 | X | B*15:01:01 | B*44:02:01 | C*03:03:01 | C*05:01:01 | DPA1*01:03:01 | X | DPB1*04:01:01 | X | DQA1*03:01:01 | DQA1*03:02 | DRB1*04:01:01 | DRB1*09:01:02 | DRB4*01:03:01 | DRB4*01:03:02 |
| D290_CMV | A*02:01:01 | A*68:02:01 | B*07:02:01 | B*44:02:01 | C*05:01:01 | C*07:02:01 | DPA1*01:03:01 | DPA1*02:01:01 | DPB1*04:01:01 | DPB1*30:01 | DQA1*01:02:01 | DQA1*03:01:01 | DRB1*15:03:01 | DRB1*04:01:01 | DRB4*01:03:01 | DRB5*01:01:01 |
| D400_CMV | A*02:01:01 | A*68:01:02 | B*39:01:01 | B*40:01:02 | C*03:19 | C*07:02:01 | DPA1*01:03:01 | DPA1*02:01:02 | DPB1*01:01:01 | DPB1*04:01:01 | DQA1*03:01:01 | DQA1*04:01:01 | DRB1*08:01:01 | DRB1*04:03:01 | DRB4*01:03:01 | X |
| D493_CMV | A*02:01:01 | A*33:03:01 | B*08:01:01 | B*39:10:01 | C*07:18 | C*12:03:01 | | | DPB1*18:01 | DPB1*85:01 | DQA1*01:02:01 | X | DRB1*15:03:01 | DRB1*13:02:01 | DRB3*03:01:01 | DRB5*01:01:01 |
| D494_CMV | A*02:01:01 | A*23:01:01 | B*35:01:01 | B*44:02:01 | C*04:01:01 | C*05:01:01 | | | DPB1*04:01:01 | DPB1*85:01 | DQA1*03:03:01 | DQA1*05:05:01 | DRB1*08:04:01 | DRB1*04:01:01 | DRB4*01:03:01 | X |
| nonCovid 1 | A*02:02:01 | A*23:01:01 | B*07:02:01 | B*53:01:01 | C*04:01:01 | C*07:02:01 | DPA1*02:01:01 | X | DPB1*17:01 | DPB1*131:01 | DQA1*01:05:01 | DQA1*03:03:01 | DRB1*10:01:01 | DRB1*09:01:02 | | |
| nonCovid_2 | A*24:02:01 | A*30:01:01 | B*13:02:01 | B*35:02:01 | C*04:01:01 | C*06:02:01 | DPA1*01:03:01 | X | DPB1*04:01:01 | DPB1*04:02:01 | DQA1*02:01 | X | DRB1*07:01:01 | X | DRB4*01:03:01 | DRB4*01:03:0IN |
| nonCovid_3 | A*01:01:01 | A*11:01:01 | B*14:02:01 | B*57:01:01 | C*06:02:01 | C*08:02:01 | DPA1*01:03:01 | DPA1*02:01:01 | DPB1*03:01:01 | DPB1*05:01:01 | DQA1*01:02:01 | DQA1*05:01:01 | DRB1*03:01:01 | DRB1*13:02:01 | DRB3*01:01:02 | DRB3*03:01:01 |
| nonCovid_4 | A*01:01:01 | A*26:01:01 | B*08:01:01 | B*40:01:02 | C*03:04:01 | C*07:01:01 | DPA1*01:03:01 | DPA1*02:01:01 | DPB1*04:01:01 | DPB1*10:01:01 | DQA1*04:01:01 | X | DRB1*08:01:01 | X | DRB3*01:0103 | X |

This approach leveraged an antigen discovery platform coupled with a newly designed comprehensive SARS-CoV-2 library to identify T cell targets directly from patient memory T cells in an unbiased way. T cell targets were profiled in a cohort of patients who successfully cleared their SARS-CoV-2 infection.

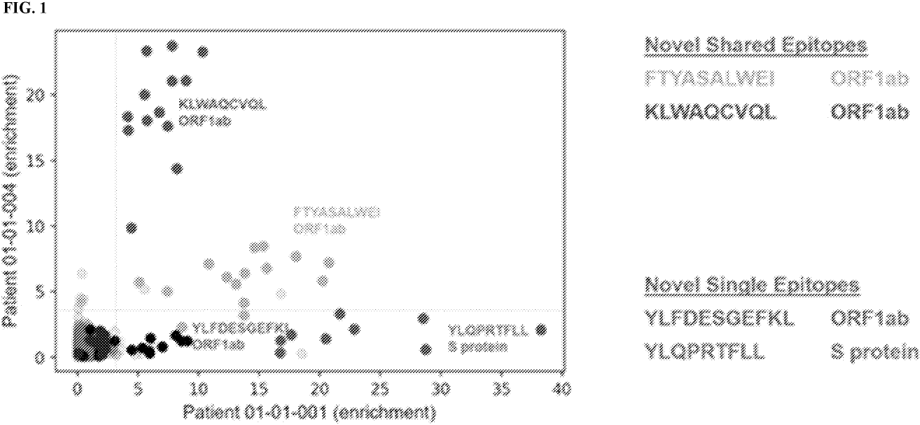

First, sample COVID functional epitope targets were identified from patients. For example, sample screen data in FIG. 1 illustrate the identification of common shared epitopes and epitopes that are unique to individual patients. Targets FTYASALWEI and KLWAQCVQL were identified in both patients. Targets YLQPRTFLL and YLFDESGEFKL were identified in patient 01-01-001 only. This figure also demonstrates the robustness of the epitope discovery approach. Identified epitopes are present in multiple distinct protein fragment tiles that serve as independent reagents. In most cases, all or nearly all of these tiles score in the screen, thereby confirming the proper mapping of the T cell response and helping to quantify its strength.

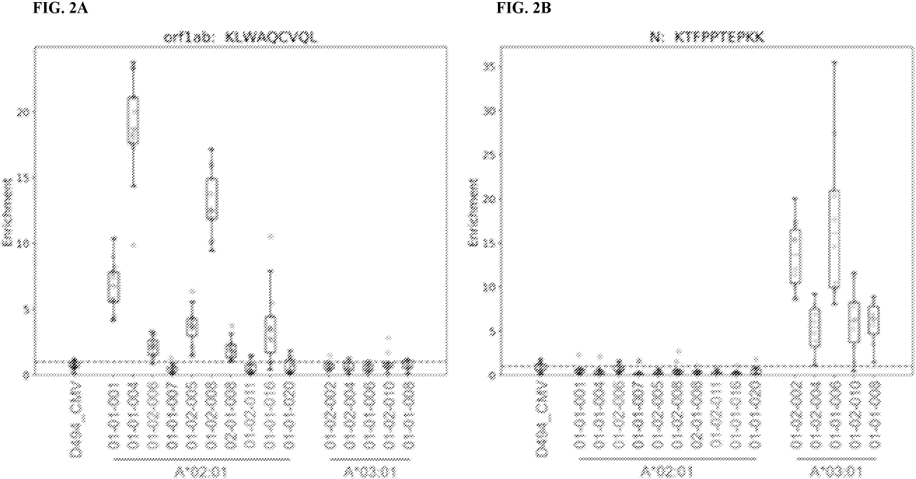

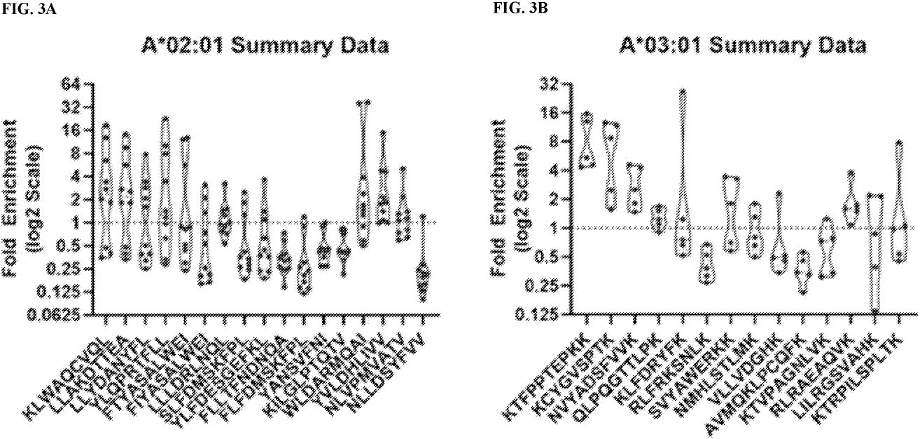

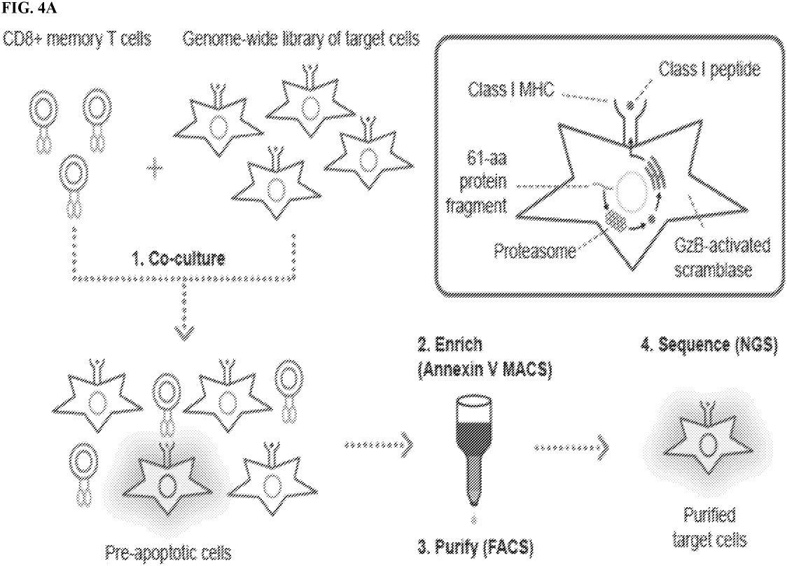

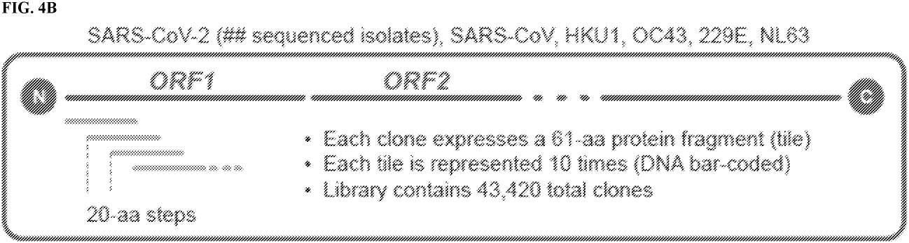

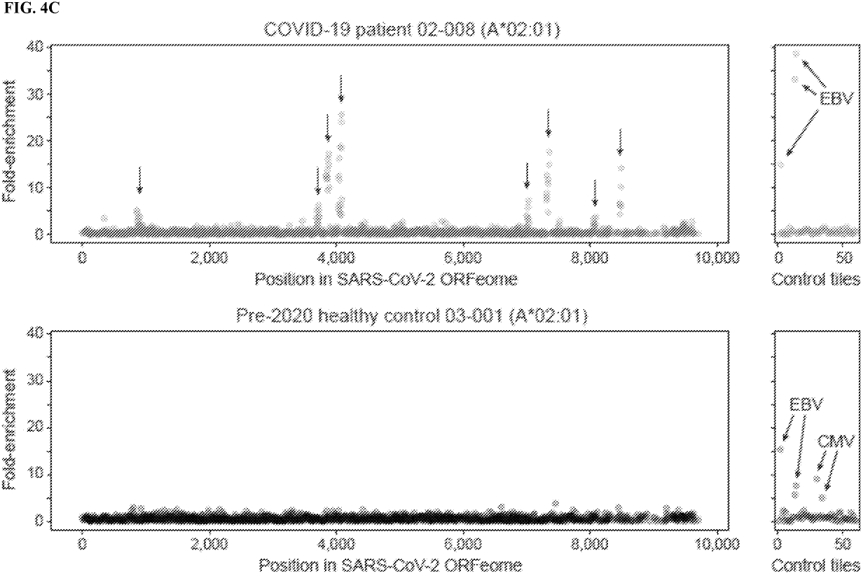

It was also found that identified T cell epitopes are shared across multiple patients. For example, KLWAQCVQL was identified in 7 out of 9 HLA-A*02:01 patients (FIG. 2A). KTFPPTEPKK was identified in all five patients with HLA-A*03:01 allele (FIG. 2B).

FIGS. 2A and 2B show that the identified HLA allele-restricted T cell epitope targets are shared across multiple patients. Similarly, FIGS. 1A through 1F provide a summary of T cell epitopes shared across multiple patients.

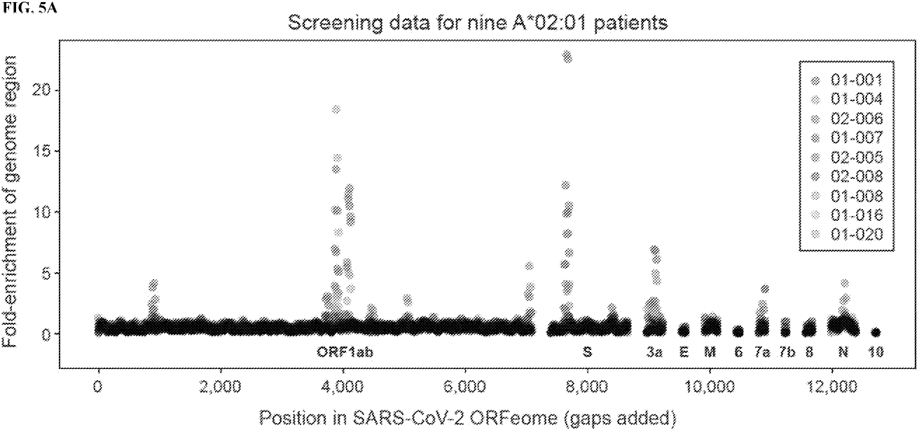

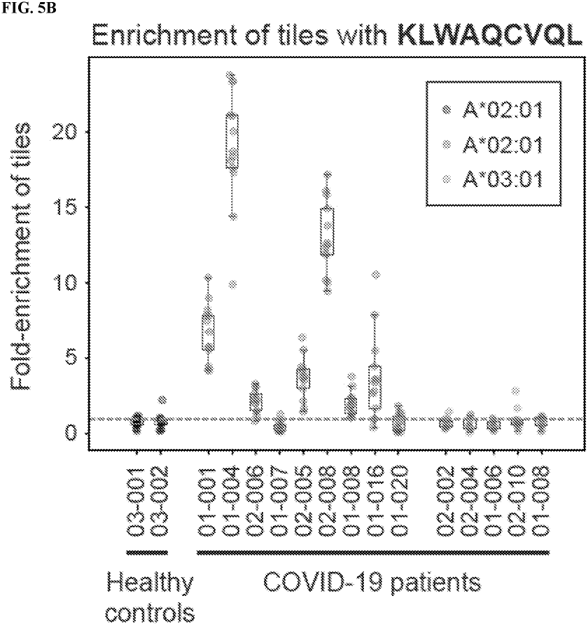

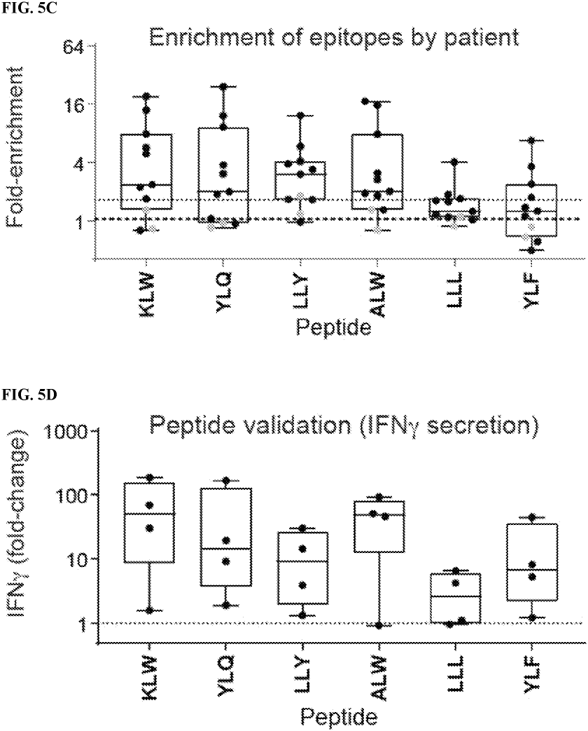

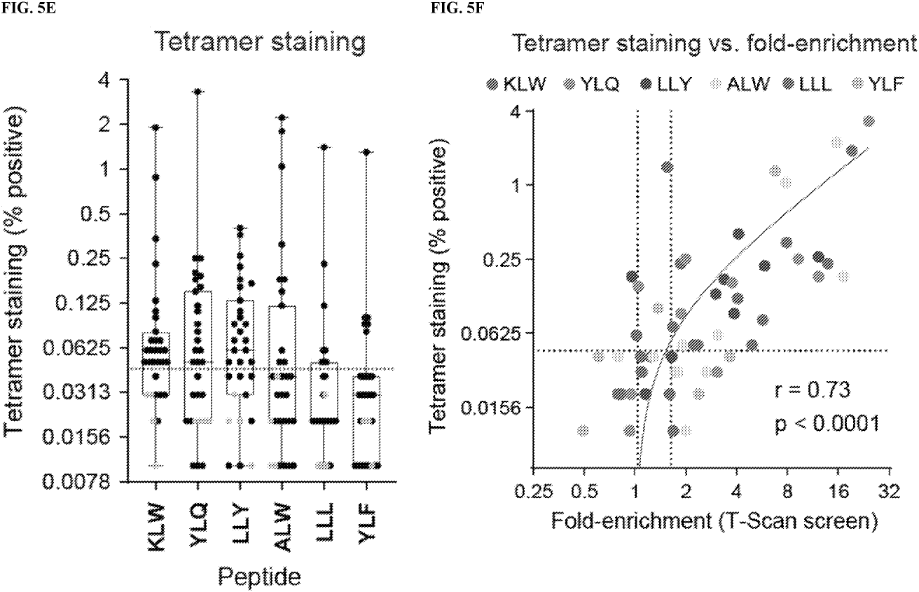

Multiple peptides that elicit COVID-specific T-cell response across patients were identified (FIG. 3 and Table 3). For example, Table 3 lists T cell epitopes identified in SARS-CoV-2 patients. Each row represents a single epitope, grouped based on HLA-A02, HLA-A03, HLA-A01, HLA-A11, HLA-A24, or HLA-B07 presentation, and indicates inter alia the epitope sequence, the open reading frame (ORF) from which it was derived, and the number of screened patients recognizing that epitope. The columns on the right (F-L) indicate the patients who had reactivity to each identified epitope.

TABLE 3

| HLA Allel e | Epitope (N- to C-terminus) | Derived from SARS-CoV-2 Protein | A01-01-01-030 | A01-01-02-019 | A0101_01 -01-003 | A0101_01 -01-007 | A0101_01 -01-009 | # of patients enriching epitope >1.5 (moderate stringency ) | # of patients enriching epitope >5 (high stringency ) |
|---|---|---|---|---|---|---|---|---|---|
| A01 | VPTDNYITTY | ORF1a b | 16.4 9 | 7.92 | 9.22 | 0.59 | 2.96 | 4 | 3 |
| A01 | FTSDYYQLYS | ORF3a | 4.61 | 56.3 4 | 7.96 | 1.76 | 14.15 | 5 | 3 |
| A01 | CTDDNALAY | ORF1a b | 6.71 | 4.08 | 4.00 | 1.55 | 3.23 | 5 | 1 |
| A01 | SSPDDQIGYY | N | 1.21 | 1.98 | 2.32 | 2.46 | 8.83 | 4 | 1 |
| A01 | HTTDPSFLGRY | ORF1a b | 11.9 9 | 26.2 2 | 6.70 | 4.48 | 8.93 | 5 | 4 |
| A01 | TACTDDNALAYY | ORF1a b | 6.71 | 4.08 | 4.00 | 1.55 | 3.23 | 5 | 1 |
| A01 | TDDNALAY | ORF1a b | 6.79 | 4.35 | 4.10 | 1.85 | 3.29 | 5 | 1 |
| A01 | GTDLEGNFY | ORF1a b | 4.35 | 0.48 | 4.69 | 0.46 | 1.00 | 2 | 0 |
| A01 | PTDNYITTY | ORF1a b | 16.5 6 | 7.81 | 9.23 | 0.59 | 2.99 | 4 | 3 |
| A01 | TCDGTTFTY | ORF1a b | 7.01 | 1.78 | 7.34 | 0.72 | 1.02 | 3 | 2 |
| A01 | SMDNSPNLA | ORF1a b | 3.53 | 1.28 | 4.05 | 0.58 | 0.93 | 2 | 0 |
| A01 | YHTTDPSFLGRY | ORF1a b | 11.9 9 | 26.2 2 | 6.70 | 4.48 | 8.93 | 5 | 4 |
| A01 | LTTAAKLMVVIPD Y | ORF1a b | 4.65 | 1.20 | 4.46 | 0.72 | 0.92 | 2 | 0 |
| A01 | VDTDFVNEFY | ORF1a b | 4.88 | 0.96 | 4.05 | 0.74 | 1.58 | 3 | 0 |
| A01 | ACTDDNALAYY | ORF1a b | 6.71 | 4.08 | 4.00 | 1.55 | 3.23 | 5 | 1 |
| A01 | FTSDYYQLY | ORF3a | 4.61 | 56.3 4 | 7.96 | 1.76 | 14.15 | 5 | 3 |
| A01 | YFTSDYYQLY | ORF3a | 4.61 | 56.3 4 | 7.96 | 1.76 | 14.15 | 5 | 3 |
| A01 | DTDFVNEFY | ORF1a b | 4.88 | 0.96 | 4.05 | 0.74 | 1.58 | 3 | 0 |
| A01 | SSDNIALLV | M | 2.34 | 11.2 5 | 0.99 | 1.63 | 2.11 | 4 | 1 |
| A01 | CTDDNALAYY | ORF1a b | 6.71 | 4.08 | 4.00 | 1.55 | 3.23 | 5 | 1 |
| A01 | TTDPSFLGRY | ORF1a b | 11.9 9 | 26.2 2 | 6.70 | 4.48 | 8.93 | 5 | 4 |
| A01 | LSPRWYFYY | N | 0.92 | 1.00 | 2.56 | 3.43 | 5.36 | 3 | 1 |
| A01 | YYHTTDPSFLGRY | ORF1a b | 11.9 9 | 26.2 2 | 6.70 | 4.48 | 8.93 | 5 | 4 |
| A01 | EYYHTTDPSFLGRY | ORF1a b | 11.9 9 | 26.2 2 | 6.70 | 4.48 | 8.93 | 5 | 4 |
| A01 | TSDYYQLY | ORF3a | 4.61 | 56.3 4 | 7.96 | 1.76 | 14.15 | 5 | 3 |
| A01 | ACTDDNALAY | ORF1a b | 6.71 | 4.08 | 4.00 | 1.55 | 3.23 | 5 | 1 |
| A01 | VATSRTLSYY | M | 0.95 | 14.5 9 | 0.92 | 1.02 | 2.14 | 2 | 1 |
| A01 | ATSRTLSYY | M | 0.95 | 14.5 9 | 0.92 | 1.02 | 2.14 | 2 | 1 |
| A01 | NTCDGTTFTY | ORF1a b | 7.09 | 1.49 | 7.26 | 0.62 | 1.00 | 2 | 2 |

| HLA Allele | Epitope (N- to C-terminus) | Derived from SARS-CoV-2 Protein | A02_01-01-001 | A02_01-01-004 | A02_01-02-006 | A02_01-01-007 | A02_01-02-005 | A02_01-02-008 | A02_01-01-008 | A02_01_02_011 | A02_01-01-016 | A02_01-01-020 | # of patients enriching epitope >1.5 (moderate stringency) | # of patients enriching epitope >5 (high stringency) |
|---|---|---|---|---|---|---|---|---|---|---|---|---|---|---|
| A02 | ALWEIQQVV | ORF1ab | 13.21 | 5.50 | 1.02 | 0.39 | 1.44 | 13.10 | 0.93 | 0.35 | 0.57 | 0.50 | 3 | 3 |
| A02 | YLQPRTFLLK | S | 23.55 | 1.75 | 3.59 | 0.49 | 8.44 | 10.73 | 1.35 | 1.08 | 0.51 | 1.71 | 6 | 3 |
| A02 | SAL WEIQQVV | ORF1ab | 13.21 | 5.50 | 1.02 | 0.39 | 1.44 | 13.10 | 0.93 | 0.35 | 0.57 | 0.50 | 3 | 3 |
| A02 | ATYYLFDESGEFKL | ORF1ab | 5.05 | 0.92 | 1.54 | 0.37 | 0.64 | 2.18 | 0.83 | 0.31 | 0.42 | 0.59 | 3 | 1 |
| A02 | PLLYDANYFL | ORF3a | 2.65 | 0.54 | 2.92 | 0.57 | 3.70 | 8.29 | 0.81 | 0.28 | 2.33 | 0.43 | 5 | 1 |
| A02 | LLYDANYFL | ORF3a | 2.65 | 0.54 | 2.92 | 0.57 | 3.70 | 8.29 | 0.81 | 0.28 | 2.33 | 0.43 | 5 | 1 |
| A02 | RLANECAQV | ORF1ab | 0.46 | 0.58 | 1.37 | 0.49 | 3.04 | 1.03 | 0.52 | 0.34 | 0.37 | 0.40 | 1 | 0 |
| A02 | QLSSYSLFDM | ORF1ab | 0.49 | 0.38 | 1.48 | 0.42 | 4.98 | 2.74 | 0.60 | 0.91 | 0.27 | 0.58 | 2 | 0 |
| A02 | YLFDESGEFKL | ORF1ab | 4.75 | 0.87 | 1.45 | 0.36 | 0.60 | 2.06 | 0.78 | 0.29 | 0.40 | 0.56 | 2 | 0 |
| A02 | FLIVAAIVFI | ORF7a | 3.69 | 0.57 | 0.32 | 1.07 | 0.29 | 0.22 | 0.75 | 0.10 | 0.76 | 0.16 | 1 | 0 |
| A02 | YANSVFNI | ORF1ab | 0.52 | 0.53 | 1.25 | 0.54 | 1.66 | 0.65 | 0.56 | 0.41 | 0.36 | 0.42 | 1 | 0 |
| A02 | FLCWHTNCYDYCI | ORF3a | 1.81 | 0.58 | 2.67 | 0.70 | 4.93 | 6.82 | 1.06 | 0.23 | 1.50 | 0.32 | 5 | 1 |
| A02 | SMWALIISV | ORF1ab | 0.53 | 0.59 | 1.58 | 0.34 | 0.86 | 3.23 | 0.72 | 0.30 | 0.34 | 0.64 | 2 | 0 |
| A02 | LLLDRLNQL | N | 1.76 | 1.08 | 1.46 | 1.03 | 3.89 | 1.42 | 1.15 | 0.98 | 0.74 | 1.13 | 2 | 0 |
| A02 | FAFACPDGV | ORF7a | 2.45 | 0.66 | 0.78 | 0.63 | 0.40 | 0.28 | 0.91 | 0.24 | 0.72 | 0.55 | 1 | 0 |
| A02 | YRLANECAQV | ORF1ab | 0.44 | 0.57 | 1.36 | 0.46 | 2.97 | 0.96 | 0.51 | 0.33 | 0.38 | 0.37 | 1 | 0 |
| A02 | GYLQPRTFLL | S | 23.55 | 1.75 | 3.59 | 0.49 | 8.44 | 10.73 | 1.35 | 1.08 | 0.51 | 1.71 | 6 | 3 |
| A02 | YLQPRTFLL | S | 23.55 | 1.75 | 3.59 | 0.49 | 8.44 | 10.73 | 1.35 | 1.08 | 0.51 | 1.71 | 6 | 3 |
| A02 | KLWAQCVQL | ORF1ab | 6.72 | 19.01 | 2.16 | 0.45 | 3.68 | 12.99 | 1.98 | 0.54 | 3.73 | 0.69 | 7 | 3 |
| A02 | ALWEIQQV | ORF1ab | 12.46 | 5.46 | 1.01 | 0.40 | 1.31 | 12.29 | 0.96 | 0.35 | 0.54 | 0.58 | 3 | 3 |
| A02 | ALDQAISMWA | ORF1ab | 0.49 | 0.53 | 1.34 | 0.36 | 0.88 | 2.60 | 0.73 | 0.18 | 0.37 | 0.59 | 1 | 0 |
| A02 | SLFDMSKFPL | ORF1ab | 0.52 | 0.34 | 1.73 | 0.38 | 5.53 | 2.95 | 0.54 | 0.91 | 0.26 | 0.47 | 3 | 1 |
| A02 | LLAKDTTEA | ORF1ab | 6.46 | 17.45 | 2.01 | 0.43 | 3.40 | 11.98 | 1.98 | 0.48 | 3.78 | 0.70 | 7 | 3 |
| A02 | MDLFMRIFTI | ORF3a | 0.16 | 0.10 | 0.26 | 0.10 | 0.10 | 0.10 | 2.03 | 0.50 | 0.10 | 0.10 | 1 | 0 |
| A02 | KILGLPTQTV | ORF1ab | 0.58 | 0.77 | 0.88 | 0.57 | 0.53 | 0.54 | 0.96 | 0.25 | 0.45 | 0.65 | 0 | 0 |
| A02 | SLQTYVTQQL | S | 0.74 | 1.09 | 1.38 | 0.64 | 0.83 | 1.99 | 1.18 | 0.59 | 0.41 | 0.68 | 1 | 0 |
| A02 | ALSKGVHFV | ORF3a | 0.36 | 0.86 | 2.42 | 0.20 | 2.48 | 0.40 | 0.29 | 0.28 | 0.41 | 0.20 | 2 | 0 |
| A02 | VMCGGSLYV | ORF1ab | 0.39 | 0.49 | 1.35 | 0.42 | 2.38 | 0.76 | 0.46 | 0.34 | 0.36 | 0.39 | 1 | 0 |
| A02 | TYASALWEIQQVV | ORF1ab | 13.87 | 5.94 | 1.05 | 0.42 | 1.60 | 14.65 | 0.98 | 0.37 | 0.64 | 0.56 | 4 | 3 |
| A02 | LLYDANYFLC | ORF3a | 2.65 | 0.54 | 2.92 | 0.57 | 3.70 | 8.29 | 0.81 | 0.28 | 2.33 | 0.43 | 5 | 1 |
| A02 | FDMSKFPLKL | ORF1ab | 0.43 | 0.33 | 1.71 | 0.30 | 4.60 | 2.16 | 0.52 | 0.66 | 0.23 | 0.39 | 3 | 0 |
| A02 | TYYLFDESGEFKL | ORF1ab | 5.05 | 0.92 | 1.54 | 0.37 | 0.64 | 2.18 | 0.83 | 0.31 | 0.42 | 0.59 | 3 | 1 |
| A02 | YSLFDMSKFPL | ORF1ab | 0.49 | 0.36 | 1.82 | 0.40 | 5.92 | 3.09 | 0.57 | 0.97 | 0.28 | 0.50 | 3 | 1 |
| A02 | YASALWEIQQVV | ORF1ab | 13.21 | 5.50 | 1.02 | 0.39 | 1.44 | 13.10 | 0.93 | 0.35 | 0.57 | 0.50 | 3 | 3 |
| A02 | FLLKYNENGTI | S | 27.06 | 1.62 | 4.28 | 0.30 | 9.90 | 11.96 | 1.07 | 1.01 | 0.62 | 1.73 | 6 | 3 |
| A02 | FTYASALWEI | ORF1ab | 13.44 | 5.87 | 1.04 | 0.44 | 1.52 | 14.08 | 0.98 | 0.36 | 0.61 | 0.65 | 4 | 3 |
| A02 | YYLFDESGEFKL | ORF1ab | 5.05 | 0.92 | 1.54 | 0.37 | 0.64 | 2.18 | 0.83 | 0.31 | 0.42 | 0.59 | 3 | 1 |
| A02 | RLWLCWKCRSKNPL | ORF3a | 1.16 | 1.27 | 2.53 | 0.73 | 4.14 | 5.94 | 0.66 | 0.17 | 1.07 | 0.26 | 3 | 1 |

| HLA Allele | Epitope (N- to C-terminus) | Derived from SARS-CoV-2 Protein | A03_01-02-002 | A03_01-02-004 | A03_01-01-006 | A03_01-02-010 | A03_01-01-008 | # of patients enriching epitope >1.5 (moderate stringency) | # of patients enriching epitope >5 (high stringency) |
|---|---|---|---|---|---|---|---|---|---|
| A03 | TVIEVQGYK | ORF1ab | 19.57 | 0.90 | 0.93 | 1.56 | 0.82 | 2 | 1 |
| A03 | QIAPGQTGK | S | 7.06 | 1.46 | 5.98 | 1.78 | 5.07 | 4 | 3 |
| A03 | MMVTNNTFTLK | ORF1ab | 28.12 | 1.09 | 1.20 | 1.96 | 0.72 | 2 | 1 |
| A03 | RLFRKSNLK | S | 0.57 | 0.32 | 0.94 | 0.40 | 0.69 | 0 | 0 |
| A03 | YNSASFSTFK | S | 9.14 | 1.91 | 9.50 | 2.44 | 6.77 | 5 | 3 |
| A03 | VTNNTFTLK | ORF1ab | 28.12 | 1.09 | 1.20 | 1.96 | 0.72 | 2 | 1 |
| A03 | RQIAPGQTGK | S | 7.22 | 1.40 | 6.04 | 1.88 | 5.36 | 4 | 3 |
| A03 | KLFDRYFKY | ORF1ab | 26.97 | 1.40 | 0.76 | 1.02 | 0.80 | 1 | 1 |
| A03 | KTIQPRVEK | ORF1ab | 5.83 | 0.51 | 0.69 | 0.77 | 0.62 | 1 | 1 |
| A03 | CVADYSVLY | S | 7.38 | 1.67 | 7.82 | 2.03 | 5.13 | 5 | 3 |
| A03 | RLKLFDRYFK | ORF1ab | 27.41 | 1.42 | 0.81 | 1.08 | 0.85 | 1 | 1 |
| A03 | KTFPPTEPK | N | 13.59 | 5.24 | 17.45 | 6.20 | 6.03 | 5 | 5 |
| A03 | STFKCYGVSPTK | S | 12.60 | 2.17 | 12.94 | 3.05 | 9.24 | 5 | 3 |
| A03 | KCYGVSPTK | S | 12.60 | 2.17 | 12.94 | 3.05 | 9.24 | 5 | 3 |
| A03 | VLYNSASFSTFK | S | 9.14 | 1.91 | 9.50 | 2.44 | 6.77 | 5 | 3 |
| A03 | MVTNNTFTLK | ORF1ab | 28.12 | 1.09 | 1.20 | 1.96 | 0.72 | 2 | 1 |
| A03 | KTFPPTEPKK | N | 13.59 | 5.24 | 17.45 | 6.20 | 6.03 | 5 | 5 |
| A03 | KLFDRYFK | ORF1ab | 26.97 | 1.40 | 0.76 | 1.02 | 0.80 | 1 | 1 |
| A03 | QLPQGTTLPK | N | 1.14 | 1.96 | 1.16 | 1.35 | 1.57 | 2 | 0 |

| HLA Allele | Epitope (N- to C-terminus) | Derived from SARS-CoV-2 Protein | A1101_01-01-003 | A1101_01-02-007 | A1101_01-02-008 | A11-01-01-039 | A11-01-02-012 | # of patients enriching epitope >1.5(moderate stringency) | # of patients enriching epitope >5 (high stringency) |
|---|---|---|---|---|---|---|---|---|---|
| A11 | VTDTPKGPK | ORF1ab | 4.86 | 0.88 | 18.80 | 0.89 | 0.27 | 2 | 1 |
| A11 | VTNNTFTLK | ORF1ab | 4.26 | 0.59 | 0.71 | 0.43 | 0.60 | 1 | 0 |
| A11 | TVATSRTLSYYK | M | 2.51 | 5.06 | 0.51 | 0.79 | 1.15 | 2 | 1 |
| A11 | ASAFFGMSR | N | 0.93 | 5.84 | 1.13 | 0.94 | 0.35 | 1 | 1 |
| A11 | LIRQGTDYK | N | 1.09 | 4.14 | 1.74 | 1.20 | 0.65 | 2 | 0 |
| A11 | LLNKHIDAYK | N | 5.27 | 3.94 | 2.86 | 0.77 | 14.96 | 4 | 2 |
| A11 | AVILRGHLR | M | 1.76 | 3.09 | 0.82 | 0.70 | 1.06 | 2 | 0 |
| A11 | QDLKWARFPK | ORF1ab | 1.80 | 1.10 | 9.03 | 0.94 | 0.33 | 2 | 1 |
| A11 | VTLACFVLAAVYR | M | 0.76 | 4.27 | 0.67 | 0.73 | 2.53 | 2 | 0 |
| A11 | KVKYLYFIK | ORF1ab | 4.74 | 0.95 | 17.30 | 0.78 | 0.28 | 2 | 1 |
| A11 | STMTNRQFHQKLLK | ORF1ab | 0.69 | 3.75 | 1.16 | 0.92 | 0.37 | 1 | 0 |
| A11 | KTFPPTEPK | N | 6.23 | 2.36 | 3.74 | 0.85 | 21.00 | 4 | 2 |
| A11 | QQQGQTVTK | N | 1.69 | 3.60 | 2.01 | 0.99 | 0.56 | 3 | 0 |
| A11 | ATSRTLSYYK | M | 2.51 | 5.06 | 0.51 | 0.79 | 1.15 | 2 | 1 |
| A11 | ATEGALNTPK | N | 5.24 | 1.91 | 6.08 | 0.76 | 0.30 | 3 | 2 |
| A11 | KSAAEASKK | N | 1.67 | 4.14 | 2.22 | 1.00 | 0.53 | 3 | 0 |
| A11 | KAYNVTQAFGR | N | 1.71 | 4.44 | 2.20 | 0.98 | 0.38 | 3 | 0 |

| HLA Allele | Epitope (N- to C-terminus) | Derived from SARS-CoV-2 Protein | A24-01-01-015 | A24-01-01-030 | A2402_01-01-006 | A2402_01-01-007 | A2402_01-01-011 | # of patients enriching epitope >1.5 (moderate stringency) | # of patients enriching epitope >5 (high stringency) |
|---|---|---|---|---|---|---|---|---|---|
| A24 | QYIKWPWYI | S | 1.45 | 11.23 | 0.52 | 1.15 | 2.06 | 2 | 1 |
| A24 | VYIGDPAQL | ORF1ab | 0.19 | 3.06 | 0.92 | 0.57 | 1.42 | 1 | 0 |
| A24 | VYFLQSINF | ORF3a | 0.23 | 5.45 | 0.50 | 0.95 | 1.08 | 1 | 1 |
| A24 | YYRRATRRI | N | 0.47 | 0.73 | 6.30 | 3.54 | 3.49 | 3 | 1 |
| A24 | RWYFYYLGTG | N | 0.44 | 0.69 | 8.12 | 4.61 | 4.26 | 3 | 1 |
| A24 | QYIKWPWYIW | S | 1.45 | 11.23 | 0.52 | 1.15 | 2.06 | 2 | 1 |
| A24 | KYEQYIKWPW | S | 1.46 | 10.64 | 0.62 | 1.13 | 2.07 | 2 | 1 |
| A24 | KWPWYIWLGF | S | 1.45 | 11.23 | 0.52 | 1.15 | 2.06 | 2 | 1 |
| A24 | LYLYALVYF | ORF3a | 0.23 | 5.45 | 0.50 | 0.95 | 1.08 | 1 | 1 |
| A24 | LYALVYFLQSINFV | ORF3a | 0.23 | 5.45 | 0.50 | 0.95 | 1.08 | 1 | 1 |
| A24 | YLYALVYFLQSINF | ORF3a | 0.23 | 5.45 | 0.50 | 0.95 | 1.08 | 1 | 1 |
| A24 | QYIKWPWYIWLGF | S | 1.45 | 11.23 | 0.52 | 1.15 | 2.06 | 2 | 1 |
| A24 | LYALVYFLQSINF | ORF3a | 0.23 | 5.45 | 0.50 | 0.95 | 1.08 | 1 | 1 |

| HLA Allele | Epitope (N- to C-terminus) | Derived from SARS-CoV-2 Protein | B0702_01-02-002 | B0702_01-02-004 | B0702_01-01-021 | B0702_01-01-030 | B07_01-01-22 | # of patients enriching epitope >1.5 (moderate stringency) | # of patients enriching epitope >5 (high stringency) |
|---|---|---|---|---|---|---|---|---|---|
| B07 | SPRWYFYYLG | N | 17.70 | 16.68 | 5.03 | 7.12 | 0.29 | 4 | 4 |
| B07 | IPRRNVATL | ORF1ab | 3.56 | 0.80 | 0.52 | 1.26 | 0.13 | 1 | 0 |
| B07 | RPDTRYVL | ORF1ab | 10.65 | 1.74 | 1.69 | 1.71 | 0.28 | 4 | 1 |
| B07 | SPRWYFYYL | N | 17.70 | 16.68 | 5.03 | 7.12 | 0.29 | 4 | 4 |
| B07 | RPDTRYVLM | ORF1ab | 10.65 | 1.74 | 1.69 | 1.71 | 0.28 | 4 | 1 |
| B07 | IPRRNVATLQ | ORF1ab | 3.83 | 0.86 | 0.56 | 1.30 | 0.13 | 1 | 0 |
| B07 | EIPRRNVATL | ORF1ab | 3.56 | 0.80 | 0.52 | 1.26 | 0.13 | 1 | 0 |
| B07 | PRWYFYYL | N | 16.86 | 14.58 | 5.74 | 7.41 | 0.33 | 4 | 4 |
| B07 | LSPRWYFYYL | N | 17.70 | 16.68 | 5.03 | 7.12 | 0.29 | 4 | 4 |
| B07 | RIRGGDGKM | N | 11.87 | 12.54 | 2.83 | 4.78 | 0.34 | 4 | 2 |
| B07 | SLEIPRRNVATLQA | ORF1ab | 4.07 | 0.92 | 0.58 | 1.13 | 0.13 | 1 | 0 |

Such peptides can: (1) serve as the basis for vaccine strategies that elicit protective T cell response; (2) be utilized to identify COVID-reactive T cell receptors for therapeutic applications; (3) be utilized for measuring COVID-specific T cell response as a diagnostic tool.

### Example 3: Analysis of Highly Immunodominant Peptides for SARS-CoV-2

Analyses were performed to further confirm the results presented in Example 1.

As described above, a recently-developed high-throughput screening technology, termed T-Scan (Kula et al. (2019) Cell 178:1016-1028), was used to simultaneously screen all the memory CD8+ T cells of 25 convalescent patients against every possible MHC class I epitope in SARS-CoV-2, as well as SARS-CoV and the four coronaviruses that cause the common cold (HKU1, OC43, 229E, and NL63). Because T cells recognize viral peptide targets in the context of MHC proteins, which are defined by an individual’s HLA type, patients were selected who were positive for each of the six most prevalent HLA types (A*02:01, A*01:01, A*03:01, A* 11:01, A*24:02, and B*07:02). Collectively, ~90% of the U.S. population and ~85% of the world population are positive for at least one of these six alleles (Maiers et al. (2007) Hum. Immunol. 68:779-788; Gonzalez-Galarza (2020) Nucl. Acids Res. 48:D783-D788). Efforts were focused on patients with relatively mild, disease (primarily non-hospitalized patients) in order to discover the most protective epitopes, but also included patients with moderate to severe disease to determine if T cell responses correlate with disease severity.

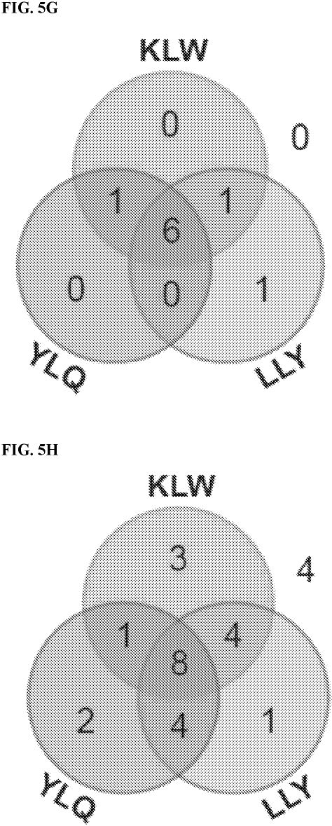

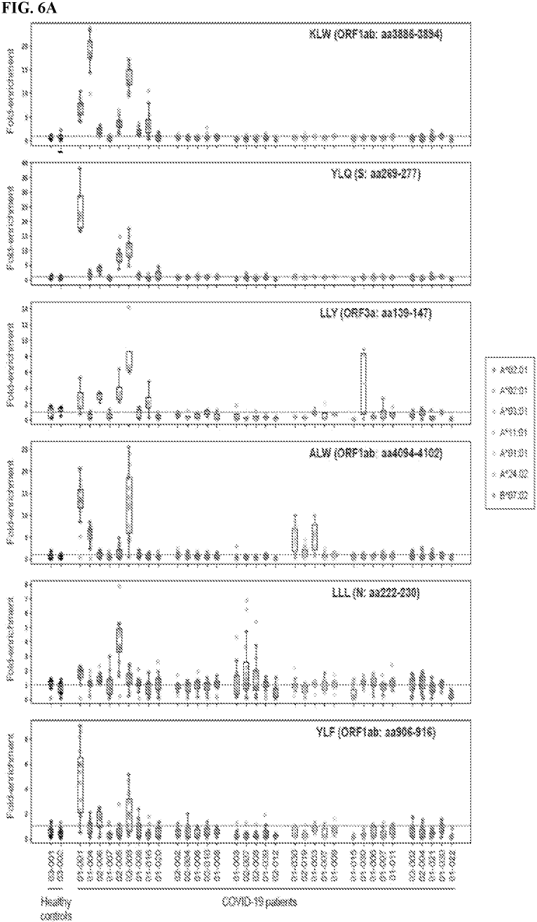

This strategy allowed for the determination of the precise epitopes in SARS-CoV-2 that are recognized by the memory CD8+ T cells of patients who have recovered from COVID-19. To do this, high-throughput cell-based screening technology (T-Scan) described above that enables simultaneous identification of the natural targets of CD8+ T cells in an unbiased, genome-wide fashion (FIG. 4A). Briefly, CD8+ T cells were co-cultured with a genome-wide library of target cells (HEK 293 cells). Each target cell in the library expresses a different 61-amino acid (61-aa) protein fragment. These fragments are processed naturally by the target cells and the appropriate peptide epitopes are displayed on class I MHCs on the cell surface. If a CD8+ T cell encounters its target in the co-culture, it secretes cytotoxic granules into the target cell, inducing apoptosis. Early apoptotic cells are then isolated from the co-culture and the expression cassettes are sequenced, thereby revealing the identity of the protein fragment. Because the assay is non-competitive, hundreds to thousands of T cells can be screened against tens of thousands of targets simultaneously.

To address the bottleneck of extensive sorting needed to isolate rare recognized target cells in high complexity libraries (Kula et al. (2019) Cell 178:1016-1028), target cells were engineered to express a granzyme B (GzB)-activated version of the scramblase enzyme, XKR8, which drives the rapid and efficient transfer of phosphatidylserine to the outer membrane of early apoptotic cells. Early apoptotic cells were then enriched by magnetic-activated cell sorting with Annexin V (see the methods and FIG. 1A). This modification increased throughput of the T-Scan assay 20-fold, enabling the rapid processing of a large number of patient samples.

To comprehensively map responses to SARS-CoV-2, a library of 61-aa protein fragments that tiled across all 11 open reading frames (ORFs) of SARS-CoV-2 in 20-aa steps, as described above (FIG. 4B). To capture the known genetic diversity of SARS-CoV-2, all protein-coding variants from the 104 isolates that had been reported as of Mar. 15, 2020 were included. In addition, the complete set of ORFs (ORFeome) of SARS-CoV and the four endemic coronaviruses that cause the common cold (betacoronaviruses HKU1 and OC43, and alphacoronaviruses NL63 and 229E) were included. As positive controls, known immunodominant antigens from cytomegalovirus, Epstein-Barr virus, and influenza virus were included. Finally, each protein fragment was represented ten times, each encoded with a unique nucleotide barcode to provide internal replicates in our screens, for a final library size of 43,420 clones.

To understand the scope and nature of acquired immunity, the focus was placed on the memory CD8+ T cells of convalescent COVID-19 patients, as described above. In total, peripheral blood mononuclear cells (PBMCs) were collected from 78 adult patients who had tested positive by viral PCR (swab test), had recovered from their disease, and had been out of quarantine according to Centers for Disease Control and Prevention (CDC) guidelines by at least two weeks. Patients were recruited at either of two centers: Atlantic Heath System in Morristown, NJ and Ochsner Medical Center in New Orleans, LA. All patients were HLA-typed, and a summary of their characteristics are provided in Table 4.

TABLE 4

COVID-19 patient characteristics and HLA types

| Patient ID | Age Range | Ethnicity | Gender | SYMPTOM DURATION (Days): | HOSPITALIZATION REQUIRED: | OXYGEN USE REQUIRED: | VENTILATOR USE REQUIRED: | Days after diagnosis until blood draw |
|---|---|---|---|---|---|---|---|---|
| 01-001 | 50 - 54 | Caucasian | M | 2 | No | No | No | 24 |
| 01-002 | 35 - 39 | Asian/Indian | F | 3 | No | No | No | 11 |
| 01-003 | 50 - 54 | Caucasian | M | 7 | No | No | No | 24 |
| 01-004 | 20 - 24 | Hispanic | F | 4 | No | No | No | 13 |
| 01-005 | 30 - 34 | Caucasian | F | 2 | No | No | No | 28 |
| 01-006 | 45 - 49 | Caucasian | F | 14 | No | No | No | 41 |
| 01-007 | 60 - 64 | Caucasian | F | 19 | Yes | Yes | Yes | 34 |
| 01-008 | 30 - 34 | Caucasian | F | 13 | No | No | No | 39 |
| 01-009 | 30 - 34 | Asian/Indian | F | 5 | No | No | No | 35 |
| 01-010 | 30 - 34 | Middle East | F | 3 | No | No | No | 28 |
| 01-011 | 50 - 54 | Caucasian | F | 21 | No | No | No | 29 |
| 01-012 | 60 - 64 | Caucasian | F | 5 | No | No | No | 37 |
| 01-013 | 65 - 69 | Caucasian | F | 14 | No | No | No | 44 |
| 01-014 | 55 - 59 | Hispanic | F | 19 | No | No | No | 30 |
| 01-015 | 40 - 45 | Caucasian | M | 19 | No | No | No | 52 |
| 01-016 | 55 - 59 | Caucasian | M | 21 | Yes | Yes | No | 46 |
| 01-019 | 45 - 49 | Caucasian | F | 15 | No | No | No | 45 |
| 01-020 | 50 - 54 | Caucasian | F | 21 | No | No | No | 47 |
| 01-021 | 55 - 59 | White | F | 22 | Yes | No | No | 56 |
| 01-022 | 25 - 29 | White | F | 33 | No | No | No | 51 |
| 01-023 | 25 - 29 | White | F | 23 | No | No | No | 46 |
| 01-024 | 45 - 49 | White | M | 5 | No | No | No | 62 |
| 01-025 | 60 - 64 | White | F | 22 | No | No | No | 53 |
| 01-026 | 45 - 49 | White | M | 0 | No | No | No | 52 |
| 01-027 | 55 - 59 | White | F | 15 | No | No | No | 52 |
| 01-028 | 35 - 39 | White | F | 10 | No | No | No | 57 |
| 01-029 | 45 - 49 | White | F | 12 | No | No | No | 59 |
| 01-030 | 20 - 24 | Hispanic | F | 15 | No | No | No | 55 |
| 01-031 | 45 - 49 | White | F | 18 | No | No | No | 60 |
| 01-032 | 30 - 35 | White | F | 16 | No | No | No | 56 |
| 01-033 | 55 - 59 | White | M | 28 | No | No | No | 49 |
| 01-034 | 45 - 49 | White | F | 14 | No | No | No | 43 |
| 01-035 | 65 - 69 | White | F | 16 | No | No | No | 55 |
| 01-036 | 60 - 64 | White | F | 21 | No | No | No | 51 |
| 01-037 | 50-54 | White | F | 11 | White | No | No | 48 |
| 01-036 | 35-39 | White | F | 17 | White | No | No | 53 |
| 01-039 | 30-34 | White | F | 11 | White | No | No | 74 |
| 01-040 | 40-44 | White | F | 5 | No | No | No | 78 |
| 01-041 | 60-64 | Hispanic | F | 36 | Yes | Yes | No | 57 |
| 01-042 | 60-64 | White | M | 30 | Yes | Yes | Yes | 60 |
| 01-043 | 45-49 | Hispanic | M | 21 | Yes | Yes | No | 63 |
| 01-044 | 55-59 | Asian | M | 29 | Yes | Yes | Yes | 81 |
| 01-045 | 30-34 | Caucasian | M | 15 | Yes | Yes | No | 89 |
| 01-046 | 50-54 | White | M | 7 | Yes | Yes | No | 79 |
| 01-047 | 50-54 | Caucasian | M | 14 | Yes | Yes | No | 80 |
| 01-048 | 50-54 | Non-Hispanic | M | 30 | Yes | Yes | No | 87 |
| 01-049 | 65-69 | Non-Hispanic | M | 24 | Yes | Yes | No | 96 |
| 01-050 | 55-59 | Caucasian | M | 24 | Yes | Yes | No | 92 |
| 01-051 | 65-69 | White | F | 22 | Yes | Yes | Yes | 111 |
| 02-001 | 50-54 | Black | F | 10 | No | No | No | 39 |
| 02-002 | 55-59 | White | F | 7 | No | No | No | 17 |
| 02-003 | 70-74 | White | M | 21 | Yes | Yes | No | 43 |
| 02-004 | 65-69 | White | F | 14 | No | No | No | 45 |
| 02-005 | 30-34 | White | F | 38 | No | No | No | 44 |
| 02-006 | 45-49 | Other | M | 18 | Yes | Yes | No | 45 |
| 02-007 | 40-44 | White | F | Unknown | No | No | No | 44 |
| 02-008 | 25-29 | White | M | Unknown | No | No | No | 44 |
| 02-009 | 45-49 | White | F | 38 | No | No | No | 44 |
| 02-010 | 35-39 | Black | F | 3 | No | No | No | 49 |
| 02-011 | 55-59 | Black | F | 9 | Yes | Yes | No | 49 |
| 02-012 | 65-69 | White | M | 10 | Yes | Yes | No | 51 |
| 02-013 | 40-44 | Other | F | 6 | No | No | No | 45 |
| 02-014 | 45-49 | Black | F | 15 | No | No | No | 37 |
| 02-015 | 35-39 | Other | F | 1 | No | No | No | 13 |
| 02-016 | 45-49 | Black | F | 20 | Yes | Yes | No | 59 |
| 02-017 | 75-79 | Black | F | 30 | Yes | Yes | No | 49 |
| 02-018 | 70-74 | White | F | 12 | No | No | No | 25 |
| 02-019 | 65-69 | White | M | 42 | Yes | Yes | No | 48 |
| 02-020 | 35-39 | Black | F | 1 | No | No | No | 44 |
| 02-021 | 35-39 | White | F | 22 | No | No | No | 50 |
| 02-022 | 70-74 | White | F | 14 | No | No | No | 64 |
| 02-022 | 70-74 | White | F | 14 | No | No | No | 64 |
| 02-023 | 65-69 | White | F | 3 | No | No | No | 53 |
| 02-024 | 70-74 | White | M | 76 | Yes | Yes | No | 72 |
| 02-025 | 60-64 | White | F | 72 | No | No | No | 75 |
| 02-026 | 60-64 | White | F | 34 | Yes | Yes | No | 30 |
| 02-027 | 65-69 | Black | F | 80 | Yes | Yes | No | 85 |

| | | | | | | |
|---|---|---|---|---|---|---|
| Patient ID | HLA-A | HLA-A | HLA-B | HLA-B | HLA-C | HLA-C |
| 01-001 | A*02:01:01 | A*23:01:01 | B*49:01:01 | B*50:01:01 | C*06:02:01 | C*07:01:01 |
| 01-002 | A*24:02:01 | A*32:01:01 | B*15:17:01 | B*35:03:01 | C*07:01:02 | C*12:03:01 |
| 01-003 | A*01:01:01 | A*11:01:01 | B*40:02:01 | B*57:01:01 | C*02:02:02 | C*06:02:01 |
| 01-004 | A*02:01:01 | A*74:01:01 | B*15:03:01 | B*35:12:01 | C*02:10:01 | C*04:01:01 |
| 01-005 | A*01:01:01 | A*32:01:01 | B*08:01:01 | B*35:189 | C*04:01:01 | C*07:01:01 |
| 01-006 | A*03:01:01 | A*24:02:01 | B*18:01:01 | B*35:01:01 | C*04:01:01 | C*07:01:01 |
| 01-007 | A*01:01:01 | A*02:01:01 | B*07:04 | B*08:01:01 | C*07:01:01 | C*07:02:01 |
| 01-008 | A*02:01:01 | A*03:01:01 | Unknown | Unknown | C*03:03:01 | C*12:03:01 |
| 01-009 | A*01:01:01 | X | B*37:01:01 | B*57:01:01 | C*06:02:01 | X |
| 01-010 | A*01:01:01 | A*24:02:01 | B*49:01:01 | X | C*07:01:01 | X |
| 01-001 | A*24:02:01 | X | B*18:01:01 | B*35:03:01 | C:04:01:01 | C*05:01:01 |
| 01-012 | Unknown | Unknown | B*15:01:01 | B*40:01:02 | C*03:03:01 | C*03:04:01 |
| 01-013 | A*24:02:01 | A*26:01:01 | B*15:01:01 | B*40:01:02 | C*03:03:01 | C*03:04:01 |
| 01-014 | A*02:06:01 | A*30:04:01 | B*35:03:01 | B*51:01:01 | C*04:01:01 | C*16:01:01 |
| 01-015 | A*02:01:01 | A*24:02:01 | B*18:01:01 | B*35:03:01 | C*04:01:01 | C*07:01:01 |
| 01-016 | A*02:01:01 | A*32:01:01 | B*18:01:01 | B*50:01:01 | C*06:02:01 | C*12:03:01 |
| 01-019 | A*03:01:01 | A*11:01:01 | B*35:03:01 | B*51:01:01 | C*12:03:01 | C*14:02:01 |
| 01-020 | A*02:01:01 | A*03:01:01 | B*07:02:01 | B*27:02:01 | C*02:02:02 | C*07:02:01 |
| 01-021 | A*03:01:01 | A*30:01:01 | B*07:02:01 | B:13:02:01 | C*06:02:01 | C*07:02:01 |
| 01-022 | A*03:01:01 | A*33:03:01 | B*07:02:01 | B*58:01:01 | C*03:02:02 | C*07:02:01 |
| 01-023 | A*11:01:01 | A*68:01:01 | B*35:01:01 | B*51:01:01 | C*04:01:01 | C*15:04:01 |
| 01-024 | A*24:02:01 | A*33:03:01 | B*35:01:01 | B*40:01:02 | C*03:04:01 | C*04:01:01 |
| 01-025 | A*01:01:01 | A*02:01:01 | B*08:01:01 | B*38:06:02 | C*07:01:01 | C*07:02:01 |
| 01-026 | A*02:120 | A*32:01:01 | B*07:02:01 | B*18:01:01 | C*07:02:01 | C*12:03:01 |
| 01-027 | A*01:01:01 | A*03:01:01 | B*39:06:02 | B*56:01:01 | C*01:02:01 | C*07:02:01 |
| 01-028 | A*01:01:01 | A*68:02:01 | B*15:17:01 | B*57:01:01 | C*06:02:01 | C*07:01:02 |
| 01-029 | A*01:01:01 | A*33:01:01 | B*14:02:01 | B*15:01:01 | C*03:04:01 | C*08:02:01 |
| 01-030 | A*01:01:01 | A*24:02:01 | B*07:02:01 | B*08:01:01 | C*07:01:01 | C*07:02:01 |
| 01-031 | A*03:01:01 | A*24:02:01 | B*35:03:01 | B*39:06:02 | C*04:01:01 | C*07:02:01 |
| 01-032 | A*02:01:01 | A68:01:01 | B*41:02:01 | B*51:01:01 | C*02:02:02 | C*17:03 |
| 01-033 | A*02:01:01 | A*24:02:01 | B*44:03:01 | B*50:01:01 | C*06:02:01 | C*16:01:01 |
| 01-034 | A*01:01:01 | A*11:01:01 | B*18:01:01 | B*35:01:01 | C*04:01:01 | C*07:01:01 |
| 01-035 | A*02:01:01 | A*30:01:01 | B*13:02:01 | B*35:02:01 | C*04:01:01 | C*06:02:01 |
| 01-036 | A*01:01:01 | A*23:01:01 | B*49:01:01 | B*52:01:01 | C*07:01:01 | C*12:02:02 |
| 01-037 | A*02:01:01 | X | B*07:02:01 | B*13:02:01 | C*06:02:01 | C*07:02:01 |
| 01-038 | A*02:01:01 | X | B*18:01:01 | B*49:01:01 | C*07:01:01 | X |
| 01-039 | A*01:01:01 | A*11:01:01 | B*35:01:01 | B*57:01:01 | C*04:01:01 | C*06:02:01 |
| 01-040 | A*01:01:01 | A*02:01:01 | B*15:01:01 | B*57:01:01 | C*03:04:01 | C*06:02:01 |
| 01-041 | A*01:01:01 | A*11:01:01 | B*08:01:01 | B*35:01:01 | C*04:01:01 | C*07:01:01 |
| 01-042 | A*01:01:01 | A*02:01:01 | B*37:01:01 | B*51:05 | C*04.01.01 | C*06:02:01 |
| 01-043 | A*02:01:01 | A*68:01:02 | B*07:02:01 | B*40:02:01 | C*03:06:01 | C*07:02:01 |
| 01-044 | A*28:01:01 | A*34:01:01 | B*40:01:02 | X | C*03:03:01 | C*03:04:01 |
| 01-045 | A*03:01:01 | A*11:01:01 | B*52:01:01 | B*57:01:01 | C*06:02:01 | C*12:02:02 |
| 01-046 | A*03:02:01 | A*30:01:01 | B*13:02:01 | B*55:01:01 | C*03:03:01 | C*06:02:01 |
| 01-047 | A*24:02:01 | A*68:01:01 | B*15:01:01 | B*36:02:01 | C*03:03:01 | C*04:01:01 |
| 01-048 | A*24:02:01 | A*31:01:02 | B*18:01:01 | B*40:04 | C*03:04:01 | C*12:03:01 |
| 01-049 | A*02:01:01 | A*03:01:01 | B*35:01:01 | B*51:01:01 | C*02:02:02 | C*04:01:01 |
| 01-050 | A*01:01:01 | A*23:01:01 | B*08:01:01 | B*49:01:01 | C*07:01:01 | X |
| 01-051 | Unknown | Unknown | Unknown | Unknown | Unknown | Unknown |
| 02-001 | A*29:02:01 | A*30:02:01 | B*91:01:01 | B*57:01:01 | C*02:10:01 | C*16:01:01 |
| 02-002 | A*03:01:01 | A*23::01:01 | B*07:02:01 | B*49:01:01 | C*07:01:01 | C*07:02:01 |
| 02-003 | A*26:01:01 | A*33:01:01 | B*14:02:01 | B*38:01:01 | C*08:02:01 | C*12:03:01 |
| 02-004 | A*03:01:01 | X | B*07:02:01 | B*14:02:01 | C*07:02:01 | C*08:02:01 |
| 02-005 | A*02:01:01 | X | B*41:02:01 | B*44:02:01 | C*05:01:01 | C*17:03 |
| 02-006 | A*02:01:01 | A*25:01:01 | B*15:01:01 | B*44:03:01 | C*03:03:01 | C*16:01:01 |
| 02-007 | A*11:01:01 | A*24:02:01 | B*38:02:01 | X | C*07:02:01 | C*07:27:01 |
| 02-008 | A*02:01:01 | A*11:01:01 | B*44:02:01 | B*52:01:01 | C*03:04:01 | C*12:02:02 |
| 02-009 | A*01:01:01 | A*24:02:13 | B*40:06:01 | B*44:03:02 | C*07:06 | C*15:02:01 |
| 02-010 | A*03:01:01 | A*23:01:01 | B*15:17:01 | B*53:01:01 | C*08:02:01 | C*16:01:01 |
| 02-011 | A*02:02:01 | A*30:02:01 | B*15:16:01 | B*42:01:01 | C*14:02:01 | C*17:01:01 |
| 02-012 | A*01:01:01 | A*11:01:01 | B*35:01:01 | B*35:03:01 | C*04:01:01 | X |
| 02-013 | A*24:02:01 | A*29:02:01 | B*14:02:01 | B*44:03:01 | C*02:02:02 | C*16:01:01 |
| 02-014 | A*30:01:01 | A*74:01:01 | B*15:03:01 | B*42:01:01 | C*02:10:01 | C*17:01:01 |
| 02-015 | A*02:01:01 | A*31:01:02 | B*35:01:01 | B*48:01:01 | C*04:01:01 | C*08:03:01 |
| 02-016 | A*30:02:01 | A*33:03:01 | B*15:03:01 | B*57:02:01 | C*02:10:01 | C*18:02 |
| 02-017 | A*02:01:01 | A*29:02:01 | B*13:02:01 | B*40:01:02 | C*03:04:01 | C*06:02:01 |
| 02-018 | A*33:03:01 | A*68:02:01 | B*13:02:01 | B*44:03:01 | C*06:02:01 | X |
| 02-019 | A*01:01:01 | A*02:01:01 | B*40:01:02 | B*57:01:01 | C*03:04:01 | C*06:02:01 |
| 02-020 | A*01:01:01 | A*11:01:01 | B*35:01:01 | B*57:01:01 | C*04:01:01 | C*06:02:01 |
| 02-021 | A*02:01:01 | X | B*15:01:01 | B*57:01:01 | C*03:03:01 | C*06:02:01 |
| 02-022 | A*02:01:01 | X | B*40:01:02 | B*56:01:01 | C*01:02:01 | C*03:04:01 |
| 02-022 | A*02:01:01 | X | B*40:01:02 | B*56:01:01 | C*01:02:01 | C*03:04:01 |
| 02-023 | A*02:01:01 | X | B*15:01:01 | B*44:02:01 | C*03:03:01 | C*05:01:01 |
| 02-024 | A*02:01:01 | A*68:01:02 | B*15:01:01 | B*44:02:01 | C*03:04:01 | C*07:04:01 |
| 02-025 | A*02:01:01 | A*29:02:01 | B*44:03:01 | B*51:01:01 | C*14:02:01 | C*16:01:01 |
| 02-026 | A*02:01:01 | X | B*35:01:01 | B*40:01:02 | C*03:04:01 | C*04:01:01 |
| 02-027 | A*02:02:01 | A*33:03:01 | B*49:01:01 | B*53:01:01 | C*04:01:01 | C*07:01:01 |

X: No additional haplotype (presumed homozygous)

As HLA A*02:01 is the most common MHC allele world-wide, nine HLA-A*02:01 patients were selected with a broad range of clinical presentations: six had mild symptoms and were not hospitalized, two required supplemental oxygen, and one required invasive ventilation. In each case, bulk memory CD8+ T cells (CD8+, CD45RO+, CD45RA-, CD57-) were collected by negative selection, the cells were expanded with antigen-independent stimulation (anti-CD3), and the cells were screened against the SARS-CoV-2 library. Target cells expressing only HLA-A*02:01 were used to provide unambiguous MHC restriction of discovered antigens.

SARS-CoV-2 screening results for one representative patient and one COVID-19-negative healthy control (blood collected prior to 2020) are shown in FIG. 4C. Reactivity to at least eight regions of SARS-CoV-2 proteins in the convalescent patient was found and none in the control. Importantly, reproducible performance of four technical screen replicates, internal nucleic acid barcodes, and overlapping protein fragments, collectively, was observed, indicating robust screen performance. Additionally, reactivity to the control CMV epitope (NLVPMVATV) was detected in the healthy control, who was known to be CMV-positive, and reactivity to two EBV epitopes in both the COVID-19 patient and the healthy control were detected (FIG. 4C).

Next, the screen results for the full set of HLA-A* 02:01 patients was examined and reactivity to specific segments of SARS-CoV-2 ORFs was detected in 8 of 9 patients (FIG. 5A). Strikingly, it was found that specific fragments are recurrently recognized by the T cells of multiple patients. For example, ORF1ab aa 3881-3900 and S aa 261-280 were each recognized by 7 of 9 patients (FIG. 5A). Overall, six regions were identified that were targeted by CD8+ T cells from at least three different patients. In addition to being shared across patients, these regions were among the strongest responses observed in each patient. Based on the results, it is believed that the CD8+ T cell response to SARS-CoV-2 is largely shaped by a limited number of recurrently targeted, immunodominant epitopes.

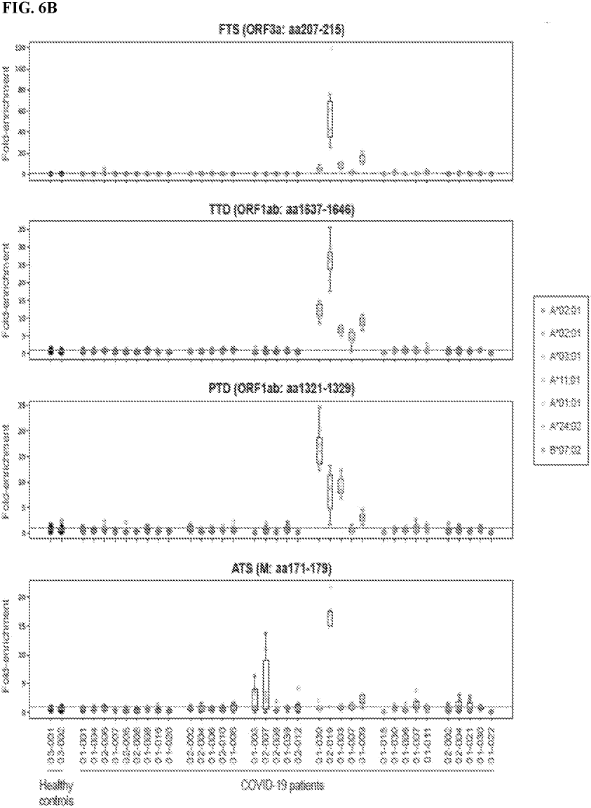

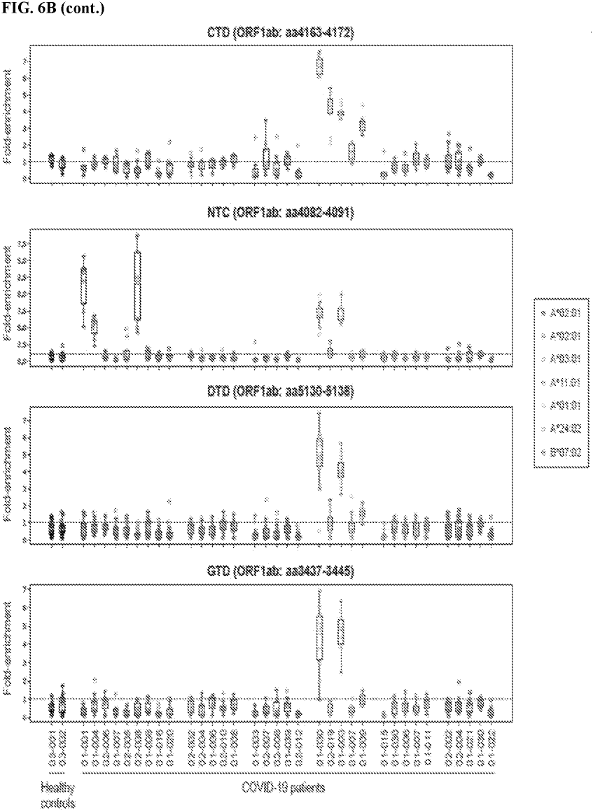

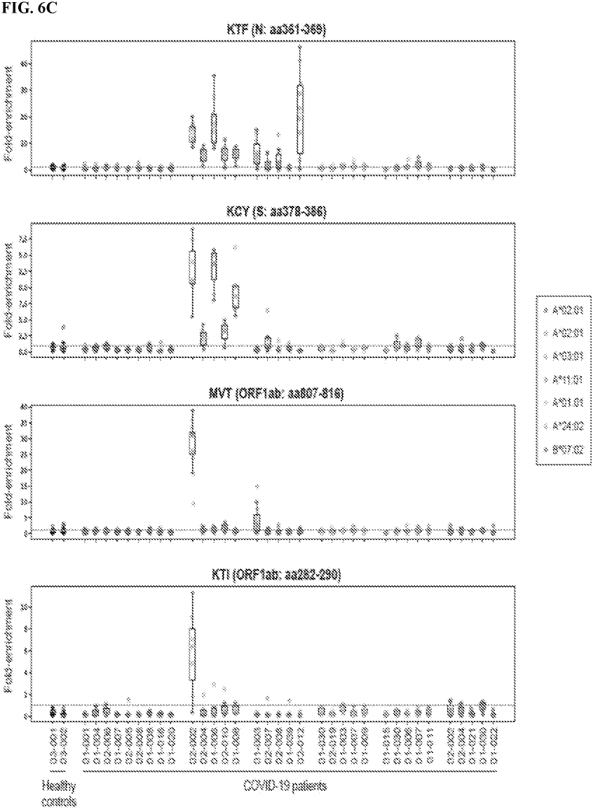

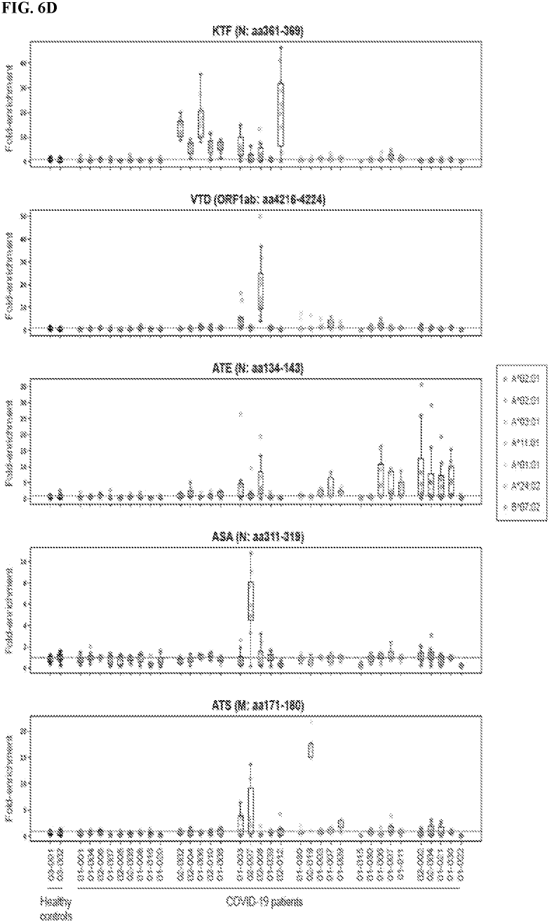

It was next sought to identify the precise peptide epitopes underlying the shared T cell reactivities detected in the screens. The overlapping design of the antigen library allowed the mapping of T cell reactivities to specific 20-aa segments. The NetMHC4.0 prediction algorithm (Andreatta and Nielsen (2016) Bioinform. 32:511-517; Nielsen et al. (2003) Prot. Sci. 12:1007-1017) was then used to identify high-affinity HLA-A*02:01 peptides in each pre-identified 20-aa stretch. A representative example of a predicted epitope and the corresponding screen data are shown in FIG. 5B. Additional epitopes are shown in FIG. 6.

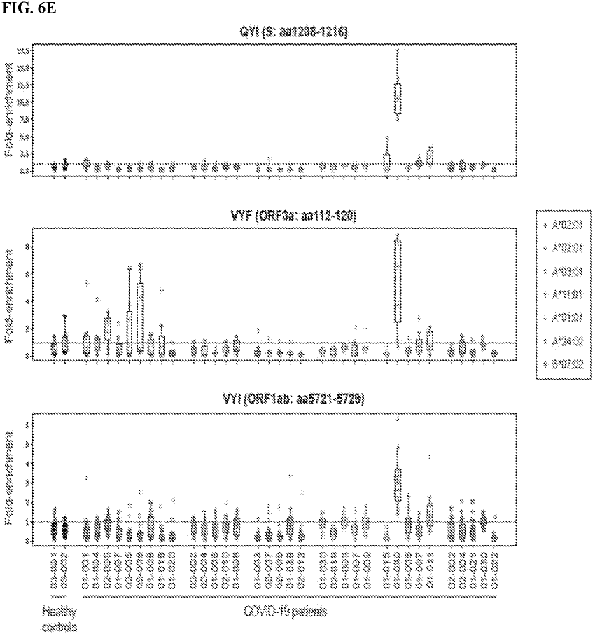

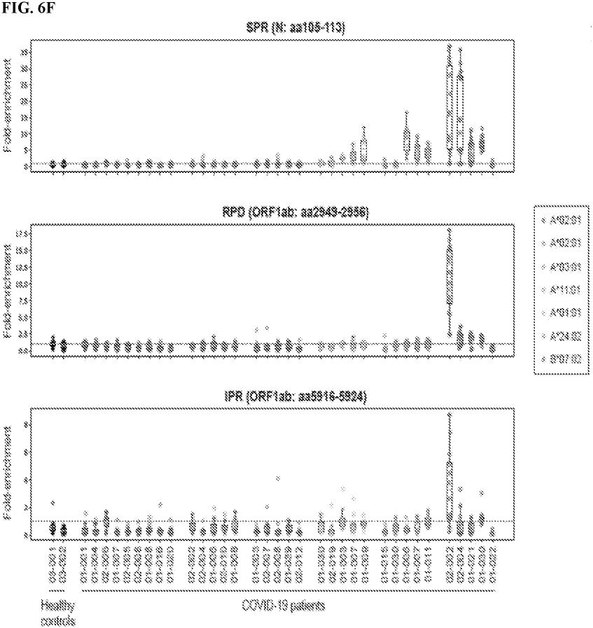

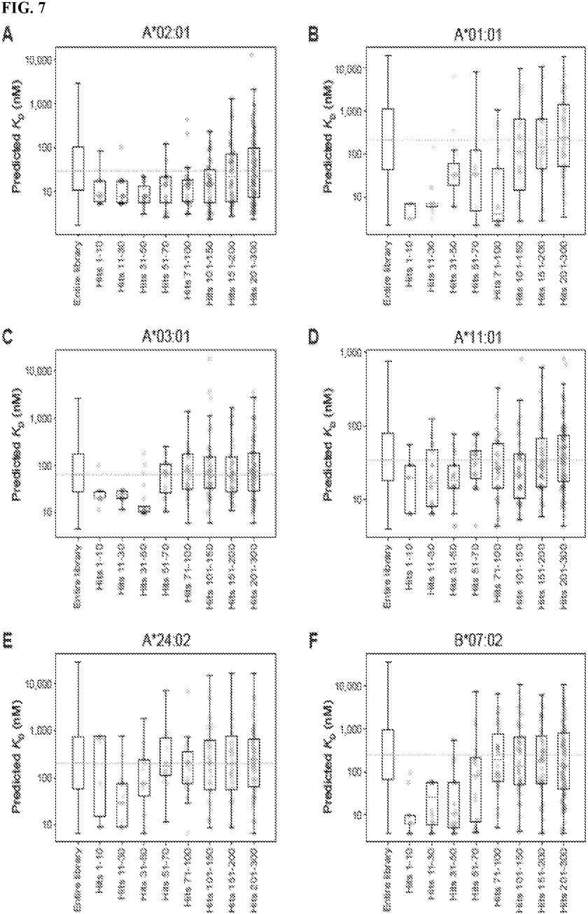

Notably, the fragments scoring in the screens were enriched for high-affinity HLA-binding peptides compared to the library as a whole, further verifying their biological relevance (FIG. 7). To visualize the results across all nine patients, the screening data were collapsed into a single value (mean of screen replicates and redundant tiles), revealing a set of six predicted epitopes that were recurrently recognized by three or more patients (FIG. 5C and Table 5).

TABLE 5

List of immunodominant T cell epitopes identified in convalescent COVID-19 patients

| | Allele | Peptide Name | Full Peptide | Parent Protein | Start | End | Affinity (nM) | % of Pts (Screen) | % of Pts (Tetramer) |
|---|---|---|---|---|---|---|---|---|---|
| 1 | A*02 | KLW | KLWAGCVCL | ORFtab | 3886 | 3894 | 17.7 | 88.9 | 77.8 |
| 2 | A*02 | YLQ | YLQPRTFLL | S | 269 | 277 | 5.4 | 77.8 | 44.4 |
| 3 | A*02 | LLY | LLYDANYFL | ORF3a | 139 | 147 | 3.1 | 88.9 | 55.6 |
| 4 | A*02 | ALW | ALWEIQQVV | ORFtab | 4094 | 4102 | 7.8 | 88.9 | 25.9 |
| 5 | A*02 | LLL | LLLDRLNQL | N | 222 | 230 | 14.8 | 33.3 | 22.2 |
| 6 | A*02 | YLF | YLFDESGEFKL | ORFtab | 906 | 916 | 22.2 | 44.4 | 18.5 |
| 7 | A*01 | FTS | FTSDYYQLY | ORF3a | 207 | 215 | 3.2 | 100 | |
| 8 | A*01 | TTD | TTDPSFLGRY | ORFtab | 1637 | 1646 | 7.2 | 100 | |
| 9 | A*01 | PID | PTDNYITTY | ORFtab | 1321 | 1329 | 6.1 | 80 | |
| 10 | A*01 | ATS | ATSRTLSYY | M | 171 | 179 | 16.7 | 80 | |
| 11 | A*01 | CTD | CTDDNALAYY | ORFtab | 4163 | 4172 | 5.3 | 100 | |
| 12 | A*01 | NTC | NTCOGTTFTY | ORFtab | 4082 | 4091 | 121.8 | 60 | |
| 13 | A*01 | DTD | DTDFVNEFY | ORFtab | 6130 | 5138 | 2.8 | 40 | |
| 14 | A*01 | GTD | GTDLEGNFY | ORFtab | 3437 | 3445 | 6 | 40 | |
| 15 | A*03 | KTF | KTFPPTEPK | N | 361 | 368 | 20.8 | 100 | |
| 16 | A*03 | KCY | KCYGVSPTKY | S | 378 | 386 | 152.6 | 100 | |
| 17 | A*03 | MVT | MVTNNTFTLK | ORFtab | 807 | 816 | 19.8 | 40 | |
| 18 | A*03 | KTI | KTIQPRVEK | ORFtab | 282 | 230 | 113.2 | 40 | |
| 19 | A*11 | KTF | KTFPPTEPK | N | 361 | 369 | 6.3 | 100 | |
| 20 | A*11 | VTD | VTDTPKGPK | ORFtab | 4216 | 4224 | 160.5 | 60 | |
| 21 | A*11 | ATE | ATEGALNTPK | N | 134 | 143 | 55.5 | 80 | |
| 22 | A*11 | ASA | ASAFFGMSR | N | 311 | 319 | 14.4 | 40 | |
| 23 | A*11 | ATS | ATSRTLSYYK | M | 171 | 180 | 7.9 | 60 | |
| 24 | A*24 | QYI | QYIKWPWYI | S | 1208 | 1216 | 13.2 | 60 | |
| 25 | A*24 | VYF | VYFLQSINF | ORF3a | 112 | 120 | 47.4 | 80 | |
| 26 | A*24 | VYI | VYIGDPAQL | ORFtab | 5721 | 5729 | 206 | 40 | |
| 27 | B*07 | SPR | SPRWYFYYL | N | 106 | 113 | 6.3 | 80 | |
| 28 | B*07 | RPD | RPDTRYVL | ORFtab | 2949 | 2996 | 56.9 | 80 | |
| 29 | B*07 | IPR | IPRRNVATL | ORFtab | 5916 | 5924 | 5.1 | 20 | |

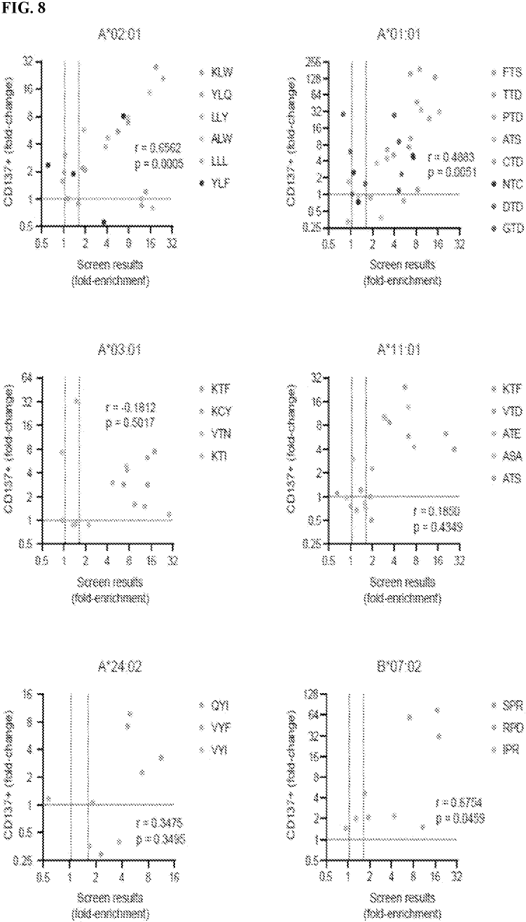

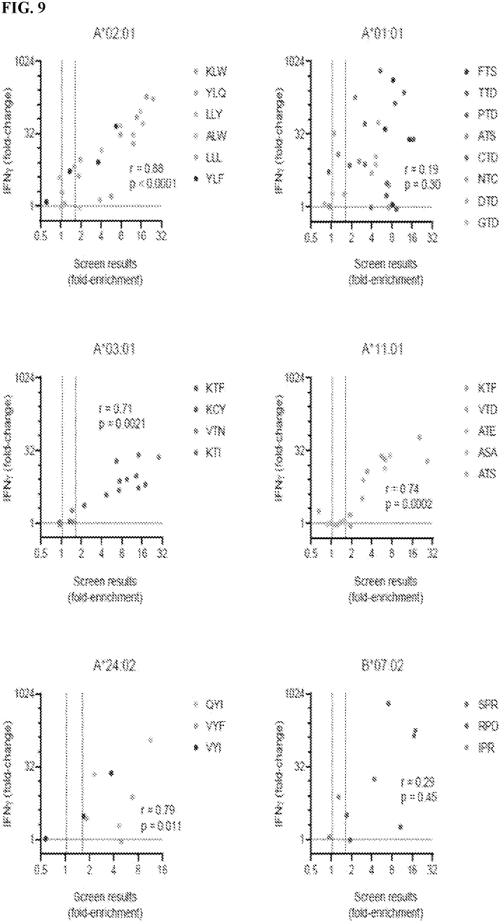

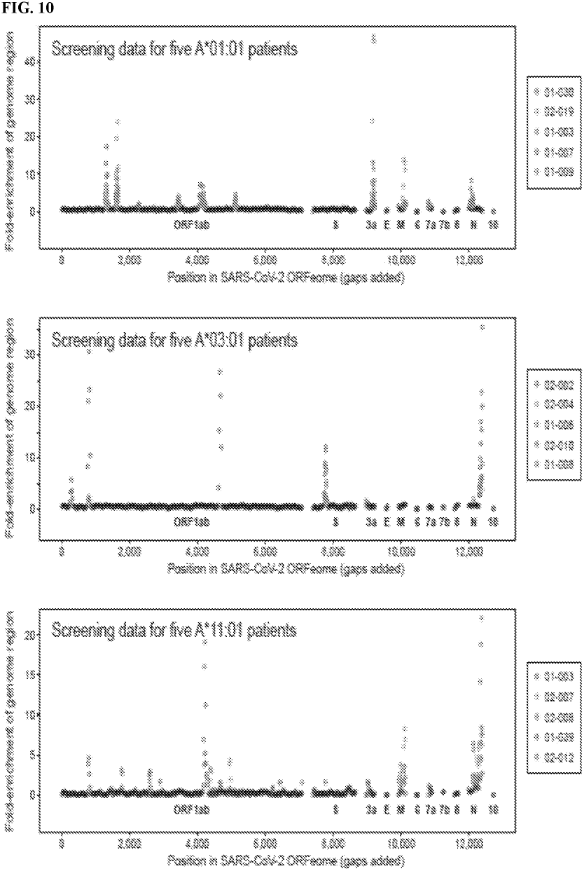

Peptides corresponding to each predicted epitope were then synthesized to further validate the results. All six epitopes induced peptide-dependent T-cell activation as determined by interferon-gamma (IFNg) secretion (FIG. 5D) and CD137 upregulation (FIG. 8). Both IFN gamma (IFNg) secretion and CD 137 upregulation correlate with the fold enrichment in the TScan screen (FIG. 8 and FIG. 9). As further validation, MHC tetramers with the six peptides were constructed and used to stain the memory CD8+ T cells of all nine A*02:01 patients, as well as an additional 18 A*02:01 patients that had not been previously screened. Positive tetramer staining was observed in a subset of patients for all six peptides, including patients who had not been screened (FIG. 5E). Notably, the magnitude of enrichment in the screens correlated well with the frequency of cognate T cells in the patient samples (r = 0.73, p < 0.0001) (FIG. 5F), indicating that the screens detected the targets of T cells that are present at ≥0.1% frequency in the memory CD8+ T cell pool. Remarkably, the three most commonly recognized epitopes discovered - KLW, YLQ, and LLY - are each recognized by 67% of the patients screened, and all nine patients had a detectable response to at least one of the top three epitopes (FIG. 5G). A similar analysis of the tetramer staining data in all 27 A*02:01 patients showed recognition of at least one of these epitopes in 23 of 27 patients (85% of patients) (FIG. 5H). Taken together, the analysis of HLA-A*02:01 patients demonstrates the utility of the T-Scan approach in mapping SARS-CoV-2 T cell epitopes and reveals that patient T cells largely target a limited set of shared immunodominant epitopes.

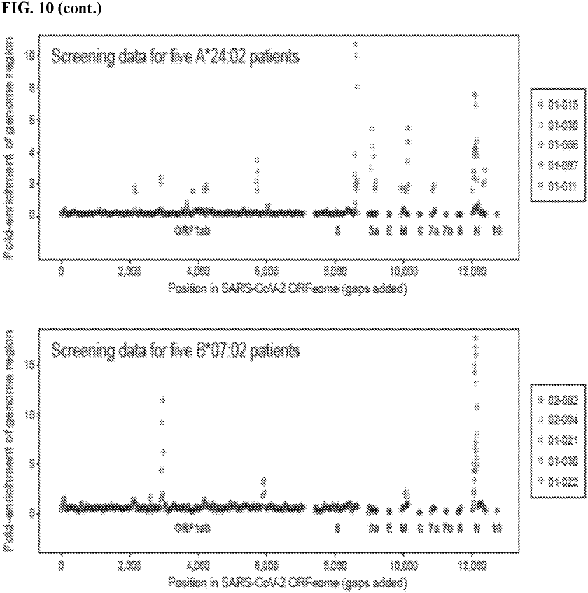

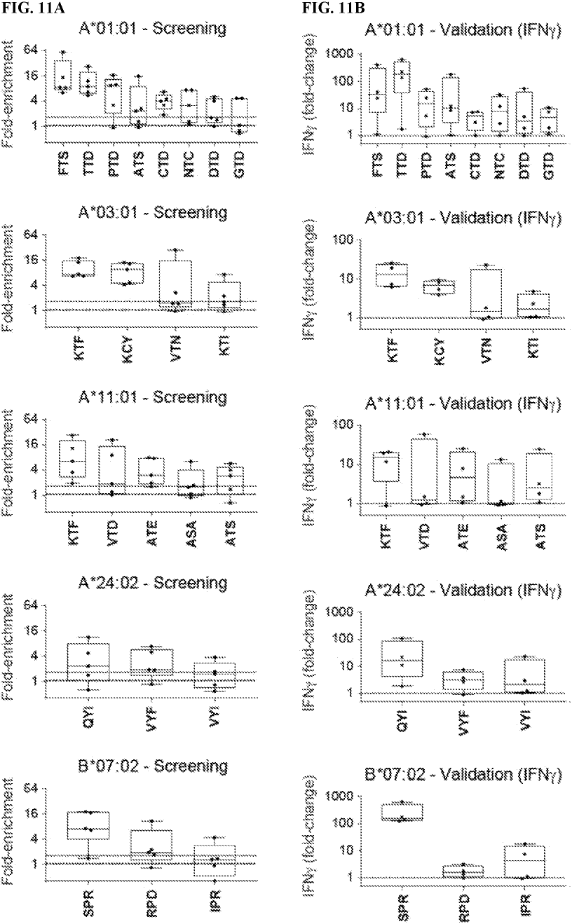

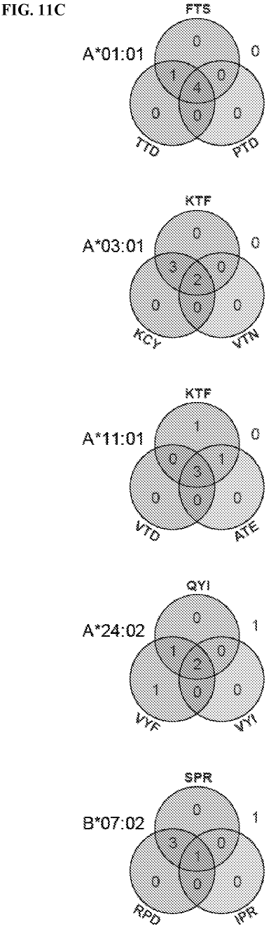

CD8+ T cell responses are profoundly shaped by host MHC alleles, which restrict the scope of displayed peptides that serve as potential antigens. To determine whether the narrow set of immunodominant epitopes identified for HLA-A*02:01 reflects a general feature of anti-SARS-CoV-2 CD8+ T cell responses, memory CD8+ T cell reactivities were mapped for five additional common MHC alleles: HLA-A*01:01, HLA-A*03:01, HLA-A*11:01, HLA-A*24:02, and HLA-B*07:02. Analysis of this set of HLA alleles provides a broad perspective on the nature of anti-SARS-CoV-2 CD8+ T cell immunity, as ~90% of the U.S. population and ~85% of the world population is positive for at least one of the six alleles examined (Maiers et al. (2007) Hum. Immunol.. 68:779-788; Gonzalez-Galarza (2020) Nucl. Acids Res. 48:D783-D788). For each allele, five HLA+ convalescent COVID-19 patients were selected and their memory CD8+ T cells were screened against the SARS-CoV-2 library in target cells expressing only the single HLA of interest. As with A*02:01 patients, robust T cell recognition of multiple regions in the SARS-CoV-2 ORFeome for patients with each HLA allele was found (FIG. 10) and it was confirmed that the scoring fragments were enriched for predicted high-affinity MHC binders for each respective allele (FIG. 7). Strikingly, recurrent recognition of specific protein fragments by most or all patients for each allele was again observed (FIG. 11A), indicating a narrow set of shared immunodominant responses. As described above, screening data and NetMHC4.0 MHC binding analyses were combined to map the precise epitopes underlying the top hits from the screens, and these peptides were validated using representative IFNg secretion assays (FIG. 11B) and CD137 upregulation assays (FIG. 8). Three or more recurrently recognized epitopes on each screened MHC allele were identified and it was determined that 92% of patients recognized at least one of the top three allele-specific epitopes (FIG. 11C). Collectively, a set of 29 CD8+ T cell epitopes that were shared among COVID-19 patients with the same HLA type were mapped and validated (Table 5). Most strikingly, it was found that the CD8+ T cell response restricted by each of six common HLA alleles contained a limited number of recurrently targeted, immunodominant epitopes.

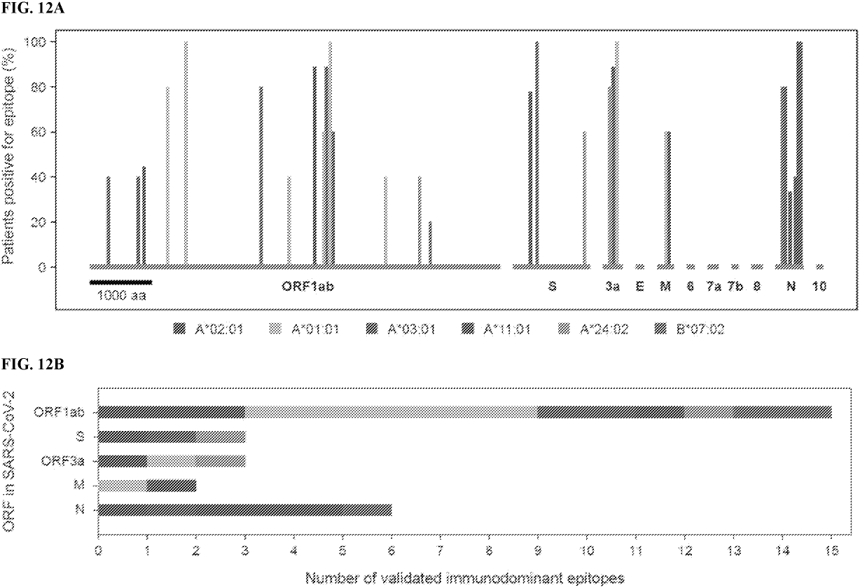

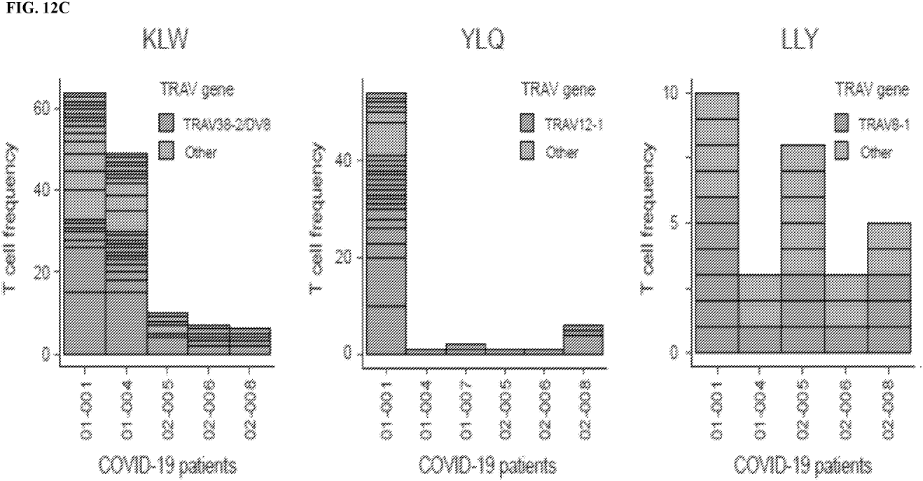

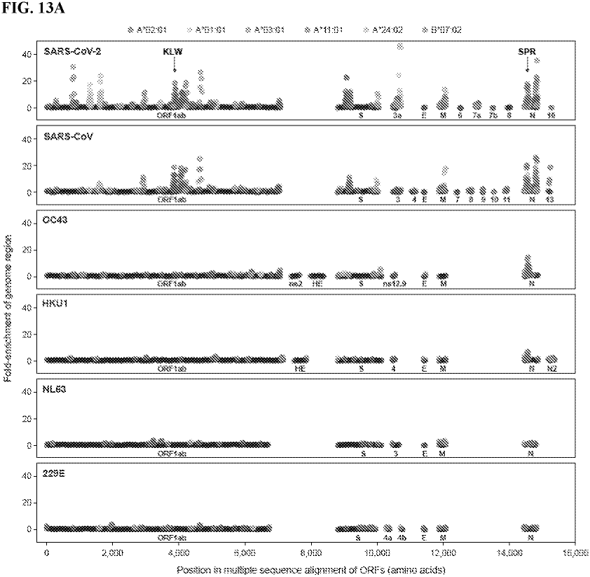

The unbiased antigen mapping performed allowed for the interrogation of various features of CD8+ T cell immunity to SARS-CoV-2. First, the scope of recognized viral proteins was examined. Broad reactivity to many SARS-CoV-2 proteins, including ORF1ab, S, N, M, and ORF3a, was observed (FIG. 12A). Notably, only three of the 29 epitopes were located in the S protein, with most (15 of 29) located in ORF1ab and the highest density of epitopes located in the N protein (FIG. 12A and FIG. 12B). When taken in aggregate, the results are largely consistent with previous ORF-level analyses using peptide pools (Grifoni et al. (2020) Cell 181:1489-1501; Le Bert et al. (2020) “SARS-CoV-2-specific T cell immunity in cases of COVID-19 and SARS, and uninfected controls” Nature (doi: 10.1038/s41586-020-2550-z) available at nature.com/articles/s41586-020-2550-z; Braun et al. (2020) “Presence of SARS-CoV-2 reactive T cells in COVID-19 patients and healthy donors” medRxiv (doi.org/10.1101/2020.04.17.20061440) available at medrxiv.org/content/10.1101/2020.04.17.20061440v1; Thieme et al. (2020) “The SARS-CoV-2 T-cell immunity is directed against the spike, membrane, and nucleocapsid protein and associated with COVID 19 severity” medRxiv (doi.org/10.1101/2020.05.13.20100636) available at medrxiv.org/content/10.1101/2020.05.13.20100636v1; Altmann and Boyton (2020) Science Immunol. 5:eabd6160). However, the approaches described and carried out herein provided an increased level of granularity that allowed for the identification of specific epitope sequences and highlighted allele-specific differences. For example, immunodominant epitopes in the S protein were observed for HLA-A*02:01, HLA-A*03:01, and HLA-A*24:02, but not for HLA-A*01:01, HLA-A*11:01, or HLA-B*07:02. Only one recurrent response in the receptor-binding domain (RBD) of the S protein (KCY on HLA-A*03:01) was detected.

It was next asked how the CD8+ T cell response to SARS-CoV-2 intersected with the emerging genetic diversity of the virus. Recent analyses, which examined the genome sequences of over 10,000 isolates of SARS-CoV-2 sampled from 68 different countries, identified a set of 28 non-synonymous coding mutations detected in at least 1% of strains (Koyama et al. (2020) Bulletin of the World Health Organization (WHO) 98:495-504). Only one of these mutations (M protein T175M; detected in 2% of strains) was found in the immunodominant epitope identified (HLA-A*01:01 ATS and HLA-A*11:01 ATS). These results indicate that the recognition of the epitopes identified and described herein are not significantly influenced by the SARS-CoV-2 genetic diversity observed thus far.

Identifying specific SARS-CoV-2 epitopes allowed for the examination of the features of the T cell receptors (TCRs) recognizing these immunodominant epitopes. Tetramers loaded with three HLA-A*02:01 epitopes (KLW, YLQ, and LLY) were used to stain and sort antigen-specific memory CD8+ T cells from the initial nine HLA-A*02:01-positive convalescent COVID-19 patients. 10X Genomics single-cell sequencing was then used to identify the paired TCR alpha and TCR beta chains expressed by these T cells. Paired clonotypes reactive to each antigen in 5/9 (KLW, ALW) or 6/9 (YLQ) patients. For a majority of responses (9/16), oligoclonal recognition by five or more distinct clonotypes was detected. Next, the TCR sequences themselves were identified. Striking similarity among the TCRs recognizing each antigen in terms of Vα gene segment usage and, to a lesser extent, Vβ usage (FIG. 12C) was observed. Specifically, 26/61 KLW-reactive clonotypes used TRAV38-2/DV8, 24/31 YLQ-reactive clonotypes used TRAV12-1, and 14/29 LLY-reactive clonotypes used TRAV8-1. Notably, these dominant Vα genes were used across all of the patients for whom reactive clonotypes were identified. Taken together, these data indicate that the epitopes identified are recognized by TCRs with shared sequence features and raise the possibility that their immunodominance is shaped by the structural requirements for high-affinity TCR binding to these peptide-MHC complexes.

Another important question is how pre-existing immunity to other coronaviruses shapes the CD8+ T cell response to SARS-CoV-2. There are four commonly circulating coronaviruses, OC43, HKU1, NL63, and 229E, and cross-reactive responses to these viruses have been theorized as a potential protective factor during SARS-CoV-2 infection (Cui et al. (2019) Nat. Rev. Microbiol. 17:181-192).

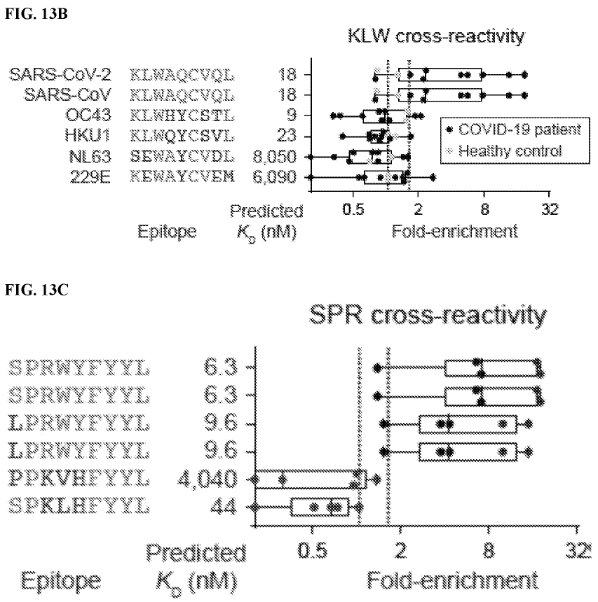

Moreover, understanding the extent of cross-reactivity has implications for accurately monitoring T cell responses to SARS-CoV-2 and for optimizing vaccine design. If the immune response to SARS-CoV-2 is shaped by pre-existing CD8 T cells that recognize other coronaviruses, it was hypothesized that COVID-19 patients should have reactivity to the regions of the other coronaviruses that correspond to the SARS-CoV-2 immunodominant epitopes identified and described herein. Accordingly, T-cell reactivity to SARS-CoV-2, SARS-CoV, and all four endemic coronaviruses was examined in all 34 genome-wide screens conducted - across all patients and all MHC alleles (FIG. 13A). Broad reactivity to the corresponding epitopes in SARS-CoV in over half of cases was observed, which is consistent with a recent study reporting the existence of long-lasting memory T cells cross-reactive to SARS-CoV-2 in patients that had been infected in SARS-CoV during the 2002/2003 SARS outbreak (Le Bert et al. (2020) “SARS-CoV-2-specific T cell immunity in cases of COVID-19 and SARS, and uninfected controls” Nature (doi: 10.1038/s41586-020-2550-z) available at nature.com/articles/s41586-020-2550-z). In contrast, however, almost no reactivity to OC43 and HKU1 (2/29 dominant epitopes) and no reactivity to NL63 and 229E was detected. Beyond the 29 epitopes, no reproducible cross-reactivity to any other regions of the four endemic coronaviruses was detected, further indicating that prior exposure to these viruses is unlikely to provide T cell-based protection from SARS-CoV-2.

Identification and description herein of specific immunodominant epitopes in SARS-CoV-2 allowed for the provision of an explanation for this lack of cross-reactivity. In some cases, the corresponding region is poorly conserved in the other coronaviruses and high-affinity binding to MHC is lost (see, for example, the corresponding regions of the KLW epitope in NL63 and 229E) (FIG. 13B). In other cases, the corresponding epitopes are still predicted to bind with high affinity to MHC, but SARS-CoV-2-reactive T cells do not recognize them (see, for example, the corresponding regions of the KLW epitope in OC43 and HKU10 (FIG. 13B).

In one case, a strong cross-reactive response was identified. The HLA B*07:02 epitope SPR, which lies in the N protein, is highly conserved across betacoronaviruses and all four of the patients that demonstrated reactivity to SPR also exhibited reactivity to the corresponding epitopes in OC43 and HKU1 (FIG. 13C). Overall, however, it was determined herein that the CD8+ T cell response to SARS-CoV-2 is not significantly shaped by pre-existing immunity to endemic coronaviruses.

Based on the foregoing, natural CD8+ T cell response to SARS-CoV-2 were analyzed using an unbiased, genome-wide method that enabled identification of the precise epitopes presented on MHC and functionally recognized by memory CD8+ T cells in convalescent patient blood. All 29 epitopes identified were validated using independent functional assays, and the A* 02:01-restricted epitopes were further validated in an independent test set of 18 patients. Overall, a core set of 3-8 immunodominant epitopes for each MHC allele was found. These epitopes were recurrently targeted across patients, but also represented the strongest hits in the screens within each patient, indicating that they are both shared and dominant. Moreover, these epitopes are almost entirely specific to SARS-CoV-2/SARS-CoV, indicating that the T cell response to SARS-CoV-2 is not significantly shaped by pre-existing immunity to the four endemic coronaviruses that cause the common cold.

The results described herein contrast with in silico studies predicting epitopes presented by HLA alleles. For example, hundreds of SARS-CoV-2-derived peptides are predicted to bind with high affinity to HLA-A*02:01 (Nguyen et al. (2020) “Human leukocyte antigen susceptibility map for SARS-CoV-2” J. Virol. (10.1128/JVI.00510-20) available at vi.asm.org/content/94/13/e00510-20), yet the results of actual T-cell responses described herein reveal eight or fewer dominant A*02:01-restricted targets per patient. Based on the strong correlation observed between the screening data and tetramer staining, it is estimated that the screens detect T cell specificity that is present at a frequency of ≥0.1% in the pool of memory cells. Although there may be other virus-specific T cells below this frequency, those detected represent the most expanded clones and so are likely to be most important in providing protection from future infection. Generating a T cell response depends not only on high-affinity binding of the peptide to the MHC, but also on efficient processing and loading of the peptide, as well as efficient recognition of the peptide by TCRs in the naive repertoire of the patient. Indeed, our clonotype analysis of the three most dominant A*02:01 epitopes (KLW, YLQ, and LLY) revealed that the T cell response is oligoclonal, but dominated by specific T cell receptor Va and Vb chains that are similarly shared across patients. This highlights the importance of experimentally identifying immunodominant epitopes in an unbiased fashion.

The results described herein also highlighted differences across MHC alleles in the total number of recognized epitopes and the proteins in which they reside. This emphasizes the importance of searching for MHC associations with disease outcome and of detailed tracking of MHC alleles in immune monitoring of vaccine trials. Previous studies using megapools of peptides spanning each of the ORFs in SARS-CoV-2 showed CD4+ and CD8+ T cell responses in all COVID-19 convalescent patients (Grifoni et al. (2020) Cell 181:1489-1501; Le Bert et al. (2020) “SARS-CoV-2-specific T cell immunity in cases of COVID-19 and SARS, and uninfected controls” Nature (doi: 10.1038/s41586-020-2550-z) available at nature.com/articles/s41586-020-2550-z; Braun et al. (2020) “Presence of SARS-CoV-2 reactive T cells in COVID-19 patients and healthy donors” medRxiv (doi.org/10.1101/2020.04.17.20061440) available at medrxiv.org/content/10.1101/2020.04.17.20061440v1; Thieme et al. (2020) “The SARS-CoV-2 T-cell immunity is directed against the spike, membrane, and nucleocapsid protein and associated with COVID 19 severity” medRxiv (doi.org/10.1101/2020.05.13.20100636) available at medrxiv.org/content/10.1101/2020.05.13.20100636v1; Altmann and Boyton (2020) Science Immunol. 5:eabd6160). Although most of the reactivity to the S protein came from CD4+ T cells, some reactivity to the S protein was also observed in CD8+ T cells. Consistent with these findings, 3 immunodominant epitopes in the S protein were identified. Overall, however, it was found that 90% of the CD8+ T cell reactivity was directed at epitopes outside the S protein. Grifoni et al. (2020) Cell 181:1489-1501 also showed reactivity to the M protein, while Le Bert et al. (2020) “SARS-CoV-2-specific T cell immunity in cases of COVID-19 and SARS, and uninfected controls” Nature (doi: 10.1038/s41586-020-2550-z) available at nature.com/articles/s41586-020-2550-z found reactivity to nsp7 and nsp13, which derive from ORF1ab. Specific epitopes within these proteins, as well as their MHC restriction, are now described herein. In contrast to peptide pool studies that found T cells in unexposed individuals that were cross reactive to SARS-CoV-2, however, the results described herein demonstrate that the immunodominant epitopes are largely specific for SARS-CoV-2 and are not shared with other coronaviruses. If pre-existing memory responses to other coronaviruses were able to efficiently recognize SARS-CoV-2, then the reacting T cells would be expected to expand and their targets would be detected in the screens described herein. As a result, the paucity of cross-reactive responses found argues against substantial protection against SARS-CoV-2 stemming from CD8+ T cell immunity to the four coronaviruses that cause the common cold.

The additional level of granularity provided by identifying the specific epitopes as described herein also provides the necessary tools for tracking SARS-CoV-2-specific CD8+ T cell responses in exposed individuals or in subjects participating in vaccine trials. Diagnosis of previous exposure to SARS-CoV-2 currently relies on serological testing for antibodies that wane with time. A recent study found that IgG responses to SARS-CoV-2 decline rapidly in >90% of infected individuals in the 2-3-month period post infection, with 40% of asymptomatic individuals turning seronegative (Long et al. (2020) “Clinical and immunological assessment of asymptomatic SARS-CoV-2 infections” Nat. Med. (10.1038/s41591-020-0965-6) available atnature.com/articles/s41591-020-0965-6). In contrast, there are indications that memory T cells may persist longer, as T cells specific for SARS-CoV were detected 11 or even 17 years after the 2003 SARS outbreak (Le Bert et al. (2020) “SARS-CoV-2-specific T cell immunity in cases of COVID-19 and SARS, and uninfected controls” Nature (doi: 10.1038/s41586-020-2550-z) available at nature.com/articles/s41586-020-2550-z; Ng et al. (2016) Vaccine 34:2008-2014). Based on the results described herein, it is believed that detecting SARS-CoV-2-specific CD8+ T cells potentially can be performed at large scale using an IFNg release assay similar to commercial assays used for tuberculosis testing (Albert-Vega et al. (2018) Front. Immunol. 9:2367). Although the frequency of SAR-CoV-2-specific memory T cells decreases in the weeks following recovery from an acute infection, the remaining pool of memory T cells can be expanded in vitro by stimulation with peptide epitopes, as previously demonstrated for the detection of T cells to SARS-CoV (Le Bert et al. (2020) “SARS-CoV-2-specific T cell immunity in cases of COVID-19 and SARS, and uninfected controls” Nature (doi: 10.1038/s41586-020-2550-z) available at nature.com/articles/s41586-020-2550-z; Ng et al. (2016) Vaccine 34:2008-2014). In contrast to serological testing for antibodies, this allows for a diagnostic test that can detect prior exposure to COVID-19 for a prolonged period following viral infection. It also allows for determination of T cell reactivity to any or all of the immunodominant epitopes as an indicator of disease severity or protection against future infection.

The results described herein also have significant implications for vaccine development. A majority of the T cell responses described herein fall outside of the S protein. Only one is in the receptor binding domain of S. Accordingly, it is believed that more robust CD8+ T cell responses across diverse patients could be generated by incorporating additional antigens into vaccine designs. For example, specific regions of the ORF lab protein that could be used are provided. The smaller proteins, N, M, and ORF3a, are also believed to be strongly and broadly immunogenic. The epitopes identified and described herein carry the additional benefit that they occur in regions that have thus far been subject to minimal genetic variation. While there does not appear to be significant cross-reactivity with other coronaviruses, the few regions identified that are highly conserved and immunogenic are believed to be of specific interest because they are believed to confer protection across different coronaviruses. Studying these epitopes in prospective tracking studies can further confirm whether previous exposure to other coronaviruses elicits protective or pathological immune responses.

The determination that the immunodominant epitopes for CD8+ T cells reside largely outside the spike protein raises the possibility that many of the S protein-directed vaccines currently under development may elicit an insufficient CD8+ T cell response. It should be noted that a recent vaccine candidate, BNT162bl, an RNA vaccine encoding the receptor binding domain of the S protein, elicited CD8+ T cell responses in 80% of participants (Mulligan et al. (2020) “Phase 1/2 study to describe the safety and immunogenicity of a COVID-19 RNA vaccine candidate (BNT162b1) in adults 18 to 55 years of age: interim report” medRxiv ( doi.org/10.1101/2020.06.30.20142570) available at medrxiv.org/content/10.1101/2020.06.30.20142570v1). Given that only a single A*03:01-restricted immunodominant epitope in the RBD was observed, it is unlikely that the observed responses in this study are all directed at this epitope. Additional immunodominant epitopes may be presented by MHC alleles not examined, although it is unlikely that a large number of rare alleles display RBD-derived immunodominant epitopes while the six most prevalent alleles collectively feature only one. A more likely explanation is that vaccinating with a high dose of an RNA-based vaccine encoding a single protein domain could potentially elicit CD8+ T cells that recognize subdominant epitopes. It is believed that vaccines like this would benefit from additional peptides/proteins that elicit the naturally occurring shared epitopes.

Overall, the results described herein indicate that memory CD8+ T cell responses in convalescent COVID-19 patients are directed against a small set of immunodominant epitopes that are shared across the majority of patients with the same HLA types. These epitopes are largely outside the spike protein, the current target of the most advanced vaccines against SARS-CoV-2. These findings allow for the development of diagnostic tests for previous exposure to SARS-CoV-2 and support the inclusion of other antigens in vaccines against this virus that are more likely to mimic the natural CD8+ T cell response to SARS-CoV-2.

### Example 4: Applications of Identified Immunodominant Peptides

Strikingly, this study revealed a limited set of highly immunodominant peptide antigens that are recurrently recognized across patients, including several that appear to be universally recognized. In addition to shedding light on the nature and scope of immunity to the pathogen, this discovery enables a series of important applications. For example, in one embodiment, specific reagents to monitor T cell responses to SARS-CoV-2 are generated. These reagents may take the form of peptide-MHC tetramers displaying the discovered peptide antigens. In another embodiment, diagnostics to determine past exposure to and future protection from SARS-CoV-2 are developed. For example, the discovered peptide antigens may be used to stimulate PBMCs from test subjects. Higher levels of reactivity, indicated by T cell activation or effector function (for example, as read out by FACS or ELISA), reveals the presence of a past exposure and existing immunity to SARS-CoV-2. In still another embodiment, T-cell based therapeutics to combat SARS-CoV-2 are developed. These may include adoptive TCR therapy using TCRs against the discovered peptide antigens, or allogeneic products where donor T cells are expanded against the discovered peptide antigens. In yet another embodiment, improved vaccines that incorporate the viral proteins or specific peptides that were found to be immunodominant are designed. Current vaccines focus primarily on the S protein of SARS-CoV-2, whereas a majority of the T cell responses that have been discovered are to other proteins. Thus, the data described herein indicate the targets recognized by patients who have successfully overcome the SARS-CoV-2 virus, making the discovered targets especially attractive for inclusion in vaccines.

### Incorporation by Reference

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the World Wide Web at tigr.org and/or the National Center for Biotechnology Information (NCBI) on the World Wide Web at ncbi.nlm.nih.gov.

### Equivalents

Those skilled in the art will recognize, or be able to ascertain using no more than routine experimentation, many equivalents to the specific embodiments encompassed by the present invention described herein. Such equivalents are intended to be encompassed by the following claims.

What is claimed is:

1. An immunogenic peptide comprising a peptide epitope selected from Table 1A, 1B, 1C, 1D, 1E, and/or 1F.

2. An immunogenic peptide consisting of a peptide epitope selected from Table 1A, 1B, 1C, 1D, 1E, and/or 1F.

3. The immunogenic peptide of claim 1 or 2, wherein the immunogenic peptide is derived from a SARS-CoV-2 protein, optionally wherein the immunogenic peptide is 8, 9, 10, 11, 12, 13, 14, or 15 amino acids in length.

4. The immunogenic peptide of claim 3, wherein the SARS-CoV-2 protein is selected from the group consisting of orf1a/b, S protein, N protein, M protein, orf3a, and orf7a.

5. The immunogenic peptide of any one of claims 1-4, wherein the immunogenic peptide is capable of eliciting a T cell response in a subject.

6. An immunogenic composition comprising at least one immunogenic peptide of any one of claims 1-5.

7. The immunogenic composition of claim 6, further comprising an adjuvant.

8. The immunogenic composition of claim 6 or 7, wherein the immunogenic composition is capable of eliciting a T cell response in a subject.

9. A composition comprising a peptide epitope selected from Table 1A, 1B, 1C, 1D, 1E, and/or 1F, and an MHC molecule.

10. The composition of claim 9, wherein the MHC molecule is a MHC multimer, optionally wherein the MHC multimer is a tetramer.

11. The composition of claim 9 or 10, wherein the MHC molecule is an MHC class I molecule.

12. The composition of any one of claims 9-11, wherein the MHC molecule comprises an MHC alpha chain that is an HLA serotype selected from the group consisting of HLA-A*02, HLA-A*03, HLA-A *01, HLA-A*11, HLA-A*24, and/or HLA-B*07, optionally wherein the HLA allele is selected from the group consisting of HLA-A* 0201, HLA-A*0202, HLA-A*0203, HLA-A*0204, HLA-A*0205, HLA-A*0206, HLA-A*0207, HLA-A*0210, HLA-A*0211, HLA-A*0212, HLA-A*0213, HLA-A*0214, HLA-A*0216, HLA-A*0217, HLA-A*0219, HLA-A*0220, HLA-A*0222, HLA-A*0224, HLA-A*0230, HLA-A*0242, HLA-A*0253, HLA-A*0260, HLA-A*0274 allele, HLA-A*0301, HLA-A*0302, HLA-A*0305, HLA-A*0307, HLA-A*0101, HLA-A*0102, HLA-A*0103, HLA-A*0116 allele, HLA-A*1101, HLA-A*1102, HLA-A*1103, HLA-A*1104, HLA-A*1105, HLA-A*1119 allele, HLA-A*2402, HLA-A*2403, HLA-A*2405, HLA-A*2407, HLA-A*2408, HLA-A*2410, HLA-A*2414, HLA-A*2417, HLA-A*2420, HLA-A*2422, HLA-A*2425, HLA-A*2426, HLA-A*2458 allele, HLA-B*0702, HLA-B*0704, HLA-B*0705, HLA-B*0709, HLA-B*0710, HLA-B*0715, and HLA-B*0721 allele.

13. A stable MHC-peptide complex, comprising a peptide epitope selected from Table 1A, 1B, 1C, 1D, 1E, and/or 1F in the context of an MHC molecule.

14. The stable MHC-peptide complex of claim 13, wherein the MHC molecule is a MHC multimer, optionally wherein the MHC multimer is a tetramer.

15. The stable MHC-peptide complex of claim 13 or 14, wherein the MHC molecule is a MHC class I molecule.

16. The stable MHC-peptide complex of any one of claims 13-15, wherein the MHC molecule comprises an MHC alpha chain that is an HLA serotype selected from the group consisting of HLA-A*02, HLA-A*03, HLA-A*01, HLA-A*11, HLA-A*24, and/or HLA-B*07, optionally wherein the HLA allele is selected from the group consisting of HLA-A*0201, HLA-A*0202, HLA-A*0203, HLA-A*0204, HLA-A*0205, HLA-A*0206, HLA-A*0207, HLA-A*0210, HLA-A*0211, HLA-A*0212, HLA-A*0213, HLA-A*0214, HLA-A*0216, HLA-A*0217, HLA-A*0219, HLA-A*0220, HLA-A*0222, HLA-A*0224, HLA-A*0230, HLA-A*0242, HLA-A*0253, HLA-A*0260, HLA-A*0274 allele, HLA-A*0301, HLA-A*0302, HLA-A*0305, HLA-A*0307, HLA-A*0101, HLA-A*0102, HLA-A*0103, HLA-A*0116 allele, HLA-A*1101, HLA-A*1102, HLA-A*1103, HLA-A*1104, HLA-A*1105, HLA-A*1119 allele, HLA-A*2402, HLA-A*2403, HLA-A*2405, HLA-A*2407, HLA-A*2408, HLA-A*2410, HLA-A*2414, HLA-A*2417, HLA-A*2420, HLA-A*2422, HLA-A*2425, HLA-A*2426, HLA-A*2458 allele, HLA-B*0702, HLA-B*0704, HLA-B*0705, HLA-B*0709, HLA-B*0710, HLA-B*0715, and HLA-B*0721 allele.

17. The stable MHC-peptide complex of any one of claims 13-16, wherein the peptide epitope and the MHC molecule are covalently linked and/or wherein the alpha and beta chains of the MHC molecule are covalently linked.

18. The stable MHC-peptide complex of any one of claims 13-17, wherein the stable MHC-peptide complex comprises a detectable label, optionally wherein the detectable label is a fluorophore.

19. An immunogenic composition comprising the stable MHC-peptide complex of any one of claims 13-18, and an adjuvant.

20. An isolated nucleic acid that encodes the immunogenic peptide of any one of claims 1-5, or a complement thereof.

21. A vector comprising the isolated nucleic acid of claim 20.

22. A cell that a) comprises the isolated nucleic acid of claim 20, b) comprises the vector of claim 21, and/or c) produces one or more immunogenic peptides of any one of claims 1-5 and/or presents at the cell surface one or more stable MHC-peptide complexes of any one of claims 13-18, optionally wherein the cell is genetically engineered.

23. A binding moiety that specifically binds an immunogenic peptide of any one of claims 1-5 and/or the stable MHC-peptide complex of any one of claims 13-18, optionally wherein the binding moiety is an antibody, an antigen-binding fragment of an antibody, a TCR, an antigen-binding fragment of a TCR, a single chain TCR (scTCR), a chimeric antigen receptor (CAR), or a fusion protein comprising a TCR and an effector domain.

24. A device or kit comprising a) one or more immunogenic peptides of any one of claims 1-5 and/or b) one or more stable MHC-peptide complexes of any one of claims 13-18, said device or kit optionally comprising a reagent to detect binding of a) and/or b) to a T cell receptor.

25. A method of detecting T cells that bind a stable MHC-peptide complex comprising:
a) contacting a sample comprising T cells with a stable MHC-peptide complex of any one of claims 13-18; and
b) detecting binding of T cells to the stable MHC-peptide complex, optionally further determining the percentage of stable MHC-peptide-specific T cells that bind to the stable MHC-peptide complex, optionally wherein the sample comprises peripheral blood mononuclear cells (PBMCs).

26. The method of claim 25, wherein the T cells are CD8+ T cells.

27. The method of any one of claims 24-27, wherein the detecting and/or determining is performed using fluorescence activated cell sorting (FACS), enzyme linked immunosorbent assay (ELISA), radioimmune assay (RIA), immunochemically, Western blot, or intracellular flow assay.

28. The method of any one of claims 24-27, wherein the sample comprises T cells contacted with, or suspected of having been contacted with, one or more SARS-CoV-2 proteins or fragments thereof.

29. A method of determining whether a subject has exposure to and/or protection from SARS-CoV-2 comprising:
a) incubating a cell population comprising T cells obtained from the subject with an immunogenic peptide of any one of claims 1-5 or a stable MHC-peptide complex of any one of claims 13-18; and
b) detecting the presence or level of reactivity,
wherein the presence of or a higher level of reactivity compared to a control level indicates that the subject has exposure to and/or protection from SARS-CoV-2.

30. A method for predicting the clinical outcome of a subject afflicted with SARS-CoV-2 infection comprising:
a) determining the presence or level of reactivity between T cells obtained from the subject and one more immunogenic peptides of any one of claims 1-5 or one or more stable MHC-peptide complexes of any one of claims 13-18; and
b) comparing the presence or level of reactivity to that from a control, wherein the control is obtained from a subject having a good clinical outcome;
wherein the presence or a higher level of reactivity in the subject sample as compared to the control indicates that the subject has a good clinical outcome.

31. A method of assessing the efficacy of a SARS-CoV-2 therapy comprising:
a) determining the presence or level of reactivity between T cells obtained from the subject and one more immunogenic peptides of any one of claims 1-5 or one or more stable MHC-peptide complexes of any one of claims 13-18, in a first sample obtained from the subject prior to providing at least a portion of the SARS-CoV-2 therapy to the subject, and
b) determining the presence or level of reactivity between the one more immunogenic peptides of any one of claims 1-5, or the one or more stable MHC-peptide complexes of any one of claims 13-18, and T cells obtained from the subject present in a second sample obtained from the subject following provision of the portion of the SARS-CoV-2 therapy,
wherein the presence or a higher level of reactivity in the second sample, relative to the first sample, is an indication that the therapy is efficacious for treating SARS-CoV-2 in the subject.

32. The method of any one of claims 29-31, wherein the level of reactivity is indicated by a) the presence of binding and/or b) T cell activation and/or effector function, optionally wherein the T cell activation or effector function is T cell proliferation, killing, or cytokine release.

33. The method of any one of claims 29-32, further comprising repeating steps a) and b) at a subsequent point in time, optionally wherein the subject has undergone treatment to ameliorate SARS-CoV-2 infection between the first point in time and the subsequent point in time.

34. The method of any one of claims 29-33, wherein the T cell binding, activation, and/or effector function is detected using fluorescence activated cell sorting (FACS), enzyme linked immunosorbent assay (ELISA), radioimmune assay (RIA), immunochemically, Western blot, or intracellular flow assay.

35. The method of any one of claims 29-34, wherein the control level is a reference number.

36. The method of any one of claims 29-35, wherein the control level is a level of a subject without exposure to SARS-CoV-2.

37. A method of preventing and/or treating SARS-CoV-2 infection in a subject comprising administering to the subject a therapeutically effective amount of an immunogenic composition comprising one or more immunogenic peptides, wherein the immunogenic peptides comprise a peptide epitope selected from Table 1A, 1B, 1C, 1D, 1E, and/or 1F.

38. The method of claim 37, wherein the immunogenic peptide consists of a peptide epitope selected from Table 1A, 1B, 1C, 1D, 1E, and/or 1F.

39. The method of claim 37 or 38, wherein the immunogenic peptide is derived from a SARS-CoV-2 protein, optionally wherein the immunogenic peptide is 8, 9, 10, 11, 12, 13, 14, or 15 amino acids in length.

40. The method of any one of claims 37-39, wherein the SARS-CoV-2 protein is selected from the group consisting of orfla/b, S protein, N protein, M protein, orf3a, and orf7a.

41. The method of any one of claims 37-39, wherein the immunogenic peptide is capable of eliciting a T cell response in a subject.

42. The method of any one of claims 37-40, wherein the immunogenic composition comprises more than one immunogenic peptide.

43. The method of any one of claims 37-42, wherein the immunogenic composition further comprises an adjuvant.

44. The method of any one of claims 37-43, wherein the immunogenic composition is capable of eliciting a T cell response in a subject.

45. The method of any one of claims 37-44, wherein the administered immunogenic composition induces an immune response against the SARS-CoV-2 in the subject.

46. The method of any one of claims 37-45, wherein the administered immunogenic composition induces a T cell immune response against the SARS-CoV-2 in the subject.

47. The method of any one of claims 37-46, wherein the T cell immune response is a CD8+ T cell immune response.

48. A method of identifying a peptide-binding molecule, or antigen-binding fragment thereof, that binds to a peptide epitope selected from Table 1A, 1B, 1C, 1D, 1E, and/or 1F comprising:
a) providing a cell presenting a peptide epitope selected from Table 1A, 1B, 1C, 1D, 1E, and/or 1F in the context of a MHC molecule on the surface of the cell;
b) determining binding of a plurality of candidate peptide-binding molecules or antigen-binding fragments thereof to the peptide epitope in the context of the MHC molecule on the cell; and
c) identifying one or more peptide-binding molecules or antigen-binding fragments thereof that bind to the peptide epitope in the context of the MHC molecule.

49. The method of claim 48, wherein the step a) comprises contacting the MHC molecule on the surface of the cell with a peptide epitope selected from Table 1A, 1B, 1C, 1D, 1E, and/or 1F.

50. The method of claim 48, wherein the step a) comprises transfecting the cell with a vector comprising a heterologous sequence encoding a peptide epitope selected from Table 1A, 1B, 1C, 1D, 1E, and/or 1F.

51. A method of identifying a peptide-binding molecule or antigen-binding fragment thereof that binds to a peptide epitope selected from Table 1A, 1B, 1C, 1D, 1E, and/or 1F comprising:
a) providing a peptide epitope either alone or in a stable MHC-peptide complex, comprising a peptide epitope selected from Table 1A, 1B, 1C, 1D, 1E, and/or 1F either alone or in the context of an MHC molecule;
b) determining binding of a plurality of candidate peptide-binding molecules or antigen-binding fragments thereof to the peptide or stable MHC-peptide complex; and
c) identifying one or more peptide-binding molecules or antigen-binding fragments thereof that bind to the peptide epitope or the stable MHC-peptide complex.

52. The method of claim 51, wherein the MHC molecule is a MHC multimer, optionally wherein the MHC multimer is a tetramer.

53. The method of claim 51 or 52, wherein the MHC molecule is a MHC class I molecule.

54. The method of any one of claims 51-53, wherein the MHC molecule comprises an MHC alpha chain that is an HLA serotype selected from the group consisting of HLA-A*02, HLA-A*03, HLA-A*01, HLA-A*11, HLA-A*24, and/or HLA-B*07, optionally wherein the HLA allele is selected from the group consisting of HLA-A*0201, HLA-A*0202, HLA-A*0203, HLA-A*0204, HLA-A*0205, HLA-A*0206, HLA-A*0207, HLA-A*0210, HLA-A*0211, HLA-A*0212, HLA-A*0213, HLA-A*0214, HLA-A*0216, HLA-A*0217, HLA-A*0219, HLA-A*0220, HLA-A*0222, HLA-A*0224, HLA-A*0230, HLA-A*0242, HLA-A*0253, HLA-A*0260, HLA-A*0274 allele, HLA-A*0301, HLA-A*0302, HLA-A*0305, HLA-A*0307, HLA-A*0101, HLA-A*0102, HLA-A*0103, HLA-A*0116 allele, HLA-A*1101, HLA-A*1102, HLA-A*1103, HLA-A*1104, HLA-A*1105, HLA-A*1119 allele, HLA-A*2402, HLA-A*2403, HLA-A*2405, HLA-A*2407, HLA-A*2408, HLA-A*2410, HLA-A*2414, HLA-A*2417, HLA-A*2420, HLA-A*2422, HLA-A*2425, HLA-A*2426, HLA-A*2458 allele, HLA-B*0702, HLA-B*0704, HLA-B*0705, HLA-B*0709, HLA-B*0710, HLA-B*0715, and HLA-B*0721 allele.

55. The method of any one of claims 51-54, wherein the peptide epitope and the MHC molecule are covalently linked and/or wherein the alpha and beta chains of the MHC molecule are covalently linked.

56. The method of any one of claims 51-55, wherein the stable MHC-peptide complex comprises a detectable label, optionally wherein the detectable label is a fluorophore.

57. The method of any of claims 48-56, wherein the plurality of candidate peptide binding molecules comprises one or more T cell receptors (TCRs), or one or more antigen-binding fragments of a TCR.

58. The method of any of claims 48-57, wherein the plurality of candidate peptide binding molecules comprises at least 2, 5, 10, 100, 10³, 10⁴, 10⁵, 10⁶, 10⁷, 10⁸, 10⁹, or more, different candidate peptide binding molecules.

59. The method of any of claims 46-58, wherein the plurality of candidate peptide binding molecules comprises one or more candidate peptide binding molecules that are obtained from a sample from a subject or a population of subjects; or the plurality of candidate peptide binding molecules comprises one or more candidate peptide binding molecules that comprise mutations in a parent scaffold peptide binding molecule obtained from a sample from a subject.

60. The method of claim 59, wherein the subject or population of subjects are a) not infected with SARS-CoV-2 and/or have recovered from COVID-19 or b) infected with SARS-CoV-2 and/or have COVID-19.

61. The method of any of claims 59 or 60, wherein the subject or population of subjects has been vaccinated with one or more immunogenic peptides, wherein the immunogenic peptides comprise a peptide epitope selected from Table 1A, 1B, 1C, 1D, 1E, and/or 1F.

62. The method of any of claims 56-61, wherein the subject is a mammal, optionally wherein the mammal is a human, a primate, or a rodent.

63. The method of any one of claims 59-62, wherein the subject is an HLA-transgenic mouse and/or is a human TCR transgenic mouse.

64. The method of any of claims 59-63, wherein the sample comprises T cells.

65. The method of claim 64, wherein the sample comprises peripheral blood mononuclear cells (PBMCs) or CD8+ memory T cells.

66. The peptide-binding molecule or antigen-binding fragment thereof identified according to any one of claims 48-65, optionally wherein the binding moiety is an antibody, an antigen-binding fragment of an antibody, a TCR, an antigen-binding fragment of a TCR, a single chain TCR (scTCR), a chimeric antigen receptor (CAR), or a fusion protein comprising a TCR and an effector domain.

67. A method of treating SARS-CoV-2 infection in a subject comprising administering to the subject a therapeutically effective amount of genetically engineered T cells that express a TCR identified by the method of any one of claims 51-66.

68. A method of treating SARS-CoV-2 infection in a subject comprising administering to the subject a therapeutically effective amount of genetically engineered T cells that express a TCR that binds to a peptide epitope selected from Table 1A, 1B, 1C, 1D, 1E, and/or 1F.

69. A method of treating SARS-CoV-2 infection in a subject comprising administering to the subject a therapeutically effective amount of genetically engineered T cells that express a TCR that binds to a stable MHC-peptide complex comprising a peptide epitope selected from Table 1A, 1B, 1C, 1D, 1E, and/or 1F in the context of an MHC molecule.

70. The method of claim 69, wherein the MHC molecule is a MHC multimer, optionally wherein the MHC multimer is a tetramer.

71. The method of any one of claims 67-70, wherein the MHC molecule is a MHC class I molecule.

72. The method of any one of claims 67-71, wherein the MHC molecule comprises an MHC alpha chain that is an HLA serotype selected from the group consisting of HLA-A*02, HLA-A*03, HLA-A*01, HLA-A*11, HLA-A*24, and/or HLA-B*07, optionally wherein the HLA allele is selected from the group consisting of HLA-A*0201, HLA-A*0202, HLA-A*0203, HLA-A*0204, HLA-A*0205, HLA-A*0206, HLA-A*0207, HLA-A*0210, HLA-A*0211, HLA-A*0212, HLA-A*0213, HLA-A*0214, HLA-A*0216, HLA-A*0217, HLA-A*0219, HLA-A*0220, HLA-A*0222, HLA-A*0224, HLA-A*0230, HLA-A*0242, HLA-A*0253, HLA-A*0260, HLA-A*0274 allele, HLA-A*0301, HLA-A*0302, HLA-A*0305, HLA-A*0307, HLA-A*0101, HLA-A*0102, HLA-A*0103, HLA-A*0116 allele, HLA-A*1101, HLA-A*1102, HLA-A*1103, HLA-A*1104, HLA-A*1105, HLA-A*1119 allele, HLA-A*2402, HLA-A*2403, HLA-A*2405, HLA-A*2407, HLA-A*2408, HLA-A*2410, HLA-A*2414, HLA-A*2417, HLA-A*2420, HLA-A*2422, HLA-A*2425, HLA-A*2426, HLA-A*2458 allele, HLA-B*0702, HLA-B*0704, HLA-B*0705, HLA-B*0709, HLA-B*0710, HLA-B*0715, and HLA-B*0721 allele.

73. The method of any one of claims 67-72, wherein the peptide epitope and the MHC molecule are covalently linked and/or wherein the alpha and beta chains of the MHC molecule are covalently linked.

74. The method of any one of claims 67-73, wherein the stable MHC-peptide complex comprises a detectable label, optionally wherein the detectable label is a fluorophore.

75. The method of any one of claims 67-74, wherein the T cells are isolated from a) the subject, b) a donor not infected with SARS-CoV-2, or c) a donor recovered from COVID-19.

76. A method of treating SARS-CoV-2 infection in a subject comprising transfusing antigen-specific T cells to the subject, wherein the antigen-specific T cells are generated by:
a) stimulating PBMCs or T cells from a subject with a peptide epitope selected from Table 1A and 1B, a stable MHC-peptide complex comprising a peptide epitope selected from Table 1A, 1B, 1C, 1D, 1E, and/or 1F in the context of an MHC molecule, or a cell that presents a peptide epitope selected from Table 1A, 1B, 1C, 1D, 1E, and/or 1F in the context of a MHC molecule on its cell surface; and
b) expanding antigen-specific T cells in vitro, optionally isolating PBMCs or T cells from the subject before stimulating the PBMCs or T cells.

77. The method of claim 76, wherein the T cell is a naive T cell, a central memory T cell, or an effector memory T cell.

78. The method of claim 77, wherein the T cell is a CD8+ memory T cell.

79. The method of any one of claims 24-78, wherein the agents are placed in contact under conditions and for a time suitable for the formation of at least one immune complex between the peptide epitope, immunogenic peptide, stable MHC-peptide complex, T cell receptor, and/or T cell.

80. The method of any one of claims 24-79, wherein the peptide epitope, immunogenic peptide, stable MHC-peptide complex, and/or T cell receptor are expressed by cells and the cells are expanded and/or isolated during one or more steps.

81. The method of any one of claims 24-80, wherein the subject is a mammal, optionally wherein the mammal is a human, a primate, or a rodent.


SEQUENCE LISTING (157 sequences)

SEQ ID NO: 1 - protein, 9 aa, Severe acute respiratory syndrome coronavirus 2
KLWAQCVQL

SEQ ID NO: 2 - protein, 10 aa, Severe acute respiratory syndrome coronavirus 2
KTFPPTEPKK

SEQ ID NO: 3 - protein, 9 aa, Severe acute respiratory syndrome coronavirus 2
ALWEIQQVV

SEQ ID NO: 4 - protein, 10 aa, Severe acute respiratory syndrome coronavirus 2
YLQPRTFLLK

SEQ ID NO: 5 - protein, 10 aa, Severe acute respiratory syndrome coronavirus 2
SALWEIQQVV

SEQ ID NO: 6 - protein, 14 aa, Severe acute respiratory syndrome coronavirus 2
ATYYLFDESGEFKL

SEQ ID NO: 7 - protein, 10 aa, Severe acute respiratory syndrome coronavirus 2
PLLYDANYFL

SEQ ID NO: 8 - protein, 9 aa, Severe acute respiratory syndrome coronavirus 2
LLYDANYFL

SEQ ID NO: 9 - protein, 9 aa, Severe acute respiratory syndrome coronavirus 2
RLANECAQV

SEQ ID NO: 10 - protein, 10 aa, Severe acute respiratory syndrome coronavirus 2
QLSSYSLFDM

SEQ ID NO: 11 - protein, 11 aa, Severe acute respiratory syndrome coronavirus 2
YLFDESGEFKL

SEQ ID NO: 12 - protein, 10 aa, Severe acute respiratory syndrome coronavirus 2
FLIVAAIVFI

SEQ ID NO: 13 - protein, 8 aa, Severe acute respiratory syndrome coronavirus 2
YANSVFNI

SEQ ID NO: 14 - protein, 13 aa, Severe acute respiratory syndrome coronavirus 2
FLCWHTNCYDYCI

SEQ ID NO: 15 - protein, 9 aa, Severe acute respiratory syndrome coronavirus 2
SMWALIISV

SEQ ID NO: 16 - protein, 9 aa, Severe acute respiratory syndrome coronavirus 2
LLLDRLNQL

SEQ ID NO: 17 - protein, 9 aa, Severe acute respiratory syndrome coronavirus 2
FAFACPDGV

SEQ ID NO: 18 - protein, 10 aa, Severe acute respiratory syndrome coronavirus 2
YRLANECAQV

SEQ ID NO: 19 - protein, 10 aa, Severe acute respiratory syndrome coronavirus 2
GYLQPRTFLL

SEQ ID NO: 20 - protein, 9 aa, Severe acute respiratory syndrome coronavirus 2
YLQPRTFLL

SEQ ID NO: 21 - protein, 8 aa, Severe acute respiratory syndrome coronavirus 2
ALWEIQQV

SEQ ID NO: 22 - protein, 10 aa, Severe acute respiratory syndrome coronavirus 2
ALDQAISMWA

SEQ ID NO: 23 - protein, 10 aa, Severe acute respiratory syndrome coronavirus 2
SLFDMSKFPL

SEQ ID NO: 24 - protein, 9 aa, Severe acute respiratory syndrome coronavirus 2
LLAKDTTEA

SEQ ID NO: 25 - protein, 10 aa, Severe acute respiratory syndrome coronavirus 2
MDLFMRIFTI

SEQ ID NO: 26 - protein, 10 aa, Severe acute respiratory syndrome coronavirus 2
KILGLPTQTV

SEQ ID NO: 27 - protein, 10 aa, Severe acute respiratory syndrome coronavirus 2
SLQTYVTQQL

SEQ ID NO: 28 - protein, 9 aa, Severe acute respiratory syndrome coronavirus 2
ALSKGVHFV

SEQ ID NO: 29 - protein, 9 aa, Severe acute respiratory syndrome coronavirus 2
VMCGGSLYV

SEQ ID NO: 30 - protein, 13 aa, Severe acute respiratory syndrome coronavirus 2
TYASALWEIQQVV

SEQ ID NO: 31 - protein, 10 aa, Severe acute respiratory syndrome coronavirus 2
LLYDANYFLC

SEQ ID NO: 32 - protein, 10 aa, Severe acute respiratory syndrome coronavirus 2
FDMSKFPLKL

SEQ ID NO: 33 - protein, 13 aa, Severe acute respiratory syndrome coronavirus 2
TYYLFDESGEFKL

SEQ ID NO: 34 - protein, 11 aa, Severe acute respiratory syndrome coronavirus 2
YSLFDMSKFPL

SEQ ID NO: 35 - protein, 12 aa, Severe acute respiratory syndrome coronavirus 2
YASALWEIQQVV

SEQ ID NO: 36 - protein, 11 aa, Severe acute respiratory syndrome coronavirus 2
FLLKYNENGTI

SEQ ID NO: 37 - protein, 10 aa, Severe acute respiratory syndrome coronavirus 2
FTYASALWEI

SEQ ID NO: 38 - protein, 12 aa, Severe acute respiratory syndrome coronavirus 2
YYLFDESGEFKL

SEQ ID NO: 39 - protein, 14 aa, Severe acute respiratory syndrome coronavirus 2
RLWLCWKCRSKNPL

SEQ ID NO: 40 - protein, 9 aa, Severe acute respiratory syndrome coronavirus 2
TVIEVQGYK

SEQ ID NO: 41 - protein, 9 aa, Severe acute respiratory syndrome coronavirus 2
QIAPGQTGK

SEQ ID NO: 42 - protein, 11 aa, Severe acute respiratory syndrome coronavirus 2
MMVTNNTFTLK

SEQ ID NO: 43 - protein, 9 aa, Severe acute respiratory syndrome coronavirus 2
RLFRKSNLK

SEQ ID NO: 44 - protein, 10 aa, Severe acute respiratory syndrome coronavirus 2
YNSASFSTFK

SEQ ID NO: 45 - protein, 9 aa, Severe acute respiratory syndrome coronavirus 2
VTNNTFTLK

SEQ ID NO: 46 - protein, 10 aa, Severe acute respiratory syndrome coronavirus 2
RQIAPGQTGK

SEQ ID NO: 47 - protein, 9 aa, Severe acute respiratory syndrome coronavirus 2
KLFDRYFKY

SEQ ID NO: 48 - protein, 9 aa, Severe acute respiratory syndrome coronavirus 2
KTIQPRVEK

SEQ ID NO: 49 - protein, 9 aa, Severe acute respiratory syndrome coronavirus 2
CVADYSVLY

SEQ ID NO: 50 - protein, 10 aa, Severe acute respiratory syndrome coronavirus 2
RLKLFDRYFK

SEQ ID NO: 51 - protein, 9 aa, Severe acute respiratory syndrome coronavirus 2
KTFPPTEPK

SEQ ID NO: 52 - protein, 12 aa, Severe acute respiratory syndrome coronavirus 2
STFKCYGVSPTK

SEQ ID NO: 53 - protein, 9 aa, Severe acute respiratory syndrome coronavirus 2
KCYGVSPTK

SEQ ID NO: 54 - protein, 12 aa, Severe acute respiratory syndrome coronavirus 2
VLYNSASFSTFK

SEQ ID NO: 55 - protein, 10 aa, Severe acute respiratory syndrome coronavirus 2
MVTNNTFTLK

SEQ ID NO: 56 - protein, 8 aa, Severe acute respiratory syndrome coronavirus 2
KLFDRYFK

SEQ ID NO: 57 - protein, 10 aa, Severe acute respiratory syndrome coronavirus 2
QLPQGTTLPK

SEQ ID NO: 58 - protein, 10 aa, Severe acute respiratory syndrome coronavirus 2
VPTDNYITTY

SEQ ID NO: 59 - protein, 10 aa, Severe acute respiratory syndrome coronavirus 2
FTSDYYQLYS

SEQ ID NO: 60 - protein, 9 aa, Severe acute respiratory syndrome coronavirus 2
CTDDNALAY

SEQ ID NO: 61 - protein, 10 aa, Severe acute respiratory syndrome coronavirus 2
SSPDDQIGYY

SEQ ID NO: 62 - protein, 11 aa, Severe acute respiratory syndrome coronavirus 2
HTTDPSFLGRY

SEQ ID NO: 63 - protein, 12 aa, Severe acute respiratory syndrome coronavirus 2
TACTDDNALAYY

SEQ ID NO: 64 - protein, 8 aa, Severe acute respiratory syndrome coronavirus 2
TDDNALAY

SEQ ID NO: 65 - protein, 9 aa, Severe acute respiratory syndrome coronavirus 2
GTDLEGNFY

SEQ ID NO: 66 - protein, 9 aa, Severe acute respiratory syndrome coronavirus 2
PTDNYITTY

SEQ ID NO: 67 - protein, 9 aa, Severe acute respiratory syndrome coronavirus 2
TCDGTTFTY

SEQ ID NO: 68 - protein, 9 aa, Severe acute respiratory syndrome coronavirus 2
SMDNSPNLA

SEQ ID NO: 69 - protein, 12 aa, Severe acute respiratory syndrome coronavirus 2
YHTTDPSFLGRY

SEQ ID NO: 70 - protein, 14 aa, Severe acute respiratory syndrome coronavirus 2
LTTAAKLMVVIPDY

SEQ ID NO: 71 - protein, 10 aa, Severe acute respiratory syndrome coronavirus 2
VDTDFVNEFY

SEQ ID NO: 72 - protein, 11 aa, Severe acute respiratory syndrome coronavirus 2
ACTDDNALAYY

SEQ ID NO: 73 - protein, 9 aa, Severe acute respiratory syndrome coronavirus 2
FTSDYYQLY

SEQ ID NO: 74 - protein, 10 aa, Severe acute respiratory syndrome coronavirus 2
YFTSDYYQLY

SEQ ID NO: 75 - protein, 9 aa, Severe acute respiratory syndrome coronavirus 2
DTDFVNEFY

SEQ ID NO: 76 - protein, 9 aa, Severe acute respiratory syndrome coronavirus 2
SSDNIALLV

SEQ ID NO: 77 - protein, 10 aa, Severe acute respiratory syndrome coronavirus 2
CTDDNALAYY

SEQ ID NO: 78 - protein, 10 aa, Severe acute respiratory syndrome coronavirus 2
TTDPSFLGRY

SEQ ID NO: 79 - protein, 9 aa, Severe acute respiratory syndrome coronavirus 2
LSPRWYFYY

SEQ ID NO: 80 - protein, 13 aa, Severe acute respiratory syndrome coronavirus 2
YYHTTDPSFLGRY

SEQ ID NO: 81 - protein, 14 aa, Severe acute respiratory syndrome coronavirus 2
EYYHTTDPSFLGRY

SEQ ID NO: 82 - protein, 8 aa, Severe acute respiratory syndrome coronavirus 2
TSDYYQLY

SEQ ID NO: 83 - protein, 10 aa, Severe acute respiratory syndrome coronavirus 2
ACTDDNALAY

SEQ ID NO: 84 - protein, 10 aa, Severe acute respiratory syndrome coronavirus 2
VATSRTLSYY

SEQ ID NO: 85 - protein, 9 aa, Severe acute respiratory syndrome coronavirus 2
ATSRTLSYY

SEQ ID NO: 86 - protein, 10 aa, Severe acute respiratory syndrome coronavirus 2
NTCDGTTFTY

SEQ ID NO: 87 - protein, 9 aa, Severe acute respiratory syndrome coronavirus 2
VTDTPKGPK

SEQ ID NO: 88 - protein, 12 aa, Severe acute respiratory syndrome coronavirus 2
TVATSRTLSYYK

SEQ ID NO: 89 - protein, 9 aa, Severe acute respiratory syndrome coronavirus 2
ASAFFGMSR

SEQ ID NO: 90 - protein, 9 aa, Severe acute respiratory syndrome coronavirus 2
LIRQGTDYK

SEQ ID NO: 91 - protein, 10 aa, Severe acute respiratory syndrome coronavirus 2
LLNKHIDAYK

SEQ ID NO: 92 - protein, 9 aa, Severe acute respiratory syndrome coronavirus 2
AVILRGHLR

SEQ ID NO: 93 - protein, 10 aa, Severe acute respiratory syndrome coronavirus 2
QDLKWARFPK

SEQ ID NO: 94 - protein, 13 aa, Severe acute respiratory syndrome coronavirus 2
VTLACFVLAAVYR

SEQ ID NO: 95 - protein, 9 aa, Severe acute respiratory syndrome coronavirus 2
KVKYLYFIK

SEQ ID NO: 96 - protein, 14 aa, Severe acute respiratory syndrome coronavirus 2
STMTNRQFHQKLLK

SEQ ID NO: 97 - protein, 9 aa, Severe acute respiratory syndrome coronavirus 2
QQQGQTVTK

SEQ ID NO: 98 - protein, 10 aa, Severe acute respiratory syndrome coronavirus 2
ATSRTLSYYK

SEQ ID NO: 99 - protein, 10 aa, Severe acute respiratory syndrome coronavirus 2
ATEGALNTPK

SEQ ID NO: 100 - protein, 9 aa, Severe acute respiratory syndrome coronavirus 2
KSAAEASKK

SEQ ID NO: 101 - protein, 11 aa, Severe acute respiratory syndrome coronavirus 2
KAYNVTQAFGR

SEQ ID NO: 102 - protein, 9 aa, Severe acute respiratory syndrome coronavirus 2
QYIKWPWYI

SEQ ID NO: 103 - protein, 9 aa, Severe acute respiratory syndrome coronavirus 2
VYIGDPAQL

SEQ ID NO: 104 - protein, 9 aa, Severe acute respiratory syndrome coronavirus 2
VYFLQSINF

SEQ ID NO: 105 - protein, 9 aa, Severe acute respiratory syndrome coronavirus 2
YYRRATRRI

SEQ ID NO: 106 - protein, 10 aa, Severe acute respiratory syndrome coronavirus 2
RWYFYYLGTG

SEQ ID NO: 107 - protein, 10 aa, Severe acute respiratory syndrome coronavirus 2
QYIKWPWYIW

SEQ ID NO: 108 - protein, 10 aa, Severe acute respiratory syndrome coronavirus 2
KYEQYIKWPW

SEQ ID NO: 109 - protein, 10 aa, Severe acute respiratory syndrome coronavirus 2
KWPWYIWLGF

SEQ ID NO: 110 - protein, 9 aa, Severe acute respiratory syndrome coronavirus 2
LYLYALVYF

SEQ ID NO: 111 - protein, 14 aa, Severe acute respiratory syndrome coronavirus 2
LYALVYFLQSINFV

SEQ ID NO: 112 - protein, 14 aa, Severe acute respiratory syndrome coronavirus 2
YLYALVYFLQSINF

SEQ ID NO: 113 - protein, 13 aa, Severe acute respiratory syndrome coronavirus 2
QYIKWPWYIWLGF

SEQ ID NO: 114 - protein, 13 aa, Severe acute respiratory syndrome coronavirus 2
LYALVYFLQSINF

SEQ ID NO: 115 - protein, 10 aa, Severe acute respiratory syndrome coronavirus 2
SPRWYFYYLG

SEQ ID NO: 116 - protein, 9 aa, Severe acute respiratory syndrome coronavirus 2
IPRRNVATL

SEQ ID NO: 117 - protein, 8 aa, Severe acute respiratory syndrome coronavirus 2
RPDTRYVL

SEQ ID NO: 118 - protein, 9 aa, Severe acute respiratory syndrome coronavirus 2
SPRWYFYYL

SEQ ID NO: 119 - protein, 9 aa, Severe acute respiratory syndrome coronavirus 2
RPDTRYVLM

SEQ ID NO: 120 - protein, 10 aa, Severe acute respiratory syndrome coronavirus 2
IPRRNVATLQ

SEQ ID NO: 121 - protein, 10 aa, Severe acute respiratory syndrome coronavirus 2
EIPRRNVATL

SEQ ID NO: 122 - protein, 8 aa, Severe acute respiratory syndrome coronavirus 2
PRWYFYYL

SEQ ID NO: 123 - protein, 10 aa, Severe acute respiratory syndrome coronavirus 2
LSPRWYFYYL

SEQ ID NO: 124 - protein, 9 aa, Severe acute respiratory syndrome coronavirus 2
RIRGGDGKM

SEQ ID NO: 125 - protein, 14 aa, Severe acute respiratory syndrome coronavirus 2
SLEIPRRNVATLQA

SEQ ID NO: 126 - protein, 7096 aa, Severe acute respiratory syndrome coronavirus 2 [sequence omitted]

SEQ ID NO: 127 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 128 - protein, 419 aa, Severe acute respiratory syndrome coronavirus 2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 129 - protein, 275 aa, Severe acute respiratory syndrome coronavirus 2
MDLFMRIFTIGTVTLKQGEIKDATPSDFVRATATIPIQASLPFGWLIVGVALLAVFQSASKIITLKKRWQLALSKGVHFVCNLLLLFVTVYSHLLLVAAGLEAPFLYLYALVYFLQSINFVRIIMRLWLCWKCRSKNPLLYDANYFLCWHTNCYDYCIPYNSVTSSIVITSGDGTTSPISEHDYQIGGYTEKWESGVKDCVVLHSYFTSDYYQLYSTQLSTDTGVEHVTFFIYNKIVDEPEEHVQIHTIDGSSGVVNPVMEPIYDEPTTTTSVPL

SEQ ID NO: 130 - protein, 222 aa, Severe acute respiratory syndrome coronavirus 2
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 131 - protein, 121 aa, Severe acute respiratory syndrome coronavirus 2
MKIILFLALITLATCELYHYQECVRGTTVLLKEPCSSGTYEGNSPFHPLADNKFALTCFSTQFAFACPDGVKHVYQLRARSVSPKLFIRQEEVQELYSPIFLIVAAIVFITLCFTLKRKTE

SEQ ID NO: 132 - protein, 10 aa, Artificial Sequence
  Description of Artificial Sequence: Synthetic peptide
GGGGSGGGGS

SEQ ID NO: 133 - protein, 8 aa, Artificial Sequence
  Description of Artificial Sequence: Synthetic peptide
GGGSGGGS

SEQ ID NO: 134 - protein, 11 aa, Artificial Sequence
  Description of Artificial Sequence: Synthetic peptide
GGGSGGGSGGS

SEQ ID NO: 135 - protein, 9 aa, Severe acute respiratory syndrome coronavirus 2
NLVPMVATV

SEQ ID NO: 136 - protein, 9 aa, Unknown
  Description of Unknown: SPR epitope sequence
SPKLHFYYL

SEQ ID NO: 137 - protein, 10 aa, Severe acute respiratory syndrome coronavirus 2
FIYASALWEI

SEQ ID NO: 138 - protein, 10 aa, Severe acute respiratory syndrome coronavirus 2
FLLTFNDNQA

SEQ ID NO: 139 - protein, 10 aa, Severe acute respiratory syndrome coronavirus 2
FLFDMSKFPL

SEQ ID NO: 140 - protein, 9 aa, Severe acute respiratory syndrome coronavirus 2
WLDARMQAI

SEQ ID NO: 141 - protein, 9 aa, Severe acute respiratory syndrome coronavirus 2
YVLDHLIVV

SEQ ID NO: 142 - protein, 9 aa, Severe acute respiratory syndrome coronavirus 2
NLLDSYFVV

SEQ ID NO: 143 - protein, 10 aa, Severe acute respiratory syndrome coronavirus 2
NVYADSFVVK

SEQ ID NO: 144 - protein, 9 aa, Severe acute respiratory syndrome coronavirus 2
SVYAWERKK

SEQ ID NO: 145 - protein, 9 aa, Severe acute respiratory syndrome coronavirus 2
NMHLSTLMK

SEQ ID NO: 146 - protein, 8 aa, Severe acute respiratory syndrome coronavirus 2
VLLVDGHK

SEQ ID NO: 147 - protein, 11 aa, Severe acute respiratory syndrome coronavirus 2
AVMQKLPCQFK

SEQ ID NO: 148 - protein, 10 aa, Severe acute respiratory syndrome coronavirus 2
KTVPAGNLVK

SEQ ID NO: 149 - protein, 9 aa, Severe acute respiratory syndrome coronavirus 2
RLRAEAQVK

SEQ ID NO: 150 - protein, 10 aa, Severe acute respiratory syndrome coronavirus 2
LILRGSVAHK

SEQ ID NO: 151 - protein, 11 aa, Severe acute respiratory syndrome coronavirus 2
KTRPILSPLTK

SEQ ID NO: 152 - protein, 9 aa, Unknown
  Description of Unknown: KLW epitope sequence
KLWHYCSTL

SEQ ID NO: 153 - protein, 9 aa, Unknown
  Description of Unknown: KLW epitope sequence
KLWQYCSVL

SEQ ID NO: 154 - protein, 9 aa, Unknown
  Description of Unknown: KLW epitope sequence
SEWAYCVDL

SEQ ID NO: 155 - protein, 9 aa, Unknown
  Description of Unknown: KLW epitope sequence
KEWAYCVEM

SEQ ID NO: 156 - protein, 9 aa, Unknown
  Description of Unknown: SPR epitope sequence
LPRWYFYYL

SEQ ID NO: 157 - protein, 9 aa, Unknown
  Description of Unknown: SPR epitope sequence
PPKVHFYYL